(19) United States
(12) Patent Application Publication
(10) Pub. No.: US 2026/0176319 A1
(43) Pub. Date: Jun. 25, 2026

(54) PEPTIDE ANALOGS AND METHODS OF USING THE SAME

(71) Applicant: Kallyope, Inc., New York, NY (US)

(72) Inventors: Santhosh F. NEELAMKAVIL, Edison, NJ (US); Christopher MOYES, Westfield, NJ (US)

(21) Appl. No.: 19/415,046

(22) Filed: Dec. 10, 2025

Related U.S. Application Data
(63) Continuation of application No. PCT/US2025/027364, filed on May 1, 2025.
(60) Provisional application No. 63/641,660, filed on May 2, 2024.
(60) Provisional application No. 63/713,191, filed on Oct. 29, 2024.
(60) Provisional application No. 63/757,920, filed on Feb. 13, 2025.

(51) Int. Cl. C07K 14/575; A61K 38/00; A61P 3/04

(52) U.S. Cl. CPC C07K 14/57572; A61P 3/04; A61K 38/00

(57) ABSTRACT

Provided herein are peptide analogs of gastrin releasing peptide (GRP), corresponding pharmaceutical compositions, and methods of using such peptide analogs of GRP in the treatment of a disease or disorder, such as gastrointestinal disease or obesity.

### CROSS-REFERENCE TO RELATED APPLICATIONS

This application is a continuation of International Application No. PCT/US2025/027364, filed May 1, 2025, which claims the benefit of U.S. Provisional Patent Application No. 63/641,660, filed May 2, 2024, U.S. Provisional Patent Application No. 63/713,191, filed Oct. 29, 2024, and U.S. Provisional Patent Application No. 63/757,920, filed Feb. 13, 2025, each of which are incorporated herein by reference in their entireties.

### SEQUENCE LISTING

This application contains a Sequence Listing which has been submitted electronically in XML format. The Sequence Listing XML is incorporated herein by reference. Said XML file, created on Jan. 7, 2025, is named 50339-746_301_SL.xml and is 855,146 bytes in size.

### BACKGROUND

Gastrin releasing peptide (GRP) is a 27 amino acid peptide that stimulates release of gastrin and regulates gastric acid secretion. GRP₁₋₂₇ can be further processed to give GRP₁₈₋₂₇, also known as GRP-10 or neuromedin C. GRP receptors are widely distributed in mammals, especially in the central nervous system (CNS) and peripheral nervous system. GRP receptors regulate several functions including release of gastrointestinal hormones, smooth muscle cell contraction, and epithelial cell proliferation.

### SUMMARY

Described herein are peptide analogs of gastrin releasing peptide (GRP). In some embodiments, a peptide described herein is conjugated to a lipid. In some embodiments, a peptide described herein is a gastrin releasing peptide receptor (GRPR) agonist. In some embodiments, a peptide described herein is useful in the treatment of a disease or disorder. In some embodiments, the disease or disorder is gastrointestinal disease or a weight-related condition.

In one aspect, described herein is a peptide comprising an amino acid sequence of Formula (I): X1-X2-Ar-X4-X5-V-X7-X8-L-X10-X11-X12,
or a pharmaceutically acceptable salt, solvate, or isotope thereof, wherein:
X4, X5, X7, and X8 are each independently a natural or non-natural amino acid or amino acid analog;
Ar is an aromatic amino acid;
X1, X2, X10, X11, and X12 are each independently a natural or non-natural amino acid or amino acid analog, or absent;
wherein the peptide is conjugated to one or more lipid moieties.

In one aspect, described herein is a peptide comprising an amino acid sequence of Formula (V): X0-X1-X2-Ar-X4-X5-V-X7-X8-L-X10-X11-X12, or a pharmaceutically acceptable salt, solvate, or isotope thereof, wherein:
X4, X5, X7, and X8 are each independently a natural or non-natural amino acid or amino acid analog;
Ar is an aromatic amino acid;
X0, X1, X2, X10, X11, and X12 are each independently a natural or non-natural amino acid or amino acid analog, or absent;
wherein the peptide is conjugated to one or more lipid moieties.

In one aspect, described herein is a peptide comprising an amino acid sequence of Formula (V):
X0-X1-X2-Ar-X4-X5-V-X7-X8-L-X10-X11-X12, or a pharmaceutically acceptable salt, solvate, or isotope thereof, wherein:
X0, X1, X2, X4, X5, X7, and X8 are each independently a natural or non-natural amino acid or amino acid analog;
Ar is an aromatic amino acid;
X10, X11, and X12 are each independently a natural or non-natural amino acid or amino acid analog, or absent;
wherein the peptide is conjugated to one or more lipid moieties.

In some embodiments, X0 is absent, Glu, Ser, Arg, or Gly.

In some embodiments, X0 is Glu.

In some embodiments, X1 is absent, Gly, Ala, D-alanine, N-acetylglycine, N-ε-acetyllysine, N-α-acetyllysine, α-aminoisobutyric acid (Aib), Arg, Phe, D-serine, or D-phenylalanine

In some embodiments, X1 is absent, Gly, Ala, D-alanine, N-acetylglycine, N-ε-acetyllysine, N-α-acetyllysine, α-aminoisobutyric acid (Aib), Arg, Phe, or D-phenylalanine.

In some embodiments, X1 is absent, Gly, Ala, N-acetylglycine, D-alanine, N-ε-acetyllysine, or N-α-acetyllysine.

In some embodiments, X1 is Phe, D-phenylalanine, or Gly.

In some embodiments, X1 is Phe.

In some embodiments, X1 is D-phenylalanine.

In some embodiments, X1 is D-serine.

In some embodiments, X1 is Gly.

In some embodiments, X2 is absent, Ala, D-alanine, α-aminoisobutyric acid (Aib), Gly, Lys, N-ε-acetyllysine, N-α-acetyllysine, Asn, Glu, Gln, Arg, or Asp.

In some embodiments, X2 is Asn, Glu, Arg, Ala, Asp, Gln, Lys, or Lys(Ac).

In some embodiments, X2 is Asn, Arg, or Gln.

In some embodiments, X2 is Arg.

In some embodiments, X2 is Asn.

In some embodiments, X2 is Gln.

In some embodiments, X2 is Glu.

In some embodiments, Ar is an aromatic amino acid selected from Phe, Trp, Tyr, His, 1-methylhistidine, 3-methylhistidine, 3-(3-pyridyl)alanine (3PAl), 3-(2-pyridyl)alanine (2PAl), 3-(4-pyridyl)alanine (4PAl), 3-(1-pyrazolyl)alanine, 2-amino-3-(pyrazin-2-yl)propanoic acid, 2-amino-3-(furan-2-yl)propanoic acid, 2-amino-3-(naphthalen-2-yl)propanoic acid, 2-methylphenylalanine, 3-methylphenylalanine, 4-methylphenylalanine, 2-fluorophenylalanine, 3-fluorophenylalanine, 4-fluorophenylalanine, 2-methyltryptophan (2MeW), 4-methyltryptophan (4MeW), 5-methyltryptophan (5MeW), 6-methyltryptophan (6MeW), or 7-methyltryptophan (7MeW).

In some embodiments, Ar is histidine, 3-(3-pyridyl)alanine (3Pal), 3-(4-pyridyl)alanine (4Pal), or 3-(2-pyridyl)alanine (2Pal).

In some embodiments, Ar is histidine or 3-(3-pyridyl)alanine (3PAl).

In some embodiments, Ar is histidine.

In some embodiments, Ar is 3-(3-pyridyl)alanine (3PAl).

In some embodiments, X4 is Arg, Phe, Trp, Tyr, His, 1-methylhistidine, 3-methylhistidine, 3-(3-pyridyl)alanine (3PAl), 3-(2-pyridyl)alanine (2PAl), 3-(4-pyridyl)alanine (4PAl), 3-(1-pyrazolylalanine, 2-amino-3-(pyrazin-2-yl)propanoic acid, 2-amino-3-(furan-2-yl)propanoic acid, 2-amino-3-(naphthalen-2-yl)propanoic acid, 2-methylphenylalanine, 3-methylphenylalanine, 4-methylphenylalanine, 2-fluorophenylalanine, 3-fluorophenylalanine, 4-fluorophenylalanine, 2-methyltryptophan (2MeW), 4-methyltryptophan (4MeW), 5-methyltryptophan (5MeW), 6-methyltryptophan (6MeW), 7-methyltryptophan (7MeW), 7-fluorotryptophan (7FW), 6-fluorotryptophan (6FW), or 3-(2-naphthyl)alanine (2Nal).

In some embodiments, X4 is Phe, Trp, Tyr, His, 1-methylhistidine, 3-methylhistidine, 3-(3-pyridyl)alanine (3PAl), 3-(2-pyridyl)alanine (2PAl), 3-(4-pyridyl)alanine (4PAl), 3-(1-pyrazolylalanine, 2-amino-3-(pyrazin-2-yl)propanoic acid, 2-amino-3-(furan-2-yl)propanoic acid, 2-amino-3-(naphthalen-2-yl)propanoic acid, 2-methylphenylalanine, 3-methylphenylalanine, 4-methylphenylalanine, 2-fluorophenylalanine, 3-fluorophenylalanine, 4-fluorophenylalanine, 2-methyltryptophan (2MeW), 4-methyltryptophan (4MeW), 5-methyltryptophan (5MeW), 6-methyltryptophan (6MeW), or 7-methyltryptophan (7MeW).

In some embodiments, X4 is Arg, His, Trp, 7-methyltryptophan (7MeW), 6-methyltryptophan (6MeW), 7-fluorotryptophan (7FW), 6-fluorotryptophan (6FW), or 3-(2-naphthyl)alanine (2Nal).

In some embodiments, X4 is 7-methyltryptophan (7MeW) or 7-fluorotryptophan (7FW).

In some embodiments, X4 is Trp or 7-methyltryptophan (7MeW).

In some embodiments, X4 is Trp.

In some embodiments, X4 is 7-methyltryptophan (7MeW).

In some embodiments, X5 is Ala, α-aminoisobutyric acid (Aib), Gly, Leu, Val, Ile, Lys, Lys(Ac), Lys(Me)₃, D-alanine, or Ne.

In some embodiments, X5 is Ala, α-aminoisobutyric acid (Aib), or D-alanine.

In some embodiments, X5 is Ala.

In some embodiments, X7 is Gly, D-alanine, β-alanine, Ala, Aib, Leu, Val, Ile, Lys, Lys(Ac), Lys(Me)₃, S-β-aminobutanoic acid (BABA), or Nle.

In some embodiments, X7 is Gly, D-alanine, β-alanine, Ala, Aib, Leu, Val, Ile, Lys, Lys(Ac), Lys(Me)₃, or Nle.

In some embodiments, X7 is Gly, D-alanine, Ala, Aib, Leu, Val, Ile, Lys, Lys(Ac), Lys(Me)₃, or Ne.

In some embodiments, X7 is D-alanine, β-alanine, α-aminoisobutyric acid (Aib), Gly, or Ala.

In some embodiments, X7 is Gly, D-alanine, or β-alanine.

In some embodiments, X7 is D-alanine or β-alanine.

In some embodiments, X7 is D-alanine.

In some embodiments, X7 is β-alanine.

In some embodiments, X7 is S-β-aminobutanoic acid (BABA).

In some embodiments, X8 is Phe, Trp, Tyr, His, D-histidine, 1-methylhistidine, 3-methylhistidine, 3-(3-pyridyl)alanine (3PAl), 3-(2-pyridyl)alanine (2PAl), 3-(4-pyridyl)alanine (4PAl), 3-(1-pyrazolyl)alanine, 2-amino-3-(pyrazin-2-yl)propanoic acid, 2-amino-3-(furan-2-yl)propanoic acid, 2-amino-3-(naphthalen-2-yl)propanoic acid, 2-methylphenylalanine, 3-methylphenylalanine, 4-methylphenylalanine, 2-fluorophenylalanine, 3-fluorophenylalanine, 4-fluorophenylalanine, 2-methyltryptophan (2MeW), 4-methyltryptophan (4MeW), 5-methyltryptophan (5MeW), 6-methyltryptophan (6MeW), or 7-methyltryptophan (7MeW).

In some embodiments, X8 is Phe, Trp, Tyr, His, 1-methylhistidine, 3-methylhistidine, 3-(3-pyridyl)alanine (3PAl), 3-(2-pyridyl)alanine (2PAl), 3-(4-pyridyl)alanine (4PAl), 3-(1-pyrazolyl)alanine, 2-amino-3-(pyrazin-2-yl)propanoic acid, 2-amino-3-(furan-2-yl)propanoic acid, 2-amino-3-(naphthalen-2-yl)propanoic acid, 2-methylphenylalanine, 3-methylphenylalanine, 4-methylphenylalanine, 2-fluorophenylalanine, 3-fluorophenylalanine, 4-fluorophenylalanine, 2-methyltryptophan (2MeW), 4-methyltryptophan (4MeW), 5-methyltryptophan (5MeW), 6-methyltryptophan (6MeW), or 7-methyltryptophan (7MeW).

In some embodiments, X8 is His, D-histidine, 3-(3-pyridyl)alanine (3Pal), or F.

In some embodiments, X8 is His.

In some embodiments, X10 is absent, Met, Arg, norleucine (Nle), 3-(3-pyridyl)alanine (3Pal), Aib, Leu, Val, Lys, Lys(Ac), Lys(Me)₃, or Ile.

In some embodiments, X10 is absent, Met, Arg, norleucine (Nle), 3-(3-pyridyl)alanine (3Pal), cyclohexylalanine (Cha), or Lys(Me)₃.

In some embodiments, X10 is absent, Met, norleucine (Nle), Aib, Leu, Val, Lys, Lys(Ac), or Ile.

In some embodiments, X10 is Met or norleucine (Nle).

In some embodiments, X10 is norleucine (Nle).

In some embodiments, X11 is absent, sarcosine (Sar), Gly, Pro, Ala, D-alanine, α-aminoisobutyric acid (Aib), or Arg.

In some embodiments, X11 is absent, sarcosine (Sar), Gly, Pro, Ala, or D-alanine.

In some embodiments, X11 is absent, sarcosine (Sar), α-aminoisobutyric acid (Aib), or R

In some embodiments, X11 is sarcosine (Sar).

In some embodiments, X11 is absent.

In some embodiments, X12 is a natural or non-natural amino acid or amino acid analog.

In some embodiments, X12 is absent.

In some embodiments, the peptide comprises an amino acid sequence of Formula (II):
X1-X2-Ar-X4-X5-V-X7-X8-L-X10-X11-X12, or a pharmaceutically acceptable salt, solvate, or isotope thereof, wherein:
X1 is absent, α-aminoisobutyric acid (Aib), Arg, Phe, Gly, Ala, N-acetylglycine, or D-alanine;
X2 is Asn, Glu, Gln, Arg, Ala, Asp, Lys, or Lys(Ac);
Ar is histidine, 3-(3-pyridyl)alanine (3PAl), 3-(4-pyridyl)alanine (4Pal), or 3-(2-pyridyl)alanine (2Pal);
X4 is Arg, His, Trp, 7-methyltryptophan (7MeW), 6-methyltryptophan (6MeW), 7-fluorotryptophan (7FW), 6-fluorotryptophan (6FW), or 3-(2-naphthyl)alanine (2Nal), Phe, D-alanine, or Tyr;
X5 is Ala, α-aminoisobutyric acid (Aib), Gly, Leu, Val, Ile, D-alanine, or Nle;
X7 is Gly, D-alanine, β-alanine, Ala, Aib, Leu, Val, Ile, or Nle;
X8 is His, Phe, D-histidine, or 3-(3-pyridyl)alanine (3PAl);
X10 is absent, Met, Arg, norleucine (Nle), Aib, Leu, Val, Ile, 3-(3-pyridyl)alanine (3Pal), cyclohexylalanine (Cha), or Lys(Me)₃;
X11 is absent, sarcosine (Sar), Gly, Pro, Ala, Arg, D-alanine, or α-aminoisobutyric acid (Aib);
X12 is independently a natural or non-natural amino acid or amino acid analog, or absent.

In some embodiments, the peptide of Formula (I) is a peptide of Formula (II): X1-X2-Ar-X4-X5-V-X7-X8-L-X10-X11-X12, or a pharmaceutically acceptable salt, solvate, or isotope thereof, wherein:
X1 is Gly, Ala, N-acetylglycine, or D-alanine;
X2 is Asn, Glu, Gln, Arg, or Asp;
Ar is histidine or 3-(3-pyridyl)alanine (3PAl);
X4 is Trp, 7-methyltryptophan (7MeW), Phe, D-alanine, or Tyr;
X5 is Ala, α-aminoisobutyric acid (Aib), Gly, Leu, Val, Ile, D-alanine, or Nle;
X7 is Gly, D-alanine, Ala, Aib, Leu, Val, Ile, or Nle;
X8 is His or 3-(3-pyridyl)alanine (3PAl);
X10 is Met, Nle, Aib, Leu, Val, or Ile;
X11 is sarcosine (Sar), Gly, Pro, Ala, D-alanine, or absent; and X12 is independently a natural or non-natural amino acid or amino acid analog, or absent.

In some embodiments, X1 is absent, Gly, α-aminoisobutyric acid (Aib), Arg, Phe, or D-phenylalanine;
X2 is Asn, Glu, Arg, Ala, Asp, Gln, or Lys(Ac);
Ar is histidine, 3-(3-pyridyl)alanine (3Pal), 3-(4-pyridyl)alanine (4Pal), or 3-(2-pyridyl)alanine (2Pal);
X4 is Arg, His, Trp, 7-methyltryptophan (7MeW), 6-methyltryptophan (6MeW), 7-fluorotryptophan (7FW), 6-fluorotryptophan (6FW), or 3-(2-naphthyl)alanine (2Nal);
X5 is Ala, α-aminoisobutyric acid (Aib), or D-alanine;
X7 is D-alanine, β-alanine, α-aminoisobutyric acid (Aib), Gly, or Ala;
X8 is His, D-histidine, 3-(3-pyridyl)alanine (3Pal), or Phe;
X10 is absent, Met, Arg, norleucine (Nle), 3-(3-pyridyl)alanine (3Pal), or Lys(Me)₃;
X11 is absent, sarcosine (Sar), α-aminoisobutyric acid (Aib), or Arg; and
X12 is absent.

In some embodiments, X0 is absent or Glu; X1 is absent, Gly, Glu, α-aminoisobutyric acid (Aib), Arg, Phe, D-phenylalanine, or D-serine; X2 is Asn, Glu, Arg, Ala, Asp, Gln, or Lys(Ac); Ar is histidine, 3-(3-pyridyl)alanine (3Pal), 3-(4-pyridyl)alanine (4Pal), or 3-(2-pyridyl)alanine (2Pal); X4 is Arg, His, Trp, 7-methyltryptophan (7MeW), 6-methyltryptophan (6MeW), 7-fluorotryptophan (7FW), 6-fluorotryptophan (6FW), or 3-(2-naphthyl)alanine (2Nal); X5 is Ala, α-aminoisobutyric acid (Aib), D-alanine; X7 is D-alanine, β-alanine, α-aminoisobutyric acid (Aib), Gly, Ala, or S-β-aminobutanoic acid (BABA); X8 is His, D-histidine, 3-(3-pyridyl)alanine (3Pal), or Phe; X10 is absent, Met, Arg, norleucine (Nle), 3-(3-pyridyl)alanine (3Pal), or Lys(Me)₃; X11 is absent, sarcosine (Sar), α-aminoisobutyric acid (Aib), or Arg; and X12 is absent.

In some embodiments, X0 is absent or Glu; X1 is Gly, Phe, D-serine, or D-phenylalanine;
X2 is Glu, Asn, Arg, or Gln; Ar is histidine or 3-(3-pyridyl)alanine (3PAl); X4 is W or 7-methyltryptophan; X5 is Ala; X7 is D-alanine, S-β-aminobutanoic acid (BABA), or β-alanine;
X8 is His; X10 is norleucine (Nle); X11 is absent; and X12 is absent.

In some embodiments, X1 is Gly, Phe, or D-phenylalanine; X2 is Asn, Arg, or Gln; Ar is histidine or 3-(3-pyridyl)alanine (3PAl); X4 is W or 7-methyltryptophan; X5 is Ala; X7 is D-alanine or β-alanine; X8 is His; X10 is norleucine (Nle); X11 is absent; and X12 is absent.

In some embodiments, X1 is Phe or D-phenylalanine; X2 is Arg; Ar is histidine or 3-(3-pyridyl)alanine (3PAl); X4 is 7-methyltryptophan (7MeW) or 7-fluorotryptophan (7FW); X5 is Ala; X7 is D-alanine or β-alanine; X8 is His; X10 is norleucine (Nle); X11 is sarcosine (Sar);
and X12 is absent.

In some embodiments, the peptide comprises the amino acid sequence of any of the peptides in Tables 1, 1A, 2, or 3.

In some embodiments, the peptide comprises the amino acid sequence of any of the peptides in Table 1.

In some embodiments, the peptide comprises the amino acid sequence of any one of the peptides in Table 1A.

In some embodiments, the peptide comprises the amino acid sequence of any one of SEQ ID NOs 1-26.

In some embodiments, the peptide comprises the amino acid sequence of any one of SEQ ID NOs 1-5, 7-26, 46-112, 114-126, 128-134, 136-167, 240-245, 247, or 248.

In some embodiments, the peptide comprises an amino acid sequence comprising at least 50%, 55%, 60%, 65%, 70%, 75%, 80%, 85%, 90%, 95%, 96%, 97%, 98%, 99% or 100% sequence identity to any one of SEQ ID NOs 1-26.

In some embodiments, the peptide comprises an amino acid sequence comprising at least 50%, 55%, 60%, 65%, 70%, 75%, 80%, 85%, 90%, 95%, 96%, 97%, 98%, 99% or 100% sequence identity to any one of SEQ ID NOs 1-5, 7-26, 46-112, 114-126, 128-134, 136-167, 240-245, 247, or 248.

In some embodiments, the peptide comprises an amino acid sequence comprising at least 50%, 55%, 60%, 65%, 70%, 75%, 80%, 85%, 90%, 95%, 96%, 97%, 98%, 99% or 100% sequence identity to any one of SEQ ID NOs 1-5, 7-26, 46-112, 114-126, 128-134, 136-167, or 240-245.

In some embodiments, the peptide comprises an amino acid sequence comprising at least 50%, 55%, 60%, 65%, 70%, 75%, 80%, 85%, 90%, 95%, 96%, 97%, 98%, 99% or 100% sequence identity to any one of the peptides in Table 3.

In some embodiments, the peptide comprises the amino acid sequence of any one of SEQ ID NOs: 6, 27-43, 166-177, 179-212, 214-218, 220-239, or 246.

In some embodiments, the peptide comprises the amino acid sequence of any one of the SEQ ID NOs: 33, 223, 237, 238, 246, 249, or 250.

In some embodiments, the peptide comprises the amino acid sequence of any one of the SEQ ID NOs: 33, 223, 237, 238, or 246.

In some embodiments, the sequence identity is determined by a BLASTP, CLUSTALW, MUSCLE, MAFFT algorithm, or a CLUSTALW algorithm with the Smith-Waterman homology search algorithm parameters.

In some embodiments, the one or more lipid moieties are conjugated to the N-terminus of the peptide.

In some embodiments, the one or more lipid moieties are conjugated to the C-terminus of the peptide.

In some embodiments, the one or more lipid moieties are conjugated to the C-terminus of the peptide and the N-terminus of the peptide is substituted with an acetyl group.

In some embodiments, the one or more lipid moieties are conjugated to any one of the amino acid residues of Formula (I) or (V) having a suitable functional group for attachment to a lipid moiety.

In some embodiments, the one or more lipid moieties are conjugated to any one of the amino acid residues of Formula (I) having a suitable functional group for attachment to a lipid moiety.

In some embodiments, the one or more lipid moieties are conjugated to any one of the amino acid residues of Formula (I) or (V) through the amino acid side chain.

In some embodiments, the one or more lipid moieties are conjugated to any one of the amino acid residues of Formula (I) through the amino acid side chain.

In some embodiments, the one or more lipid moieties are conjugated to any one of the lysine residues of Formula (I).

In some embodiments, the one or more lipid moieties are conjugated to the N-terminus of the peptide and any one of the lysine residues of Formula (I).

In some embodiments, the lipid moiety or a lipid moiety of the two or more lipid moieties comprises a C₂-C₂₆ fatty acyl group.

In some embodiments, the lipid moiety or a lipid moiety of the two or more lipid moieties comprises a saturated fatty acyl group.

In some embodiments, the fatty acyl group comprises caprylyl, caprinoyl, lauroyl, myristoyl, palmitoyl, stearoyl, arachidonoyl, behenoyl, lignoceroyl, ceritoyl, octadecanedioyl, or eicosanedioyl.

In some embodiments, the fatty acyl group comprises palmitoyl or stearoyl.

In some embodiments, the lipid moiety or a lipid moiety of the two or more lipid moieties comprises -(L¹)ₚ-(L²)_{q}-(L³)ᵣ, wherein:
each L¹ is independently —((CH₂)ᵥNR¹)_{w}—CO(CH₂)ₓO(PEG)_{y}(CH₂)_{z}NR¹—;
PEG is —CH₂CH₂O—; v is 2-6; w is 0-1; x is 1-4; y is 1-4; z is 2-24; R¹ is H or —CH₃;
each L² is independently a natural or unnatural amino acid or amino acid analog;
each L³ is independently a substituted or unsubstituted C₂-C₂₆ fatty acyl group;
p is 0-8; q is 0-6; and r is 1 or 2.

In some embodiments, each L¹ is independently —CO(CH₂)O(PEG)(CH₂)₂NH— or —((CH₂)₃NH)—CO(CH₂)O(PEG)(CH₂)₂NH—; each L² is independently glutamic acid; each L³ is independently palmitoyl or stearoyl, wherein palmitoyl and stearoyl are unsubstituted or substituted with one or more of oxo, —OH, or —C(═O)OH; p is 0-4; q is 0-2; and r is 1 or 2.

In some embodiments, the lipid moiety or a lipid moiety of the two or more lipid moieties comprises -(L¹)ₚ-(L²)_{q}-(L³)ᵣ, wherein: each L¹ is OEG; OEG is —NH-PEG-PEG-CH₂C(═O)—; PEG is —CH₂CH₂O—; each L² is independently a natural or unnatural amino acid or amino acid analog; each L³ is independently a substituted or unsubstituted C₂-C₂₆ fatty acyl group; p is 0-8; q is 0-6; and r is 1 or 2.

In some embodiments, the lipid moiety or a lipid moiety of the two or more lipid moieties comprises -(L¹)ₚ-(L²)_{q}-(L³)ᵣ, wherein: each L¹ is OEG; OEG is —NH-PEG-PEG-CH₂C(═O)—; PEG is —CH₂CH₂O—; each L² is Glu or D-Glu; each L³ is independently a substituted or unsubstituted C₂-C₂₆ fatty acyl group; p is 0-8; q is 0-6; and r is 1 or 2.

In some embodiments, the lipid moiety or a lipid moiety of the two or more lipid moieties comprises -(L¹)ₚ-(L²)_{q}-(L³)ᵣ, wherein: each L¹ is OEG; OEG is —NH-PEG-PEG-CH₂C(═O)—; PEG is —CH₂CH₂O—; each L² is Glu or D-Glu; each L³ is independently palmitoyl, octadecanedioyl, or eicosanedioyl; p is 0-4; q is 0-2; and r is 1.

In some embodiments, -(L¹)ₚ-(L²)_{q}-(L³)ᵣ is

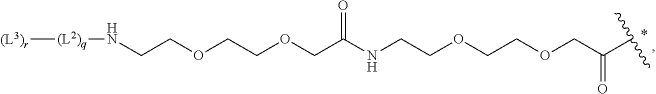

wherein * represents the attachment point to a nitrogen atom of the peptide.

In some embodiments, -(L¹)ₚ-(L²)_{q}-(L³)ᵣ is

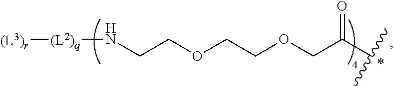

wherein * represents the attachment point to a nitrogen atom of the peptide.

In some embodiments, -(L¹)ₚ-(L²)_{q}-(L³)ᵣ is

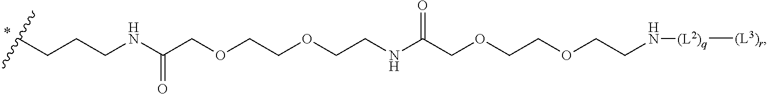

wherein * represents the attachment point to a nitrogen atom of the peptide.

In some embodiments, L¹)ₚ(L²)_{q}-(L³)ᵣ is

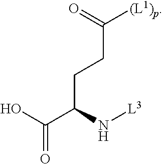

In some embodiments, -(L¹)ₚ-(L²)_{q}-(L³)ᵣ is

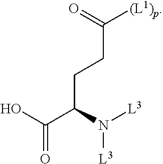

In some embodiments, L³ is

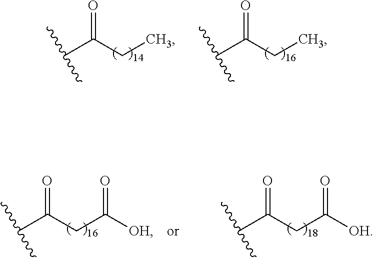

In some embodiments, L³ is

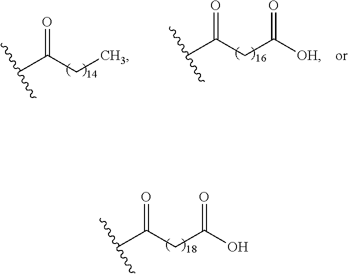

In some embodiments, -(L¹)ₚ-(L²)_{q}-(L³)ᵣ is -(L¹)₂-(L²)-(L³).

In some embodiments, -(L¹)ₚ-(L²)_{q}-(L³)ᵣ is

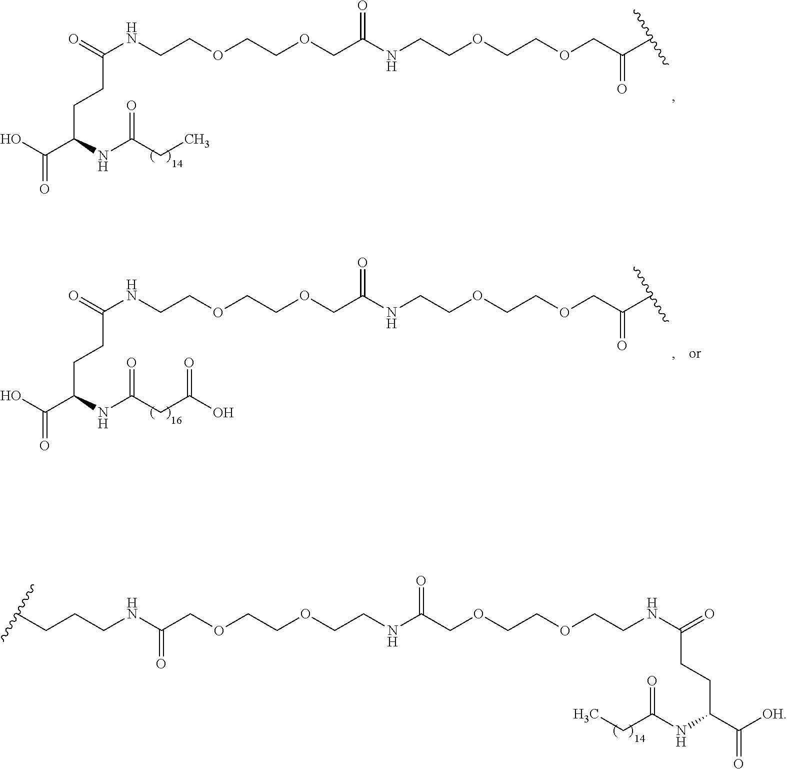

In some embodiments, -(L¹)ₚ-(L²)_{q}-(L³)ᵣ is

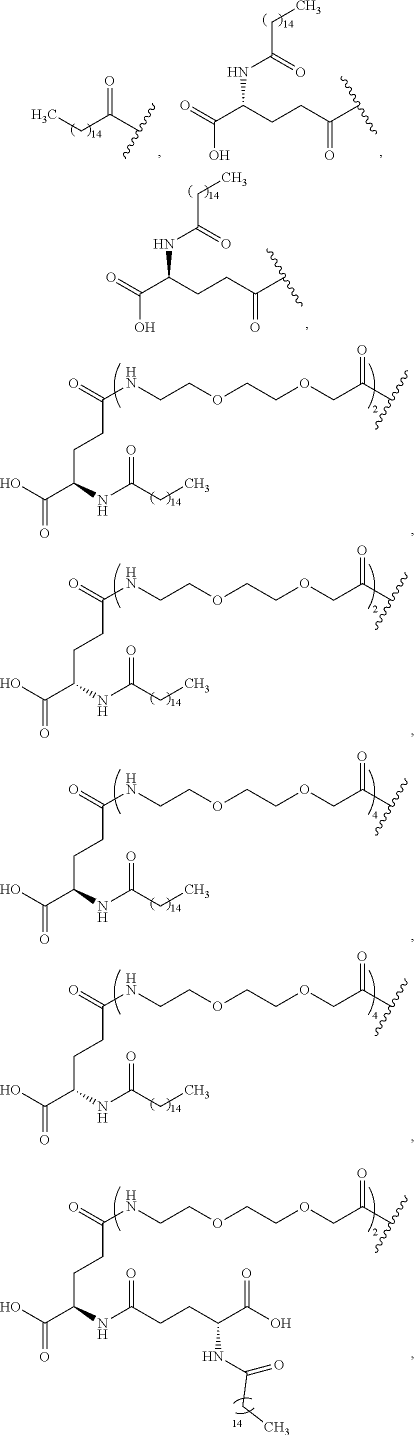

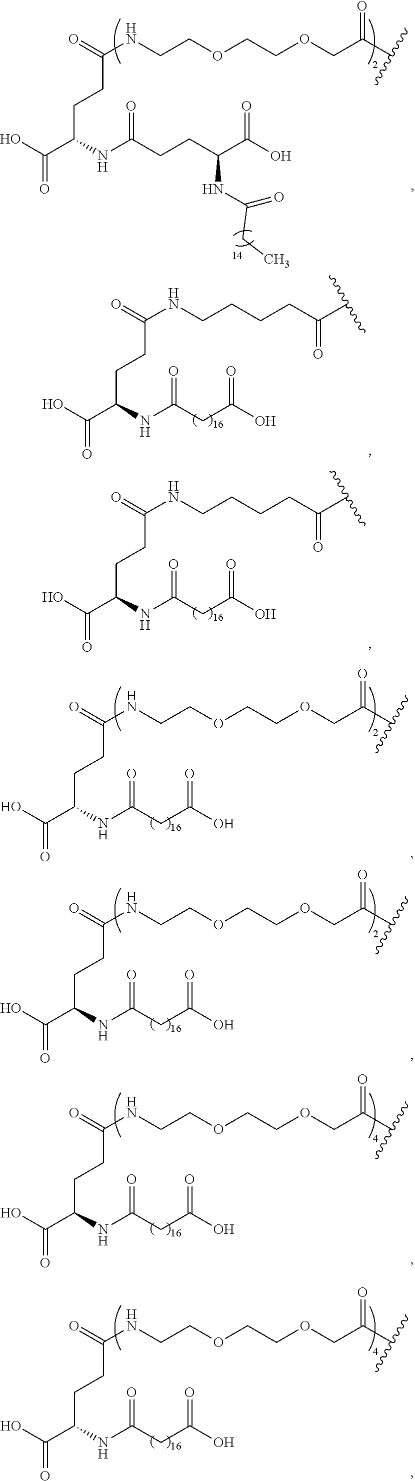

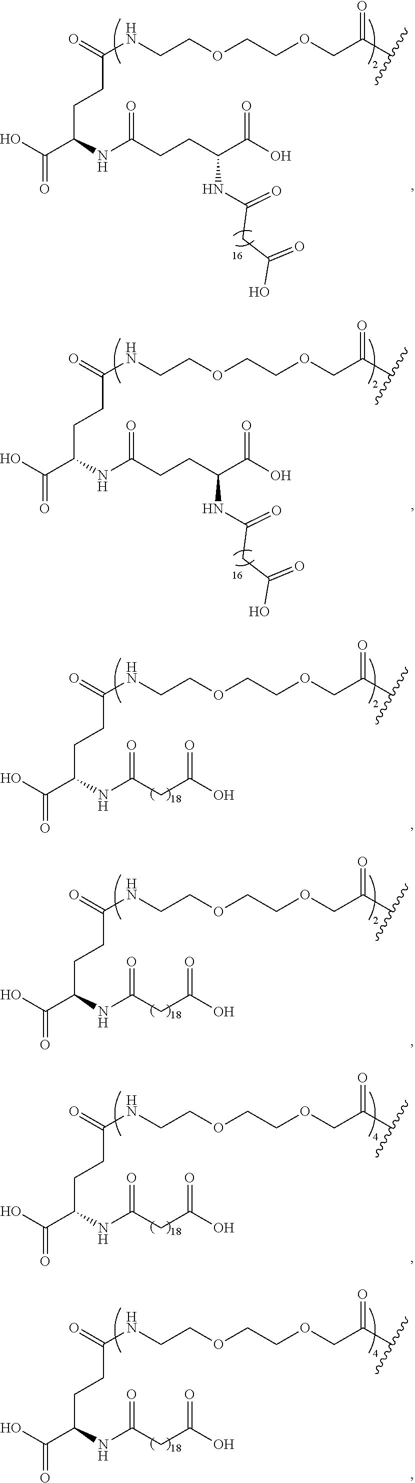

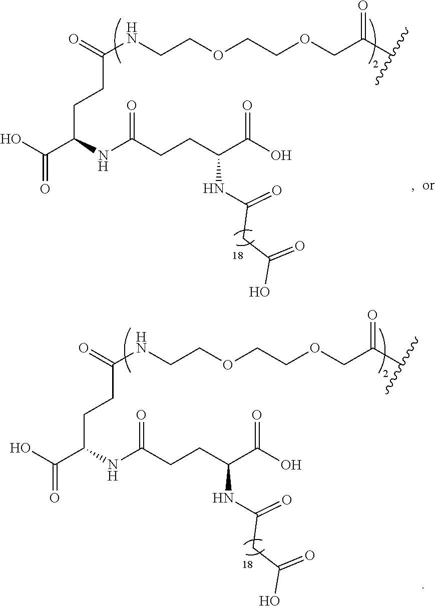

In some embodiments, -(L¹)ₚ-(L²)_{q}-(L³)ᵣ is

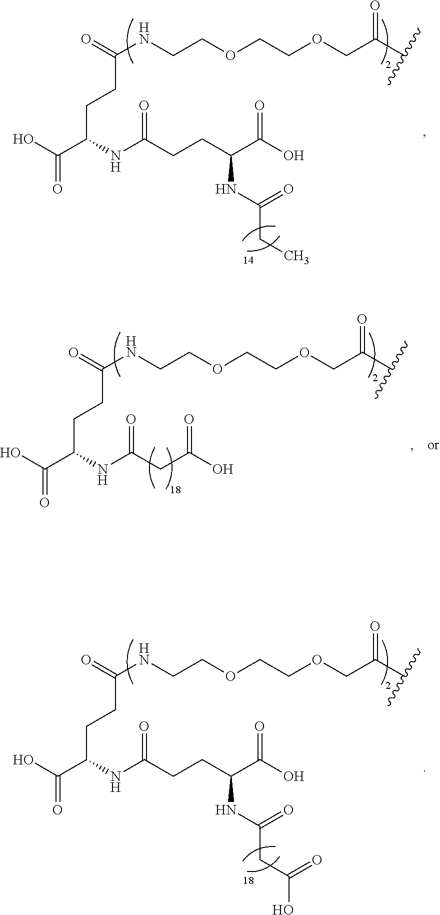

In some embodiments, -(L¹)ₚ-(L²)_{q}-(L³)ᵣ is

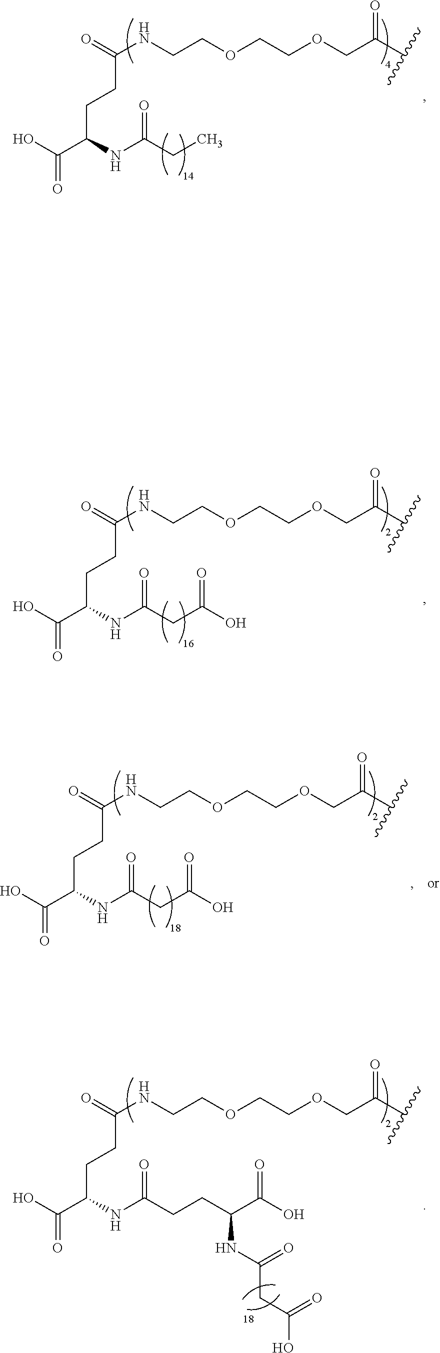

In some embodiments, the peptide is conjugated to 1 or 2 of -(L¹)ₚ-(L²)_{q}-(L³)ᵣ.

In some embodiments, the peptide is conjugated to 1 or 2 of

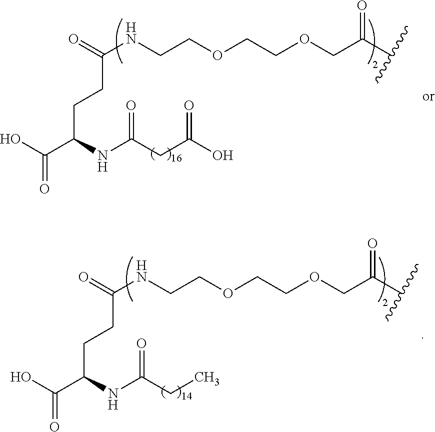

In some embodiments, the peptide is conjugated to 1 or 2 of

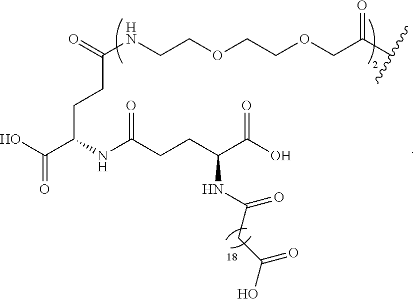

In some embodiments, the peptide is conjugated to -(L¹)ₚ-(L²)_{q}-(L³)ᵣ through the N-terminus of the peptide and a lysine residue of the peptide.

In one aspect, described herein is a peptide of Formula (VI),

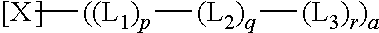

or a pharmaceutically acceptable salt, solvate, or isotope thereof, wherein:
X is a peptide comprising an amino acid sequence of Formula (V):

X0-X1-X2-Ar-X4-X5-V-X7-X8-L-X10-X11-X12,

or a pharmaceutically acceptable salt, solvate, or isotope thereof, wherein:
X4, X5, X7, and X8 are each independently a natural or non-natural amino acid or amino acid analog;
Ar is an aromatic amino acid;
X0, X1, X2, X10, X11, and X12 are each independently a natural or non-natural amino acid or amino acid analog, or absent;
each L¹ is independently —((CH₂)ᵥNR¹)_{w}—CO(CH₂)ₓO(PEG)_{y}(CH₂)_{z}NR¹—; PEG is —CH₂CH₂O—; v is 2-6; w is 0-1; x is 1-4; y is 1-4; z is 2-24; R¹ is H or —CH₃;
or each L¹ is OEG; OEG is —NH-PEG-PEG-CH₂C(═O)—; PEG is —CH₂CH₂O—;
each L² is independently a natural or unnatural amino acid or amino acid analog;
each L³ is independently a substituted or unsubstituted C₂-C₂₆ fatty acyl group;
a is 1, 2, 3, or 4; p is 0-8; q is 0-6; and r is 1 or 2.

In one aspect, described herein is a peptide of Formula (IV): [X [symbol] ((L₁)ₚ-(L₂)_{q}-(L₃)ᵣ)ₐ, or a pharmaceutically acceptable salt, solvate, or isotope thereof, wherein:
X is a peptide comprising an amino acid sequence of Formula (I):
X1-X2-Ar-X4-X5-V-X7-X8-L-X10-X11-X12,

X1-X2-Ar-X4-X5-V-X7-X8-L-X10-X11-X12,

or a pharmaceutically acceptable salt, solvate, or isotope thereof, wherein:
X4, X5, X7, and X8 are each independently a natural or non-natural amino acid or amino acid analog;
Ar is an aromatic amino acid;
X1, X2, X10, X11, and X12 are each independently a natural or non-natural amino acid or amino acid analog, or absent;
each L¹ is independently —((CH₂)ᵥNR¹)_{w}—CO(CH₂)ₓO(PEG)_{y}(CH₂)_{z}NR¹—; PEG is —CH₂CH₂O—; v is 2-6; w is 0-1; x is 1-4; y is 1-4; z is 2-24; R¹ is H or —CH₃;
each L² is independently a natural or unnatural amino acid or amino acid analog;
each L³ is independently a substituted or unsubstituted C₂-C₂₆ fatty acyl group;
a is 1,2,3, or 4; p is 0-8; q is 0-6; and r is 1 or 2.

In one aspect, described herein is a peptide of Formula (IV): [X [symbol] ((L¹)ₚ-(L₂)_{q}-(L₃)ᵣ)ₐ, or a pharmaceutically acceptable salt, solvate, or isotope thereof, wherein:
X is a peptide comprising an amino acid sequence of Formula (I):

X1-X2-Ar-X4-X5-V-X7-X8-L-X10-X11-X12,

or a pharmaceutically acceptable salt, solvate, or isotope thereof, wherein:
X4, X5, X7, and X8 are each independently a natural or non-natural amino acid or amino acid analog;
Ar is an aromatic amino acid;
X1, X2, X10, X11, and X12 are each independently a natural or non-natural amino acid or amino acid analog, or absent;
each L¹ is independently —((CH₂)ᵥNR¹)_{w}—CO(CH₂)ₓO(PEG)_{y}(CH₂)_{z}NR¹—; PEG is —CH₂CH₂O—; v is 2-6; w is 0-1; x is 1-4; y is 1-4; z is 2-24; R¹ is H or —CH₃;
or each L¹ is OEG;
   OEG is —NH-PEG-PEG-CH₂C(═O)—;
   PEG is —CH₂CH₂O—;
each L² is independently a natural or unnatural amino acid or amino acid analog;
each L³ is independently a substituted or unsubstituted C₂-C₂₆ fatty acyl group;
a is 1, 2, 3, or 4;
p is 0-8;
q is 0-6; and
r is 1 or 2.

In some embodiments, each (-(L¹)ₚ-(L²)_{q}-(L³)ᵣ) is independently attached to the peptide X at the N-terminus of the peptide, the C-terminus of the peptide, or any one of the amino acid residues of Formula (I) or (V) having a suitable functional group for attachment to a lipid moiety.

In some embodiments, each (-(L¹)ₚ-(L²)_{q}-(L³)ᵣ) is independently attached to the peptide X at the N-terminus of the peptide, the C-terminus of the peptide, or any one of the amino acid residues of Formula (I) having a suitable functional group for attachment to a lipid moiety.

In some embodiments, a is 2.

In some embodiments, each (-(L¹)ₚ-(L²)_{q}-(L³)ᵣ) is independently attached to the N-terminus of peptide X.

In some embodiments, each (-(L¹)ₚ-(L²)_{q}-(L³)ᵣ) is independently attached to any one of the amino acid residues of Formula (I) or (V) having a suitable functional group for attachment to a lipid moiety.

In some embodiments, each (-(L¹)ₚ-(L²)_{q}-(L³)ᵣ) is independently attached to any one of the amino acid residues of Formula (I) having a suitable functional group for attachment to a lipid moiety.

In some embodiments, a is 1.

In some embodiments, the peptide is no more than 14 amino acids in length.

In some embodiments, the peptide is no more than 13 amino acids in length.

In some embodiments, the peptide is no more than 12 amino acids in length.

In some embodiments, the peptide is no more than 11 amino acids in length.

In some embodiments, the peptide is no more than 10 amino acids in length.

In some embodiments, the peptide is no more than 9 amino acids in length.

In some embodiments, the peptide is no more than 8 amino acids in length.

In some embodiments, the peptide is no more than 7 amino acids in length.

In some embodiments, the peptide comprises the structure of any of the peptides in Table 1, or a pharmaceutically acceptable salt or solvate thereof.

Also described herein is a pharmaceutical composition comprising: (i) a peptide described herein, or a pharmaceutically acceptable salt or solvate thereof, and (ii) a pharmaceutically acceptable carrier, diluent, or excipient.

In some embodiments, the pharmaceutical composition further comprises an absorption enhancer.

In some embodiments, the absorption enhancer is for use in an oral formulation.

In some embodiments, the absorption enhancer is selected from the group consisting of sodium caprate, sodium caprylate, labrasol, and sodium N-(8-[2-hydroxylbenzoyl]amino) caprylate.

In some embodiments, described herein is a use of a peptide described herein, or a pharmaceutical composition comprising a peptide described herein, for the preparation of a medicament for the treatment of a disorder.

In some embodiments, the disorder is a gastrointestinal disease.

In some embodiments, the disorder is obesity.

In some embodiments, described herein is a method for weight management in a subject in need thereof, the method comprising: administering a therapeutically effective amount of a peptide described herein or a pharmaceutical composition comprising a peptide described herein.

In some embodiments, described herein is a method for reducing body weight of a subject in need thereof, the method comprising: administering a therapeutically effective amount of a peptide described herein or a pharmaceutical composition comprising a peptide described herein.

In some embodiments, described herein is a method for treating obesity in a subject in need thereof, the method comprising: administering a therapeutically effective amount of a peptide of described herein or a pharmaceutical composition comprising a peptide described herein.

In some embodiments, the administering the peptide occurs weekly.

In some embodiments, the administering the peptide occurs every 7-14 days, every two weeks, every three weeks, or every four weeks.

In some embodiments, the administering the peptide occurs semimonthly or monthly.

In some embodiments, the administering the peptide occurs monthly.

In some embodiments, the subject is a human.

In some embodiments, the subject is overweight.

In some embodiments, the subject is obese.

In some embodiments, the subject has at least one weight-related comorbid condition selected from the group consisting of: hypertension, type 2 diabetes mellitus, dyslipidemia, metabolic-associated steatohepatitis, sleep apnea, and urinary incontinence.

In some embodiments, the subject has received at least one previous treatment of a weight management therapy.

In some embodiments, administering the peptide reduces a body weight of the subject by at least 5%, for example, when compared to a body weight of an otherwise identical subject not administered the peptide. In some embodiments, administering the peptide reduces a body weight of the subject by at least 5%, for example, when compared to the body weight of the subject at baseline. In some embodiments, the baseline is the body weight of the subject before the subject begins administration of the peptide. For example, baseline can be the body weight of the subject on Day 0 if peptide administration begins on Day 1.

In some embodiments, the peptide is administered intravenously.

In some embodiments, the peptide is administered subcutaneously.

In some embodiments, the peptide is administered orally.

In some embodiments, the method described herein further comprises administering with an absorption enhancer.

In some embodiments, the method described herein further comprises administering a therapeutically effective amount of another weight loss agent.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

Bombesin receptors are a class of G protein-coupled receptors that regulate several functions of the gastrointestinal tract and central nervous system. The Bombesin receptor family includes gastrin releasing peptide (GRP) receptor (GRPR or BB₂ receptor), neuromedin B (NMB) receptor (NMBR or BB₁ receptor), and the orphan receptor, bombesin receptor subtype 3 (BRS-3) receptor (BB₃ receptor). Activation of GRPR induces a broad range of physiological effects, including smooth muscle cell contraction, tissue growth, gastrointestinal motility, feeding behavior, regulation of circadian rhythm, thermoregulation, and satiety.

Biologically active peptides are promising drug candidates due to high selectivity, high binding affinity, and low toxicity. However, the use of native peptides as drug candidates is limited due to their short half-lives, chemical and physical stability, and low bioavailability. For example, native GRP has a short half-life and is rapidly cleared from the bloodstream. Structural modifications of native peptides (e.g., native GRP), can provide peptide analogs having improved pharmacological properties to the parent peptide for use in therapeutics.

Peptide lipidation or conjugation of lipids to peptides can be an attractive strategy to improve the therapeutic potential of peptide drugs. The presence of fatty acyl(s) increases the lipophilicity of peptides, affecting the secondary structure and binding properties of peptides. As such, conjugation of lipids to peptides as described herein, can alter absorption, distribution, metabolism, and excretion (ADME) properties. Described herein are long-acting lipidated peptide analogs of gastrin releasing peptide (GRP) for use in the treatment of a disease or disorder. The peptide analogs of the disclosure can comprise greater potency, selectivity, half-life and/or solubility compared with native or parent GRP peptides.

### Peptides

In some embodiments, described herein are long-acting peptide analogs of GRP or GRP-10.

In some embodiments, described herein is a peptide comprising an amino acid sequence of Formula (I): X1-X2-Ar-X4-X5-V-X7-X8-L-X10-X11-X12, or a pharmaceutically acceptable salt, solvate, or isotope thereof, wherein:
X4, X5, X7, and X8 are each independently a natural or non-natural amino acid or amino acid analog;
Ar is an aromatic amino acid;
X1, X2, X10, X11, and X12 are each independently a natural or non-natural amino acid or amino acid analog, or absent;
wherein the peptide is conjugated to one or more lipid moieties.

In some embodiments of Formula (I), when X1 is a natural or non-natural amino acid or amino acid analog and X2 is absent, X4 is Ala, Arg, Asn, Asp, Cys, Glu, Gln, Gly, His, Ile, Lys, Met, Phe, Pro, Ser, Thr, Trp, Tyr, Val, or a non-natural amino acid or amino acid analog; when X1 is absent and X2 is a natural or non-natural amino acid or amino acid analog, X4 is Ala, Arg, Asn, Asp, Cys, Glu, Gln, Gly, His, Ile, Lys, Met, Phe, Pro, Ser, Thr, Trp, Tyr, Val, or a non-natural amino acid or amino acid analog; and when X1 and X2 are each absent, X5 is Ala, Arg, Asn, Asp, Cys, Glu, Gln, Gly, His, Ile, Lys, Met, Phe, Pro, Ser, Thr, Trp, Tyr, Val, or a non-natural amino acid or amino acid analog.

In some embodiments of Formula (I), when X1 is a natural or non-natural amino acid or amino acid analog and X2 is absent, X7 is Ala, Arg, Asn, Asp, Cys, Glu, Gln, Gly, His, Ile, Leu, Lys, Met, Phe, Pro, Ser, Trp, Tyr, Val, or a non-natural amino acid or amino acid analog; when X1 is absent and X2 is a natural or non-natural amino acid or amino acid analog, X7 is Ala, Arg, Asn, Asp, Cys, Glu, Gln, Gly, His, Ile, Leu, Lys, Met, Phe, Pro, Ser, Trp, Tyr, Val, or a non-natural amino acid or amino acid analog; and when X1 and X2 are each absent, X8 is Ala, Arg, Asn, Asp, Cys, Glu, Gln, Gly, His, Ile, Leu, Lys, Met, Phe, Pro, Ser, Trp, Tyr, Val, or a non-natural amino acid or amino acid analog.

In some embodiments of Formula (I), when X1 is a natural or non-natural amino acid or amino acid analog and X2 is absent, X10 is Ala, Arg, Asn, Asp, Cys, Glu, Gln, Gly, His, Ile, Leu, Lys, Met, Pro, Ser, Thr, Trp, Tyr, Val, or a non-natural amino acid or amino acid analog; when X1 is absent and X2 is a natural or non-natural amino acid or amino acid analog, X10 is Ala, Arg, Asn, Asp, Cys, Glu, Gln, Gly, His, Ile, Leu, Lys, Met, Pro, Ser, Thr, Trp, Tyr, Val, or a non-natural amino acid or amino acid analog; and when X1 and X2 are each absent, X11 is Ala, Arg, Asn, Asp, Cys, Glu, Gln, Gly, His, Ile, Leu, Lys, Met, Pro, Ser, Thr, Trp, Tyr, Val, or a non-natural amino acid or amino acid analog.

In some embodiments, described herein is a peptide comprising an amino acid sequence of Formula (V): X0-X1-X2-Ar-X4-X5-V-X7-X8-L-X10-X11-X12, or a pharmaceutically acceptable salt, solvate, or isotope thereof, wherein:
X4, X5, X7, and X8 are each independently a natural or non-natural amino acid or amino acid analog;
Ar is an aromatic amino acid;
X0, X1, X2, X10, X11, and X12 are each independently a natural or non-natural amino acid or amino acid analog, or absent;
wherein the peptide is conjugated to one or more lipid moieties. In some embodiments of Formula (V), X2 is Ala, Arg, Asn, Asp, Cys, Glu, Gln, Gly, His, Ile, Lys, Met, Phe, Pro, Ser, Thr, Trp, Tyr, Val, or a non-natural amino acid or amino acid analog.

In some embodiments of Formula (V), X5 is Ala, Arg, Asn, Asp, Cys, Glu, Gln, Gly, His, Ile, Leu, Lys, Met, Phe, Pro, Ser, Trp, Tyr, Val, or a non-natural amino acid or amino acid analog.

In some embodiments of Formula (V), X8 is Ala, Arg, Asn, Asp, Cys, Glu, Gln, Gly, His, Ile, Leu, Lys, Met, Pro, Ser, Thr, Trp, Tyr, Val, or a non-natural amino acid or amino acid analog.

In some embodiments, described herein is a peptide comprising an amino acid sequence of Formula (II): X1-X2-Ar-X4-X5-V-X7-X8-L-X10-X11-X12, or a pharmaceutically acceptable salt, solvate, or isotope thereof, wherein:
X1 is Gly, Ala, N-acetylglycine, or D-alanine;
X2 is Asn, Glu, Gln, Arg, or Asp;
Ar is histidine or 3-(3-pyridyl)alanine (3PAl);
X4 is Trp, 7-methyltryptophan (7MeW), Phe, D-alanine, or Tyr;
X5 is Ala, α-aminoisobutyric acid (Aib), Gly, Leu, Val, Ile, D-alanine, or Nle;
X7 is Gly, D-alanine, Ala, Aib, Leu, Val, Ile, or Nle;
X8 is His or 3-(3-pyridyl)alanine (3PAl);
X10 is Met, Nle, Aib, Leu, Val, or Ile;
X11 is sarcosine (Sar), Gly, Pro, Ala, D-alanine, or absent; and
X12 is independently a natural or non-natural amino acid or amino acid analog, or absent.

In some embodiments, the peptide comprises an amino acid sequence of Formula (II):
X1-X2-Ar-X4-X5-V-X7-X8-L-X10-X11-X12, or a pharmaceutically acceptable salt, solvate, or isotope thereof, wherein:
X1 is absent, α-aminoisobutyric acid (Aib), Arg, Phe, Gly, Ala, N-acetylglycine, or D-alanine;
X2 is Asn, Glu, Gln, Arg, Ala, Asp, Lys, or Lys(Ac);
Ar is histidine, 3-(3-pyridyl)alanine (3PAl), 3-(4-pyridyl)alanine (4Pal), or 3-(2-pyridyl)alanine (2Pal);
X4 is Arg, His, Trp, 7-methyltryptophan (7MeW), 6-methyltryptophan (6MeW), 7-fluorotryptophan (7FW), 6-fluorotryptophan (6FW), or 3-(2-Naphthyl)alanine (2Nal), Phe, D-alanine, or Tyr;
X5 is Ala, α-aminoisobutyric acid (Aib), Gly, Leu, Val, Ile, D-alanine, or Nle;
X7 is Gly, D-alanine, β-alanine, Ala, Aib, Leu, Val, Ile, or Nle;
X8 is His, Phe, D-histidine, or 3-(3-pyridyl)alanine (3PAl);
X10 is absent, Met, Arg, norleucine (Nle), Aib, Leu, Val, Ile, 3-(3-pyridyl)alanine (3Pal), cyclohexylalanine (Cha), or Lys(Me)₃;
X11 is absent, sarcosine (Sar), Gly, Pro, Ala, Arg, D-alanine, or α-aminoisobutyric acid (Aib);
X12 is independently a natural or non-natural amino acid or amino acid analog, or absent.

In some embodiments, X1 is absent, Gly, α-aminoisobutyric acid (Aib), Arg, Phe, or D-phenylalanine; X2 is Asn, Glu, Arg, Ala, Asp, Gln, or Lys(Ac); Ar is histidine, 3-(3-pyridyl)alanine (3Pal), 3-(4-pyridyl)alanine (4Pal), or 3-(2-pyridyl)alanine (2Pal); X4 is Arg, His, Trp, 7-methyltryptophan (7MeW), 6-methyltryptophan (6MeW), 7-fluorotryptophan (7FW), 6-fluorotryptophan (6FW), or 3-(2-naphthyl)alanine (2Nal); X5 is Ala, α-aminoisobutyric acid (Aib), or D-alanine; X7 is D-alanine, β-alanine, α-aminoisobutyric acid (Aib), Gly, or Ala; X8 is His, D-histidine, 3-(3-pyridyl)alanine (3Pal), or Phe; X10 is absent, Met, Arg, norleucine (Nle), 3-(3-pyridyl)alanine (3Pal), or Lys(Me)₃; X11 is absent, sarcosine (Sar), α-aminoisobutyric acid (Aib), or Arg; and X12 is absent.

In some embodiments, X0 is absent or Glu; X1 is absent, Gly, Glu, α-aminoisobutyric acid (Aib), Arg, Phe, D-phenylalanine, or D-serine; X2 is Asn, Glu, Arg, Ala, Asp, Gln, or Lys(Ac); Ar is histidine, 3-(3-pyridyl)alanine (3Pal), 3-(4-pyridyl)alanine (4Pal), or 3-(2-pyridyl)alanine (2Pal); X4 is Arg, His, Trp, 7-methyltryptophan (7MeW), 6-methyltryptophan (6MeW), 7-fluorotryptophan (7FW), 6-fluorotryptophan (6FW), or 3-(2-naphthyl)alanine (2Nal); X5 is Ala, α-aminoisobutyric acid (Aib), D-alanine; X7 is D-alanine, β-alanine, α-aminoisobutyric acid (Aib), Gly, Ala, or S-β-aminobutanoic acid (BABA); X8 is His, D-histidine, 3-(3-pyridyl)alanine (3Pal), or Phe; X10 is absent, Met, Arg, norleucine (Nle), 3-(3-pyridyl)alanine (3Pal), or Lys(Me)₃; X11 is absent, sarcosine (Sar), α-aminoisobutyric acid (Aib), or Arg; and X12 is absent.

In some embodiments, X0 is absent or Glu; X1 is Gly, Phe, D-serine, or D-phenylalanine; X2 is Glu, Asn, Arg, or Gln; Ar is histidine or 3-(3-pyridyl)alanine (3PAl); X4 is W or 7-methyltryptophan; X5 is Ala; X7 is D-alanine, S-β-aminobutanoic acid (BABA), β-alanine; X8 is His; X10 is norleucine (Nle); X11 is absent; and X12 is absent.

In some embodiments, X1 is Gly, Phe, or D-phenylalanine; X2 is Asn, Arg, or Gln; Ar is histidine or 3-(3-pyridyl)alanine (3PAl); X4 is W or 7-methyltryptophan; X5 is Ala; X7 is D-alanine or β-alanine; X8 is His; X10 is norleucine (Nle); X11 is absent; and X12 is absent.

In some embodiments, X1 is Phe or D-phenylalanine; X2 is Arg; Ar is histidine or 3-(3-pyridyl)alanine (3PAl); X4 is 7-methyltryptophan (7MeW) or 7-fluorotryptophan (7FW); X5 is Ala; X7 is D-alanine or β-alanine; X8 is His; X10 is norleucine (Nle); X11 is sarcosine (Sar);
and X12 is absent. In some embodiments, described herein is a peptide comprising an amino acid sequence of Formula (III): G-X2-Ar-X4-X5-V-X7-X8-L-X10-X11, or a pharmaceutically acceptable salt, solvate, or isotope thereof, wherein:
X2 is Asn or Glu;
Ar is His or 3-(3-pyridyl)alanine (3PAl);
X4 is W or 7-methyltryptophan (7MeW);
X5 is Ala or α-aminoisobutyric acid (Aib);
X7 is Gly, D-alanine or α-aminoisobutyric acid (Aib);
X8 is His or 3-(3-pyridyl)alanine (3PAl);
X10 is Met or norleucine (Nle); and
X11 is sarcosine (Sar) or absent.

In some embodiments, a peptide of Formula (I) is a peptide of Formula (II) or Formula (III).

In some embodiments, X0 is absent, Glu, Ser, Arg, or Gly. In some embodiments, X0 is Glu, Arg, or Ser. In some embodiments, X0 is absent. In some embodiments, X0 is Glu. In some embodiments, X0 is Ser. In some embodiments, X0 is Arg. In some embodiments, X0 is Gly.

In some embodiments, X1 is absent, Gly, Ala, D-alanine, N-acetylglycine, Lys(Ac), N-α-acetyllysine, α-aminoisobutyric acid (Aib), Arg, Phe, D-serine, or D-phenylalanine. In some embodiments, X1 is absent, Gly, Ala, D-alanine, N-acetylglycine, Lys(Ac), N-α-acetyllysine, α-aminoisobutyric acid (Aib), Arg, Phe, or D-phenylalanine. In some embodiments, X1 is absent, Gly, Ala, D-alanine, Lys(Ac), or N-α-acetyllysine. In some embodiments, X1 is absent. In some embodiments, X1 is Gly, Ala, or D-alanine. In some embodiments, X1 is Gly. In some embodiments, X1 is N-acetylglycine. In some embodiments, X1 is Phe, D-phenylalanine, or Gly. In some embodiments, X1 is Phe or Gly. In some embodiments, X1 is D-Ser, Ser, Gly. In some embodiments, X1 is Ser. In some embodiments, X1 is Phe. In some embodiments, X1 is D-phenylalanine. In some embodiments, X1 is Gly. In some embodiments, X1 is D-serine.

In some embodiments, X2 is absent, Ala, D-alanine, α-aminoisobutyric acid (Aib), Gly, Lys(Ac), N-α-acetyllysine, Asn, Glu, Gln, Arg, or Asp. In some embodiments, X2 is absent, Ala, D-alanine, α-aminoisobutyric acid (Aib), Gly, Lys, Lys(Ac), N-α-acetyllysine, Asn, Glu, Gln, Arg, or Asp. In some embodiments, X2 is Asn, Glu, Arg, Ala, Asp, Gln, Lys, or Lys(Ac). In some embodiments, X2 is Asn, Glu, Gln, Arg, or Asp. In some embodiments, X2 is Asn or Glu. In some embodiments, X2 is Asn, Arg, or Gln. In some embodiments, X2 is Glu or Asp. In some embodiments, X2 is Glu. In some embodiments, X2 is Asn. In some embodiments, X2 is Arg. In some embodiments, X2 is Gln. In some embodiments, X2 is Asp.

In some embodiments, Ar is an aromatic amino acid selected from Phe, Trp, Tyr, His, 1-methylhistidine, 3-methylhistidine, 3-(3-pyridyl)alanine (3PAl), 3-(2-pyridyl)alanine (2PAl), 3-(4-pyridyl)alanine (4PAl), 3-(1-pyrazolyl)alanine, 2-amino-3-(pyrazin-2-yl)propanoic acid, 2-amino-3-(furan-2-yl)propanoic acid, 2-amino-3-(naphthalen-2-yl)propanoic acid, 2-methylphenylalanine, 3-methylphenylalanine, 4-methylphenylalanine, 2-fluorophenylalanine, 3-fluorophenylalanine, 4-fluorophenylalanine, 2-methyltryptophan (2MeW), 4-methyltryptophan (4MeW), 5-methyltryptophan (5MeW), 6-methyltryptophan (6MeW), or 7-methyltryptophan (7MeW). In some embodiments, Ar is histidine. In some embodiments, Ar is Trp. In some embodiments, Ar is histidine, 1-methylhistidine, or 3-methylhistidine. In some embodiments, Ar is 2-amino-3-(pyrazin-2-yl)propanoic acid, 2-amino-3-(furan-2-yl)propanoic acid, or 2-amino-3-(naphthalen-2-yl)propanoic acid. In some embodiments, Ar is phenylalanine, 2-methylphenylalanine, 3-methylphenylalanine, 4-methylphenylalanine, 2-fluorophenylalanine, 3-fluorophenylalanine, or 4-fluorophenylalanine. In some embodiments, Ar is tryptophan, 2-methyltryptophan (2MeW), 4-methyltryptophan (4MeW), 5-methyltryptophan (5MeW), 6-methyltryptophan (6MeW), or 7-methyltryptophan (7MeW). In some embodiments, Ar is 7-methyltryptophan (7MeW). In some embodiments, Ar is 3-(3-pyridyl)alanine, 3-(2-pyridyl)alanine (2PAl), or 3-(4-pyridyl)alanine (4PAl). In some embodiments, Ar is 3-(3-pyridyl)alanine (3PAl). In some embodiments, Ar is histidine, 3-(3-pyridyl)alanine (3Pal), 3-(4-pyridyl)alanine (4Pal), or 3-(2-pyridyl)alanine (2Pal). In some embodiments, Ar is histidine or 3-(3-pyridyl)alanine (3PAl). In some embodiments, Ar is histidine. In some embodiments, Ar is 3-(3-pyridyl)alanine (3PAl).

In some embodiments, X4 is Arg, Phe, Trp, Tyr, His, 1-methylhistidine, 3-methylhistidine, 3-(3-pyridyl)alanine (3PAl), 3-(2-pyridyl)alanine (2PAl), 3-(4-pyridyl)alanine (4PAl), 3-(1-pyrazolylalanine, 2-amino-3-(pyrazin-2-yl)propanoic acid, 2-amino-3-(furan-2-yl)propanoic acid, 2-amino-3-(naphthalen-2-yl)propanoic acid, 2-methylphenylalanine, 3-methylphenylalanine, 4-methylphenylalanine, 2-fluorophenylalanine, 3-fluorophenylalanine, 4-fluorophenylalanine, 2-methyltryptophan (2MeW), 4-methyltryptophan (4MeW), 5-methyltryptophan (5MeW), 6-methyltryptophan (6MeW), 7-methyltryptophan (7MeW), 7-fluorotryptophan (7FW), 6-fluorotryptophan (6FW), or 3-(2-naphthyl)alanine (2Nal). In some embodiments, X4 is Phe, Trp, Tyr, His, 1-methylhistidine, 3-methylhistidine, 3-(3-pyridyl) alanine (3PAl), 3-(2-pyridyl)alanine (2PAl), 3-(4-pyridyl)alanine (4PAl), 3-(1-pyrazolyl)alanine, 2-amino-3-(pyrazin-2-yl)propanoic acid, 2-amino-3-(furan-2-yl)propanoic acid, 2-amino-3-(naphthalen-2-yl)propanoic acid, 2-methylphenylalanine, 3-methylphenylalanine, 4-methylphenylalanine, 2-fluorophenylalanine, 3-fluorophenylalanine, 4-fluorophenylalanine, 2-methyltryptophan (2MeW), 4-methyltryptophan (4MeW), 5-methyltryptophan (5MeW), 6-methyltryptophan (6MeW), or 7-methyltryptophan (7MeW). In some embodiments, X4 is histidine. In some embodiments, X4 is Trp. In some embodiments, X4 is histidine, 1-methylhistidine, or 3-methylhistidine. In some embodiments, X4 is 2-amino-3-(pyrazin-2-yl)propanoic acid, 2-amino-3-(furan-2-yl)propanoic acid, or 2-amino-3-(naphthalen-2-yl)propanoic acid. In some embodiments, X4 is phenylalanine, 2-methylphenylalanine, 3-methylphenylalanine, 4-methylphenylalanine, 2-fluorophenylalanine, 3-fluorophenylalanine, or 4-fluorophenylalanine. In some embodiments, X4 is tryptophan, 2-methyltryptophan, 4-methyltryptophan, 5-methyltryptophan, 6-methyltryptophan, or 7-methyltryptophan. In some embodiments, X4 is 3-(2-pyridyl)alanine, 3-(3-pyridyl)alanine, or 3-(4-pyridyl)alanine. In some embodiments, X4 is 3-(3-pyridyl) alanine. In some embodiments, X4 is Arg, His, Trp, 7-methyltryptophan (7MeW), 6-methyltryptophan (6MeW), 7-fluorotryptophan (7FW), 6-fluorotryptophan (6FW), or 3-(2-naphthyl)alanine (2Nal). In some embodiments, X4 is 7-methyltryptophan (7MeW) or 7-fluorotryptophan (7FW). In some embodiments, X4 is W or 7-methyltryptophan (7MeW). In some embodiments, X4 is Trp. In some embodiments, X4 is 7-methyltryptophan.

In some embodiments, X5 is Ala, α-aminoisobutyric acid (Aib), Gly, Leu, Val, Ile, Lys, Lys(Ac), Lys(Me)₃, D-alanine, or Ne. In some embodiments, X5 is Ala, α-aminoisobutyric acid (Aib), Gly, Leu, Val, Ile, D-alanine, or Ne. In some embodiments, X5 is Ala, D-alanine, or Aib. In some embodiments, X5 is Ala. In some embodiments, X5 is Aib.

In some embodiments, X7 is Gly, D-alanine, β-alanine, Ala, Aib, Leu, Val, Ile, Lys, Lys(Ac), Lys(Me)₃, or Nle. In some embodiments, X7 is Gly, D-alanine, Ala, Aib, Leu, Val, Ile, Lys, Lys(Ac), Lys(Me)₃, or Ne. In some embodiments, X7 is Gly, D-alanine, Ala, Aib, Leu, Val, Ile, or Ne. In some embodiments, X7 is Gly, D-alanine, or β-alanine. In some embodiments, X7 is D-alanine or β-alanine. In some embodiments, X7 is β-alanine or BABA. In some embodiments, X7 is Gly, Ala, D-alanine, or Aib. In some embodiments, X7 is Gly, D-alanine, or Aib. In some embodiments, X7 is Gly. In some embodiments, X7 is D-alanine. In some embodiments, X7 is Aib. In some embodiments, X7 is β-alanine. In some embodiments, X7 is S-β-aminobutanoic acid (BABA).

In some embodiments, X8 is Phe, Trp, Tyr, His, 1-methylhistidine, 3-methylhistidine, 3-(3-pyridyl)alanine (3PAl), 3-(2-pyridyl)alanine (2PAl), 3-(4-pyridyl)alanine (4PAl), 3-(1-pyrazolyl)alanine, 2-amino-3-(pyrazin-2-yl)propanoic acid, 2-amino-3-(furan-2-yl)propanoic acid, 2-amino-3-(naphthalen-2-yl)propanoic acid, 2-methylphenylalanine, 3-methylphenylalanine, 4-methylphenylalanine, 2-fluorophenylalanine, 3-fluorophenylalanine, 4-fluorophenylalanine, 2-methyltryptophan (2MeW), 4-methyltryptophan (4MeW), 5-methyltryptophan (5MeW), 6-methyltryptophan (6MeW), or 7-methyltryptophan (7MeW). In some embodiments, X8 is histidine. In some embodiments, X8 is Trp. In some embodiments, X8 is histidine, 1-methylhistidine, or 3-methylhistidine. In some embodiments, X8 is 2-amino-3-(pyrazin-2-yl)propanoic acid, 2-amino-3-(furan-2-yl)propanoic acid, or 2-amino-3-(naphthalen-2-yl)propanoic acid. In some embodiments, X8 phenylalanine, 2-methylphenylalanine, 3-methylphenylalanine, 4-methylphenylalanine, 2-fluorophenylalanine, 3-fluorophenylalanine, or 4-fluorophenylalanine. In some embodiments, X8 is Trp, 2-methyl tryptophan, 4-methyltryptophan, 5-methyltryptophan, 6-methyl tryptophan, or 7-methyltryptophan. In some embodiments, X8 is W or 7-methyltryptophan. In some embodiments, X8 is 7-methyltryptophan. In some embodiments, X8 is 3-(2-pyridyl)alanine, 3-(3-pyridyl)alanine, or 3-(4-pyridyl)alanine. In some embodiments, X8 is His or 3-(3-pyridyl)alanine. In some embodiments, X8 is 3-(3-pyridyl)alanine. In some embodiments, X8 is His, D-histidine, 3-(3-pyridyl)alanine (3Pal), or Phe. In some embodiments, X8 is His or NMeHis. In some embodiments, X8 is NMeHis. In some embodiments, X8 is His.

In some embodiments, X10 is absent, Met, norleucine (Nle), Aib, Leu, Val, Lys, Lys(Ac), or Ile. In some embodiments, X10 is Met, norleucine (Nle), Aib, Leu, Val, or Ile. In some embodiments, X10 is Met or Nle. In some embodiments, X10 is Met. In some embodiments, X10 is Nle. In some embodiments, X10 is absent, Met, Arg, norleucine (Nle), 3-(3-pyridyl)alanine (3Pal), Aib, Leu, Val, Lys, Lys(Ac), Lys(Me)₃, or Ile. In some embodiments, X10 is absent, Met, Arg, norleucine (Nle), 3-(3-pyridyl)alanine (3Pal), cyclohexylalanine (Cha), or Lys(Me)₃.

In some embodiments, X11 is absent, sarcosine (Sar), Gly, Pro, Ala, D-alanine, α-aminoisobutyric acid (Aib), or Arg. In some embodiments, X11 is absent, sarcosine (Sar), Gly, Pro, Ala, or D-alanine. In some embodiments, X11 is absent, sarcosine (Sar), α-aminoisobutyric acid (Aib), or Arg. In some embodiments, X11 is Sar or absent. In some embodiments, X11 is absent. In some embodiments, X11 is Sar.

In some embodiments, X12 is absent. In some embodiments, X12 is a natural amino acid. In some embodiments, X12 is a non-natural amino acid.

In some embodiments, described herein is a peptide comprising the amino acid sequence of any of the peptides in Tables 1-3.

In some embodiments, described herein is a peptide comprising the amino acid sequence of any one of SEQ ID NOs: 1-44.

In some embodiments, described herein is a peptide comprising an amino acid sequence comprising at least 50%, 55%, 60%, 65%, 70%, 75%, 80%, 85%, 90%, 95%, 96%, 97%, 98%, 99% or 100% sequence identity to any one of SEQ ID NOs: 1-44.

In some embodiments, a peptide of the disclosure consists of the amino acid sequence of any one of SEQ ID NOs: 1-44.

A peptide of the disclosure can comprise a length of at least 7, 8, 9, 10, 11, 12, 13, or 14 amino acids. A peptide of the disclosure can comprise a length of at least 7, 8, 9, 10, 11, or 12 amino acids. In some embodiments, described herein is a peptide that is no more than 14 amino acids in length. In some embodiments, described herein is a peptide that is no more than 13 amino acids in length. In some embodiments, the peptide is no more than 12 amino acids in length. In some embodiments, the peptide is no more than 11 amino acids in length. In some embodiments, the peptide is no more than 10 amino acids in length. In some embodiments, the peptide is no more than 9 amino acids in length. In some embodiments, the peptide is no more than 8 amino acids in length. In some embodiments, the peptide is no more than 7 amino acids in length.

In some embodiments, peptides described herein can comprise a linker, a lipid moiety, or both a linker and lipid moiety.

### Lipid Moiety

In some embodiments, a peptide of the disclosure is conjugated to one or more lipid moieties. In some embodiments, the peptide is conjugated to two or more lipid moieties. For example, the peptide can be conjugated to at least 1, 2, 3, or 4 lipid moieties.

In some embodiments, the one or more lipid moieties are conjugated to the N-terminus of the peptide. In some embodiments, the one or more lipid moieties are conjugated to the C-terminus of the peptide.

In some embodiments, the one or more lipid moieties are conjugated to any one of the amino acid residues of Formula (I) having a suitable functional group for attachment to a lipid moiety. In some embodiments, the one or more lipid moieties are conjugated to any one of the amino acid residues of Formula (V) having a suitable functional group for attachment to a lipid moiety. In some embodiments, the one or more lipid moieties are conjugated to any one of the amino acid residues of Formula (I) through an amino acid side chain. In some embodiments, the one or more lipid moieties are conjugated to any one of the amino acid residues of Formula (V) through an amino acid side chain. In some embodiments, the one or more lipid moieties are conjugated to any one of the amino acid residues of Formula (I) having an amino acid side chain comprising an amino group. In some embodiments, the one or more lipid moieties are conjugated to any one of the amino acid residues of Formula (V) having an amino acid side chain comprising an amino group. In some embodiments, the one or more lipid moieties are conjugated to any one of the lysine residues of Formula (I). In some embodiments, the one or more lipid moieties are conjugated to any one of the lysine residues of Formula (V). In some embodiments, the one or more lipid moieties are conjugated to the N-terminus of the peptide and any one of the lysine residues of Formula (I). In some embodiments, the one or more lipid moieties are conjugated to the N-terminus of the peptide and any one of the lysine residues of Formula (V).

In some embodiments, the lipid moiety or a lipid moiety of the two or more lipid moieties comprises a C₂-C₂₆ fatty acyl group.

In some embodiments, the lipid moiety or a lipid moiety of the two or more lipid moieties comprises a saturated fatty acyl group. In some embodiments, the fatty acyl group comprises acetyl, propionyl, butyryl, pentanoyl, hexanoyl, heptanoyl, caprylyl, caprinoyl, lauroyl, myristoyl, palmitoyl, stearoyl, arachidonoyl, behenoyl, lignoceroyl, ceritoyl, octadecanedioyl, or eicosanedioyl. In some embodiments, the fatty acyl group comprises caprylyl, caprinoyl, lauroyl, myristoyl, palmitoyl, stearoyl, arachidonoyl, behenoyl, lignoceroyl, or ceritoyl. In some embodiments, the fatty acyl group comprises palmitoyl or stearoyl. In some embodiments, the fatty acyl group comprises acetyl.

In some embodiments, the lipid moiety or a lipid moiety of the two or more lipid moieties comprises -(L¹)ₚ-(L²)_{q}-(L³)ᵣ, wherein: each L¹ is independently —((CH₂)ᵥNR¹)_{w}—CO(CH₂)ₓO(PEG)_{y}(CH₂)_{z}NR¹—; PEG is —CH₂CH₂O—; v is 2-6; w is 0-1; x is 1-4; y is 1-4; z is 2-24; R¹ is H or —CH₃; each L² is independently a natural or unnatural amino acid or amino acid analog; each L³ is independently a substituted or unsubstituted C₂-C₂₆ fatty acyl group; p is 0-8; q is 0-6; and r is 1 or 2.

In some embodiments, each L¹ is independently —((CH₂)ᵥNR¹)_{w}—CO(CH₂)ₓO(PEG)_{y}(CH₂)_{z}NR¹—; each L² is independently a natural or unnatural amino acid or amino acid analog; each L³ is independently a C₂-C₂₆ fatty acyl group, wherein the C₂-C₂₆ fatty acyl group is unsubstituted or substituted with one or more of oxo, —OH, —C(═O)OH, tetrazolyl, benzoic acid, or naphthoic acid; p is 0-4; q is 0-2; and r is 1 or 2.

In some embodiments, each L¹ is independently —CO(CH₂)O(PEG)(CH₂)₂NH— or —((CH₂)ᵥNH)_{w}O(CH₂)O(PEG)(CH₂)₂NH—; each L² is independently glutamic acid, proline, aspartic acid, or 2,4-diaminobutyric acid; each L³ is independently lauroyl, myristoyl, palmitoyl, stearoyl, or arachidonoyl, wherein lauroyl, myristoyl, palmitoyl, stearoyl, and arachidonoyl are unsubstituted or substituted with one or more of oxo, —OH, —C(═O)OH, tetrazolyl, benzoic acid, or naphthoic acid; p is 0-4; q is 0-2; and r is 1 or 2.

In some embodiments, each L¹ is independently —CO(CH₂)O(PEG)(CH₂)₂NH— or —((CH₂)₃NH)—CO(CH₂)O(PEG)(CH₂)₂NH—; each L² is independently glutamic acid; each L³ is independently palmitoyl or stearoyl, wherein palmitoyl and stearoyl are unsubstituted or substituted with one or more of oxo, —OH, or —C(═O)OH; p is 0-4; q is 0-2; and r is 1 or 2.

In some embodiments, the lipid moiety or a lipid moiety of the two or more lipid moieties comprises -(L¹)ₚ-(L²)_{q}-(L³)ᵣ, wherein: each L¹ is OEG; OEG is —NH-PEG-PEG-CH₂C(═O)—; PEG is —CH₂CH₂O—; each L² is independently a natural or unnatural amino acid or amino acid analog; each L³ is independently a substituted or unsubstituted C₂-C₂₆ fatty acyl group; p is 0-8; q is 0-6; and r is 1 or 2.

In some embodiments, the lipid moiety or a lipid moiety of the two or more lipid moieties comprises -(L¹)ₚ-(L²)_{q}-(L³)ᵣ, wherein: each L¹ is OEG; OEG is —NH-PEG-PEG-CH₂C(═O)—; PEG is —CH₂CH₂O—; each L² is Glu or D-Glu; each L³ is independently a substituted or unsubstituted C₂-C₂₆ fatty acyl group; p is 0-8; q is 0-6; and r is 1 or 2.

In some embodiments, the lipid moiety or a lipid moiety of the two or more lipid moieties comprises -(L¹)ₚ-(L²)_{q}-(L³)ᵣ, wherein: each L¹ is OEG; OEG is —NH-PEG-PEG-CH₂C(═O)—; PEG is —CH₂CH₂O—; each L² is Glu or D-Glu; each L³ is independently palmitoyl, octadecanedioyl, or eicosanedioyl; p is 0-4; q is 0-2; and r is 1.

In some embodiments, -(L¹)ₚ-(L²)_{q}-(L³)ᵣ is

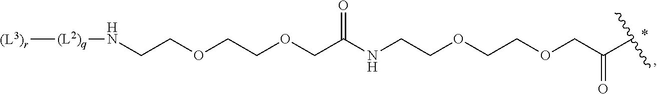

wherein * represents the attachment point to a nitrogen atom of the peptide.

In some embodiments, -(L¹)ₚ-(L²)_{q}-L³)ᵣ is

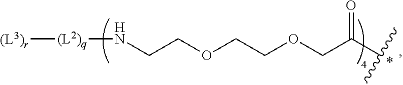

wherein * represents the attachment point to a nitrogen atom of the peptide.

In some embodiments, -(L¹)ₚ-(L²)_{q}-(L³)ᵣ is

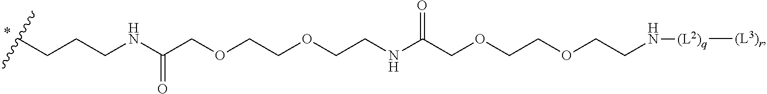

wherein * represents the attachment point to a nitrogen atom of the peptide.

In some embodiments, -(L¹)ₚ-(L²)_{q}-(L³)ᵣ is

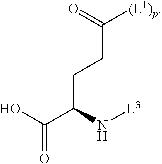

In some embodiments, -(L¹)ₚ-(L²)_{q}-(L³)ᵣ is

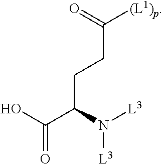

In some embodiments, L³ is

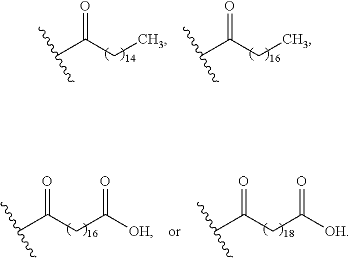

In some embodiments, L³ is

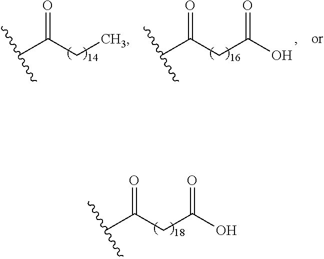

In some embodiments, -(L¹)ₚ-(L²)_{q}-L³)ᵣ is -(L¹)₂-(L²)-(L³).

In some embodiments, -(L¹)ₚ-(L²)_{q}-(L³)ᵣ is

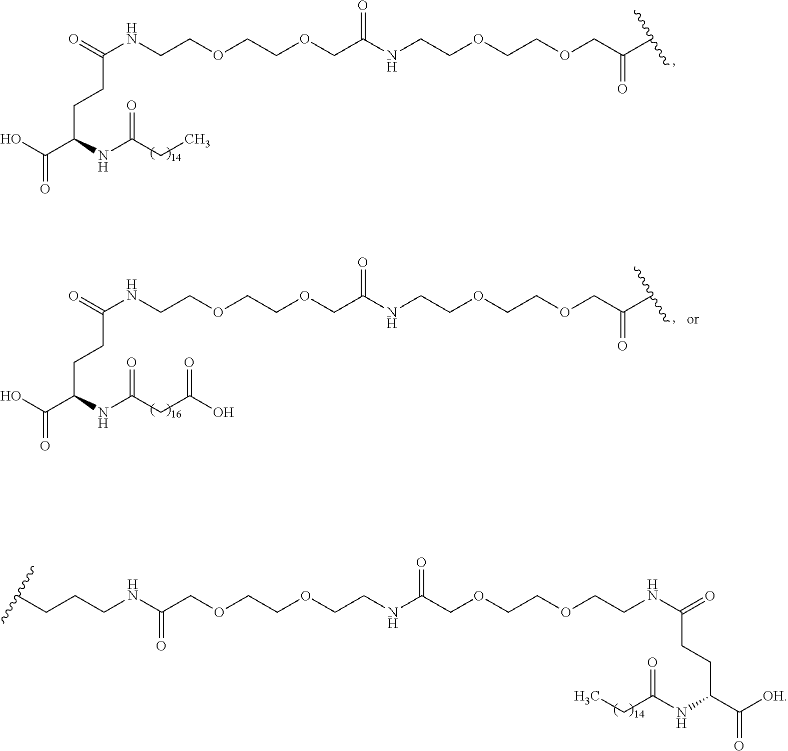

In some embodiments, -(L¹)ₚ-(L²)_{q}-(L³)ᵣ is

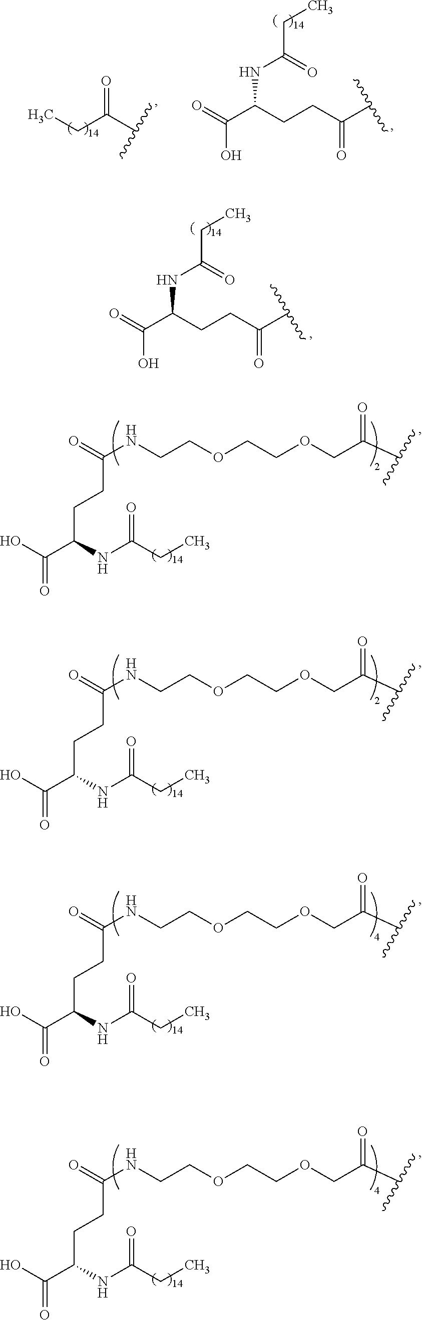

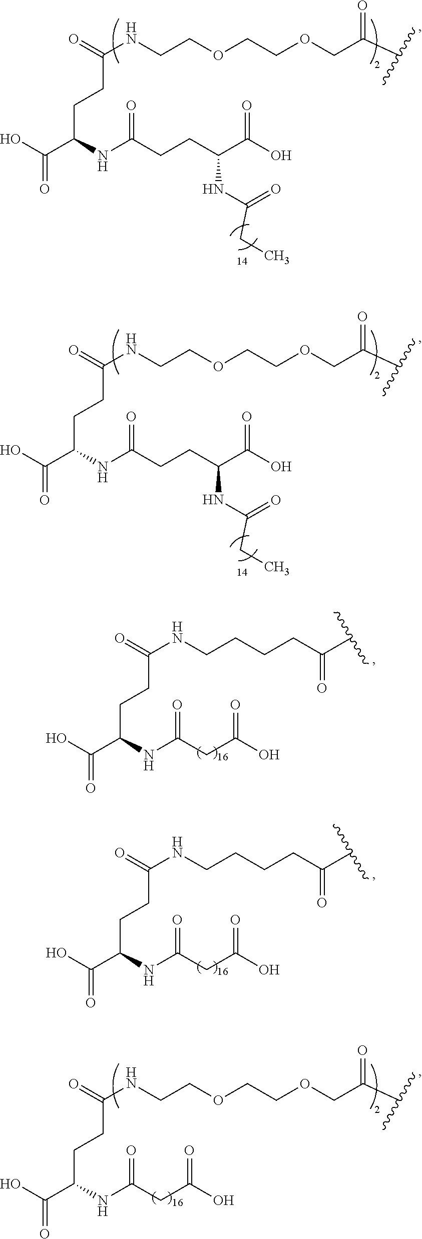

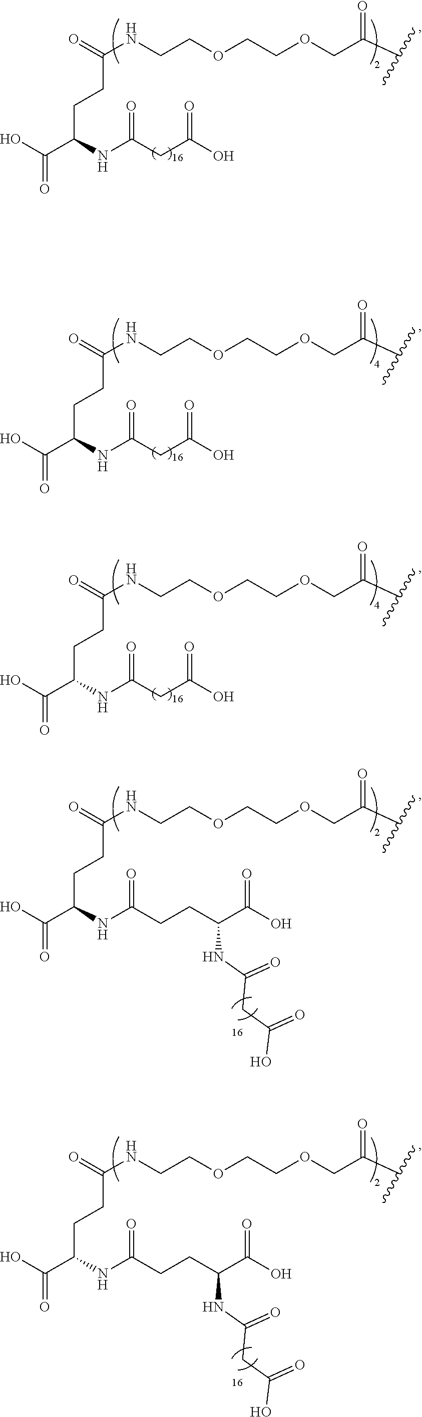

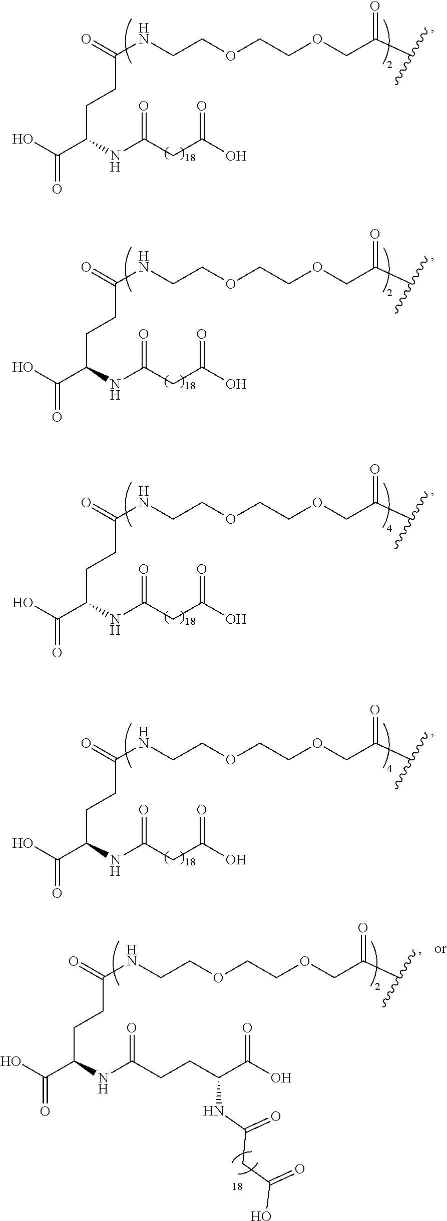

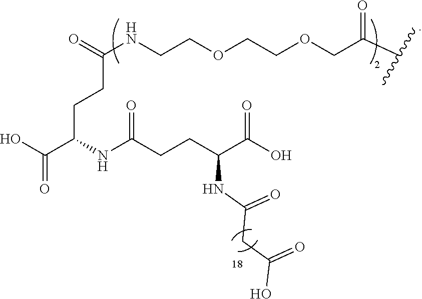

In some embodiments, -(L¹)ₚ-(L²)_{q}-(L³)ᵣ is

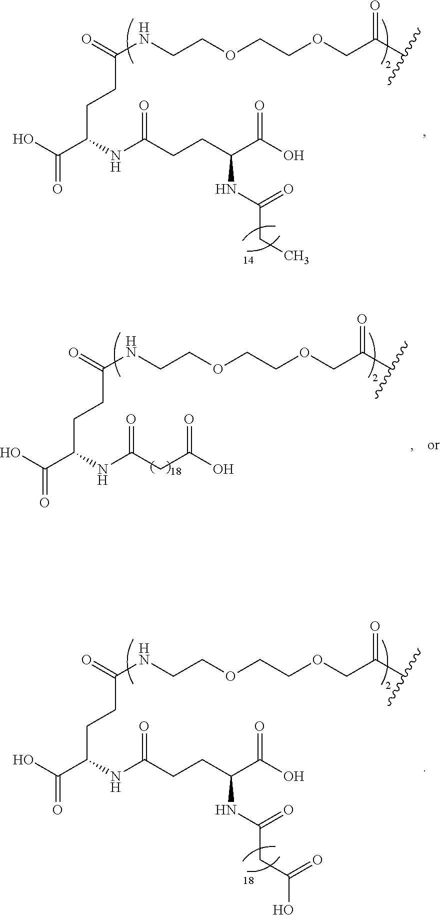

In some embodiments, -(L¹)ₚ-(L²)_{q}-(L³)ᵣ is

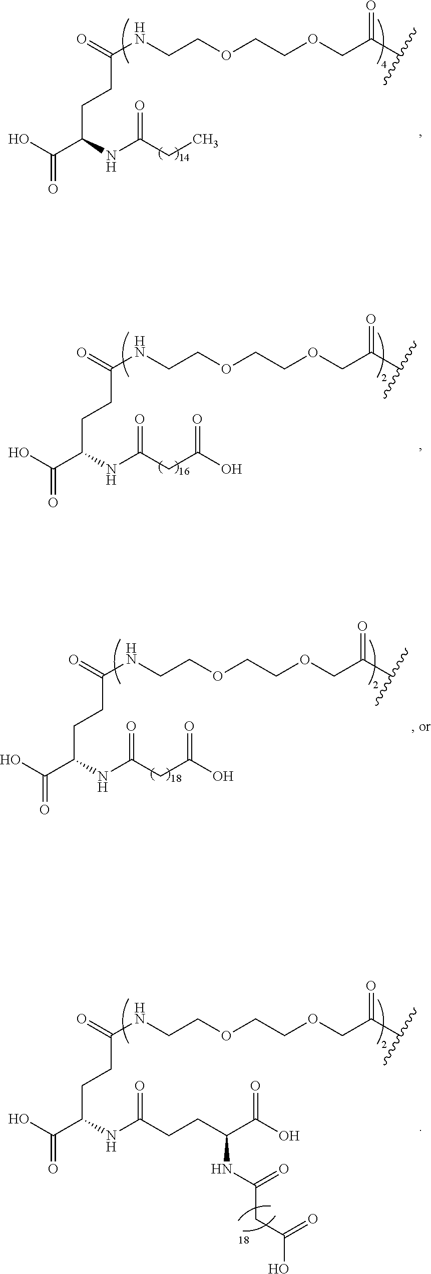

In some embodiments, the peptide is conjugated to 1 or 2 of -(L¹)ₚ-(L²)_{q}-(L³)ᵣ.

In some embodiments, the peptide is conjugated to 1 or 2 of

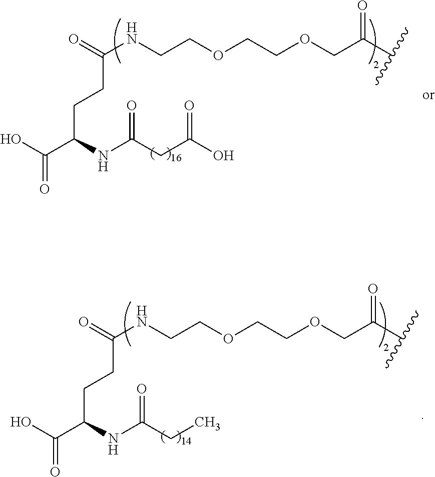

In some embodiments, the peptide is conjugated to 1 or 2 of

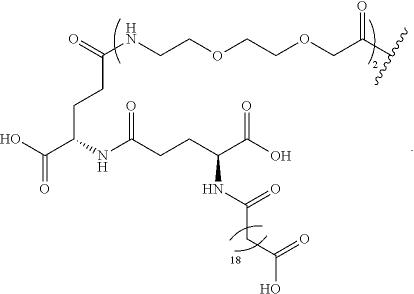

In some embodiments, the peptide is conjugated to -(L¹)ₚ-(L²)_{q}-(L³)ᵣ through the N-terminus of the peptide and a lysine residue of the peptide.

### Linker

In some embodiments, the lipid moiety or a lipid moiety of the two or more lipid moieties comprises a linker attached to a fatty acyl.

In some embodiments, the linker is flexible. In some embodiments, the linker is rigid. In some embodiments, the linker comprises a linear structure. In some embodiments, the linker comprises a non-linear structure. In some embodiments, the linker comprises a branched structure. In some embodiments, the linker comprises a cyclic structure.

In some embodiments, the linker comprises one or more linear structures, one or more non-linear structures, one or more branched structures, one or more cyclic structures, one or more flexible moieties, one or more rigid moieties, or combinations thereof.

In some embodiments, a linker comprises one or more amino acid residues. In some embodiments, the linker comprises 1 to 3, 1 to 5, 1 to 10, 5 to 10, or 5 to 20 amino acid residues. In some embodiments, one or more amino acids of the linker are unnatural amino acids.

In some embodiments, the linker comprises a peptide linkage. In some embodiments, the peptide linkage comprises L-amino acids and/or D-amino acids.

In some embodiments, the linker is 1 to 100 atoms, 1 to 50 atoms, 1 to 30 atoms, 1 to 20 atoms, 1 to 15 atoms, 1 to 10 atoms, or 1 to 5 atoms in length. In some embodiments, the linker has 1 to 20 atoms in length. In some embodiments, the linker is 1 to 10 atoms in length.

In some embodiments, the linker comprises flexible and/or rigid regions. Exemplary flexible linker regions include those comprising Gly and Ser residues (“GS” linker), glycine residues, alkylene chain, PEG chain, and the like. Exemplary rigid linker regions include those comprising α-helix-forming sequences, proline-rich sequences, and regions rich in double and/or triple bonds.

In some embodiments, the linker comprises one or more of substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene. In some embodiments, the linker comprises one or more of substituted or unsubstituted alkylene or substituted or unsubstituted heteroalkylene.

In some embodiments, the linker comprises a click chemistry residue. In some embodiments, the linker is attached to a peptide via click chemistry. For example, in some embodiments, a peptide comprises an azide group that reacts with an alkyne moiety of the linker. For example, in some embodiments, a peptide comprises an alkyne group that reacts with an azide of the linker. In some embodiments, the linker comprises an azide moiety, an alkyne moiety, or both. In some embodiments, the linker comprises a triazole moiety.

In some embodiments, the linker is hydrophilic. In some embodiments, the linker is hydrophobic.

In some embodiments, described herein is a peptide of Formula (VI):

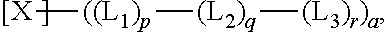

or a pharmaceutically acceptable salt, solvate, or isotope thereof, wherein:
X is a peptide comprising an amino acid sequence of Formula (V): X0-X1-X2-Ar-X4-X5-V-X7-X8-L-X10-X11-X12,
or a pharmaceutically acceptable salt, solvate, or isotope thereof, wherein: X4, X5, X7, and X8 are each independently a natural or non-natural amino acid or amino acid analog; Ar is an aromatic amino acid; X0, X1, X2, X10, X11, and X12 are each independently a natural or non-natural amino acid or amino acid analog, or absent;
each L¹ is independently —((CH₂)ᵥNR¹)_{w}—CO(CH₂)ₓO(PEG)_{y}(CH₂)_{z}NR¹—;
   PEG is —CH₂CH₂O—; v is 2-6; w is 0-1; x is 1-4; y is 1-4; z is 2-24; R¹ is H or —CH₃;
or each L¹ is OEG; OEG is —NH-PEG-PEG-CH₂C(═O)—; PEG is —CH₂CH₂O—;
each L² is independently a natural or unnatural amino acid or amino acid analog;
each L³ is independently a substituted or unsubstituted C₂-C₂₆ fatty acyl group;
a is 1, 2, 3, or 4; p is 0-8; q is 0-6; and r is 1 or 2.

In some embodiments, described herein is a peptide of Formula (IV):

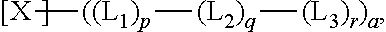

wherein:
X is a peptide comprising an amino acid sequence of Formula I:
X1-X2-Ar-X4-X5-V-X7-X8-L-X10-X11-X12, wherein:
X4, X5, X7, and X8 are each independently a natural or non-natural amino acid or amino acid analog;
Ar is an aromatic amino acid;
X1, X2, X10, X11, and X12 are each independently a natural or non-natural amino acid or amino acid analog, or absent;
each L¹ is independently —((CH₂)ᵥNR¹)_{w}—CO(CH₂)ₓO(PEG)_{y}(CH₂)_{z}NR¹—;
   PEG is —CH₂CH₂O—;
   v is 2-6;
   w is 0-1;
   x is 1-4;
   y is 1-4;
   z is 2-24;
   R¹ is H or —CH₃;
each L² is independently a natural or unnatural amino acid or amino acid analog;
each L³ is independently a substituted or unsubstituted C₂-C₂₆ fatty acyl group;
a is 1, 2, 3 or 4;
p is 0-8;
q is 0-6; and
r is 1 or 2.

In some embodiments, each (-(L¹)ₚ-(L²)_{q}-(L³)ᵣ) is independently attached to the peptide X at the N-terminus of the peptide, the C-terminus of the peptide, or any one of the amino acid residues of Formula (I) or (V) having a suitable functional group for attachment to a lipid moiety. In some embodiments, each (-(L¹)ₚ-(L²)_{q}-(L³)ᵣ) is independently attached to the peptide X at the N-terminus of the peptide, the C-terminus of the peptide, or any one of the amino acid residues of Formula (I) having a suitable functional group for attachment to a lipid moiety. In some embodiments, each (-(L¹)ₚ-(L²)_{q}-(L³)ᵣ) is independently attached to the peptide X at the N-terminus of the peptide, the C-terminus of the peptide, or any one of the amino acid residues of Formula (V) having a suitable functional group for attachment to a lipid moiety.

In some embodiments, a is 2. In some embodiments, each (-(L¹)ₚ-(L²)_{q}-(L³)ᵣ) is independently attached to the N-terminus of peptide X. In some embodiments, each (-(L¹)ₚ-(L²)_{q}-L³)ᵣ) is independently attached to any one of the amino acid residues of Formula (I) or (V) having a suitable functional group for attachment to a lipid moiety.

In some embodiments, a is 1. In some embodiments, (-(L¹)ₚ-(L²)_{q}-(L³)ᵣ) is attached to the N-terminus of peptide X. In some embodiments, (-(L¹)ₚ-(L²)_{q}-(L³)ᵣ) is attached to any one of the amino acid residues of Formula (I) or (V) having a suitable functional group for attachment to a lipid moiety.

In some embodiments, a is 2. In some embodiments, each (-(L¹)ₚ-(L²)_{q}-(L³)ᵣ) is independently attached to the N-terminus of peptide X. In some embodiments, each (-(L¹)ₚ-(L²)_{q}-L³)ᵣ) is independently attached to any one of the amino acid residues of Formula (I) having a suitable functional group for attachment to a lipid moiety. In some embodiments, each (-(L¹)ₚ-(L²)_{q}-(L³)ᵣ) is independently attached to any one of the amino acid residues of Formula (V) having a suitable functional group for attachment to a lipid moiety.

In some embodiments, a is 1.

In some embodiments, described herein is a peptide of Formula (IV), [X [symbol] ((L¹)ₚ-(L²)_{q}-(L³)ᵣ)ₐ or a pharmaceutically acceptable salt, solvate, or isotope thereof, wherein: X is a peptide comprising an amino acid sequence of Formula (II): X1-X2-Ar-X4-X5-V-X7-X8-L-X10-X11-X12, or a pharmaceutically acceptable salt, solvate, or isotope thereof, wherein:
X1 is Gly, Ala, N-acetylglycine, or D-alanine;
X2 is Asn, Glu, Gln, Arg, or Asp;
Ar is histidine or 3-(3-pyridyl)alanine (3PAl);
X4 is Trp, 7-methyltryptophan (7MeW), Phe, D-alanine, or Tyr;
X5 is Ala, α-aminoisobutyric acid (Aib), Gly, Leu, Val, Ile, D-alanine, or Nle;
X7 is Gly, D-alanine, Ala, Aib, Leu, Val, Ile, or Nle;
X8 is H or 3-(3-pyridyl)alanine (3PAl);
X10 is Met, Nle, Aib, Leu, Val, or Ile;
X11 is sarcosine (Sar), Gly, Pro, Ala, D-alanine, or absent; and
X12 is independently a natural or non-natural amino acid or amino acid analog, or absent.

In one aspect, described herein is a peptide of Formula (IV): [X [symbol] ((L₁)ₚ-(L₂)_{q}-(L₃)ᵣ)ₐ wherein:
X is a peptide comprising an amino acid sequence of Formula (I):
X1-X2-Ar-X4-X5-V-X7-X8-L-X10-X11-X12, or a pharmaceutically acceptable salt, solvate, or isotope thereof, wherein:
X4, X5, X7, and X8 are each independently a natural or non-natural amino acid or amino acid analog;
Ar is an aromatic amino acid;
X1, X2, X10, X11, and X12 are each independently a natural or non-natural amino acid or amino acid analog, or absent;
each L¹ is independently —((CH₂)ᵥNR¹)_{w}—CO(CH₂)ₓO(PEG)_{y}(CH₂)_{z}NR¹—; PEG is —CH₂CH₂O—; v is 2-6; w is 0-1; x is 1-4; y is 1-4; z is 2-24; R¹ is H or —CH₃;
or each L¹ is OEG;
   OEG is —NH-PEG-PEG-CH₂C(═O)—;
   PEG is —CH₂CH₂O—;
each L² is independently a natural or unnatural amino acid or amino acid analog;
each L³ is independently a substituted or unsubstituted C₂-C₂₆ fatty acyl group;
a is 1, 2, 3, or 4;
p is 0-8;
q is 0-6; and
r is 1 or 2.

In some embodiments, a peptide disclosed herein can comprise a capping group. In some embodiments, a peptide disclosed herein is conjugated to a lipid moiety at the N-terminus and is substituted with a capping group (e.g., amino group) at the C-terminus. In some embodiments, a peptide disclosed herein is conjugated to a lipid moiety at the C-terminus and is substituted with a capping group (e.g., acetyl group) at the N-terminus.

Table 1 provides structures of illustrative peptides of the disclosure. In some embodiments, a composition or method of the disclosure comprises a peptide of Table 1, or a pharmaceutically acceptable salt or solvate thereof. In some embodiments, a peptide of the disclosure comprises an amino acid sequence comprising at least 50%, 55%, 60%, 65%, 70%, 75%, 80%, 85%, 90%, 95%, 96%, 97%, 98%, 99% or 100% sequence identity to any one of the peptides in Table 1.

TABLE 1
Exemplary Peptides

| Pep- | |
|---|---|
| tide | Structure |
| 1 (SEQ ID NO. 1) | 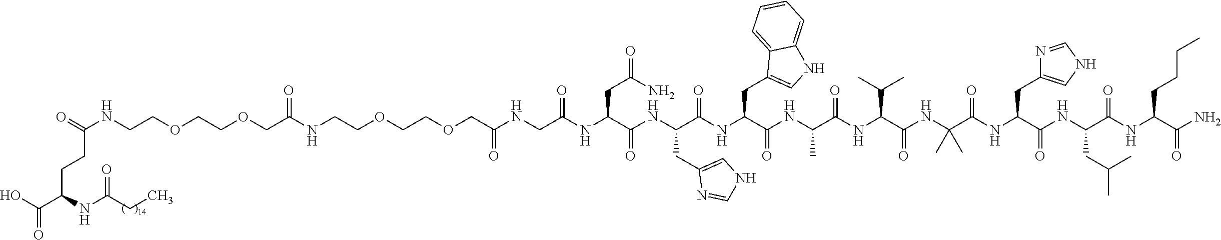 |
| 2 (SEQ ID NO. 2) | 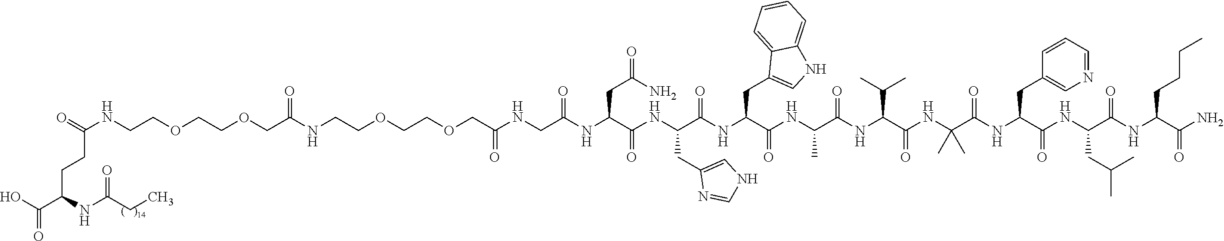 |
| 3 (SEQ ID NO. 3) | 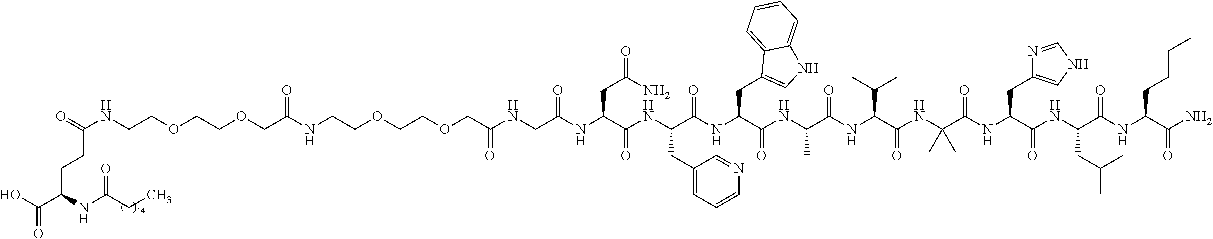 |
| 4 (SEQ ID NO. 4) | 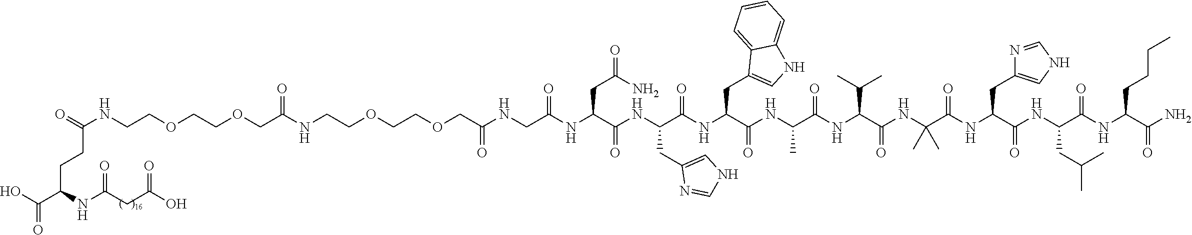 |
| 5 (SEQ ID NO. 5) | 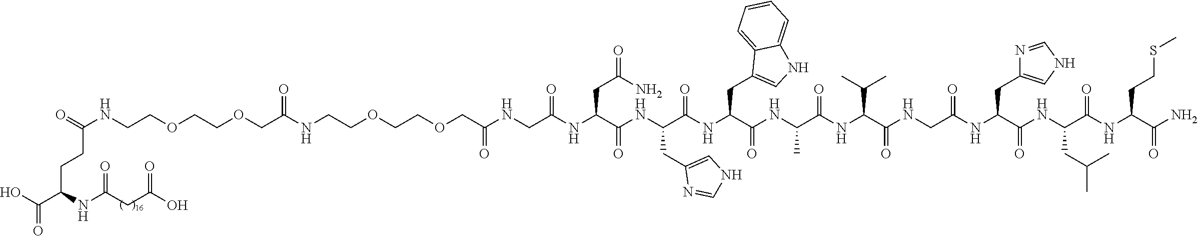 |
| 7 (SEQ ID NO. 7) | 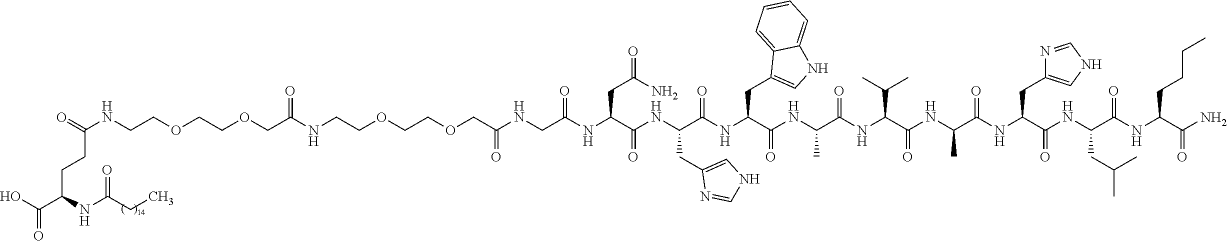 |
| 8 (SEQ ID NO. 8) | 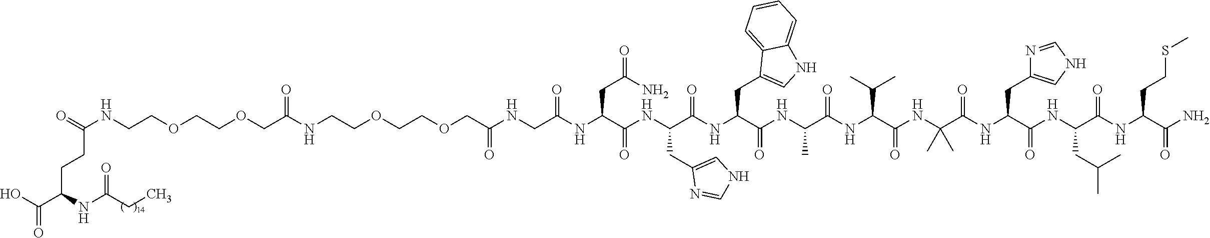 |
| 9 (SEQ ID NO. 9) | 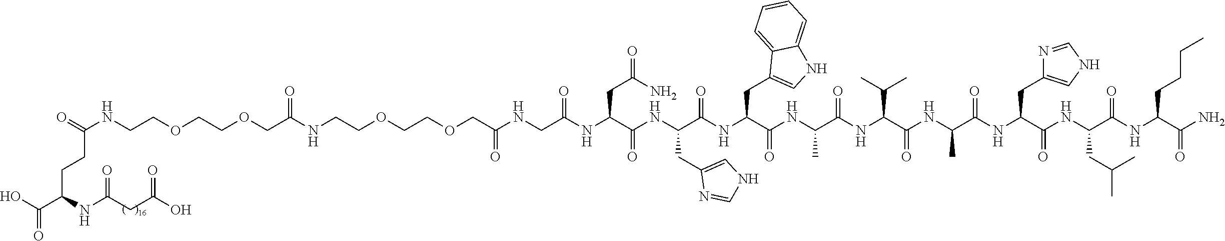 |
| 10 (SEQ ID NO. 10) | 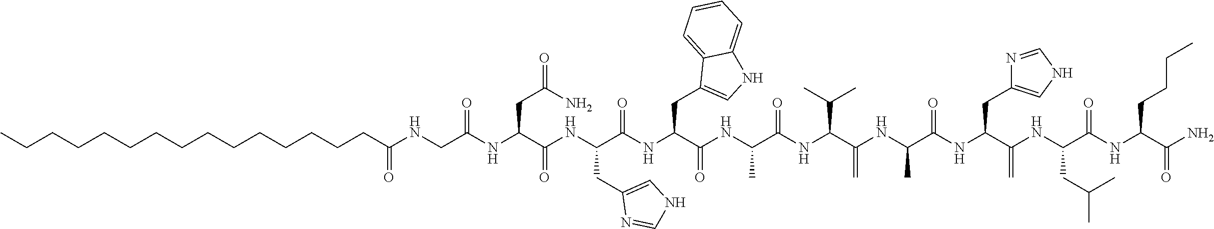 [SMILES: C=C(N[C@@H](CC(C)C)C(=O)N[C@@H](CCCC)C(N)=O)[C@H](Cc1c[nH]cn1)NC(=O)[C@@H](C)NC(=C)[C@@H](NC(=O)[C@H](C)NC(=O)[C@H](Cc1c[nH]c2ccccc12)NC(=O)[C@H](Cc1c[nH]cn1)NC(=O)[C@H](CC(N)=O)NC(=O)CNC(=O)CCCCCCCCCCCCCCC)C(C)C] |
| 11 (SEQ ID NO. 11) | 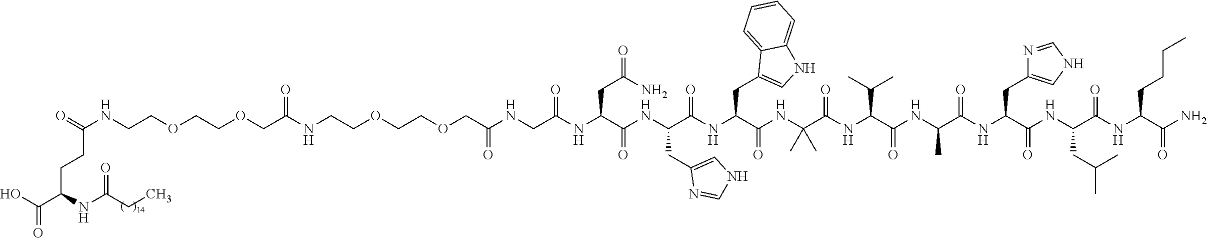 |
| 12 (SEQ ID NO. 12) | 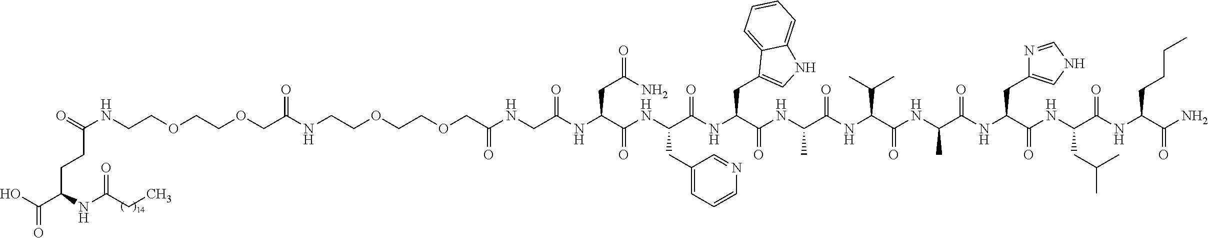 |
| 13 (SEQ ID NO. 130 | 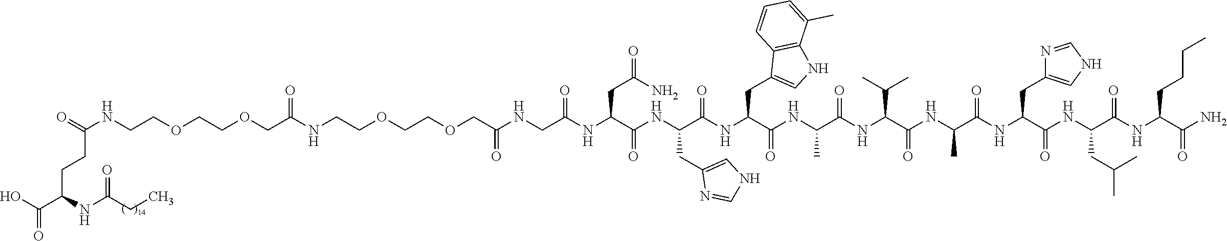 |
| 14 (SEQ ID NO. 14) | 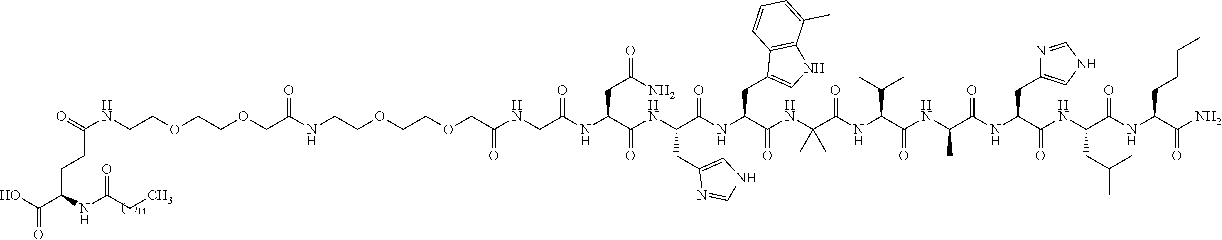 |
| 15 (SEQ ID NO. 15) | 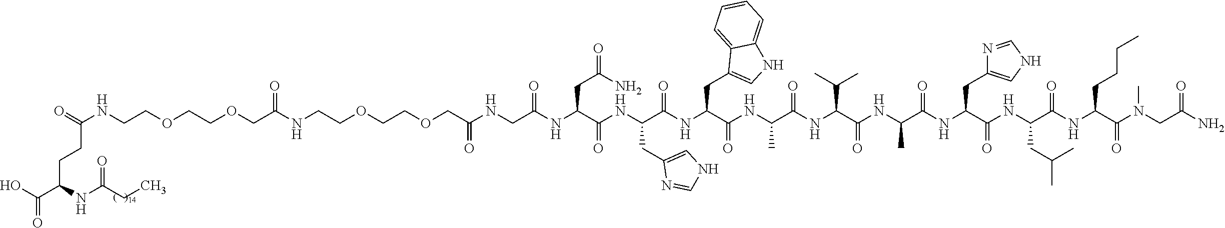 |
| 16 (SEQ ID NO. 16) | 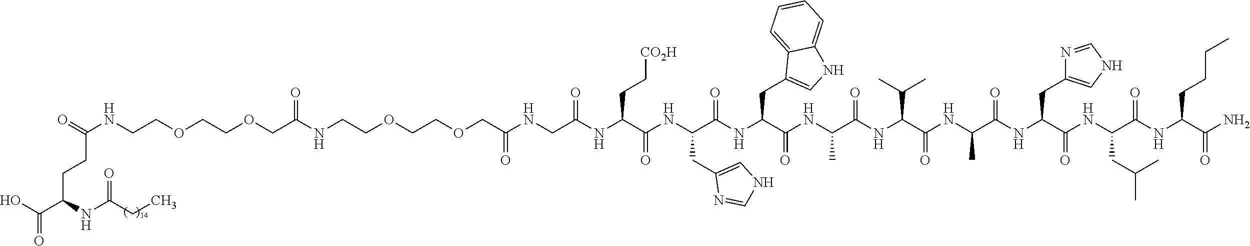 |
| 17 (SEQ ID NO. 17) | 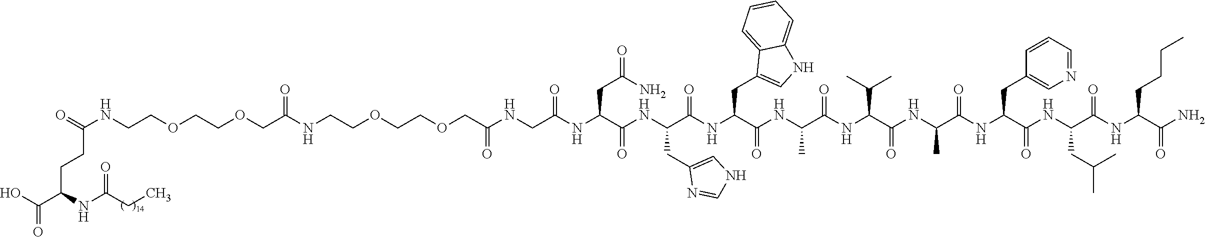 |
| 18 (SEQ ID NO. 18) | 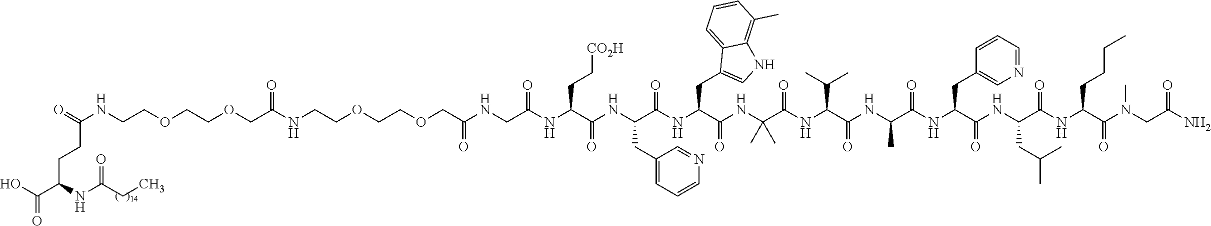 |
| 19 (SEQ ID NO. 19) | 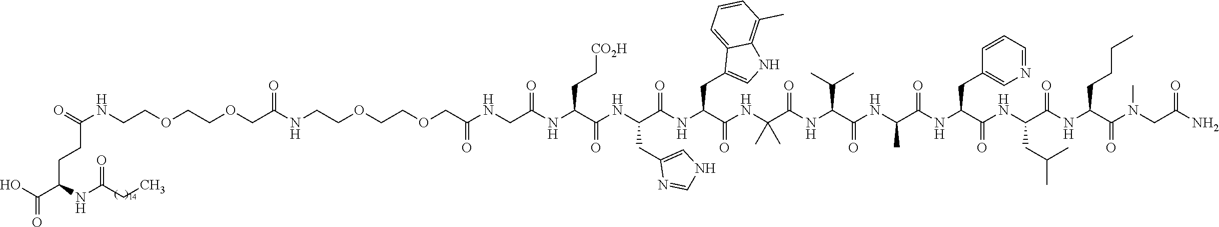 |
| 20 (SEQ ID NO. 20) | 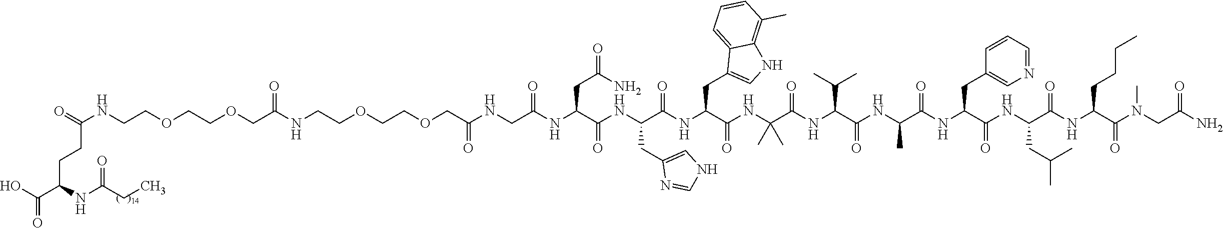 |
| 21 (SEQ ID NO. 21) | 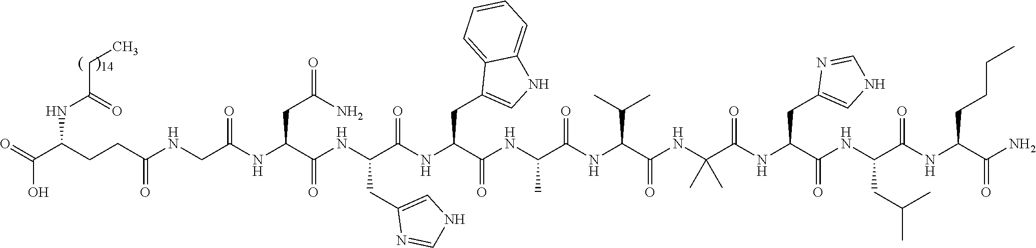 |
| 22 (SEQ ID NO. 22) | 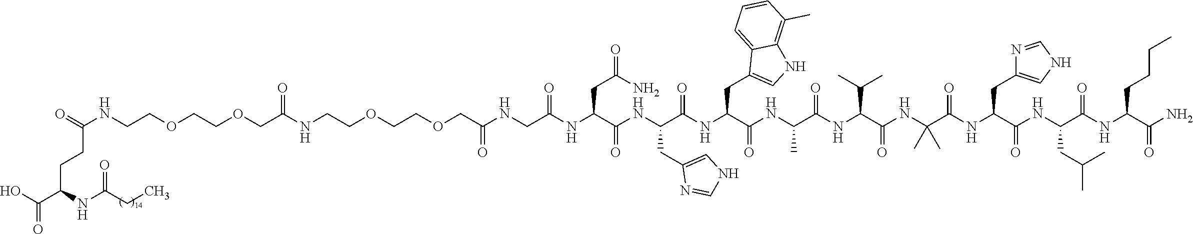 |
| 23 (SEQ ID NO. 23) | 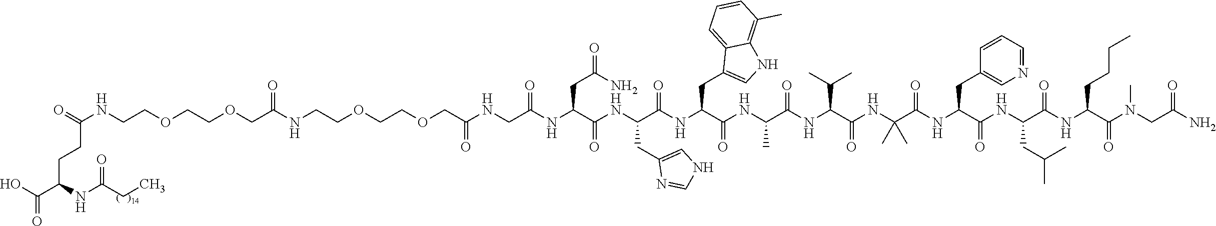 |
| 24 (SEQ ID NO. 24) | 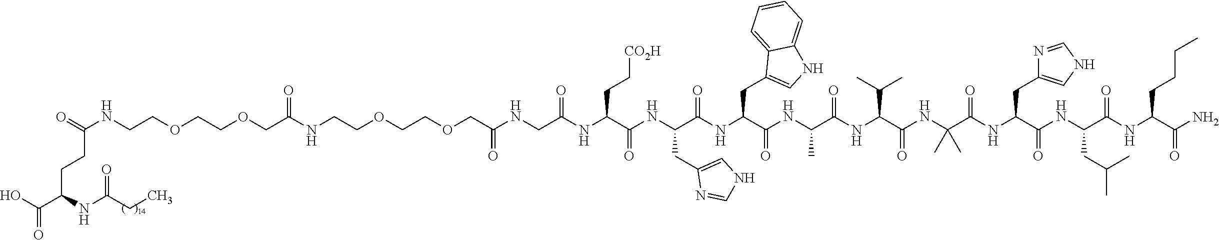 |
| 25 (SEQ ID NO. 25) | 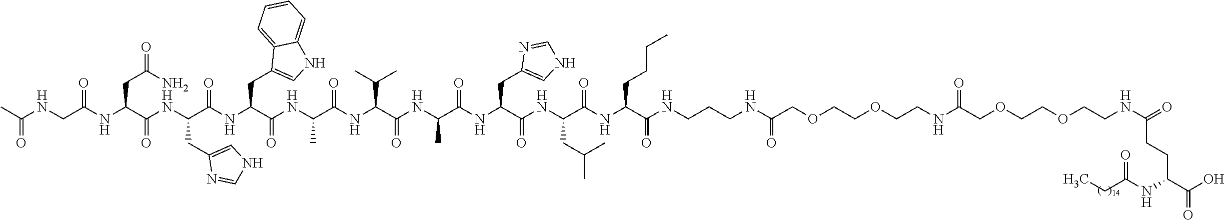 |
| 26 (SEQ ID NO. 26) | 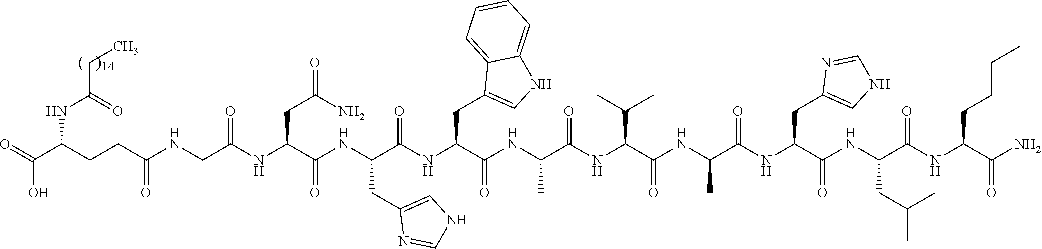 |
| 247 (SEQ ID NO. 247) | 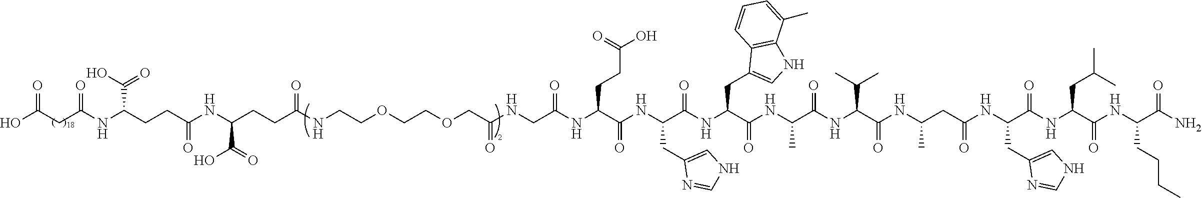 |
| 248 (SEQ ID NO. 248) | 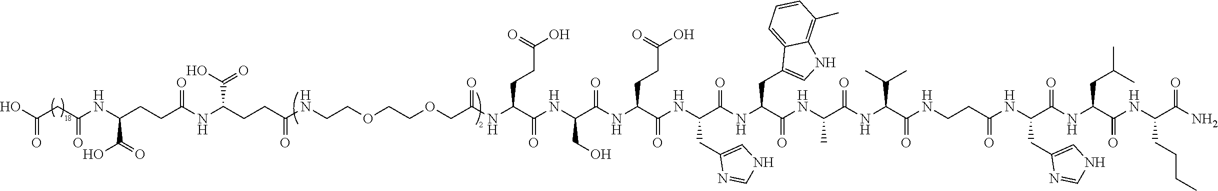 |

In some embodiments, a composition or method of the disclosure comprises a peptide selected from any one of:

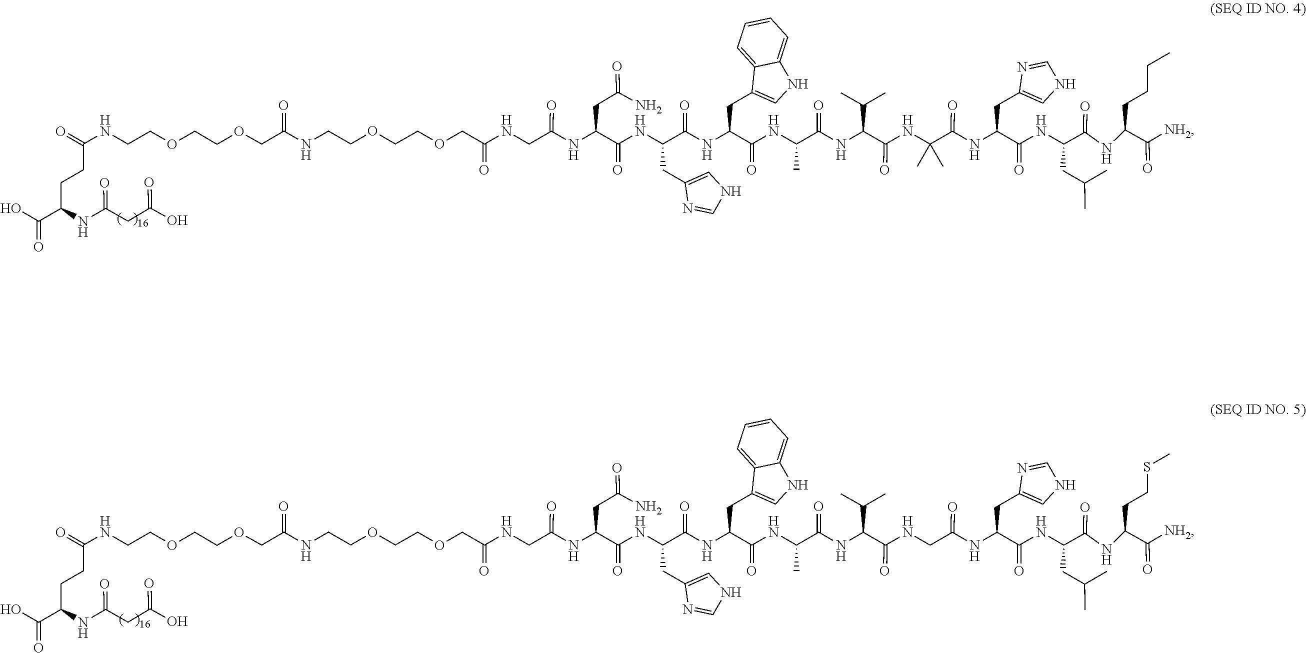

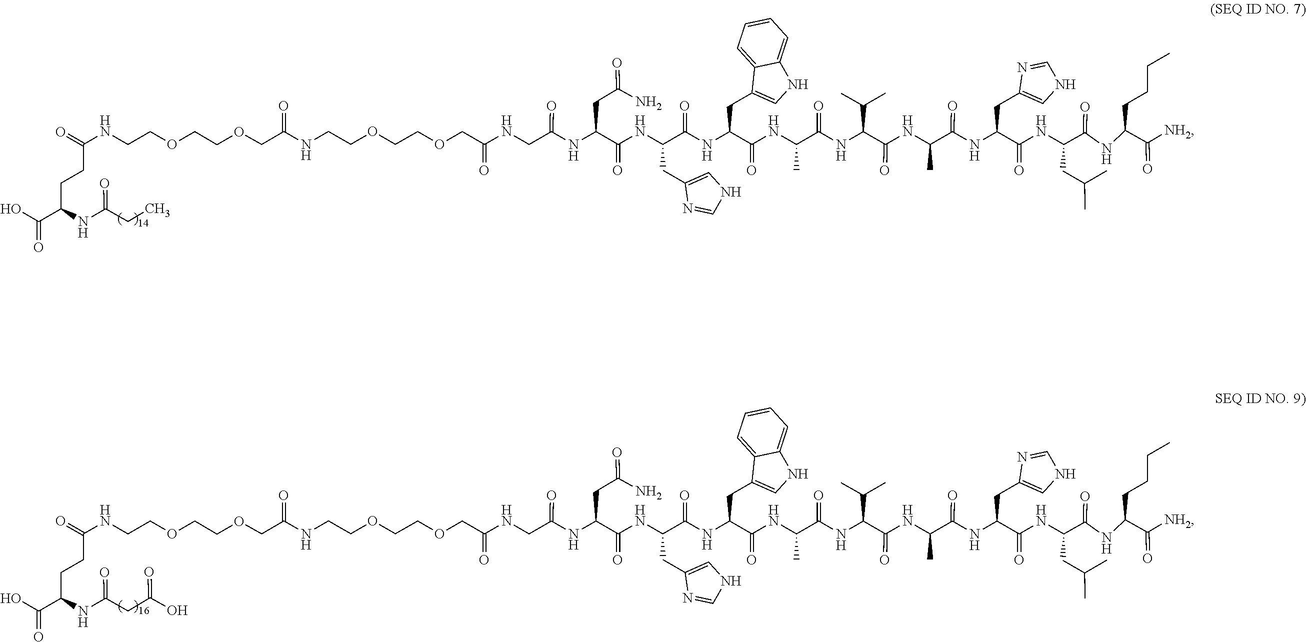

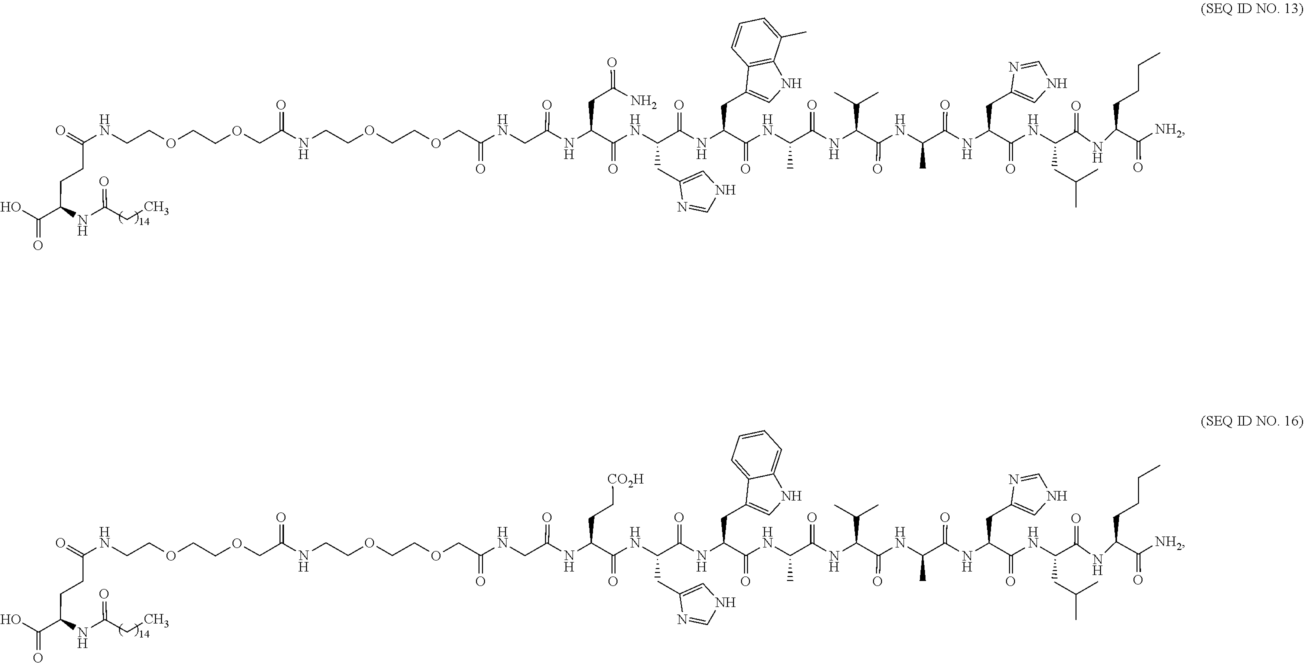

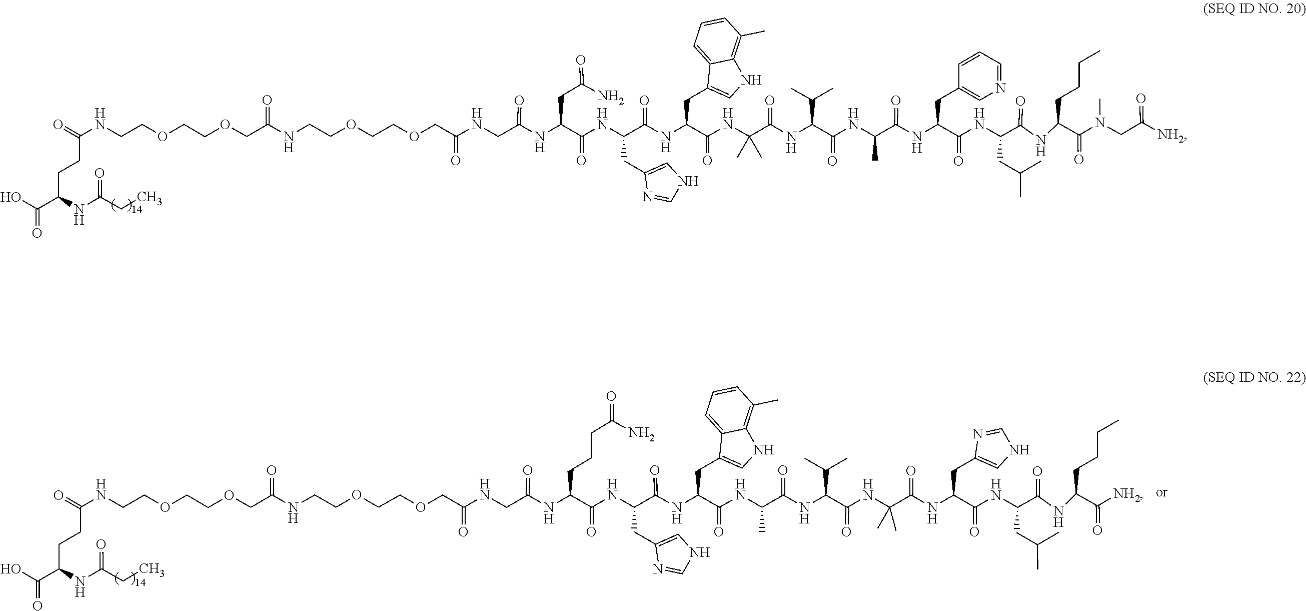

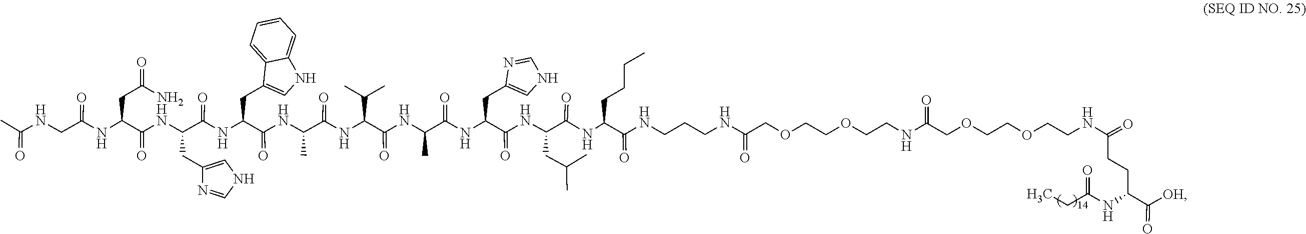

or a pharmaceutically acceptable salt or solvate thereof.

In some embodiments, a composition or method of the disclosure comprises a peptide selected from any one of:

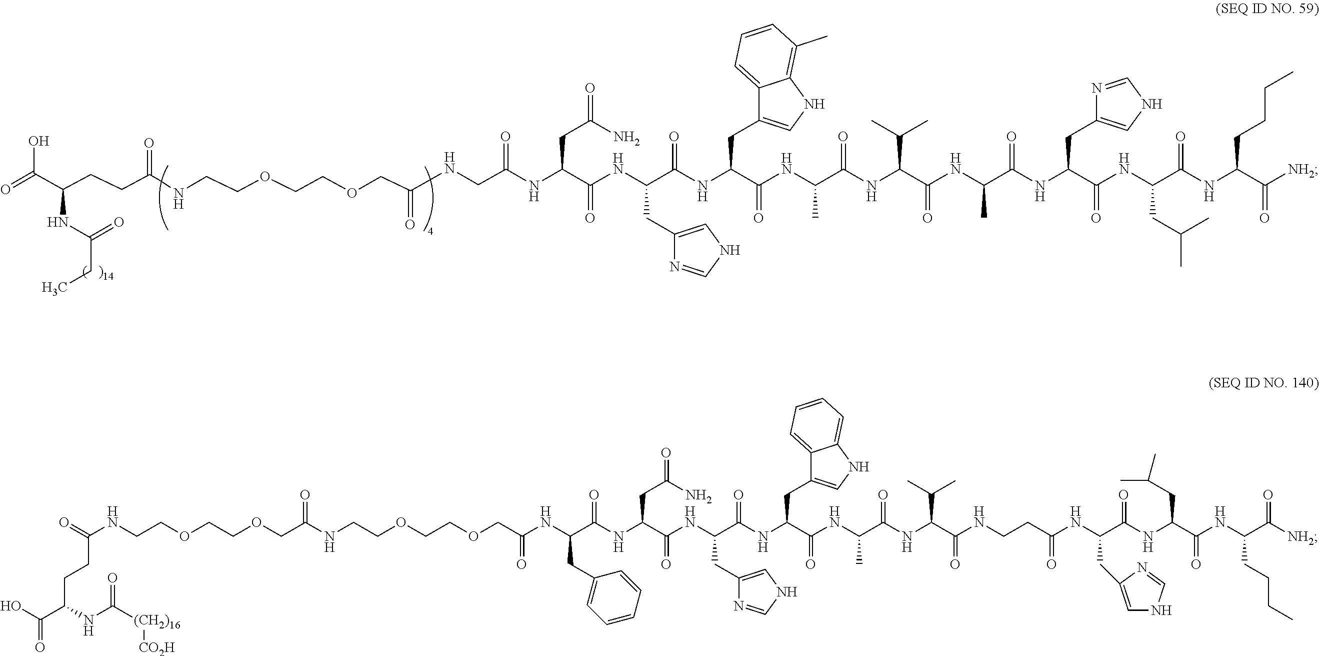

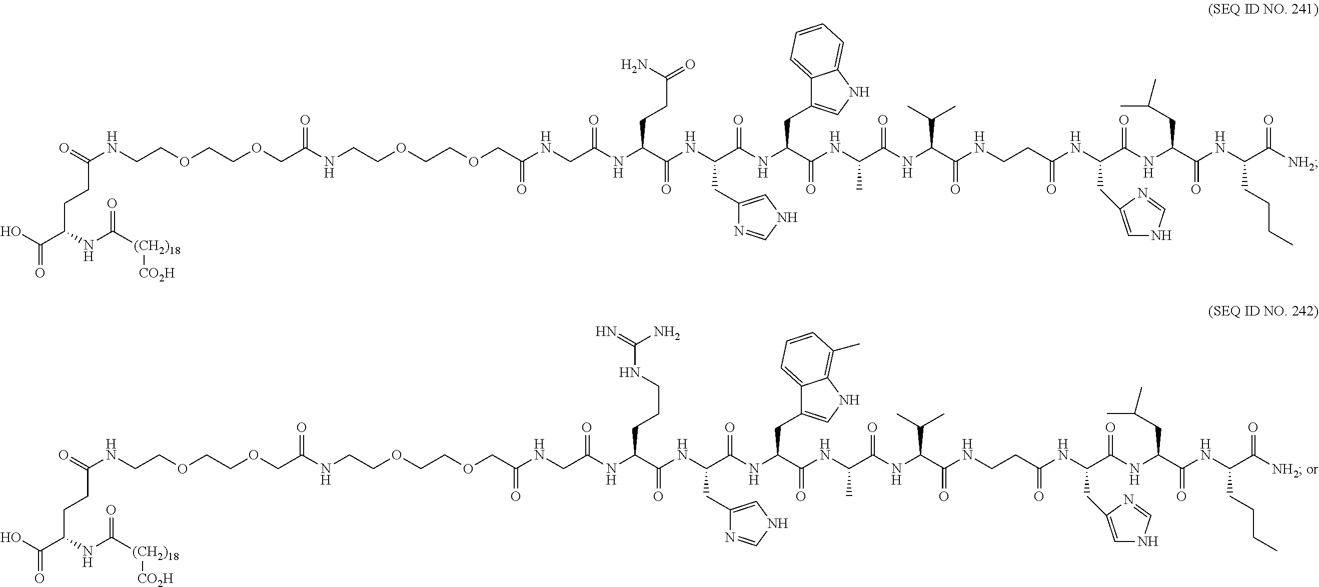

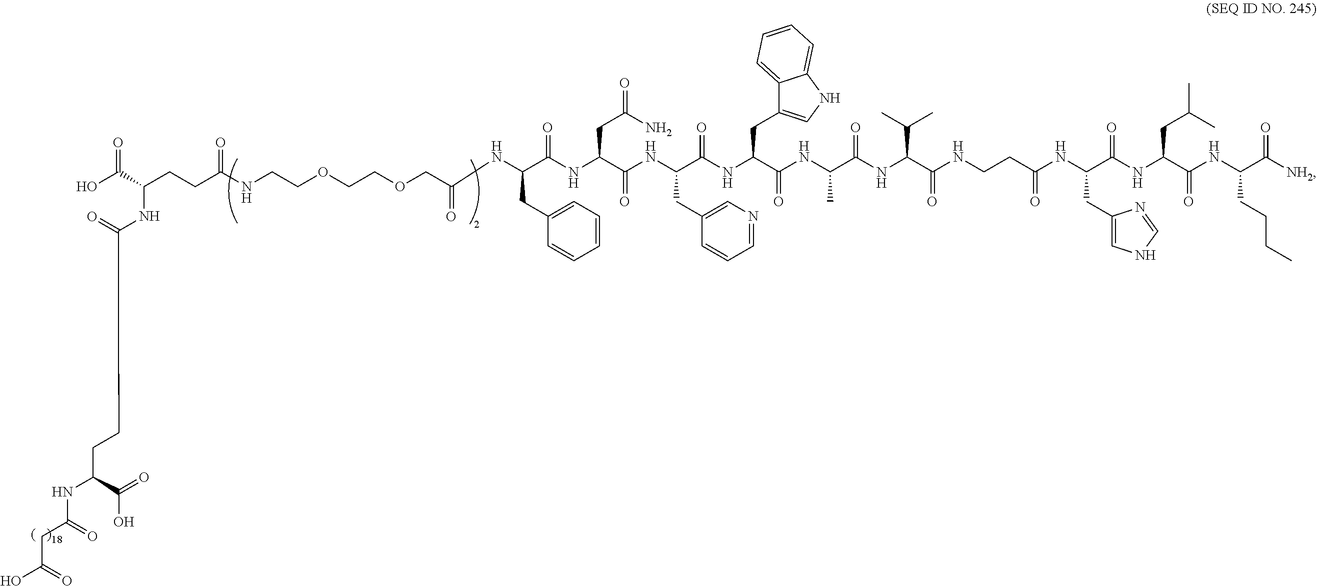

or a pharmaceutically acceptable salt or solvate thereof.

Table 1A provides amino acid sequences of illustrative lipid moiety conjugated peptides of the disclosure. In some embodiments, a composition or method of the disclosure comprises a peptide comprising an amino acid sequence having at least 50%, 55%, 60%, 65%, 70%, 75%, 80%, 85%, 90%, 95%, 96%, 97%, 98%, 99% or 100% sequence identity to any one of the sequences of Table 1A, or a pharmaceutically acceptable salt, solvate, or isotope thereof.

TABLE 1A
Amino Acid Sequences for Illustrative Lipid Moiety Conjugated Peptides

| | SEQ ID | |
|---|---|---|
| Peptide | NO. | Sequence and Lipid Moiety |
| 1 | 1 | Palmitoyl-D-γGlu-OEG-OEG-Gly-Asn-His-Trp-Ala-Val-Aib-His-Leu-Nle- |
| | | NH₂ |
| 2 | 2 | Palmitoyl-D-γGlu-OEG-OEG-Gly-Asn-His-Trp-Ala-Val-Aib-3Pal-Leu- |
| | | Nle-NH₂ |
| 3 | 3 | Palmitoyl-D-γGlu-OEG-OEG-Gly-Asn-3Pal-Trp-Ala-Val-Aib-His-Leu- |
| | | Nle-NH₂ |
| 4 | 4 | Octadecanedioyl-D-γGlu-OEG-OEG-Gly-Asn-His-Trp-Ala-Val-Aib-His- |
| | | Leu-Nle-NH₂ |
| 5 | 5 | Octadecanedioyl-D-γGlu-OEG-OEG-Gly-Asn-His-Trp-Ala-Val-Gly-His- |
| | | Leu-Met-NH₂ |
| 7 | 7 | Palmitoyl-D-γGlu-OEG-OEG-Gly-Asn-His-Trp-Ala-Val-D-Ala-His-Leu- |
| | | Nle-NH₂ |
| 8 | 8 | Palmitoyl-D-γGlu-OEG-OEG-Gly-Asn-His-Trp-Ala-Val-Gly-His-Leu- |
| | | Met-NH₂ |
| 9 | 9 | Octadecanedioyl-D-γGlu-OEG-OEG-Gly-Asn-His-Trp-Ala-Val-D-Ala-His- |
| | | Leu-Nle-NH₂ |
| 10 | 10 | Palmitoyl-Gly-Asn-His-Trp-Ala-Val-D-Ala-His-Leu-Nle-NH₂ |
| 11 | 11 | Palmitoyl-D-γGlu-OEG-OEG-Gly-Asn-His-Trp-Aib-Val-D-Ala-His-Leu- |
| | | Nle-NH₂ |
| 12 | 12 | Palmitoyl-D-γGlu-OEG-OEG-Gly-Asn-3Pal-Trp-Ala-Val-D-Ala-His-Leu- |
| | | Nle-NH₂ |
| 13 | 13 | Palmitoyl-D-γGlu-OEG-OEG-Gly-Asn-His-7MethylTrp-Ala-Val-D-Ala- |
| | | His-Leu-Nle-NH₂ |
| 14 | 14 | Palmitoyl-D-γGlu-OEG-OEG-Gly-Asn-His-7MethylTrp-Aib-Val-D-Ala- |
| | | His-Leu-Nle-NH₂ |
| 15 | 15 | Palmitoyl-D-γGlu-OEG-OEG-Gly-Asn-His-Trp-Ala-Val-D-Ala-His-Leu- |
| | | Nle-Sar-NH₂ |
| 16 | 16 | Palmitoyl-D-γGlu-OEG-OEG-Gly-Glu-His-Trp-Ala-Val-D-Ala-His-Leu- |
| | | Nle-NH₂ |
| 17 | 17 | Palmitoyl-D-γGlu-OEG-OEG-Gly-Asn-His-Trp-Ala-Val-D-Ala-3Pal-Leu- |
| | | Nle-NH₂ |
| 18 | 18 | Palmitoyl-D-γGlu-OEG-OEG-Gly-Glu-3Pal-7MethylTrp-Aib-Val-D-Ala- |
| | | 3Pal-Leu-Nle-Sar-NH₂ |
| 19 | 19 | Palmitoyl-D-γGlu-OEG-OEG-Gly-Glu-His-7MethylTrp-Aib-Val-D-Ala- |
| | | 3Pal-Leu-Nle-Sar-NH₂ |
| 20 | 20 | Palmitoyl-D-γGlu-OEG-OEG-Gly-Asn-His-7MethylTrp-Aib-Val-D-Ala- |
| | | 3Pal-Leu-Nle-Sar-NH₂ |
| 21 | 21 | Palmitoyl-D-γGlu-Gly-Asn-His-Trp-Ala-Val-Aib-His-Leu-Nle-NH₂ |
| 22 | 22 | Palmitoyl-D-γGlu-OEG-OEG-Gly-Asn-His-7MethylTrp-Ala-Val-Aib-His- |
| | | Leu-Nle-NH₂ |
| 23 | 23 | Palmitoyl-D-γGlu-OEG-OEG-Gly-Asn-His-7MethylTrp-Ala-Val-Aib-3Pal- |
| | | Leu-Nle-Sar-NH₂ |
| 24 | 24 | Palmitoyl-D-γGlu-OEG-OEG-Gly-Glu-His-Trp-Ala-Val-Aib-His-Leu-Nle- |
| | | NH₂ |
| 25 | 25 | Ac-Gly-Asn-His-Trp-Ala-Val-D-Ala-His-Leu-Nle-H₂N(CH₂)₃NH₂-OEG- |
| | | OEG-(α-Palmitoyl-D-γGlu) |
| 26 | 26 | Palmitoyl-D-γGlu-Gly-Asn-His-Trp-Ala-Val-D-Ala-His-Leu-Nle-NH₂ |
| 46 | 46 | Octadecanedioyl-D-Glu-OEG-OEG-Gly-Asn-His-7MethylTrp-Ala-Val-D- |
| | | Ala-His-Leu-Nle-NH₂ |
| 47 | 47 | Octadecanedioyl-D-γGlu-OEG-OEG-Gly-Asn-His-Trp-Ala-Val-D-Ala-His- |
| | | Leu-Nle-Sar-NH₂ |
| 48 | 48 | Palmitoyl-D-γGlu-OEG-OEG-Gly-Asn-His-Trp-Ala-Val-D-Ala-His-Leu- |
| | | Nle-Sar-NH₂ |
| 49 | 49 | Palmitoyl-D-γGlu-OEG-OEG-Gly-Asn-His-Trp-Ala-Val-D-Ala-His-Leu- |
| | | Lys(Me)₃-NH₂ |
| 50 | 50 | Ac-Gly-Asn-His-Trp-Ala-Val-D-Ala-His-Leu-EK-OEG-OEG-(α- |
| | | Palmitoyl-D-γGlu)-NH₂ |
| 51 | 51 | Palmitoyl-D-γGlu-OEG-OEG-Gly-Asn-2Pal-Trp-Ala-Val-DAla-His-Leu- |
| | | Nle-NH₂ |
| 52 | 52 | Palmitoyl-D-γGlu-OEG-OEG-Gly-Asn-4Pal-Trp-Ala-Val-D-Ala-His-Leu- |
| | | Nle-NH₂ |
| 53 | 53 | Octadecanedioyl-D-γGlu-OEG-OEG-OEG-OEG-Gly-Asn-His-Trp-Ala- |
| | | Val-D-Ala-His-Leu-Nle-NH₂ |
| 54 | 54 | Palmitoyl-D-γGlu-OEG-OEG-OEG-OEG-Gly-Asn-His-Trp-Ala-Val-D- |
| | | Ala-His-Leu-Nle-NH₂ |
| 55 | 55 | Octadecanedioyl-D-γGlu-OEG-OEG-Gly-Lys(OEG-OEG- |
| | | Octadecanedioyl-D-Glu--His-Trp-Ala-Val-D-Ala-His-Leu-Nle-NH₂ |
| 56 | 56 | Palmitoyl-D-γGlu-OEG-OEG-Gly-Asn-His-7FTrp-Ala-Val-D-Ala-His- |
| | | Leu-Nle-NH₂ |
| 57 | 57 | Palmitoyl-D-γGlu-OEG-OEG-Gly-Lys(OEG-OEG-α-Palmitoyl-D-γGlu)- |
| | | His-Trp-Ala-Val-D-Ala-His-Leu-Nle-NH₂ |
| 58 | 58 | Palmitoyl-D-γGlu-OEG-OEG-OEG-OEG-Gly-Asn-3Pal-7MethylTrp-Ala- |
| | | Val-D-Ala-His-Leu-Nle-NH₂ |
| 59 | 59 | Palmitoyl-D-γGlu-OEG-OEG-OEG-OEG-Gly-Asn-His-7MethylTrp-Ala- |
| | | Val-D-Ala-His-Leu-Nle-NH₂ |
| 60 | 60 | Octadecanedioyl-D-γGlu-OEG-OEG-Gly-Lys-His-Trp-Ala-Val-D-Ala-His- |
| | | Leu-Nle-NH₂ |
| 61 | 61 | Octadecanedioyl-D-γGlu-OEG-OEG-Gly-Arg-His-Trp-Ala-Val-D-Ala- |
| | | His-Leu-Nle-NH₂ |
| 62 | 62 | Octadecanedioyl-D-γGlu-OEG-OEG-Gly-Asn-His-Trp-Ala-Val-D-Ala- |
| | | Phe-Leu-Nle-NH₂ |
| 63 | 63 | Palmitoyl-D-γGlu-OEG-OEG-Gly-Asn-His-6MethylTrp-Ala-Val-D-Ala- |
| | | His-Leu-Nle-NH₂ |
| 64 | 64 | Octadecanedioyl-D-γGlu-OEG-OEG-Gly-Asn-3Pal-Trp-Ala-Val-D-Ala- |
| | | His-Leu-Nle-NH₂ |
| 65 | 65 | Octadecanedioyl-D-γGlu-OEG-OEG-Gly-Lys(Ac)-His-Trp-Ala-Val-D-Ala- |
| | | His-Leu-Nle-NH₂ |
| 66 | 66 | Octadecanedioyl-D-γGlu-OEG-OEG-Gly-Asn-His-Trp-Ala-Val-D-Ala- |
| | | 3Pal-Leu-Nle-NH₂ |
| 67 | 67 | Octadecanedioyl-D-γGlu-OEG-OEG-Gly-Glu-His-Trp-Ala-Val-D-Ala- |
| | | 3Pal-Leu-Nle-NH₂ |
| 68 | 68 | Octadecanedioyl-D-γGlu-OEG-OEG-Gly-Asn-His-His-Ala-Val-D-Ala-His- |
| | | Leu-Nle-NH₂ |
| 69 | 69 | Octadecanedioyl-D-γGlu-OEG-OEG-Gly-Asn-His-Trp-Ala-Val-D-Ala-His- |
| | | Leu-Arg-NH₂ |
| 70 | 70 | Octadecanedioyl-D-γGlu-OEG-OEG-Gly-Glu-His-Trp-Aib-Val-D-Ala-His- |
| | | Leu-Nle-NH₂ |
| 71 | 71 | Octadecanedioyl-D-γGlu-OEG-OEG-Lys(Ac)-His-Trp-Ala-Val-D-Ala-His- |
| | | Leu-Nle-NH₂ |
| 72 | 72 | Octadecanedioyl-D-γGlu-OEG-OEG-Gly-Asn-3Pal-Trp-Ala-Val-D-Ala- |
| | | His-Leu-Nle-Sar-NH₂ |
| 73 | 73 | Octadecanedioyl-D-γGlu-OEG-OEG-Gly-Asn-3Pal-7MethylTrp-Ala-Val- |
| | | D-Ala-His-Leu-Nle-Sar-NH₂ |
| 74 | 74 | Octadecanedioyl-D-γGlu-OEG-OEG-Aib-Asn-His-Trp-Ala-Val-D-Ala-His- |
| | | Leu-Nle-NH₂ |
| 75 | 75 | Palmitoyl-D-γGlu-OEG-OEG-Gly-Asn-His-Trp-Ala-Val-D-Ala-His-Leu- |
| | | Nle-Aib-NH₂ |
| 76 | 76 | Octadecanedioyl-D-γGlu-OEG-OEG-Gly-Asn-His-7MethylTrp-Ala-Val-D- |
| | | Ala-His-Leu-Nle-Aib-NH₂ |
| 77 | 77 | Octadecanedioyl-D-γGlu-OEG-OEG-Gly-Lys(Ac)-His-Trp-Ala-Val-D-Ala- |
| | | His-Leu-Nle-Sar-NH₂ |
| 78 | 78 | Octadecanedioyl-D-γGlu-OEG-OEG-OEG-OEG-Gly-Asn-His-7Methy Trp- |
| | | Ala-Val-D-Ala-His-Leu-NH₂ |
| 79 | 79 | Octadecanedioyl-D-γGlu-OEG-OEG-Gly-Asn-His-6MethylTrp-Ala-Val-D- |
| | | Ala-His-Leu-Nle-NH₂ |
| 80 | 80 | Octadecanedioyl-D-γGlu-Ava-Gly-Asn-His-Trp-Ala-Val-D-Ala-His-Leu- |
| | | Nle-NH₂ |
| 81 | 81 | Octadecanedioyl-D-γGlu-OEG-OEG-Gly-Asn-His-7MethylTrp-Ala-Val-D- |
| | | Ala-His-Leu-Nle-Sar-NH₂ |
| 82 | 82 | Octadecanedioyl-D-γGlu-OEG-OEG-Gly-Asn-His-Trp-Ala-Val-D-Ala-His- |
| | | Leu-Nle-Aib-NH₂ |
| 83 | 83 | Octadecanedioyl-D-γGlu-OEG-OEG-OEG-OEG-Gly-Asn-3Pal-Trp-Ala- |
| | | Val-D-Ala-His-Leu-Nle-NH₂ |
| 84 | 84 | Octadecanedioyl-D-γGlu-OEG-OEG-Gly-Asn-His-6MethylTrp-Ala-Val-D- |
| | | Ala-His-Leu-Nle-Sar-NH₂ |
| 85 | 85 | Octadecanedioyl-γGlu-OEG-OEG-Gly-Asn-His-7MethylTrp-Ala-Val-D- |
| | | Ala-His-Leu-Nle-NH₂ |
| 86 | 86 | Octadecanedioyl-γGlu-OEG-OEG-Gly-Asn-His-7FTrp-Ala-Val-D-Ala- |
| | | His-Leu-Nle-NH₂ |
| 87 | 87 | Palmitoyl-γGlu-OEG-OEG-Gly-Asn-His-7FTrp-Ala-Val-D-Ala-His-Leu- |
| | | Nle-NH₂ |
| 88 | 88 | Octadecanedioyl-γGlu-OEG-OEG-Gly-Arg-His-Trp-Ala-Val-D-Ala-His- |
| | | Leu-Nle-NH₂ |
| 89 | 89 | Palmitoyl-γGlu-OEG-OEG-Gly-Arg-His-Trp-Ala-Val-D-Ala-His-Leu-Nle- |
| | | NH₂ |
| 90 | 90 | Octadecanedioyl-γGlu-OEG-OEG-OEG-OEG-Gly-Asn-His-7MethylTrp- |
| | | Ala-Val-D-Ala-His-Leu-Nle-NH₂ |
| 91 | 91 | Palmitoyl-γGlu-OEG-OEG-OEG-OEG-Gly-Asn-3Pal-7MethylTrp-Ala- |
| | | Val-D-Ala-His-Leu-Nle-NH₂ |
| 92 | 92 | Palmitoyl-γGlu-OEG-OEG-Gly-Asn-His-Trp-Ala-Val-D-Ala-His-Leu-Nle- |
| | | Sar-NH₂ |
| 93 | 93 | Palmitoyl-γGlu-OEG-OEG-Gly-Asn-His-2Nal-Ala-Val-Ala-His-Leu-Nle- |
| | | NH₂ |
| 94 | 94 | Palmitoyl-γGlu-OEG-OEG-Gly-Asn-4Pal-Trp-Ala-Val-D-Ala-His-Leu- |
| | | Nle-NH₂ |
| 95 | 95 | Octadecanedioyl-γGlu-OEG-OEG-OEG-OEG-Gly-Asn-3Pal-7MeTrp-Ala- |
| | | Val-D-Ala-His-Leu-Nle-NH₂ |
| 96 | 96 | Palmitoyl-γGlu-OEG-OEG-Gly-Asn-His-Trp-Ala-Val-D-Ala-His-Leu-Nle- |
| | | NH₂ |
| 97 | 97 | Octadecanedioyl-γGlu-OEG-OEG-Gly-Asn-His-Trp-Ala-Val-D-Ala-His- |
| | | Leu-Nle-NH₂ |
| 98 | 98 | Octadecanedioyl-γGlu-OEG-OEG-OEG-OEG-Gly-Asn-His-Trp-Ala-Val- |
| | | D-Ala-His-Leu-Nle-NH₂ |
| 99 | 99 | Octadecanedioyl-γGlu-OEG-OEG-Gly-Asn-His-2Nal-Ala-Val-D-Ala-His- |
| | | Leu-Nle-Sar-NH₂ |
| 100 | 100 | Octadecanedioyl-γGlu-OEG-OEG-Arg-Asn-His-Trp-Ala-Val-D-Ala-His- |
| | | Leu-Nle-Sar-NH₂ |
| 101 | 101 | Octadecanedioyl-γGlu-OEG-OEG-OEG-OEG-Gly-Asn-His-7FTrp-Ala- |
| | | Val-D-Ala-His-Leu-Nle-Sar-NH₂ |
| 102 | 102 | Octadecanedioyl-γGlu-OEG-OEG-Gly-Asn-His-7FTrp-Ala-Val-D-Ala- |
| | | His-Leu-Nle-Sar-NH₂ |
| 103 | 103 | Octadecanedioyl-γGlu-OEG-OEG-OEG-OEG-Gly-Asn-His-7MethylTrp- |
| | | Ala-Val-D-Ala-His-Leu-Nle-Sar-NH₂ |
| 104 | 104 | Octadecanedioyl-γGlu-OEG-OEG-Gly-Asn-His-7MethylTrp-Ala-Val-D- |
| | | Ala-His-Leu-Nle-Sar-NH₂ |
| 105 | 105 | Octadecanedioyl-γGlu-OEG-OEG-Gly-Asn-His-2Nal-Ala-Val-D-Ala-His- |
| | | Leu-Nle-NH₂ |
| 106 | 106 | Octadecanedioyl-γGlu-OEG-OEG-Gly-Asn-His-Trp-D-Ala-Val-D-Ala-His- |
| | | Leu-Nle-NH₂ |
| 107 | 107 | Octadecanedioyl-γGlu-OEG-OEG-Gly-Asn-His-His-Ala-Val-D-Ala-His- |
| | | Leu-Nle-NH₂ |
| 108 | 108 | Octadecanedioyl-γGlu-OEG-OEG-Gly-Asn-His-Arg-Ala-Val-D-Ala-His- |
| | | Leu-Nle-NH₂ |
| 109 | 109 | Octadecanedioyl-γGlu-OEG-OEG-OEG-OEG-Gly-Arg-His-7FTrp-Ala- |
| | | Val-D-Ala-His-Leu-Nle-NH₂ |
| 110 | 110 | Octadecanedioyl-γGlu-OEG-OEG-OEG-OEG-Gly-Asn-His-7FTrp-Ala- |
| | | Val-D-Ala-His-Leu-Nle-NH₂ |
| 111 | 111 | Palmitoyl-γGlu-OEG-OEG-Gly-Asn-His-Trp-Ala-Val-D-Ala-His-Leu-Nle- |
| | | Sar-NH₂ |
| 112 | 112 | Palmitoyl-γGlu-OEG-OEG-Gly-Asn-His-7MethylTrp-Ala-Val-D-Ala-His- |
| | | Leu-Nle-Sar-NH₂ |
| 114 | 114 | Octadecanedioyl-γGlu-OEG-OEG-Gly-Asn-His-Trp-Ala-Val-D-Ala-His- |
| | | Leu-Nle-Arg-NH₂ |
| 115 | 115 | Octadecanedioyl-γGlu-OEG-OEG-Gly-Asn-His-Trp-D-Ala-Val-D-Ala-His- |
| | | Leu-Nle-Sar-NH₂ |
| 116 | 116 | Octadecanedioyl-γGlu-OEG-OEG-Gly-Arg-His-His-Ala-Val-D-Ala-His- |
| | | Leu-Nle-NH₂ |
| 117 | 117 | Octadecanedioyl-γGlu-OEG-OEG-OEG-OEG-Gly-Arg-His-7MethylTrp- |
| | | Ala-Val-D-Ala-His-Leu-Nle-NH₂ |
| 118 | 118 | Octadecanedioyl-γGlu-OEG-OEG-OEG-OEG-Gly-Arg-His-Trp-Ala-Val- |
| | | D-Ala-His-Leu-Nle-NH₂ |
| 119 | 119 | Octadecanedioyl-γGlu-OEG-OEG-Gly-Asn-His-Trp-Ala-Val-D-Ala-His- |
| | | Leu-Cha-NH₂ |
| 120 | 120 | Octadecanedioyl-γGlu-OEG-OEG-Gly-Ala-His-Trp-Ala-Val-D-Ala-His- |
| | | Leu-Cha-NH₂ |
| 121 | 121 | Octadecanedioyl-γGlu-OEG-OEG-OEG-OEG-Gly-Ala-His-7FTrp-Ala- |
| | | Val-D-Ala-His-Leu-Nle-Sar-NH₂ |
| 122 | 122 | Eicosanedioyl-γGlu-OEG-OEG-OEG-OEG-Gly-Arg-His-7MethylTrp-Ala- |
| | | Val-D-Ala-His-Leu-Nle-NH₂ |
| 123 | 123 | Eicosanedioyl-γGlu-OEG-OEG-Gly-Asn-His-7MethylTrp-Ala-Val-D-Ala- |
| | | His-Leu-Nle-Sar-NH₂ |
| 124 | 124 | Octadecanedioyl-γGlu-OEG-OEG-Gly-Ala-His-2Nal-Ala-Val-D-Ala-His- |
| | | Leu-Nle-Sar-NH₂ |
| 125 | 125 | Eicosanedioyl-γGlu-OEG-OEG-Gly-Asn-His-7MethylTrp-D-Ala-Val-D- |
| | | Ala-His-Leu-Nle-Sar-NH₂ |
| 126 | 126 | Octadecanedioyl-γGlu-OEG-OEG-Gly-Ala-His-2Nal-Ala-Val-D-Ala-His- |
| | | Leu-Nle-NH₂ |
| 128 | 128 | Octadecanedioyl-γGlu-OEG-OEG-Gly-Ala-His-Trp-D-Ala-Val-D-Ala-His- |
| | | Leu-Nle-Sar-NH₂ |
| 129 | 129 | Octadecanedioyl-γGlu-OEG-OEG-Gly-Arg-His-7Methy Trp-Ala-Val-D- |
| | | Ala-His-Leu-Nle-Sar-NH₂ |
| 130 | 130 | Octadecanedioyl-γGlu-OEG-OEG-Gly-Asn-His-Arg-Ala-Val-D-Ala-His- |
| | | Leu-Nle-Sar-NH₂ |
| 131 | 131 | Eicosanedioyl-γGlu-OEG-OEG-Gly-Asn-His-Trp-Ala-Val-D-Ala-His-Leu- |
| | | Nle-Sar-NH₂ |
| 132 | 132 | Eicosanedioyl-γGlu-OEG-OEG-OEG-OEG-Gly-Arg-His-Trp-Ala-Val-D- |
| | | Ala-His-Leu-Nle-NH₂ |
| 133 | 133 | Octadecanedioyl-γGlu-OEG-OEG-OEG-OEG-Gly-Asn-His-6FTrp-Ala- |
| | | Val-D-Ala-His-Leu-Nle-Sar-NH₂ |
| 134 | 134 | Octadecanedioyl-γGlu-OEG-OEG-Gly-Asn-His-7MeTrp-Ala-Val-D-Ala- |
| | | His-Leu-Nle-Arg-NH₂ |
| 136 | 136 | Octadecanedioyl-γGlu-OEG-OEG-OEG-OEG-Gly-Ala-His-7FTrp-Ala- |
| | | Val-D-Ala-His-Leu-Nle-NH₂ |
| 137 | 137 | Octadecanedioyl-γGlu-OEG-OEG-OEG-OEG-Gly-Arg-His-7FTrp-Ala- |
| | | Val-D-Ala-His-Leu-Nle-Sar-NH₂ |
| 138 | 138 | Eicosanedioyl-γGlu-OEG-OEG-Gly-Asn-His-7FTrp-Ala-Val-D-Ala-His- |
| | | Leu-Nle-Sar-NH₂ |
| 139 | 139 | Octadecanedioyl-γGlu-OEG-OEG-Gly-Asp-His-Trp-D-Ala-Val-D-Ala-His- |
| | | Leu-Nle-NH₂ |
| 140 | 140 | Octadecanedioyl-γGlu-OEG-OEG-Phe-Asn-His-Trp-Ala-Val-β-Ala-His- |
| | | Leu-Nle-NH₂ |
| 141 | 141 | Octadecanedioyl-γGlu-OEG-OEG-Arg-Arg-His-Trp-Ala-Val-D-Ala-His- |
| | | Leu-Nle-NH₂ |
| 142 | 142 | Octadecanedioyl-γGlu-OEG-OEG-Gly-Asn-His-Trp-Ala-Val-β-Ala-His- |
| | | Leu-Nle-NH₂ |
| 143 | 143 | Octadecanedioyl-γGlu-OEG-OEG-D-Phe-Asn-His-Trp-Ala-Val-D-Ala-His- |
| | | Leu-Nle-NH₂ |
| 144 | 144 | Octadecanedioyl-γGlu-OEG-OEG-Phe-Asn-3Pal-Trp-Ala-Val-β-Ala-His- |
| | | Leu-Nle-NH₂ |
| 145 | 145 | Octadecanedioyl-γGlu-OEG-OEG-Arg-Asn-His-Trp-Ala-Val-D-Ala-His- |
| | | Leu-Nle-NH₂ |
| 146 | 146 | Octadecanedioyl-γGlu-OEG-OEG-Gly-Asn-His-Trp-Ala-Val-D-Ala-His- |
| | | Leu-Nle |
| 147 | 147 | Octadecanedioyl-γGlu-OEG-OEG-Gly-Gln-His-Trp-Ala-Val-D-Ala-His- |
| | | Leu-Nle-NH₂ |
| 148 | 148 | Eicosanedioyl-γGlu-OEG-OEG-Gly-Asn-His-Trp-Ala-Val-D-Ala-His-Leu- |
| | | Nle |
| 149 | 149 | Eicosanedioyl-γGlu-OEG-OEG-Gly-Asn-His-Trp-Ala-Val-D-Ala-His-Leu- |
| | | Nle-NH₂ |
| 150 | 150 | Eicosanedioyl-γGlu-OEG-OEG-Gly-Arg-His-Trp-Ala-Val-D-Ala-His-Leu- |
| | | Nle-NH₂ |
| 151 | 151 | Eicosanedioyl-γGlu-OEG-OEG-Gly-Gln-His-Trp-Ala-Val-D-Ala-His-Leu- |
| | | Nle-NH₂ |
| 152 | 152 | Octadecanedioyl-γGlu-OEG-OEG-Gln-His-Trp-Ala-Val-β-Ala-His-Leu- |
| | | Nle-NH₂ |
| 153 | 153 | Eicosanedioyl-γGlu-Gly-Arg-His-Trp-Ala-Val-D-Ala-His-Leu-Nle-NH₂ |
| 154 | 154 | Eicosanedioyl-γGlu-Arg-Asn-His-Trp-Ala-Val-D-Ala-His-Leu-Nle-NH₂ |
| 155 | 155 | Eicosanedioyl-γGlu-Arg-Asn-His-Trp-Ala-Val-D-Ala-His-Leu-Nle |
| 156 | 156 | Octadecanedioyl-γGlu-OEG-OEG-Arg-Asn-His-Trp-Ala-Val-D-Ala-His- |
| | | Leu-Nle |
| 157 | 157 | Octadecanedioyl-γGlu-OEG-OEG-Arg-Arg-His-Trp-Ala-Val-D-Ala-His- |
| | | Leu-Nle |
| 158 | 158 | Eicosanedioyl-γGlu-Arg-Arg-His-Trp-Ala-Val-D-Ala-His-Leu-Nle-NH₂ |
| 159 | 159 | Octadecanedioyl-γGlu-OEG-OEG-Gly-Asn-His-Trp-Ala-Val-D-Ala-D-His- |
| | | Leu-Nle-NH₂ |
| 160 | 160 | Eicosanedioyl-γGlu-Gly-Asn-His-Trp-Ala-Val-D-Ala-D-His-Leu-Nle-NH₂ |
| 161 | 161 | Octadecanedioyl-γGlu-OEG-OEG-D-Phe-Gln-His-Trp-Ala-Val-D-Ala-His- |
| | | Leu-Nle-NH₂ |
| 162 | 162 | Eicosanedioyl-γGlu-Gly-Arg-His-Trp-Ala-Val-D-Ala-His-Leu-Nle |
| 163 | 163 | Eicosanedioyl-γGlu-Arg-Arg-His-Trp-Ala-Val-D-Ala-His-Leu-Nle |
| 164 | 164 | Eicosanedioyl-γGlu-Gly-Asn-His-Trp-Ala-Val-D-Ala-His-Leu-Nle |
| 165 | 165 | Eicosanedioyl-γGlu-Gly-Gln-His-Trp-Ala-Val-D-Ala-His-Leu-Nle-NH₂ |
| 166 | 166 | Octadecanedioyl-γGlu-OEG-OEG-Gly-Arg-His-Trp-Ala-Val-D-Ala-His- |
| | | Leu-Nle |
| 167 | 167 | Eicosanedioyl-γGlu-OEG-OEG-Gly-Arg-His-Trp-Ala-Val-D-Ala-His-Leu- |
| | | Nle |
| 240 | 240 | Palmitoyl-γGlu-γGlu-OEG-OEG-Gly-Asn-His-Trp-Ala-Val-Gly-His-Leu- |
| | | Nle-NH₂ |
| 241 | 241 | Eicosanedioyl-γGlu-OEG-OEG-Gly-Gln-His-Trp-Ala-Val-β-Ala-His-Leu- |
| | | Nle-NH₂ |
| 242 | 242 | Eicosanedioyl-γGlu-OEG-OEG-Gly-Arg-His-7MethylTrp-Ala-Val-β-Ala- |
| | | His-Leu-Nle-NH₂ |
| 243 | 243 | Octadecanedioyl-γGlu-γGlu-OEG-OEG-Gly-Arg-His-Trp-Ala-Val-β-Ala- |
| | | His-Leu-Nle-NH₂ |
| 244 | 244 | Eicosanedioyl-γGlu-γGlu-OEG-OEG-Gly-Arg-His-Trp-Ala-Val-D-Ala- |
| | | His-Leu-Nle-NH₂ |
| 245 | 245 | Eicosanedioyl-γGlu-γGlu-OEG-OEG-D-Phe-Asn-3PAl-Trp-Ala-Val-β-Ala- |
| | | His-Leu-Nle-NH₂ |
| 247 | 247 | Eicosanedioyl-γGlu-γGlu-OEG-OEG-Gly-Glu-His-7MeTrp-Ala-Val- |
| | | BABA-His-Leu-Nle-NH₂ |
| 248 | 248 | Eicosanedioyl-γGlu-γGlu-OEG-OEG-Glu-D-Ser-Glu-His-7MeTrp-Ala- |
| | | Val-β-Ala-His-Leu-Nle-NH₂ |
| 251 | 251 | Eicosanedioyl-γGlu-γGlu-OEG-OEG-Gly-Gln-His-Trp-Ala-Val-β-Ala- |
| | | His-Leu-Nle-NH₂ |
| 252 | 252 | Eicosanedioyl-γGlu-γGlu-OEG-OEG-Glu-Gly-Arg-His-7MeTrp-Ala-Val- |
| | | β-Ala-His-Leu-Nle-NH₂ |
| 253 | 253 | Eicosanedioyl-γGlu-γGlu-OEG-OEG-Arg-Arg-His-7MeTrp-Ala-Val-β- |
| | | Ala-His-Leu-Nle-NH₂ |
| 254 | 254 | Eicosanedioyl-γGlu-γGlu-OEG-OEG-Arg-Gly-Arg-His-7MeTrp-Ala-Val- |
| | | β-Ala-His-Leu-Nle-NH₂ |
| 255 | 255 | Eicosanedioyl-γGlu-Glu-OEG-OEG-Ser-Gly-Glu-His-7MeTrp-Ala-Val- |
| | | β-Ala-His-Leu-Nle-NH₂ |
| 256 | 256 | Eicosanedioyl-γGlu-Glu-OEG-OEG-Ser-Gly-Arg-His-7MeTrp-Ala-Val- |
| | | β-Ala-His-Leu-Nle-NH₂ |
| 257 | 257 | Eicosanedioyl-γGlu-γGlu-OEG-OEG-Glu-Ser-Gln-His-7MeTrp-Ala-Val- |
| | | β-Ala-His-Leu-Nle-NH₂ |
| 258 | 258 | Eicosanedioyl-γGlu-γGlu-OEG-OEG-Gly-Asn-His-7MeTrp-Ala-Val- |
| | | BABA-His-Leu-Nle-NH₂ |
| 259 | 259 | Eicosanedioyl-γGlu-γGlu-OEG-OEG-Gly-Gln-His-7MeTrp-Ala-Val- |
| | | BABA-His-Leu-Nle-NH₂ |
| 260 | 260 | Eicosanedioyl-γGlu-OEG-OEG-Gly-Dap-His-Trp-Ala-Val-β-Ala-His-Leu- |
| | | Nle-NH₂ |
| 261 | 261 | Eicosanedioyl-γGlu-OEG-OEG-Gly-Dap(5CTMPA)-His-Trp-Ala-Val-β- |
| | | Ala-His-Leu-Nle-NH₂ |
| 262 | 262 | Eicosanedioyl-γGlu-OEG-OEG-Gly-Dap(2CE-PEG-TMEA)-His-Trp-Ala- |
| | | Val-β-Ala-His-Leu-Nle-NH₂ |
| 263 | 263 | Eicosanedioyl-γGlu-OEG-OEG-Gly-Gln-His-7MeTrp-Ala-Val-BAIBA- |
| | | His-Leu-Nle-NH₂ |
| 264 | 264 | Eicosanedioyl-γGlu-OEG-OEG-Gly-Asn-His-7MeTrp-Ala-Val-3A3MBA- |
| | | His-Leu-Nle-NH₂ |
| 265 | 265 | Eicosanedioyl-γGlu-OEG-OEG-Gly-Gln-His-Trp-Ala-Val-β-Ala-His-Leu- |
| | | Hse(Me)-NH₂ |
| 266 | 266 | Eicosanedioyl-γGlu-OEG-OEG-Gly-Glu-Gly-Glu-His-Trp-Ala-Val-β-Ala- |
| | | His-Leu-Nle-NH₂ |
| 267 | 267 | Eicosanedioyl-γGlu-OEG-OEG-Gly-Gln-His-Trp-Ala-Val-β-Ala-NMeHis- |
| | | Leu-Nle-NH₂ |
| 268 | 268 | Eicosanedioyl-γGlu-OEG-OEG-Pro-Gln-His-Trp-Ala-Val-β-Ala-His-Leu- |
| | | Nle-NH₂ |
| 269 | 269 | Eicosanedioyl-γGlu-OEG-OEG-Hyp-Gln-His-Trp-Ala-Val-β-Ala-His-Leu- |
| | | Nle-NH₂ |
| 270 | 270 | Eicosanedioyl-γGlu-γGlu-OEG-OEG-Gly-Gln-His-7MeTrp-Ala-Val- |
| | | BABA-NMeHis-Leu-Nle-NH₂ |
| 271 | 271 | Eicosanedioyl-γGlu-γGlu-OEG-OEG-Gly-Gln-His-Trp-Ala-Val-ACPC- |
| | | His-Leu-Nle-NH₂ |
| 272 | 272 | Eicosanedioyl-γGlu-γGlu-OEG-OEG-Gly-Gln-His-Trp-Ala-Val-ACPA- |
| | | His-Leu-Nle-NH₂ |
| 273 | 273 | Eicosanedioyl-γGlu-Glu-OEG-OEG-Gly-Gln-His-Trp-Ala-Val-ACBA- |
| | | His-Leu-Nle-NH₂ |
| 274 | 274 | Eicosanedioyl-γGlu-γGlu-OEG-OEG-Gly-Asn-His-αMeTrp-Ala-Val-β- |
| | | Ala-His-Leu-Nle-NH₂ |
| 275 | 275 | Eicosanedioyl-γGlu-γGlu-PEG5-Gly-Glu-His-7MeTrp-Ala-Val-BABA- |
| | | His-Leu-Nle-NH₂ |
| 276 | 276 | Eicosanedioyl-γGlu-OEG-OEG-Gly-Gln-His-Trp-Ala-Val-β-Ala-His-Leu- |
| | | MetO-NH₂ |
| 277 | 277 | Eicosanedioyl-γGlu-γGlu-OEG-OEG-Ser-Glu-His-7MeTrp-Ala-Val- |
| | | BABA-NMeHis-Leu-Nle-NH₂ |
| 278 | 278 | Eicosanedioyl-γGlu-γGlu-OEG-OEG-Gly-Asp-His-7MeTrp-Ala-Val- |
| | | BABA-NMeHis-Leu-Nle-NH₂ |

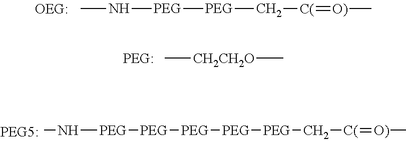

Table 2 provides structures of illustrative peptides of the disclosure. In some embodiments, a composition or method of the disclosure comprises a peptide comprising an amino acid sequence having at least 50%, 55%, 60%, 65%, 70%, 75%, 80%, 85%, 90%, 95%, 96%, 97%, 98%, 99% or 100% sequence identity to any one of the sequences of Table 2, or a pharmaceutically acceptable salt, solvate, or isotope thereof. In some embodiments, a composition or method of the disclosure comprises a peptide of Table 2, or a pharmaceutically acceptable salt, solvate, or isotope thereof.

In some embodiments, a composition or method of the disclosure comprises a peptide comprising an amino acid sequence having at least 50%, 55%, 60%, 65%, 70%, 75%, 80%, 85%, 90%, 95%, 96%, 97%, 98%, 99% or 100% sequence identity to any one of the sequences of Table 2, or a pharmaceutically acceptable salt, solvate, or isotope thereof, wherein the peptide comprises one or more lipid moieties as described herein. In some embodiments, a composition or method of the disclosure comprises a peptide of Table 2, or a pharmaceutically acceptable salt, solvate, or isotope thereof, wherein the peptide of Table 2 is conjugated to one or more lipid moieties. In some embodiments, the peptide of Table 2 is conjugated to one lipid moiety. In some embodiments, the peptide of Table 2 is conjugated to two lipid moieties. In some embodiments, the peptide of Table 2 is conjugated to at least 1, 2, 3, or 4 lipid moieties.

In some embodiments, the peptide of Table 2 is conjugated to a C₂-C₂₆ fatty acyl group. In some embodiments, the peptide of Table 2 is conjugated to a C₄-C₂₆ fatty acyl group. In some embodiments, the peptide of Table 2 is conjugated to a C₈-C₂₆ fatty acyl group. In some embodiments, the peptide of Table 2 is conjugated to a C₁₂-C₂₀ fatty acyl group. In some embodiments, the peptide of Table 2 is conjugated to palmitoyl or stearoyl, wherein palmitoyl and stearoyl are substituted or unsubstituted.

In some embodiments, the peptide of Table 2 is conjugated to -(L¹)ₚ-(L²)_{q}-(L³)ᵣ.

In some embodiments, the peptide of Table 2 is conjugated to

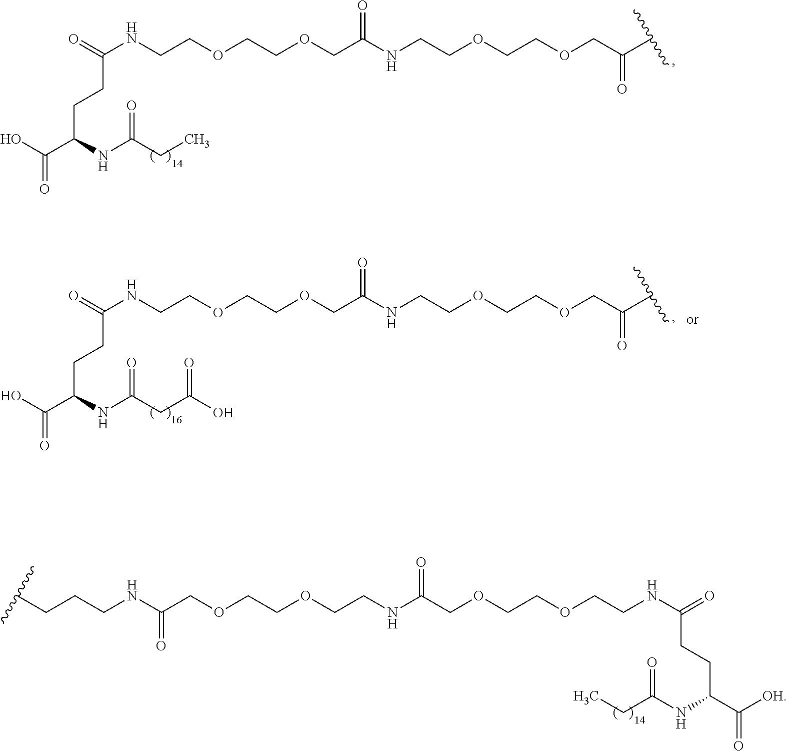

In some embodiments, the peptide of Table 2 is conjugated to

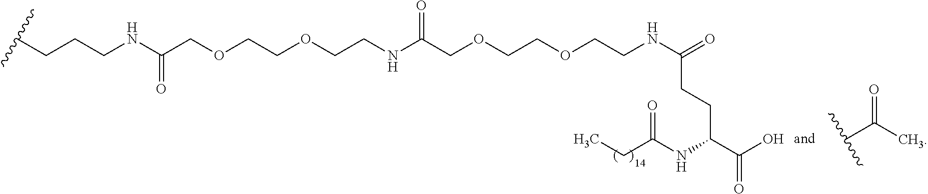

TABLE 2
Exemplary Peptides

| SEQ | |
|---|---|
| ID | |
| NO. | Structure |
| 6 | 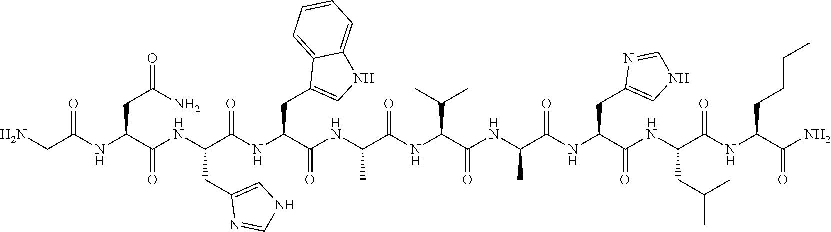 [SMILES: CCCC[C@H](NC(=O)[C@H](CC(C)C)NC(=O)[C@H](Cc1c[nH]cn1)NC(=O)[C@@H](C)NC(=O)[C@@H](NC(=O)[C@H](C)NC(=O)[C@H](Cc1c[nH]c2ccccc12)NC(=O)[C@H](Cc1c[nH]cn1)NC(=O)[C@H](CC(N)=O)NC(=O)CN)C(C)C)C(N)=O] |
| 27 | 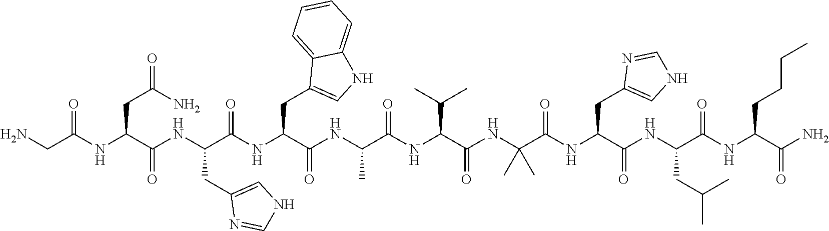 [SMILES: CCCC[C@H](NC(=O)[C@H](CC(C)C)NC(=O)[C@H](Cc1c[nH]cn1)NC(=O)C(C)(C)NC(=O)[C@@H](NC(=O)[C@H](C)NC(=O)[C@H](Cc1c[nH]c2ccccc12)NC(=O)[C@H](Cc1c[nH]cn1)NC(=O)[C@H](CC(N)=O)NC(=O)CN)C(C)C)C(N)=O] |
| 28 | 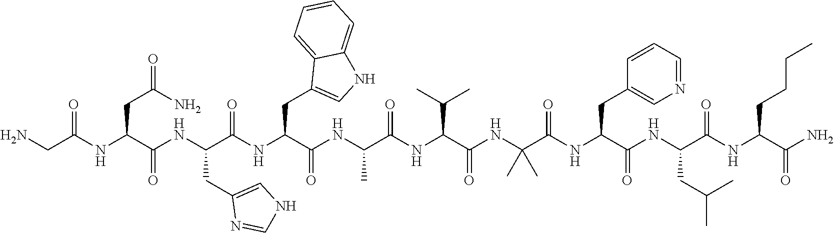 [SMILES: CCCC[C@H](NC(=O)[C@H](CC(C)C)NC(=O)[C@H](Cc1cccnc1)NC(=O)C(C)(C)NC(=O)[C@@H](NC(=O)[C@H](C)NC(=O)[C@H](Cc1c[nH]c2ccccc12)NC(=O)[C@H](Cc1c[nH]cn1)NC(=O)[C@H](CC(N)=O)NC(=O)CN)C(C)C)C(N)=O] |
| 29 | 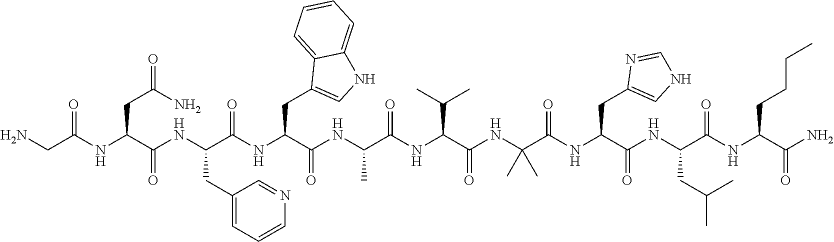 [SMILES: CCCC[C@H](NC(=O)[C@H](CC(C)C)NC(=O)[C@H](Cc1c[nH]cn1)NC(=O)C(C)(C)NC(=O)[C@@H](NC(=O)[C@H](C)NC(=O)[C@H](Cc1c[nH]c2ccccc12)NC(=O)[C@H](Cc1cccnc1)NC(=O)[C@H](CC(N)=O)NC(=O)CN)C(C)C)C(N)=O] |
| 30 | 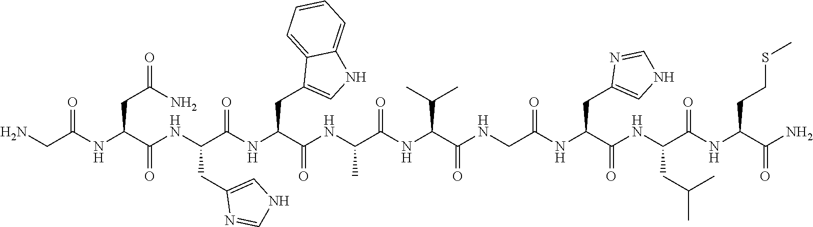 [SMILES: CSCC[C@H](NC(=O)[C@H](CC(C)C)NC(=O)[C@H](Cc1c[nH]cn1)NC(=O)CNC(=O)[C@@H](NC(=O)[C@H](C)NC(=O)[C@H](Cc1c[nH]c2ccccc12)NC(=O)[C@H](Cc1c[nH]cn1)NC(=O)[C@H](CC(N)=O)NC(=O)CN)C(C)C)C(N)=O] |
| 31 | 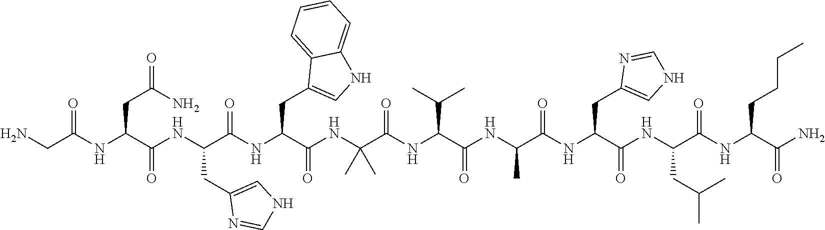 [SMILES: CCCC[C@H](NC(=O)[C@H](CC(C)C)NC(=O)[C@H](Cc1c[nH]cn1)NC(=O)[C@@H](C)NC(=O)[C@@H](NC(=O)C(C)(C)NC(=O)[C@H](Cc1c[nH]c2ccccc12)NC(=O)[C@H](Cc1c[nH]cn1)NC(=O)[C@H](CC(N)=O)NC(=O)CN)C(C)C)C(N)=O] |
| 32 | 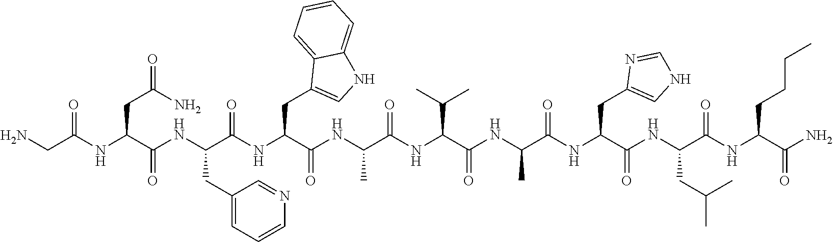 [SMILES: CCCC[C@H](NC(=O)[C@H](CC(C)C)NC(=O)[C@H](Cc1c[nH]cn1)NC(=O)[C@@H](C)NC(=O)[C@@H](NC(=O)[C@H](C)NC(=O)[C@H](Cc1c[nH]c2ccccc12)NC(=O)[C@H](Cc1cccnc1)NC(=O)[C@H](CC(N)=O)NC(=O)CN)C(C)C)C(N)=O] |
| 33 | 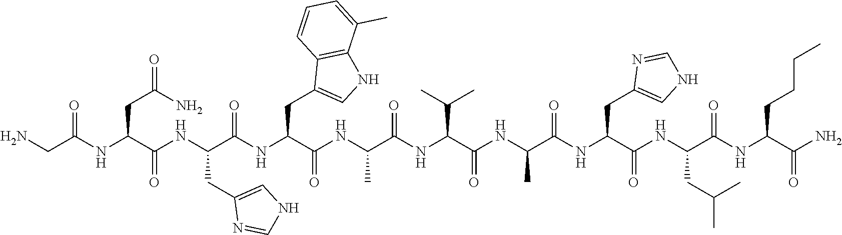 [SMILES: CCCC[C@H](NC(=O)[C@H](CC(C)C)NC(=O)[C@H](Cc1c[nH]cn1)NC(=O)[C@@H](C)NC(=O)[C@@H](NC(=O)[C@H](C)NC(=O)[C@H](Cc1c[nH]c2c(C)cccc12)NC(=O)[C@H](Cc1c[nH]cn1)NC(=O)[C@H](CC(N)=O)NC(=O)CN)C(C)C)C(N)=O] |
| 34 | 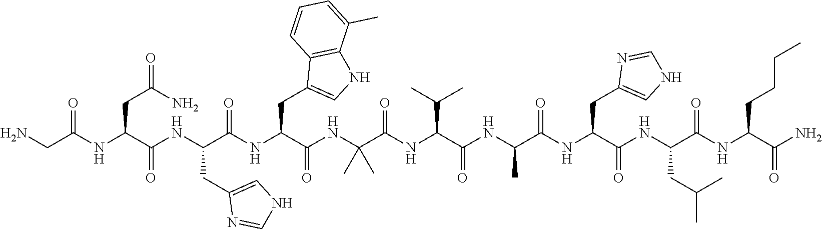 [SMILES: CCCC[C@H](NC(=O)[C@H](CC(C)C)NC(=O)[C@H](Cc1c[nH]cn1)NC(=O)[C@@H](C)NC(=O)[C@@H](NC(=O)C(C)(C)NC(=O)[C@H](Cc1c[nH]c2c(C)cccc12)NC(=O)[C@H](Cc1c[nH]cn1)NC(=O)[C@H](CC(N)=O)NC(=O)CN)C(C)C)C(N)=O] |
| 35 | 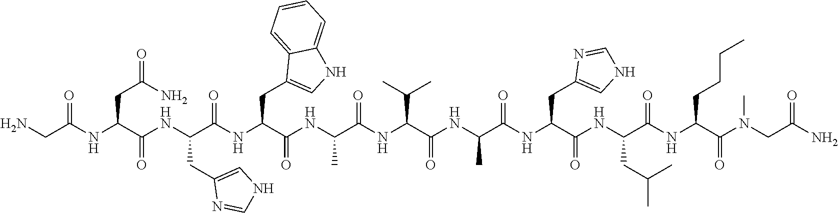 [SMILES: CCCC[C@H](NC(=O)[C@H](CC(C)C)NC(=O)[C@H](Cc1c[nH]cn1)NC(=O)[C@@H](C)NC(=O)[C@@H](NC(=O)[C@H](C)NC(=O)[C@H](Cc1c[nH]c2ccccc12)NC(=O)[C@H](Cc1c[nH]cn1)NC(=O)[C@H](CC(N)=O)NC(=O)CN)C(C)C)C(=O)N(C)CC(N)=O] |
| 36 | 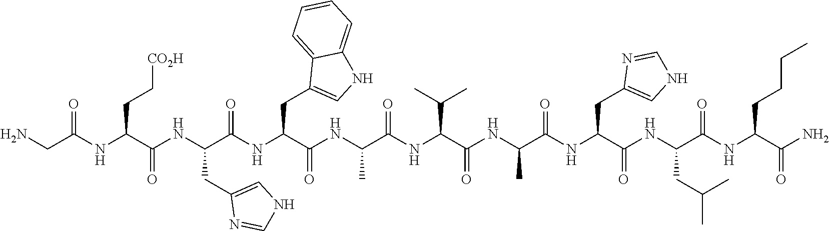 [SMILES: CCCC[C@H](NC(=O)[C@H](CC(C)C)NC(=O)[C@H](Cc1c[nH]cn1)NC(=O)[C@@H](C)NC(=O)[C@@H](NC(=O)[C@H](C)NC(=O)[C@H](Cc1c[nH]c2ccccc12)NC(=O)[C@H](Cc1c[nH]cn1)NC(=O)[C@H](CCC(=O)O)NC(=O)CN)C(C)C)C(N)=O] |
| 37 | 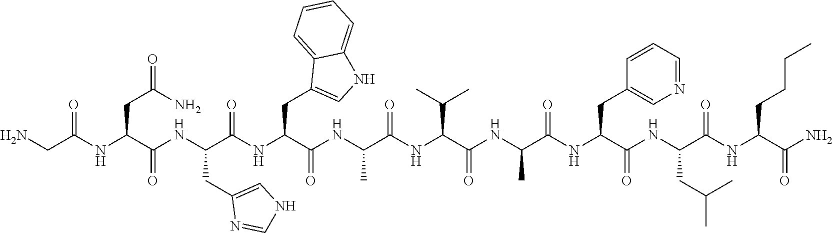 [SMILES: CCCC[C@H](NC(=O)[C@H](CC(C)C)NC(=O)[C@H](Cc1cccnc1)NC(=O)[C@@H](C)NC(=O)[C@@H](NC(=O)[C@H](C)NC(=O)[C@H](Cc1c[nH]c2ccccc12)NC(=O)[C@H](Cc1c[nH]cn1)NC(=O)[C@H](CC(N)=O)NC(=O)CN)C(C)C)C(N)=O] |
| 38 | 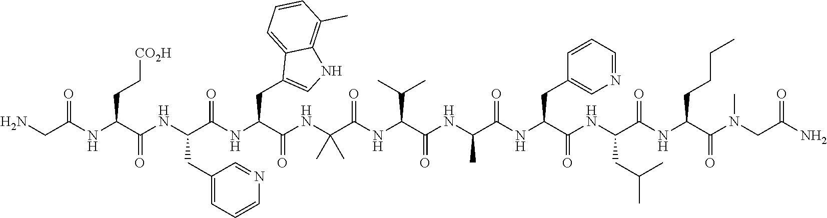 [SMILES: CCCC[C@H](NC(=O)[C@H](CC(C)C)NC(=O)[C@H](Cc1cccnc1)NC(=O)[C@@H](C)NC(=O)[C@@H](NC(=O)C(C)(C)NC(=O)[C@H](Cc1c[nH]c2c(C)cccc12)NC(=O)[C@H](Cc1cccnc1)NC(=O)[C@H](CCC(=O)O)NC(=O)CN)C(C)C)C(=O)N(C)CC(N)=O] |
| 39 | 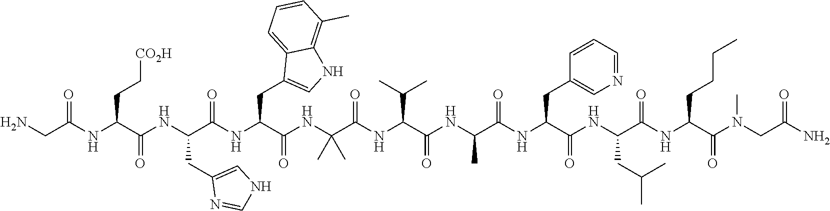 [SMILES: CCCC[C@H](NC(=O)[C@H](CC(C)C)NC(=O)[C@H](Cc1cccnc1)NC(=O)[C@@H](C)NC(=O)[C@@H](NC(=O)C(C)(C)NC(=O)[C@H](Cc1c[nH]c2c(C)cccc12)NC(=O)[C@H](Cc1c[nH]cn1)NC(=O)[C@H](CCC(=O)O)NC(=O)CN)C(C)C)C(=O)N(C)CC(N)=O] |
| 40 | 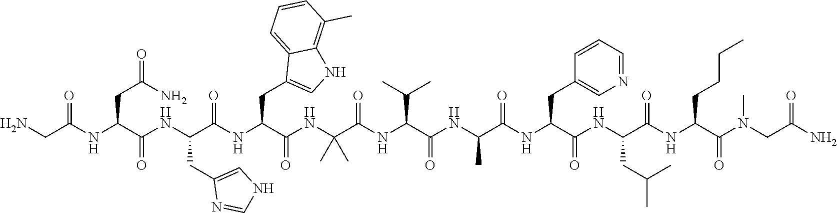 [SMILES: CCCC[C@H](NC(=O)[C@H](CC(C)C)NC(=O)[C@H](Cc1cccnc1)NC(=O)[C@@H](C)NC(=O)[C@@H](NC(=O)C(C)(C)NC(=O)[C@H](Cc1c[nH]c2c(C)cccc12)NC(=O)[C@H](Cc1c[nH]cn1)NC(=O)[C@H](CC(N)=O)NC(=O)CN)C(C)C)C(=O)N(C)CC(N)=O] |
| 41 | 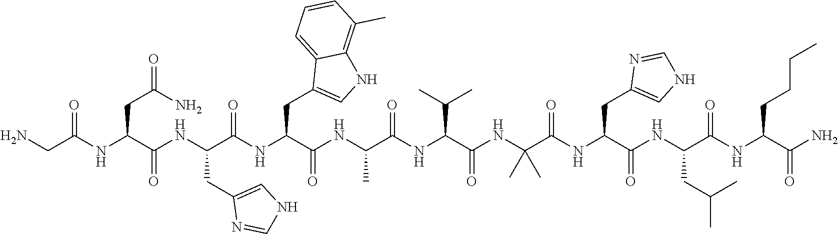 [SMILES: CCCC[C@H](NC(=O)[C@H](CC(C)C)NC(=O)[C@H](Cc1c[nH]cn1)NC(=O)C(C)(C)NC(=O)[C@@H](NC(=O)[C@H](C)NC(=O)[C@H](Cc1c[nH]c2c(C)cccc12)NC(=O)[C@H](Cc1c[nH]cn1)NC(=O)[C@H](CC(N)=O)NC(=O)CN)C(C)C)C(N)=O] |
| 42 | 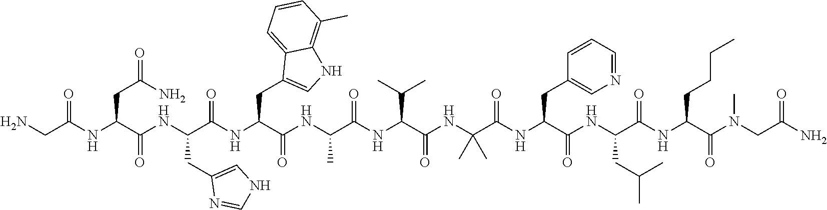 [SMILES: CCCC[C@H](NC(=O)[C@H](CC(C)C)NC(=O)[C@H](Cc1cccnc1)NC(=O)C(C)(C)NC(=O)[C@@H](NC(=O)[C@H](C)NC(=O)[C@H](Cc1c[nH]c2c(C)cccc12)NC(=O)[C@H](Cc1c[nH]cn1)NC(=O)[C@H](CC(N)=O)NC(=O)CN)C(C)C)C(=O)N(C)CC(N)=O] |
| 43 | 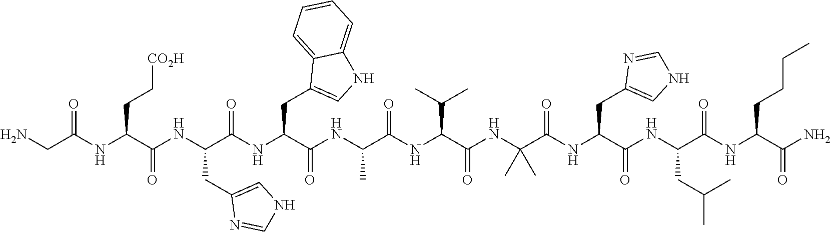 [SMILES: CCCC[C@H](NC(=O)[C@H](CC(C)C)NC(=O)[C@H](Cc1c[nH]cn1)NC(=O)C(C)(C)NC(=O)[C@@H](NC(=O)[C@H](C)NC(=O)[C@H](Cc1c[nH]c2ccccc12)NC(=O)[C@H](Cc1c[nH]cn1)NC(=O)[C@H](CCC(=O)O)NC(=O)CN)C(C)C)C(N)=O] |

Table 3 provides amino acid sequences of illustrative peptides of the disclosure. In some embodiments, a composition or method of the disclosure comprises a peptide comprising an amino acid sequence having at least 50%, 55%, 60%, 65%, 70%, 75%, 80%, 85%, 90%, 95%, 96%, 97%, 98%, 99% or 100% sequence identity to any one of the sequences of Table 3, or a pharmaceutically acceptable salt, solvate, or isotope thereof, wherein the peptide comprises one or more lipid moieties as described herein. In some embodiments, a composition or method of the disclosure comprises a peptide comprising an amino acid sequence having at least 50%, 55%, 60%, 65%, 70%, 75%, 80%, 85%, 90%, 95%, 96%, 97%, 98%, 99% or 100% sequence identity to any one of the sequences of Table 3, or a pharmaceutically acceptable salt, solvate, or isotope thereof.

TABLE 3
Amino Acid Sequences for Exemplary Peptides

| SEQ ID | |
|---|---|
| NO. | Sequence |
| 6 | Gly-Asn-His-Trp-Ala-Val-D-Ala-His-Leu-Nle-NH₂ |
| 27 | Gly-Asn-His-Trp-Ala-Val-Aib-His-Leu-Nle-NH₂ |
| 28 | Gly-Asn-His-Trp-Ala-Val-Aib-3Pal-Leu-Nle-NH₂ |
| 29 | Gly-Asn-3Pal-Trp-Ala-Val-Aib-His-Leu-Nle-NH₂ |
| 30 | Gly-Asn-His-Trp-Ala-Val-Gly-His-Leu-Met-NH₂ |
| 31 | Gly-Asn-His-Trp-Aib-Val-D-Ala-His-Leu-Nle-NH₂ |
| 32 | Gly-Asn-3Pal-Trp-Ala-Val-D-Ala-His-Leu-Nle-NH₂ |
| 33 | Gly-Asn-His-7MethylTrp-Ala-Val-D-Ala-His-Leu-Nle-NH₂ |
| 34 | Gly-Asn-His-7MethylTrp-Aib-Val-D-Ala-His-Leu-Nle-NH₂ |
| 35 | Gly-Asn-His-Trp-Ala-Val-D-Ala-His-Leu-Nle-Sar-NH₂ |
| 36 | Gly-Glu-His-Trp-Ala-Val-D-Ala-His-Leu-Nle-NH₂ |
| 37 | Gly-Asn-His-Trp-Ala-Val-D-Ala-3Pal-Leu-Nle-NH₂ |
| 38 | Gly-Glu-3Pal-7MethylTrp-Aib-Val-D-Ala-3Pal-Leu-Nle-Sar-NH₂ |
| 39 | Gly-Glu-His-7MethylTrp-Aib-Val-D-Ala-3Pal-Leu-Nle-Sar-NH₂ |
| 40 | Gly-Asn-His-7MethylTrp-Aib-Val-D-Ala-3Pal-Leu-Nle-Sar-NH₂ |
| 41 | Gly-Asn-His-7MethylTrp-Ala-Val-Aib-His-Leu-Nle-NH₂ |
| 42 | Gly-Asn-His-7MethylTrp-Ala-Val-Aib-3Pal-Leu-Nle-Sar-NH₂ |
| 43 | Gly-Glu-His-Trp-Ala-Val-Aib-His-Leu-Nle-NH₂ |
| 168 | Gly-Asn-3Pal-7MethylTrp-Ala-Val-D-Ala-His-Leu-Nle-NH₂ |
| 169 | Gly-Asn-His-Trp-Ala-Val-D-Ala-His-Leu-Lys(Me)₃-NH₂ |
| 170 | Gly-Asn-2Pal-Trp-Ala-Val-D-Ala-His-Leu-Nle-NH₂ |
| 171 | Gly-Asn-4Pal-Trp-Ala-Val-D-Ala-His-Leu-Nle-NH₂ |
| 172 | Gly-Asn-His-7FTrp-Ala-Val-D-Ala-His-Leu-Nle-NH₂ |
| 173 | Gly-Lys-His-Trp-Ala-Val-D-Ala-His-Leu-Nle-NH₂ |
| 174 | Gly-Arg-His-Trp-Ala-Val-D-Ala-His-Leu-Nle-NH₂ |
| 175 | Gly-Asn-His-Trp-Ala-Val-D-Ala-Phe-Leu-Nle-NH₂ |
| 176 | Gly-Asn-His-6MethylTrp-Ala-Val-D-Ala-His-Leu-Nle-NH₂ |
| 177 | Gly-Lys(Ac)-His-Trp-Ala-Val-D-Ala-His-Leu-Nle-NH₂ |
| 179 | Gly-Glu-His-Trp-Ala-Val-D-Ala-3Pal-Leu-Nle-NH₂ |
| 180 | Gly-Asn-His-His-Ala-Val-D-Ala-His-Leu-Nle-NH₂ |
| 181 | Gly-Asn-His-Trp-Ala-Val-D-Ala-His-Leu-Arg-NH₂ |
| 182 | Gly-Glu-His-Trp-Aib-Val-D-Ala-His-Leu-Nle-NH₂ |
| 183 | Lys(Ac)-His-Trp-Ala-Val-D-Ala-His-Leu-Nle-NH₂ |
| 184 | Gly-Asn-3Pal-Trp-Ala-Val-D-Ala-His-Leu-Nle-Sar-NH₂ |
| 185 | Gly-Asn-3Pal-7MethylTrp-Ala-Val-D-Ala-His-Leu-Nle-Sar-NH₂ |
| 186 | Aib-Asn-His-Trp-Ala-Val-D-Ala-His-Leu-Nle-NH₂ |
| 187 | Gly-Asn-His-Trp-Ala-Val-D-Ala-His-Leu-Nle-Aib-NH₂ |
| 188 | Gly-Asn-His-7MethylTrp-Ala-Val-D-Ala-His-Leu-Nle-Aib-NH₂ |
| 189 | Gly-Lys(Ac)-His-Trp-Ala-Val-D-Ala-His-Leu-Nle-Sar-NH₂ |
| 190 | Gly-Asn-His-7MethylTrp-Ala-Val-D-Ala-His-Leu-NH₂ |
| 191 | Gly-Asn-His-7MethylTrp-Ala-Val-D-Ala-His-Leu-Nle-Sar-NH₂ |
| 192 | Gly-Asn-His-6MethylTrp-Ala-Val-D-Ala-His-Leu-Nle-Sar-NH₂ |
| 193 | Gly-Arg-His-Trp-Ala-Val-D-Ala-His-Leu-Nle-NH₂ |
| 194 | Gly-Asn-His-2Nal-Ala-Val-Ala-His-Leu-Nle-NH₂ |
| 195 | Gly-Asn-3Pal-7MeTrp-Ala-Val-D-Ala-His-Leu-Nle-NH₂ |
| 196 | Gly-Asn-His-2Nal-Ala-Val-D-Ala-His-Leu-Nle-Sar-NH₂ |
| 197 | Arg-Asn-His-Trp-Ala-Val-D-Ala-His-Leu-Nle-Sar-NH₂ |
| 198 | Gly-Asn-His-7FTrp-Ala-Val-D-Ala-His-Leu-Nle-Sar-NH₂ |
| 199 | Gly-Asn-His-2Nal-Ala-Val-D-Ala-His-Leu-Nle-NH₂ |
| 200 | Gly-Asn-His-Trp-D-Ala-Val-D-Ala-His-Leu-Nle-NH₂ |
| 201 | Gly-Asn-His-Arg-Ala-Val-D-Ala-His-Leu-Nle-NH₂ |
| 202 | Gly-Arg-His-7FTrp-Ala-Val-D-Ala-His-Leu-Nle-NH₂ |
| 203 | Gly-Asn-His-Trp-Ala-Val-D-Ala-His-Leu-Nle-Arg-NH₂ |
| 204 | Gly-Asn-His-Trp-D-Ala-Val-D-Ala-His-Leu-Nle-Sar-NH₂ |
| 205 | Gly-Arg-His-His-Ala-Val-D-Ala-His-Leu-Nle-NH₂ |
| 206 | Gly-Arg-His-7MethylTrp-Ala-Val-D-Ala-His-Leu-Nle-NH₂ |
| 207 | Gly-Asn-His-Trp-Ala-Val-D-Ala-His-Leu-Cha-NH₂ |
| 208 | Gly-Ala-His-Trp-Ala-Val-D-Ala-His-Leu-Cha-NH₂ |
| 209 | Gly-Ala-His-7FTrp-Ala-Val-D-Ala-His-Leu-Nle-Sar-NH₂ |
| 210 | Gly-Ala-His-2Nal-Ala-Val-D-Ala-His-Leu-Nle-Sar-NH₂ |
| 211 | Gly-Asn-His-7MethylTrp-D-Ala-Val-D-Ala-His-Leu-Nle-Sar-NH₂ |
| 212 | Gly-Ala-His-2Nal-Ala-Val-D-Ala-His-Leu-Nle-NH₂ |
| 214 | Gly-Ala-His-Trp-D-Ala-Val-D-Ala-His-Leu-Nle-Sar-NH₂ |
| 215 | Gly-Arg-His-7MethylTrp-Ala-Val-D-Ala-His-Leu-Nle-Sar-NH₂ |
| 216 | Gly-Asn-His-Arg-Ala-Val-D-Ala-His-Leu-Nle-Sar-NH₂ |
| 217 | Gly-Asn-His-6FTrp-Ala-Val-D-Ala-His-Leu-Nle-Sar-NH₂ |
| 218 | Gly-Asn-His-7MethylTrp-Ala-Val-D-Ala-His-Leu-Nle-Arg-NH₂ |
| 220 | Gly-Ala-His-7FTrp-Ala-Val-D-Ala-His-Leu-Nle-NH₂ |
| 221 | Gly-Arg-His-7FTrp-Ala-Val-D-Ala-His-Leu-Nle-Sar-NH₂ |
| 222 | Gly-Asp-His-Trp-D-Ala-Val-D-Ala-His-Leu-Nle-NH₂ |
| 223 | Phe-Asn-His-Trp-Ala-Val-β-Ala-His-Leu-Nle-NH₂ |
| 224 | Arg-Arg-His-Trp-Ala-Val-D-Ala-His-Leu-Nle-NH₂ |
| 225 | Gly-Asn-His-Trp-Ala-Val-β-Ala-His-Leu-Nle-NH₂ |
| 226 | D-Phe-Asn-His-Trp-Ala-Val-D-Ala-His-Leu-Nle-NH₂ |
| 227 | Phe-Asn-3Pal-Trp-Ala-Val-β-Ala-His-Leu-Nle-NH₂ |
| 228 | Arg-Asn-His-Trp-Ala-Val-D-Ala-His-Leu-Nle-NH₂ |
| 229 | Gly-Asn-His-Trp-Ala-Val-D-Ala-His-Leu-Nle |
| 230 | Gly-Gln-His-Trp-Ala-Val-D-Ala-His-Leu-Nle-NH₂ |
| 231 | Arg-Asn-His-Trp-Ala-Val-D-Ala-His-Leu-Nle |
| 232 | Arg-Arg-His-Trp-Ala-Val-D-Ala-His-Leu-Nle |
| 233 | Gly-Asn-His-Trp-Ala-Val-D-Ala-D-His-Leu-Nle-NH₂ |
| 234 | D-Phe-Gln-His-Trp-Ala-Val-D-Ala-His-Leu-Nle-NH₂ |
| 235 | Gly-Arg-His-Trp-Ala-Val-D-Ala-His-Leu-Nle |
| 236 | Gly-Asn-His-Trp-Ala-Val-Gly-His-Leu-Nle-NH₂ |
| 237 | Gly-Gln-His-Trp-Ala-Val-β-Ala-His-Leu-Nle-NH₂ |
| 238 | Gly-Arg-His-7MethylTrp-Ala-Val-β-Ala-His-Leu-Nle-NH₂ |
| 239 | Gly-Arg-His-Trp-Ala-Val-β-Ala-His-Leu-Nle-NH₂ |
| 246 | D-Phe-Asn-3Pal-Trp-Ala-Val-β-Ala-His-Leu-Nle-NH₂ |
| 249 | Gly-Glu-His-7MeTrp-Ala-Val-BABA-His-Leu-Nle-NH₂ |
| 250 | Glu-D-Ser-Glu-His-7MeTrp-Ala-Val-β-Ala-His-Leu-Nle-NH₂ |
| 279 | Gly-Asn-His-Trp-Ala-Val-D-Ala-His-Leu-Nle |
| 280 | Arg-Arg-His-7MeTrp-Ala-Val-β-Ala-His-Leu-Nle-NH₂ |
| 281 | Arg-Gly-Arg-His-7MeTrp-Ala-Val-β-Ala-His-Leu-Nle-NH₂ |
| 282 | Ser-Gly-Glu-His-7MeTrp-Ala-Val-β-Ala-His-Leu-Nle-NH₂ |
| 283 | Ser-Gly-Arg-His-7MeTrp-Ala-Val-β-Ala-His-Leu-Nle-NH₂ |
| 284 | Glu-Ser-Gln-His-7MeTrp-Ala-Val-β-Ala-His-Leu-Nle-NH₂ |
| 285 | Gly-Asn-His-7MeTrp-Ala-Val-BABA-His-Leu-Nle-NH₂ |
| 286 | Gly-Gln-His-7MeTrp-Ala-Val-BABA-His-Leu-Nle-NH₂ |
| 287 | Gly-Dap-His-Trp-Ala-Val-β-Ala-His-Leu-Nle-NH₂ |
| 288 | Gly-Dap(5CTMPA)-His-Trp-Ala-Val-β-Ala-His-Leu-Nle-NH₂ |
| 289 | Gly-Dap(2CE-PEG-TMEA)-His-Trp-Ala-Val-β-Ala-His-Leu-Nle-NH₂ |
| 290 | Gly-Gln-His-7MeTrp-Ala-Val-BAIBA-His-Leu-Nle-NH₂ |
| 291 | Gly-Asn-His-7MeTrp-Ala-Val-3A3MBA-His-Leu-Nle-NH₂ |
| 292 | Gly-Gln-His-Trp-Ala-Val-β-Ala-His-Leu-HSe(Me)-NH₂ |
| 293 | Gly-Glu-Gly-Glu-His-Trp-Ala-Val-β-Ala-His-Leu-Nle-NH₂ |
| 294 | Gly-Gln-His-Trp-Ala-Val-β-Ala-NMeHis-Leu-Nle-NH₂ |
| 295 | Pro-Gln-His-Trp-Ala-Val-β-Ala-His-Leu-Nle-NH₂ |
| 296 | Hyp-Gln-His-Trp-Ala-Val-β-Ala-His-Leu-Nle-NH₂ |
| 297 | Gly-Gln-His-7MeTrp-Ala-Val-BABA-NMeHis-Leu-Nle-NH₂ |
| 298 | Gly-Gln-His-Trp-Ala-Val-ACPC-His-Leu-Nle-NH₂ |
| 299 | Gly-Gln-His-Trp-Ala-Val-ACPA-His-Leu-Nle-NH₂ |
| 300 | Gly-Gln-His-Trp-Ala-Val-ACBA-His-Leu-Nle-NH₂ |
| 301 | Gly-Asn-His-αMeTrp-Ala-Val-β-Ala-His-Leu-Nle-NH₂ |
| 302 | Gly-Glu-His-7MeTrp-Ala-Val-BABA-His-Leu-Nle-NH₂ |
| 303 | Gly-Gln-His-Trp-Ala-Val-β-Ala-His-Leu-Met(O)-NH₂ |
| 304 | Ser-Glu-His-7MeTrp-Ala-Val-BABA-NMeHis-Leu-Nle-NH₂ |
| 305 | Gly-Asp-His-7MeTrp-Ala-Val-BABA-NMeHis-Leu-Nle-NH₂ |
| 306 | Glu-Gly-Arg-His-7MeTrp-Ala-Val-β-Ala-His-Leu-Nle-NH₂ |

In Tables 1A and 3, the following abbreviations stand for:
3Pal: 3-(3-pyridyl)alanine

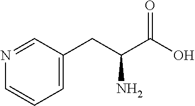 [SMILES: N[C@@H](Cc1cccnc1)C(=O)O]

7MeW: 7-methyltryptophan

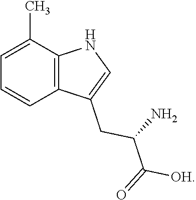 [SMILES: Cc1cccc2c(C[C@H](N)C(=O)O)c[nH]c12]

Aib: α-aminoisobutyric acid

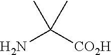

Sar: sarcosine

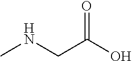

a=D-Ala=D-alanine

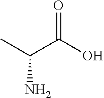

b=β-Ala=β-alanine

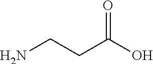

Cha=cyclohexylalanine

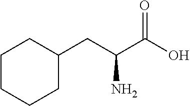 [SMILES: N[C@@H](CC1CCCCC1)C(=O)O]

s=D-Ser=D-serine

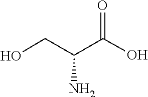

BABA=S-β-aminobutanoic acid

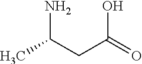

NMeHis=N-methyl-L-histidine

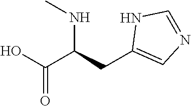

Dap=(S)-2,3-diaminopropanoic acid

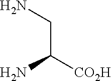

5CTMPA=5-carboxy-N,N,N-trimethylpentan-1-aminium
Dap(2CE-PEG-TMEA)=

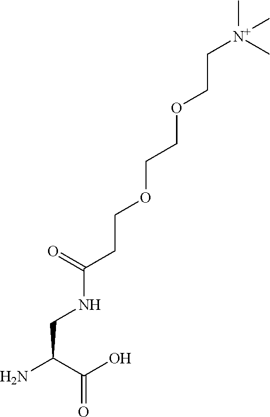 [SMILES: C[N+](C)(C)CCOCCOCCC(=O)NC[C@H](N)C(=O)O]

2CE-PEG-TMEA=2-(2-(2-carboxyethoxy)ethoxy)-N,N,N-trimethylethan-1-aminium
Dap(5CTMPA)=

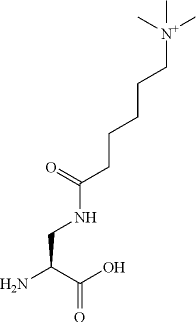 [SMILES: C[N+](C)(C)CCCCCC(=O)NC[C@H](N)C(=O)O]

BAIBA=(S)-β-aminoisobutyric acid

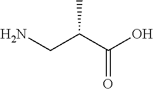

3A3MBA=3-amino-3-methylbutanoic acid

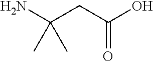

ACPC=(1S,2S)-2-aminocyclopentane-1-carboxylic acid

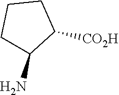

ACPA=2-(1-aminocyclopropyl)acetic acid

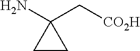

ACBA=2-(1-aminocyclobutyl)acetic acid

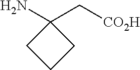

MetO=(2S)-2-amino-4-(methylsulfinyl)butanoic acid

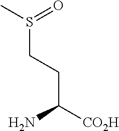

Hyp=(2S,4R)-4-hydroxyproline

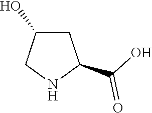

Hse(Me)=O-methyl-L-homoserine

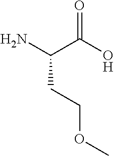

αMeTrp=α-methyltryptophan=

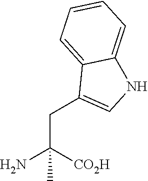 [SMILES: C[C@](N)(Cc1c[nH]c2ccccc12)C(=O)O]

In some embodiments, GRP-10 (human GRP18-27, CAS Number: 81608-30-2) has the following sequence: G-N-H-W-A-V-G-H-L-M-NH₂ (SEQ ID NO. 44).

In some embodiments, human GRP (CAS Number: 93755-85-2) has the following sequence: V-P-L-P-A-G-G-G-T-V-L-T-K-M-Y-P-R-G-N-H-W-A-V-G-H-L-M-NH₂ (SEQ ID NO. 45)

### Pharmaceutical Compositions

In some embodiments, described herein is a pharmaceutical composition comprising: (i) a peptide described herein, or a pharmaceutically acceptable salt, solvate, or isotope thereof, and (ii) a pharmaceutically acceptable carrier, diluent, or excipient.

In some embodiments, the pharmaceutical composition further comprises an absorption enhancer. In some embodiments, the absorption enhancer is for use in an oral formulation. In some embodiments, the absorption enhancer comprises sodium caprate, sodium caprylate, labrasol, or sodium N-(8-[2-hydroxylbenzoyl]amino) caprylate.

### Methods of Treating

In some embodiments, described herein is a use of a peptide described herein, or a pharmaceutical composition comprising a peptide described herein, for the preparation of a medicament for the treatment of a disorder. In some embodiments, the disorder is a gastrointestinal disease. In some embodiments, the disorder is obesity.

In some embodiments of the methods described herein, the disorder is selected from the group consisting of: central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, and diabetic neuropathy; metabolic syndrome, obesity and co-morbidities including cardiovascular disease such as hypertension, heart failure and atrial fibrillation, dyslipidemia, sleep apnea, and osteoarthritis; and metabolic-associated steatohepatitis (MASH); eating and nutritional disorders including hyperphagia, cachexia, binge eating disorder, short bowel syndrome, intestinal failure, intestinal insufficiency and other eating disorders; inflammatory disorders and autoimmune diseases such as inflammatory bowel disease, ulcerative colitis, Crohn's disease, psoriasis, and celiac disease; necrotizing enterocolitis; gastrointestinal injury resulting from toxic insults such as radiation or chemotherapy; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, other conditions involving the gut-brain axis.

In some embodiments, the disorder is a metabolic condition including diabetes and its complications such as chronic kidney disease/diabetic nephropathy, diabetic retinopathy, and diabetic neuropathy; metabolic syndrome, obesity and co-morbidities including cardiovascular disease such as hypertension, heart failure and atrial fibrillation, dyslipidemia, sleep apnea, and osteoarthritis; metabolic-associated steatohepatitis (MASH); eating and nutritional disorders including hyperphagia, cachexia, binge eating disorder, and other eating disorders. In some embodiments, the metabolic condition is type 2 diabetes, hyperglycemia, metabolic syndrome, obesity, hypercholesterolemia, or metabolic-associated steatohepatitis. In some embodiments, the metabolic condition is type 2 diabetes. In some embodiments, the metabolic condition is metabolic-associated steatohepatitis. In some embodiments, the metabolic condition is obesity.

In some embodiments, the disorder is weight loss or preventing weight gain or weight regain. In some embodiments, the disorder is weight loss or preventing weight gain or weight regain post-bariatric surgery. In some embodiments, the disorder is weight loss or preventing weight gain or weight regain, wherein the subject has had bariatric surgery.

In some embodiments, described herein is a method for treating a disorder in a subject in need thereof, the method comprising: administering a therapeutically effective amount of a peptide of described herein or a pharmaceutical composition comprising a peptide described herein. The disorder can be a gastrointestinal disease. The disorder can be obesity.

In some embodiments, described herein is a method for weight management in a subject in need thereof, the method comprising: administering a therapeutically effective amount of a peptide described herein or a pharmaceutical composition comprising a peptide described herein.

In some embodiments, described herein is a method for reducing body weight of a subject in need thereof, the method comprising: administering a therapeutically effective amount of a peptide described herein or a pharmaceutical composition comprising a peptide described herein.

In some embodiments, described herein is a method for treating obesity in a subject in need thereof, the method comprising: administering a therapeutically effective amount of a peptide of described herein or a pharmaceutical composition comprising a peptide described herein.

In some embodiments, the administering occurs weekly. In some embodiments, the administering occurs every 7-14 days, every two weeks, every three weeks, or every four weeks.

In some embodiments, the subject is a mammal. In some embodiments, the subject is a human.

In some embodiments, the subject is overweight. In some embodiments, the subject is obese.

In some embodiments, the subject has at least one weight-related comorbid condition selected from the group consisting of: hypertension, type 2 diabetes mellitus, dyslipidemia, metabolic-associated steatohepatitis, sleep apnea, and urinary incontinence.

In some embodiments, the subject has received at least one previous treatment of a weight management therapy.

In some embodiments, administering the peptide reduces a body weight of the subject by at least 5% compared to a body weight of an otherwise identical subject that is not administered the peptide. In some embodiments, administering the peptide reduces body weight of a subject by at least about 5%, at least about 10%, at least about 15%, at least about 20%, or at least about 30% compared to the body weight of an otherwise identical subject that is not administered the peptide. In some embodiments, administering the peptide reduces a body weight of the subject by at least 5% compared to the body weight of the subject at baseline. In some embodiments, administering the peptide reduces body weight of a subject by at least about 5%, at least about 10%, at least about 15%, at least about 20%, or at least about 30% compared to the body weight of the subject at baseline. In some embodiments, the baseline is the body weight of the subject before the subject begins administration of the peptide. For example, baseline can be the body weight of the subject on Day 0 if peptide administration begins on Day 1.

In some embodiments, administering the peptide reduces cumulative food intake of the subject (e.g., within a 24-hour period) by at least about: 5%, 10%, 15%, 20%, 30%, 40%, 50%, 55%, 60%, 65%, 70%, 75%, 80%, 85%, or 90% compared to food intake of the same subject when not administered the peptide.

In some embodiments, a peptide described herein comprises a half-life that is longer than the half-life of an otherwise identical peptide that is not conjugated to the lipid moiety. For example, the half-life can be at least 5-fold, 10-fold, 100-fold, 1000-fold, 1500-fold, or 2000-fold higher. In some embodiments, the half-life of a peptide described herein is at least 100-fold higher compared to a half-life of an otherwise identical peptide with no lipid conjugation. In some embodiments, the half-life of the peptide is at least 1000-fold higher compared to a half-life of an otherwise identical peptide with no lipid conjugation. In some embodiments, the half-life of the peptide is at least 1500-fold higher compared to a half-life of an otherwise identical peptide with no lipid conjugation.

In some embodiments, a peptide described herein comprises a half-life that is longer than the half-life of a reference peptide, for example a GRP peptide (SEQ ID NO. 45) or a GRP-10 peptide (SEQ ID NO. 44). For example, the half-life can be at least 5-fold, 10-fold, 100-fold, 1000-fold, 1500-fold, or 2000-fold higher. In some embodiments, the half-life of a peptide described herein is at least 100-fold higher compared to the half-life of GRP-10 (SEQ ID NO. 44). In some embodiments, the half-life of the peptide is at least 1000-fold higher compared to a half-life of a GRP peptide (SEQ ID NO. 45) or a GRP-10 peptide (SEQ ID NO. 44). In some embodiments, the half-life of the peptide is at least 1500-fold higher compared to a half-life of a GRP peptide (SEQ ID NO. 45) or a GRP-10 peptide (SEQ ID NO. 44).

In some embodiments, a peptide described herein has a reduced clearance as compared to a reference peptide. In some embodiments, the clearance is reduced by at least 2-fold, 5-fold, 10-fold, 100 fold, or 1000-fold. In some embodiments, the reference peptide is GRP (SEQ ID NO. 45), GRP-10 (SEQ ID NO. 44), or a non-lipidated analog.

In some embodiments, a peptide described herein has a solubility between about 1 mg/mL and about 100 mg/mL. In some embodiments, a peptide described herein has a solubility between about 1 mg/mL and 50 mg/mL. In some embodiments, a peptide described herein has a solubility of about 1 mg/mL, about 5 mg/mL, about 10 mg/mL, about 15 mg/mL, or about 20 mg/mL. In some embodiments, a peptide described herein has a solubility of about 25 mg/mL, about 30 mg/mL, about 35 mg/mL, about 40 mg/mL, about 45 mg/mL, or about 50 mg/mL. In some embodiments, the solubility is measured in a buffer. In some embodiments, the buffer is an aqueous buffer. In some embodiments, the buffer is an aqueous phosphate buffer. In some embodiments, the solubility is measured at a pH range of about 4-9. In some embodiments, the solubility is measured at a neutral pH (i.e., pH of about 7). In some embodiments, the solubility is measured at a physiological pH (i.e., pH of about 7.4). In some embodiments, the solubility is measured at a pH of about 8. In some embodiments, the solubility is measured at a pH of about 9. In some embodiments, the solubility is measured at a pH of about 10. In some embodiments, the solubility is measured at a pH of about 4. In some embodiments, the solubility is measured at a pH of about 5. In some embodiments, the solubility is measured at a pH of about 6.

In some embodiments, a peptide described herein is stable in a buffer for up to 2 weeks or greater. In some embodiments, a peptide described herein is stable in a buffer for up to 4 weeks or greater. In some embodiments, a peptide described herein is stable in a buffer for up to 6 weeks or greater. In some embodiments, a peptide described herein is stable in a buffer for up to 12 weeks or greater. In some embodiments, a peptide described herein is stable in a buffer for up to 6 months or greater. In some embodiments, a peptide described herein is stable in a buffer for up to 1 year or greater. In some embodiments, the buffer is an aqueous buffer. In some embodiments, the buffer is an aqueous phosphate buffer. In some embodiments, the buffer has a pH range of about 4-9. In some embodiments, the buffer is at a neutral pH (i.e., pH of about 7). In some embodiments, the buffer is at a physiological pH (i.e., pH of about 7.4). In some embodiments, the buffer has a pH of about 8. In some embodiments, the buffer has a pH of about 9. In some embodiments, the buffer has a pH of about 10. In some embodiments, the buffer has a pH of about 4. In some embodiments, the buffer has a pH of about 5. In some embodiments, the buffer has a pH of about 6.

In some embodiments, a peptide described herein is stable in a buffer at a temperature of about 25° C. In some embodiments, a peptide described herein is stable in a buffer at a temperature of about 30° C. In some embodiments, a peptide described herein is stable in a buffer at a temperature of about 35° C. In some embodiments, a peptide described herein is stable in a buffer at a temperature of about 40° C. In some embodiments, a peptide described herein is stable in a buffer at a temperature of about 45° C. In some embodiments, a peptide described herein is stable in a buffer at a temperature of about 50° C. In some embodiments, the buffer is an aqueous buffer. In some embodiments, the buffer is an aqueous phosphate buffer. In some embodiments, the buffer has a pH range of about 4-9. In some embodiments, the buffer is at a neutral pH (i.e., pH of about 7). In some embodiments, the buffer is at a physiological pH (i.e., pH of about 7.4). In some embodiments, the buffer has a pH of about 8. In some embodiments, the buffer has a pH of about 9. In some embodiments, the buffer has a pH of about 10. In some embodiments, the buffer has a pH of about 4. In some embodiments, the buffer has a pH of about 5. In some embodiments, the buffer has a pH of about 6.

In some embodiments, a peptide described herein is stable and soluble in a buffer. In some embodiments, a peptide described herein has a solubility of at least 50 mg/mL and is stable for up to 4 weeks or greater at 25° C. in a phosphate buffer at pH 8. In some embodiments, a peptide described herein has a solubility of at least 75 mg/mL and is stable for up to 4 weeks or greater at 25° C. in a phosphate buffer at pH 8. In some embodiments, a peptide described herein has a solubility of at least 50 mg/mL and is stable for up to 12 weeks or greater at 25° C. in a phosphate buffer at pH 8. In some embodiments, a peptide described herein has a solubility of at least 75 mg/mL and is stable for up to 12 weeks or greater at 25° C. in a phosphate buffer at pH 8.

In some embodiments, a peptide described herein has a stability (e.g., plasma stability) of greater than about 1 hour, about 2 hours, about 3 hours, about 4 hours, about 5 hours, about 6 hours, about 7 hours, or about 8 hours. In some embodiments, a peptide described herein has a plasma stability of greater than about 12 hours. In some embodiment, a peptide described herein has a plasma stability of greater than about 18 hours. In some embodiments, a peptide described herein has a plasma stability of greater than about 24 hours. In some embodiments, a peptide described herein has a plasma stability of greater than about 30 hours. In some embodiments, a peptide described herein is stable in plasma at a temperature of about 25° C. In some embodiments, a peptide described herein is stable in plasma at a temperature of about 30° C. In some embodiments, a peptide described herein is stable in plasma at a temperature of about 35° C. In some embodiments, a peptide described herein is stable in plasma at a temperature of about 40° C. In some embodiments, the plasma is from a subject, wherein the subject is a human. In some embodiments, the plasma is from a subject, wherein the subject is a mammal. In some embodiments, the plasma is from a subject, wherein the subject is a mouse.

In some embodiments, stability is measured as a function of the percentage peptide remaining at a given time or after a given time interval. In some embodiments, the percentage peptide remaining at a given time or after a given time interval is greater than about 25%. In some embodiments, the percentage peptide remaining at a given time or after a given time interval is greater than about 50%. In some embodiments, the percentage peptide remaining at a given time or after a given time interval is greater than about 75%. In some embodiments, the percentage peptide remaining at a given time or after a given time interval is greater than about 80%. In some embodiments, the percentage peptide remaining at a given time or after a given time interval is greater than about 85%. In some embodiments, the percentage peptide remaining at a given time or after a given time interval is greater than about 90%. In some embodiments, the percentage peptide remaining at a given time or after a given time interval is greater than about 95%. In some embodiments, the percentage peptide remaining at a given time or after a given time interval is greater than about 99%.

In some embodiments, the peptide is administered intravenously. In some embodiments, the peptide is administered subcutaneously. In some embodiments, the peptide is administered orally.

In some embodiments, a method described herein further comprises administering an absorption enhancer. In some embodiments, the absorption enhancer is for use in an oral formulation. In some embodiments, the absorption enhancer is selected from the group consisting of: sodium caprate, sodium caprylate, labrasol, and sodium N-(8-[2-hydroxylbenzoyl]amino) caprylate.

In some embodiments, a method described herein further comprises administering a therapeutically effective amount of another weight loss agent.

### Certain Terminology

Unless otherwise stated, the following terms used in this application have the definitions given below. The use of the term “including” as well as other forms, such as “include”, “includes,” and “included,” is not limiting.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e., groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

The term “acyl,” as used herein refers to the group —C(═O)—R, where R is alkyl, alkenyl, alkynyl, aryl, cycloalkyl, heteroaryl, heterocycle, or any other moiety were the atom attached to the carbonyl is carbon. An “acetyl” group refers to a —C(═O)CH₃ group.

The term “alkenyl,” as used herein refers to a straight-chain or branched-chain hydrocarbon radical having one or more double bonds and containing from 2 to 20 carbon atoms. In certain embodiments, alkenyl includes 2 to 6 carbon atoms. The term “alkenylene” refers to a divalent alkenyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkoxy” refers to a (alkyl)-O— group, where alkyl is as defined herein. In some embodiments, the alkoxy group is a C₁-C₆alkoxy, which refers to a (C₁-C₆alkyl)-O— group. Examples of alkyl groups include methoxy, ethoxy, n-propoxy, isopropoxy, n-butoxy, iso-butoxy, sec-butoxy, tert-butoxy, and the like.

An “alkyl” group refers to an aliphatic hydrocarbon group. In some embodiments, the alkyl is a straight-chain or branched-chain aliphatic hydrocarbon group containing from 1 to 20 carbon atoms. In certain embodiments, alkyl includes 1 to 10 carbon atoms. In further embodiments, the alkyl includes 1 to 8 carbon atoms. Examples of alkyl radicals include methyl, ethyl, n-propyl, isopropyl, n-butyl, isobutyl, sec-butyl, tert-butyl, pentyl, iso-amyl, hexyl, octyl, nonyl, and the like. In some embodiments, an alkyl is a C₁-C₆alkyl. In one aspect the alkyl is methyl, ethyl, propyl, iso-propyl, n-butyl, iso-butyl, sec-butyl, or t-butyl. The term “alkylene” refers to a divalent alkyl, such as methylene (—CH₂—). In some embodiments, an alkylene is a C₁-C₆alkylene. In other embodiments, an alkylene is a C₁-C₄alkylene. Typical alkylene groups include, but are not limited to, —CH₂—, —CH₂CH₂—, —CH₂CH₂CH₂—, —CH₂CH₂CH₂CH₂—, and the like.

The term “amino,” as used herein refers to —NRR′, wherein R and R′ are independently selected from hydrogen, alkyl, acyl, heteroalkyl, aryl, cycloalkyl, heteroaryl, and heterocycloalkyl, any of which may themselves be optionally substituted. Additionally, R and R′may combine to form heterocycloalkyl, either of which may be optionally substituted. In one aspect, “amino” as used herein refers to an —NH₂ group.

The term “alkynyl,” as used herein refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH. The term “alkynylene” refers to a carbon-carbon triple bond attached at two positions such as ethynylene (—C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The term “aromatic” refers to a planar ring having a delocalized π-electron system containing 4n+2 π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

The term “aryl” as used herein means a carbocyclic aromatic system containing one, two or three rings wherein such polycyclic ring systems are fused together. The term “aryl” embraces aromatic groups such as phenyl, naphthyl, anthracenyl, and phenanthryl. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a C₆-C₁₀aryl. Depending on the structure, an aryl group is a monoradical or a diradical (i.e., an arylene group).

The terms “benzo” and “benz,” as used herein refer to fused bicyclic or polycyclic ring system that is formed with benzene as one of the rings. Examples include benzofuran, benzothiophene, and benzimidazole.

The term “cycloalkyl,” as used herein refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In some embodiments, cycloalkyl groups include groups having from 3 to 10 ring atoms. In certain embodiments, said cycloalkyl will comprise from 5 to 7 carbon atoms. In certain embodiments, said cycloalkyl will comprise from 3 to 6 carbon atoms. Examples of such cycloalkyl groups include cyclopropyl, cyclobutyl, cyclopentyl, cyclohexyl, cycloheptyl, tetrahydronaphthyl, indanyl, octahydronaphthyl, 2,3-dihydro-1H-indenyl, adamantly, and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane. In some embodiments, a cycloalkyl is a C₃-C₆cycloalkyl. In some embodiments, a cycloalkyl is a C₃-C₄cycloalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen, and sulfur. In some embodiments, the term “heteroaryl,” as used herein refers to a 3 to 15 membered unsaturated heteromonocyclic ring, or a fused monocyclic, bicyclic, or tricyclic ring system in which at least one of the fused rings is aromatic, which contains at least one atom selected from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, imidazolyl, pyrazolyl, pyridyl, pyrimidinyl, pyrazinyl, pyridazinyl, triazinyl, triazolyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuranyl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl, and the like. Exemplary tricyclic heterocyclic groups include carbazolyl, benzidolyl, phenanthrolinyl, dibenzofuranyl, acridinyl, phenanthridinyl, xanthenyl, and the like. In some embodiments, a heteroaryl contains 0-4 N atoms in the ring. In some embodiments, a heteroaryl contains 1-4 N atoms in the ring. In some embodiments, a heteroaryl contains 1 O atom in the ring. In some embodiments, a heteroaryl contains 1 S atom in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” group refers to a cycloalkyl group that includes at least one heteroatom selected from nitrogen, oxygen, and sulfur. In some embodiments, the term “heterocycloalkyl” as used herein each refer to a saturated, partially unsaturated, or fully unsaturated (but nonaromatic) monocyclic, bicyclic, or tricyclic heterocyclic group containing at least one heteroatom as a ring member, wherein each said heteroatom may be independently selected from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 1-2 N atoms in the ring. In some embodiments, a heterocycloalkyl contains 1-2 O atoms. In some embodiments, a heterocycloalkyl contains 1 S atom. In some embodiments, a heterocycloalkyl contains 0-2 N atoms, 0-2 O atoms and 0-1 S atoms in the ring.

The term “carbamate,” as used herein refers to an ester of carbamic acid (—NHCOO—) which may be attached to the parent molecular moiety from either the nitrogen or acid end, and which may be optionally substituted as defined herein.

The term “carboxyl” or “carboxy,” as used herein, refers to —C(═O)OH or the corresponding “carboxylate” anion, such as is in a carboxylic acid salt.

The term “cyano,” as used herein refers to —CN.

The term “ester,” as used herein refers to a carboxy group bridging two moieties linked at carbon atoms.

The term “ether,” as used herein refers to an oxy group bridging two moieties linked at carbon atoms.

The term “halo,” or “halogen,” as used herein refers to fluorine, chlorine, bromine, or iodine. In some embodiments, halo is fluoro, chloro, or bromo.

The term “haloalkyl,” as used herein refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—), and the like. In one aspect, a haloalkyl is a C₁-C₆haloalkyl. In another aspect, a haloalkyl is a C₁-C₄haloalkyl.

The term “haloalkoxy,” as used herein refers to a haloalkyl group attached to the parent molecular moiety through an oxygen atom. In one aspect, the haloalkoxy is a C₁-C₆haloalkoxy, which refers to a (C₁-C₆haloalkyl)-O— group. In another aspect, the haloalkoxy is a C₁-C₄haloalkoxy, which refers to a (C₁-C₄haloalkyl)-O— group.

The term “heteroalkyl” refers to an alkyl wherein 1 or more carbon atoms are replaced with a heteroatom. In some embodiments, “heteroalkyl” refers to an alkyl wherein 1 or more carbon atoms are replaced with one or more heteroatoms that are independently selected from NH, —N(alkyl), O, S, S(═O) and S(═O)₂. The attachment of the heteroatom(s) to the remainder of the compound is at a carbon atoms of the heteroalkyl. In some embodiments, up to two heteroatoms may be consecutive, such as, for example, —CH₂—NH—OCH₃. In some embodiments, “heteroalkyl” is an “alkoxyalkyl”, “alkylthioalkyl”, or “alkylaminoalkyl”. “Alkoxyalkyl” refers to an alkyl in which one hydrogen atom is replaced by an alkoxy group, as defined herein. In some embodiments, an alkoxyalkyl is a (C₁-C₆alkoxy)-C₁-C₆alkyl. Typical alkoxyalkyl groups include, but are not limited to, —CH₂OCH₃, —CH₂CH₂OCH₃, —CH₂CH₂CH₂OCH₃, —CH₂CH₂CH₂CH₂OCH₃, —CH₂OCH₂CH₃, —CH₂CH₂OCH₂CH₃, —CH₂CH₂CH₂OCH₂CH₃, —CH₂CH₂CH₂CH₂OCH₂CH₃, and the like. “Alkylthioalkyl” refers to an alkyl in which one hydrogen atom is replaced by an alkylthio group, as defined herein. In some embodiments, an alkoxyalkyl is a (C₁-C₆ alkylthio)-C₁-C₆alkyl. Typical alkoxyalkyl groups include, but are not limited to, —CH₂SCH₃, —CH₂CH₂SCH₃, —CH₂CH₂CH₂SCH₃, —CH₂CH₂CH₂CH₂SCH₃, —CH₂SCH₂CH₃, —CH₂CH₂SCH₂CH₃, —CH₂CH₂CH₂SCH₂CH₃, —CH₂CH₂CH₂CH₂SCH₂CH₃, and the like. “Alkylaminoalkyl” refers to an alkyl in which one hydrogen atom is replaced by an alkylamino group, as defined herein. In some embodiments, an alkoxyalkyl is a (C₁-C₆alkylamino)-C₁-C₆alkyl. Typical alkoxyalkyl groups include, but are not limited to, —CH₂NHCH₃, —CH₂CH₂NHCH₃, —CH₂CH₂CH₂NHCH₃, —CH₂CH₂CH₂CH₂NHCH₃, —CH₂NHCH₂CH₃, —CH₂CH₂NHCH₂CH₃, —CH₂CH₂CH₂NHCH₂CH₃, —CH₂CH₂CH₂CH₂NHCH₂CH₃, and the like.

The term “hydroxy,” or “hydroxyl,” as used herein refers to —OH.

The term “hydroxyalkyl,” as used herein refers to a hydroxy group attached to the parent molecular moiety through an alkyl group. In some embodiments, a hydroxyalkyl is a C₁-C₄hydroxyalkyl. Typical hydroxyalkyl groups include, but are not limited to, —CH₂OH, —CH₂CH₂OH, —CH₂CH₂CH₂OH, —CH₂CH₂CH₂CH₂OH, and the like.

The phrase “linear chain of atoms” refers to the longest straight chain of atoms independently selected from carbon, nitrogen, oxygen, and sulfur.

The term “nitro,” as used herein refers to —NO₂.

The term “oxo,” as used herein refers to ═O.

The terms “sulfonate,” “sulfonic acid,” and “sulfonic,” as used herein refer the —SO₃H group and its anion as the sulfonic acid is used in salt formation.

The term “sulfanyl,” as used herein refers to —S—.

The term “sulfinyl,” as used herein refers to —S(═O)—.

The term “sulfonyl,” as used herein refers to a —S(═O)₂—, —S(═O)₂R, or —S(═O)₂R— group, with R as defined herein.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

In some embodiments, the term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “amino acid” as used herein refers to both natural and non-natural amino acids. The term “non-natural amino acid” as used herein refers to an amino acid other than the 20 amino acids that occur naturally in protein. The term “amino acid analog” as used herein refers to a molecule which is structurally similar to an amino acid and which can be substituted for an amino acid in a peptide disclosed herein. Amino acid analogs include, without limitation, 3-amino acids and amino acids where the amino or carboxy group is substituted by a similarly reactive group (e.g., substitution of the primary amine with a secondary or tertiary amine, or substitution of the carboxy group with an ester).

The term “peptide” as used herein refers to a compound that includes two or more amino acids. A peptide described herein can comprise one or more non-natural amino acids.

The term “lipidated” as used herein refers to a group (e.g., a peptide or an amino acid) that is conjugated to a lipid moiety.

As used herein, amino acid residue refers to an amino acid formed upon chemical digestion (hydrolysis) of a polypeptide at its peptide linkages. The amino acid residues described herein are, in certain embodiments, in the “L” isomeric form. Residues in the “D” isomeric form can be substituted for any “L” amino acid residue, as long as the desired functional property is retained by the polypeptide. “—NH₂” refers to the free amino group present at the amino terminus of a polypeptide or the free amino group of a primary amide group present at the carboxyl terminus of a polypeptide. In addition, the phrase “amino acid residue” is broadly defined to include the amino acids listed in Table A and modified and unusual amino acids, such as those listed in Table B and referred to in 37 C.F.R. §§ 1.821-1.822 and incorporated herein by reference.

TABLE A
Amino Acids and Abbreviations

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

TABLE B
Non-Natural Amino Acids or Amino Acid Analogs and Abbreviations

| ABBREVIATION | AMINO ACID | STRUCTURE |
|---|---|---|
| a or D-Ala | D-alanine | 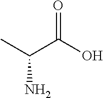 |
| b or β-Ala | β-alanine | 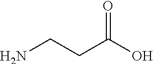 |
| D-Glu | D-glutamic acid | 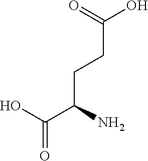 |
| | N-α-acetyllysine | 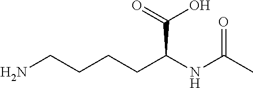 |
| | N-acetylglycine | 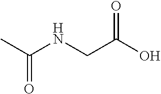 |
| Aib | α-aminoisobutyric acid | 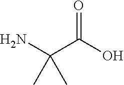 |
| 3PAl | 3-(3-pyridyl)alanine | 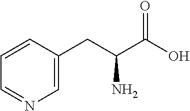 [SMILES: N[C@@H](Cc1cccnc1)C(=O)O] |
| 2PAl | 3-(2-pyridyl)alanine | 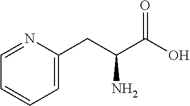 [SMILES: N[C@@H](Cc1ccccn1)C(=O)O] |
| 4PAl | 3-(4-pyridyl)alanine | 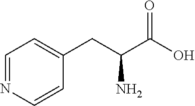 [SMILES: N[C@@H](Cc1ccncc1)C(=O)O] |
| | 3-methylhistidine | 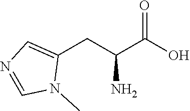 |
| | 1-methylhistidine | 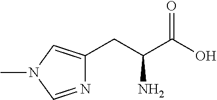 |
| | 3-(1-pyrazolyl)alanine | 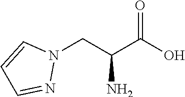 [SMILES: N[C@@H](Cn1cccn1)C(=O)O] |
| | 2-amino-3-(pyrazin-2- yl)propanoic acid | 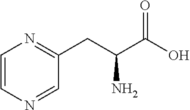 [SMILES: N[C@@H](Cc1cnccn1)C(=O)O] |
| | 2-amino-3-(furan-2- yl)propanoic acid | 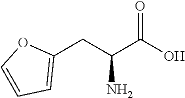 [SMILES: N[C@@H](Cc1ccco1)C(=O)O] |
| 2NAl | 3-(2-naphthyl)-alanine | 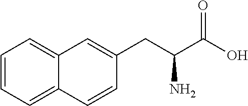 [SMILES: N[C@@H](Cc1ccc2ccccc2c1)C(=O)O] |
| | 2-methylphenylalanine | 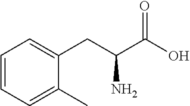 |
| | 3-methylphenylalanine | 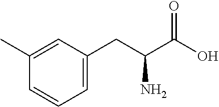 |
| | 4-methylphenylalanine | 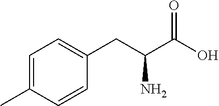 |
| | 2-fluorophenylalanine | 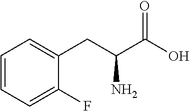 [SMILES: N[C@@H](Cc1ccccc1F)C(=O)O] |
| | 3-fluorophenylalanine | 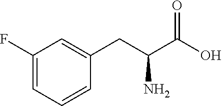 [SMILES: N[C@@H](Cc1cccc(F)c1)C(=O)O] |
| | 4-fluorophenylalanine | 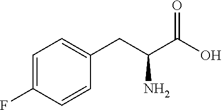 [SMILES: N[C@@H](Cc1ccc(F)cc1)C(=O)O] |
| 2MeW | 2-methyltryptophan | 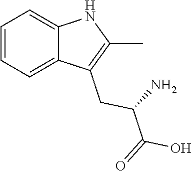 [SMILES: Cc1[nH]c2ccccc2c1C[C@H](N)C(=O)O] |
| 4MeW | 4-methyltryptophan | 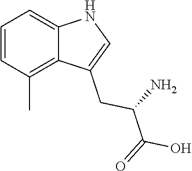 [SMILES: Cc1cccc2[nH]cc(C[C@H](N)C(=O)O)c12] |
| 5MeW | 5-methyltryptophan | 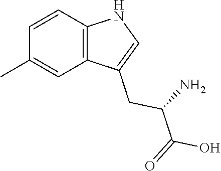 [SMILES: Cc1ccc2[nH]cc(C[C@H](N)C(=O)O)c2c1] |
| 6MeW | 6-methyltryptophan | 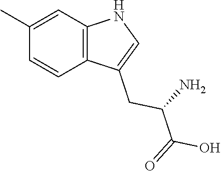 [SMILES: Cc1ccc2c(C[C@H](N)C(=O)O)c[nH]c2c1] |
| 7MeW | 7-methyltryptophan | 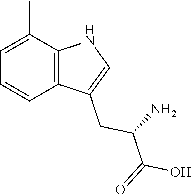 [SMILES: Cc1cccc2c(C[C@H](N)C(=O)O)c[nH]c12] |
| 7FW | 7-fluorotryptophan | 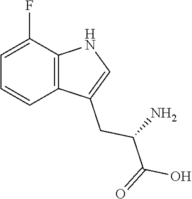 [SMILES: N[C@@H](Cc1c[nH]c2c(F)cccc12)C(=O)O] |
| 6FW | 6-fluorotryptophan | 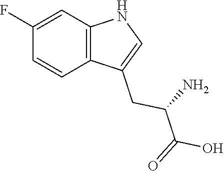 [SMILES: N[C@@H](Cc1c[nH]c2cc(F)ccc12)C(=O)O] |
| Lys(Me)₃ | N-ϵ-trimethyllysine | 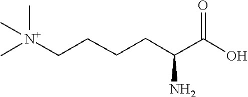 |
| Lys(Ac) | N-ϵ-acetyllysine | 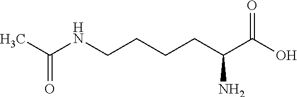 |
| Nle | norleucine | 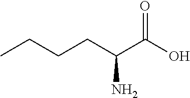 |
| Sar | sarcosine | 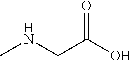 |
| f or D-Phe | D-phenylalanine | 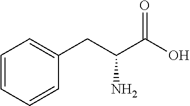 [SMILES: N[C@H](Cc1ccccc1)C(=O)O] |
| h or D-His | D-histidine | 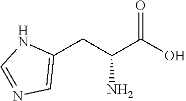 [SMILES: N[C@H](Cc1cnc[nH]1)C(=O)O] |
| Cha | cyclohexylalanine | 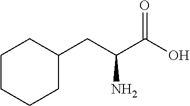 [SMILES: N[C@@H](CC1CCCCC1)C(=O)O] |
| BABA | S-β-aminobutanoic acid | 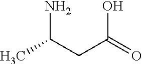 |
| s or D-Ser | D-Serine | 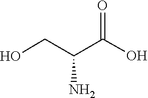 |
| Abu | (S)-2-aminobutanoic acid | 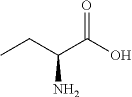 |
| 3AP | (R)-3-aminopentanoic acid | 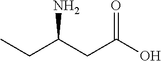 |
| Cpa | L-cyclopropylalanine | 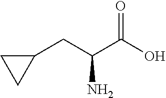 |
| Cbg | L-cyclobutylglycine | 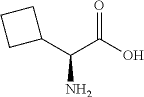 |
| Cpg | L-cyclopropylglycine | 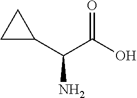 |
| Dap | (S)-2,3-diaminopropanoic acid | 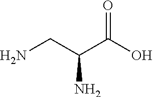 |
| AMBA | (R)-(2-aminomethyl)butanoic acid | 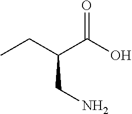 |
| Tba | beta-tert-butyl-L-alanine | 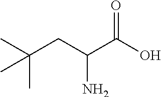 |
| Tle | L-tert-leucine | 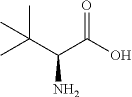 |
| Hva | beta-2-homovaline or (S)-(2-aminomethyl)-3- methylbutanoic acid | 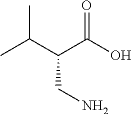 |
| HOV | L-3-hydroxyvaline | 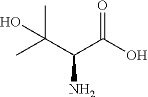 |
| NMeHis | N-methyl-L-histidine | 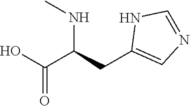 |
| Dap(2CE-PEG-TMEA) | (S)-2-(2-(3-((2-amino-2- carboxyethyl)amino)-3- oxopropoxy)ethoxy)-N,N,N- trimethylethan-1-aminium | 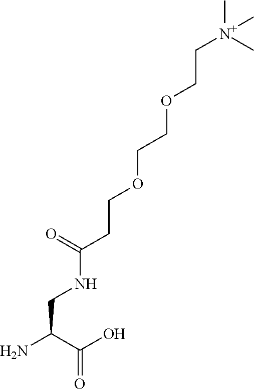 [SMILES: C[N+](C)(C)CCOCCOCCC(=O)NC[C@H](N)C(=O)O] |
| Dap(5CTMPA) | (S)-6-((2-amino-2- carboxyethyl)amino)-N,N,N- trimethyl-6-oxohexan-1- aminium | 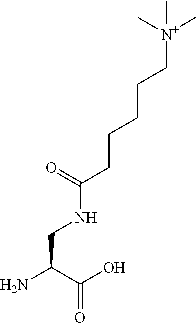 [SMILES: C[N+](C)(C)CCCCCC(=O)NC[C@H](N)C(=O)O] |
| BAIBA | (S)-β-aminoisobutyric acid | 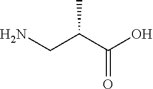 |
| 3A3MBA | 3-amino-3-methylbutanoic acid | 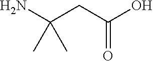 |
| ACPC | (1S,2S)-2- aminocyclopentane-1- carboxylic acid | 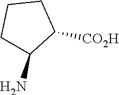 |
| ACPA | 2-(1-aminocyclopropyl)acetic acid | 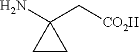 |
| ACBA | 2-(1-aminocyclobutyl)acetic acid | 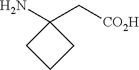 |
| MetO | (2S)-2-amino-4- (methylsulfinyl)butanoic acid or methionine sulfoxide | 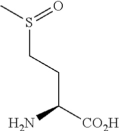 |
| Hyp | (2S,4R)-4-hydroxyproline | 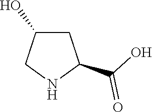 |
| Hse(Me) | O-methyl-L-homoserine | 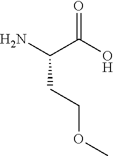 |
| αMeTrp | αMethyltryptophan | 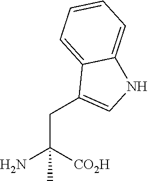 [SMILES: C[C@](N)(Cc1c[nH]c2ccccc12)C(=O)O] |

The term “identity” as used herein can refer to the subunit sequence identity between two polymeric molecules particularly between two amino acid molecules, such as, between two polypeptide molecules, two peptide molecules, or two polynucleotide molecules. When two amino acid sequences have the same residues at the same positions, e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions, e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical. In embodiments, “percent sequence identity” means that two nucleotide sequences or two amino acid sequences, when optimally aligned, such as by the programs GAP or BESTFIT using default gap weights, share at least, e.g., 70% sequence identity, or at least 80% sequence identity, or at least 85% sequence identity, or at least 90% sequence identity, or at least 95% sequence identity or more. For sequence comparison, one sequence can act as a reference sequence (e.g., parent sequence), to which test sequences are compared. When using a sequence comparison algorithm, test and reference sequences are input into a computer, subsequence coordinates are designated, if necessary, and sequence algorithm program parameters are designated. The sequence comparison algorithm can then calculate the percent sequence identity for the test sequence(s) relative to the reference sequence, based on the designated program parameters.

The term “percent identity” refers to a comparison between two nucleic acid or amino acid sequences. Such comparisons are measured using any number of alignment methods known in the art, including but not limited to global (e.g., Needleman-Wunsch algorithm) or local alignments (e.g., Smith-Waterman, Sellers, or other algorithm). Percent identity often refers to the percentage of matching positions of two sequences for a contiguous section of positions, wherein the two sequences are aligned in such a way to maximize matching positions and minimize gaps of non-matching positions. In some instances, alignments are conducted wherein there are no gaps between the two sequences. In some instances, the alignment results in less than 5% gaps, less than 3% gaps, or less than 1% gaps. Additional methods of sequence comparison or alignment are also consistent with the disclosure.

The term “substantially identical” means a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In some embodiments, such a sequence is at least 50%, least 60%, at least 70%, at least 75%, at least 80%, at least 85%, at least 90%, at least 95%, at least 97%, at least 99%, at least 99.99% or 100% identical at the amino acid level or nucleic acid to the sequence used for comparison. Sequence identity is typically measured using sequence analysis software. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). Conservative substitutions may include substitutions within the following groups: glycine, alanine; valine, isoleucine, leucine; aspartic acid, glutamic acid, asparagine, glutamine; serine, threonine; lysine, arginine; and phenylalanine, tyrosine.

The term “disease” or “disorder” as used herein refers to any condition that impairs the normal functioning of the body, such as a functional abnormality or disturbance that impairs normal functioning.

The term “combination therapy” means the administration of two or more therapeutic agents to treat a therapeutic condition or disorder described in the present disclosure.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, “treating,” “treatment,” and the like means ameliorating a disease, so as to reduce, ameliorate, or eliminate its cause, its progression, its severity, or one or more of its symptoms, or otherwise beneficially alter the disease in a subject. In certain embodiments, reference to “treating” or “treatment” of a subject at risk for developing a disease, or at risk of disease progression to a worse state, is intended to include prophylaxis. Prevention of a disease may involve complete protection from disease or may involve prevention of disease progression. Prevention of diseases may also mean prevention of progression of a disease to a later stage of the disease.

The term “patient” is generally synonymous with the term “subject” and includes all mammals including humans. Examples of patients include humans, non-human primates such as chimpanzees, and other apes and monkey species; livestock such as cows, goats, sheep, pigs, and rabbits, and companion animals such as dogs, cats, rabbits, and horses. Preferably, the patient is a human.

In some embodiments, a lipid moiety described herein comprises a fatty acid group, or corresponding fatty acyl group, as shown in Table C.

TABLE C
Fatty Acids

| FATTY ACID | STRUCTURE |
|---|---|
| Palmitic acid | 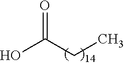 |
| Octadecanoic acid | 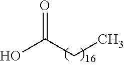 |
| Octadecanedioic acid | 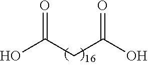 |
| Eicosanedioic acid | 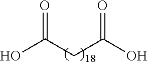 |

### Further Forms of Peptides

In one aspect, peptides described herein are in the form of pharmaceutically acceptable salts. As well, active metabolites of these peptides having the same type of activity are included in the scope of the present disclosure. In addition, the peptides described herein can exist in unsolvated as well as solvated forms with pharmaceutically acceptable solvents such as water, ethanol, and the like. The solvated forms of the peptides presented herein are also considered to be disclosed herein.

“Pharmaceutically acceptable,” as used herein, refers a material, such as a carrier or diluent, which does not abrogate the biological activity or properties of the peptide, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble than non-ionic species. In some embodiments, a pharmaceutically acceptable salt of a peptide disclosed herein is for oral administration. In some embodiments, a pharmaceutically acceptable salt of a peptide disclosed herein is for intravenous administration. In some embodiments, a pharmaceutically acceptable salt of a peptide disclosed herein is for subcutaneous administration. In some embodiments, pharmaceutically acceptable salts are obtained by reacting a peptide disclosed herein with an acid. In some embodiments, the peptide disclosed herein (i.e., free base form) is basic and is reacted with an organic acid or an inorganic acid.

In some embodiments, pharmaceutically acceptable salts are obtained by reacting a peptide disclosed herein with a base. In some embodiments, the peptide disclosed herein is acidic and is reacted with a base. In such situations, an acidic proton of the peptide disclosed herein is replaced by a metal ion, e.g., lithium, sodium, potassium, magnesium, calcium, or an aluminum ion. In some cases, peptides described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, peptides described herein form salts with amino acids such as, but not limited to, arginine, lysine, and the like. Acceptable inorganic bases used to form salts with peptides that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the peptides provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of peptides described herein are conveniently prepared or formed during the processes described herein. In addition, the peptides provided herein optionally exist in unsolvated as well as solvated forms.

In some embodiments, sites on the organic radicals (e.g., alkyl groups, aromatic rings) of peptides disclosed herein are susceptible to various metabolic reactions. Incorporation of appropriate substituents on the organic radicals will reduce, minimize, or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the peptides described herein are labeled isotopically (e.g., with a radioisotope) or by another other means, including, but not limited to, the use of chromophores or fluorescent moieties, bioluminescent labels, or chemiluminescent labels.

Peptides described herein include isotopically-labeled peptides, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present peptides include isotopes of hydrogen, carbon, nitrogen, oxygen, sulfur, fluorine chlorine, iodine, phosphorus, such as, for example, ²H, ³H, ¹³C, ¹⁴C, ⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled peptides described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the peptides disclosed herein possess one or more stereocenters and each stereocenter exists independently in either the R or S configuration. The peptides presented herein include all diastereomeric, individual enantiomers, atropisomers, and epimeric forms as well as the appropriate mixtures thereof. The peptides and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents.

### Pharmaceutical Compositions and Formulations

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tableting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound provided herein in a suitable liquid vehicle and filter sterilizing the solution before filling and sealing an appropriate vial or ampule. These are just a few examples of the many appropriate methods well known in the art for preparing dosage forms.

A compound of the present invention can be formulated into pharmaceutical compositions using techniques well known to those in the art. Suitable pharmaceutically acceptable carriers, outside those mentioned herein, are known in the art; for example, see Remington, The Science and Practice of Pharmacy, 20th Edition, 2000, Lippincott Williams & Wilkins, (Editors: Gennaro et. al.).

The compounds provided herein, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension, or liquid. The pharmaceutical composition is preferably made in the form of a dosage unit containing a particular amount of the active ingredient. Examples of such dosage units are capsules, tablets, powders, granules, or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. In some embodiments, the pharmaceutical composition further comprises an absorption enhancer. In some embodiments, the absorption enhancer comprises sodium caprate, sodium caprylate, labrasol, or sodium N-(8-[2-hydroxylbenzoyl]amino) caprylate.

The active ingredient may also be administered by injection as a composition wherein, for example, saline, dextrose, or water may be used as a suitable pharmaceutically acceptable carrier. Compounds provided herein or a salt, solvate, or hydrate thereof can be used as active ingredients in pharmaceutical compositions. The term “active ingredient”, defined in the context of a “pharmaceutical composition”, refers to a component of a pharmaceutical composition that provides the primary pharmacological effect, as opposed to an “inactive ingredient” which would generally be recognized as providing no pharmaceutical benefit.

The dose when using the compounds provided herein can vary within wide limits and as is customary and is known to the physician or other clinician, it is to be tailored to the individual conditions in each individual case. It depends, for example, on the nature and severity of the illness to be treated, on the condition of the patient, on the compound employed or on whether an acute or chronic disease state is treated, or prophylaxis conducted, or on whether further active compounds are administered in addition to the compounds provided herein. Multiple doses may be administered during the day, especially when relatively large amounts are deemed to be needed, for example 2, 3, or 4 doses. Depending on the individual and as deemed appropriate from the healthcare provider it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated, or prophylaxis conducted, or on whether further active compounds are administered in addition to the compounds provided herein and as part of a drug combination. The dosage regimen for treating a disease condition with the compounds and/or compositions provided herein is selected in accordance with a variety of factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods provided herein.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents.

Aqueous formulations suitable for oral use can be prepared by dissolving or suspending the active component in water and adding suitable colorants, flavors, stabilizing and thickening agents, as desired.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

For topical administration to the epidermis the compounds provided herein may be formulated as ointments, creams, or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

### Methods of Dosing and Treatment Regimens

In one embodiment, the compounds disclosed herein, or a pharmaceutically acceptable salt or solvate thereof, are used in the preparation of medicaments for the treatment of diseases or conditions in a mammal. Methods for treating any of the diseases or conditions described herein in a mammal in need of such treatment, involves administration of pharmaceutical compositions that include at least one compound disclosed herein, or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound disclosed herein, or a pharmaceutically acceptable salt or solvate thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound disclosed herein, or a pharmaceutically acceptable salt or solvate thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered subcutaneously to the mammal; and/or (g) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In certain instances, it is appropriate to administer at least one compound disclosed herein, or a pharmaceutically acceptable salt or solvate thereof, in combination with one or more other therapeutic agents.

### Synthesis of Compounds

Compounds described herein are synthesized using standard synthetic techniques or using methods known in the art in combination with methods described herein.

Unless otherwise indicated, conventional methods of mass spectroscopy, NMR, HPLC are employed.

Compounds are prepared using standard organic chemistry techniques such as those described in, for example, March's Advanced Organic Chemistry, 6^{th} Edition, John Wiley and Sons, Inc. Compounds may also be prepared using solid-phase peptide synthesis techniques such as those described in, for example, Solid Phase Peptide Synthesis, 2^{nd} Edition, The Pierce Chemical Co., Rockford, Ill. (1984). Alternative reaction conditions for the synthetic transformations described herein may be employed such as variation of solvent, reaction temperature, reaction time, as well as different chemical reagents and other reaction conditions.

A peptide of the present disclosure may be prepared through known methods, including solid-phase peptide synthesis (SPPS). (see Palomo, Jose M. (2014). “Solid-phase peptide synthesis: an overview focused on the preparation of biologically relevant peptides” RSC Adv. 4 (62): 32658-32672; Krchňák, V; Holladay, Mark W. (2002). “Solid Phase Heterocyclic Chemistry” Chemical Reviews. 102 (1): 61-92; Merrifield, B. (1986-04-18). “Solid phase synthesis” Science. 232 (4748): 341-347; Guillier, F; et al., (2000). “Linkers and Cleavage Strategies in Solid-Phase Organic Synthesis and Combinatorial Chemistry.” Chemical Reviews. 100 (6): 2091-2158; Amblard M, et al., “Methods and protocols of modern solid phase Peptide synthesis.” Mol Biotechnol. 2006 July; 33(3):239-54).

In some embodiments, the solid-phase peptide synthesis is Fmoc solid-phase peptide synthesis. (see, e.g., Behrendt, R., et al., (2016) Advances in Fmoc solid-phase peptide synthesis. J. Pept. Sci., 22: 4-27).

### NUMBERED EMBODIMENTS

Embodiment 1. A peptide comprising an amino acid sequence of Formula (I):

X1-X2-Ar-X4-X5-V-X7-X8-L-X10-X11-X12

or a pharmaceutically acceptable salt, solvate, or isotope thereof, wherein:
X4, X5, X7, and X8 are each independently a natural or non-natural amino acid or amino acid analog;
Ar is an aromatic amino acid;
X1, X2, X10, X11, and X12 are each independently a natural or non-natural amino acid or amino acid analog, or absent;
wherein the peptide is conjugated to one or more lipid moieties.

Embodiment 2. The peptide of embodiment 1, or a pharmaceutically acceptable salt, solvate, or isotope thereof, wherein:
when X1 is absent and X2 is a natural or non-natural amino acid or amino acid analog, X4 is Ala, Arg, Asn, Asp, Cys, Glu, Gln, Gly, His, Ile, Lys, Met, Phe, Pro, Ser, Thr, Trp, Tyr, Val, or a non-natural amino acid or amino acid analog; and
when X1 and X2 are each absent, X5 is Ala, Arg, Asn, Asp, Cys, Glu, Gln, Gly, His, Ile, Lys, Met, Phe, Pro, Ser, Thr, Trp, Tyr, Val, or a non-natural amino acid or amino acid analog.

Embodiment 3. The peptide of embodiment 1, or a pharmaceutically acceptable salt, solvate, or isotope thereof, wherein:
when X1 is absent and X2 is a natural or non-natural amino acid or amino acid analog, X7 is Ala, Arg, Asn, Asp, Cys, Glu, Gln, Gly, His, Ile, Leu, Lys, Met, Phe, Pro, Ser, Trp, Tyr, Val, or a non-natural amino acid or amino acid analog; and
when X1 and X2 are each absent, X8 is Ala, Arg, Asn, Asp, Cys, Glu, Gln, Gly, His, Ile, Leu, Lys, Met, Phe, Pro, Ser, Trp, Tyr, Val, or a non-natural amino acid or amino acid analog.

Embodiment 4. The peptide of embodiment 1, or a pharmaceutically acceptable salt, solvate, or isotope thereof, wherein:
when X1 is absent and X2 is a natural or non-natural amino acid or amino acid analog, X10 is Ala, Arg, Asn, Asp, Cys, Glu, Gln, Gly, His, Ile, Leu, Lys, Met, Pro, Ser, Thr, Trp, Tyr, Val, or a non-natural amino acid or amino acid analog; and
when X1 and X2 are each absent, X11 is Ala, Arg, Asn, Asp, Cys, Glu, Gln, Gly, His, Ile, Leu, Lys, Met, Pro, Ser, Thr, Trp, Tyr, Val, or a non-natural amino acid or amino acid analog.

Embodiment 5. A peptide comprising an amino acid sequence of Formula (V):

X0-X1-X2-Ar-X4-X5-V-X7-X8-L-X10-X11-X12

or a pharmaceutically acceptable salt, solvate, or isotope thereof, wherein:
X0, X1, X2, X4, X5, X7, and X8 are each independently a natural or non-natural amino acid or amino acid analog;
Ar is an aromatic amino acid;
X10, X11, and X12 are each independently a natural or non-natural amino acid or amino acid analog, or absent;
wherein the peptide is conjugated to one or more lipid moieties.

Embodiment 6. The peptide of embodiment 5, or a pharmaceutically acceptable salt, solvate, or isotope thereof, wherein:
X2 is Ala, Arg, Asn, Asp, Cys, Glu, Gln, Gly, His, Ile, Lys, Met, Phe, Pro, Ser, Thr, Trp, Tyr, Val, or a non-natural amino acid or amino acid analog.

Embodiment 7. The peptide of embodiment 5, or a pharmaceutically acceptable salt, solvate, or isotope thereof, wherein:
X5 is Ala, Arg, Asn, Asp, Cys, Glu, Gln, Gly, His, Ile, Leu, Lys, Met, Phe, Pro, Ser, Trp, Tyr, Val, or a non-natural amino acid or amino acid analog.

Embodiment 8. The peptide of embodiment 5, or a pharmaceutically acceptable salt, solvate, or isotope thereof, wherein:
X8 is Ala, Arg, Asn, Asp, Cys, Glu, Gln, Gly, His, Ile, Leu, Lys, Met, Pro, Ser, Thr, Trp, Tyr, Val, or a non-natural amino acid or amino acid analog.

Embodiment 9. The peptide of any one of embodiments 5-8, or a pharmaceutically acceptable salt, solvate, or isotope thereof, wherein:
X0 is Glu, Arg, or Ser.

Embodiment 10. The peptide of any one of embodiments 5-8, or a pharmaceutically acceptable salt, solvate, or isotope thereof, wherein:
X0 is Glu.

Embodiment 11. The peptide of any one of embodiments 1-4, or a pharmaceutically acceptable salt, solvate, or isotope thereof, wherein:
X1 is absent, Gly, Ala, D-alanine, N-acetylglycine, Lys(Ac), N-α-acetyllysine, α-aminoisobutyric acid (Aib), Arg, Phe, D-serine, or D-phenylalanine.

Embodiment 12. The peptide of any one of embodiments 1-4, or a pharmaceutically acceptable salt, solvate, or isotope thereof, wherein:
X1 is absent,
Embodiment 13. The peptide of any one of embodiments 1-10, or a pharmaceutically acceptable salt, solvate, or isotope thereof, wherein:
X1 is Gly, Aib, Arg, Phe, D-Phe, D-Ser, Ser, Pro, Hyp.

Embodiment 14. The peptide of any one of embodiments 1-10, or a pharmaceutically acceptable salt, solvate, or isotope thereof, wherein:
X1 is Gly, α-aminoisobutyric acid (Aib), Arg, Phe, or D-phenylalanine.

Embodiment 15. The peptide of any one of embodiments 1-10, or a pharmaceutically acceptable salt, solvate, or isotope thereof, wherein:
X1 is Phe, D-phenylalanine, or Gly.

Embodiment 16. The peptide of any one of embodiments 1-10, or a pharmaceutically acceptable salt, solvate, or isotope thereof, wherein:
X1 is D-Ser, Ser, Gly.

Embodiment 17. The peptide of any one of embodiments 1-10, or a pharmaceutically acceptable salt, solvate, or isotope thereof, wherein:
X1 is Phe.

Embodiment 18. The peptide of any one of embodiments 1-10, or a pharmaceutically acceptable salt, solvate, or isotope thereof, wherein:
X1 is D-phenylalanine.

Embodiment 19. The peptide of any one of embodiments 1-10, or a pharmaceutically acceptable salt, solvate, or isotope thereof, wherein:
X1 is D-serine.

Embodiment 20. The peptide of any one of embodiments 1-10, or a pharmaceutically acceptable salt, solvate, or isotope thereof, wherein:
X1 is Ser.

Embodiment 21. The peptide of any one of embodiments 1-10, or a pharmaceutically acceptable salt, solvate, or isotope thereof, wherein:
X1 is Gly.

Embodiment 22. The peptide of any one of embodiments 1-21, or a pharmaceutically acceptable salt, solvate, or isotope thereof, wherein:
X2 is Asn, Glu, Lys, Arg, Lys(Ac), Ala, Asp, Gln, Dap, Dap(5CTMPA), Dap(2CE-PEG-TMEA).

Embodiment 23. The peptide of any one of embodiments 1-21, or a pharmaceutically acceptable salt, solvate, or isotope thereof, wherein:
X2 is Ala, D-alanine, α-aminoisobutyric acid (Aib), Lys, N-ε-acetyl-lysine, N-α-acetyl-lysine, Gly, Asn, Glu, Gln, Arg, or Asp.

Embodiment 24. The peptide of any one of embodiments 1-21, or a pharmaceutically acceptable salt, solvate, or isotope thereof, wherein:
X2 is Asn, Glu, Arg, Ala, Asp, Gln, Lys, or Lys(Ac).

Embodiment 25. The peptide of any one of embodiments 1-21, or a pharmaceutically acceptable salt, solvate, or isotope thereof, wherein:
X2 is Glu or Asp.

Embodiment 26. The peptide of any one of embodiments 1-21, or a pharmaceutically acceptable salt, solvate, or isotope thereof, wherein:
X2 is Asn, Arg, or Gln.

Embodiment 27. The peptide of any one of embodiments 1-21, or a pharmaceutically acceptable salt, solvate, or isotope thereof, wherein:
X2 is Asn.

Embodiment 28. The peptide of any one of embodiments 1-21, or a pharmaceutically acceptable salt, solvate, or isotope thereof, wherein:
X2 is Arg.

Embodiment 29. The peptide of any one of embodiments 1-21, or a pharmaceutically acceptable salt, solvate, or isotope thereof, wherein:
X2 is Gln.

Embodiment 30. The peptide of any one of embodiments 1-21, or a pharmaceutically acceptable salt, solvate, or isotope thereof, wherein:
X2 is Glu.

Embodiment 31. The peptide of any one of embodiments 1-30, or a pharmaceutically acceptable salt, solvate, or isotope thereof, wherein:
Ar is an aromatic amino acid selected from Phe, Trp, Tyr, His, 1-methylhistidine, 3-methylhistidine, 3-(3-pyridyl)alanine (3PAl), 3-(2-pyridyl)alanine (2PAl), 3-(4-pyridyl)alanine (4PAl), 3-(1-pyrazolyl)alanine, 2-amino-3-(pyrazin-2-yl)propanoic acid, 2-amino-3-(furan-2-yl)propanoic acid, 3-(2-naphthyl)alanine (2Nal), 2-methylphenylalanine, 3-methylphenylalanine, 4-methylphenylalanine, 2-fluorophenylalanine, 3-fluorophenylalanine, 4-fluorophenylalanine, 2-methyltryptophan (2MeW), 4-methyltryptophan (4MeW), 5-methyltryptophan (5MeW), 6-methyltryptophan (6MeW), or 7-methyltryptophan (7MeW).

Embodiment 32. The peptide of any one of embodiments 1-30, or a pharmaceutically acceptable salt, solvate, or isotope thereof, wherein:
Ar is histidine, 3-(3-pyridyl)alanine (3Pal), 3-(4-pyridyl)alanine (4Pal), or 3-(2-pyridyl)alanine (2Pal).

Embodiment 33. The peptide of any one of embodiments 1-30, or a pharmaceutically acceptable salt, solvate, or isotope thereof, wherein:
Ar is histidine or 3-(3-pyridyl)alanine (3PAl).

Embodiment 34. The peptide of any one of embodiments 1-30, or a pharmaceutically acceptable salt, solvate, or isotope thereof, wherein:
Ar is histidine.

Embodiment 35. The peptide of any one of embodiments 1-30, or a pharmaceutically acceptable salt, solvate, or isotope thereof, wherein:
Ar is 3-(3-pyridyl)alanine (3PAl).

Embodiment 36. The peptide of any one of embodiments 1-35, or a pharmaceutically acceptable salt, solvate, or isotope thereof, wherein:
X4 is Arg, Phe, Trp, Tyr, His, 1-methylhistidine, 3-methylhistidine, 3-(3-pyridyl)alanine (3PAl), 3-(2-pyridyl)alanine (2PAl), 3-(4-pyridyl)alanine (4PAl), 3-(1-pyrazolylalanine, 2-amino-3-(pyrazin-2-yl)propanoic acid, 2-amino-3-(furan-2-yl)propanoic acid, 2-amino-3-(naphthalen-2-yl)propanoic acid, 2-methylphenylalanine, 3-methylphenylalanine, 4-methylphenylalanine, 2-fluorophenylalanine, 3-fluorophenylalanine, 4-fluorophenylalanine, 2-methyltryptophan (2MeW), 4-methyltryptophan (4MeW), 5-methyltryptophan (5MeW), 6-methyltryptophan (6MeW), 7-methyltryptophan (7MeW), 7-fluorotryptophan (7FW), 6-fluorotryptophan (6FW), α-methyltryptophan (αMeW), or 3-(2-naphthyl)alanine (2Nal).

Embodiment 37. The peptide of any one of embodiments 1-35, or a pharmaceutically acceptable salt, solvate, or isotope thereof, wherein:
X4 is Trp, 7MeW, 7FW, 6MeW, 6FW, His, 2Nal, Arg, or αMeW.

Embodiment 38. The peptide of any one of embodiments 1-35, or a pharmaceutically acceptable salt, solvate, or isotope thereof, wherein:
X4 is Arg, His, Trp, 7-methyltryptophan (7MeW), 6-methyltryptophan (6MeW), 7-fluorotryptophan (7FW), 6-fluorotryptophan (6FW), or 3-(2-naphthyl)alanine (2Nal).

Embodiment 39. The peptide of any one of embodiments 1-25, or a pharmaceutically acceptable salt, solvate, or isotope thereof, wherein:
X4 is 7-methyltryptophan (7MeW) or 7-fluorotryptophan (7FW).

Embodiment 40. The peptide of any one of embodiments 1-35, or a pharmaceutically acceptable salt, solvate, or isotope thereof, wherein:
X4 is Trp or 7-methyltryptophan (7MeW).

Embodiment 41. The peptide of any one of embodiments 1-35, or a pharmaceutically acceptable salt, solvate, or isotope thereof, wherein:
X4 is Trp.

Embodiment 42. The peptide of any one of embodiments 1-35, or a pharmaceutically acceptable salt, solvate, or isotope thereof, wherein:
X4 is 7-methyltryptophan (7MeW).

Embodiment 43. The peptide of any one of embodiments 1-42, or a pharmaceutically acceptable salt, solvate, or isotope thereof, wherein:
X5 is Ala, α-aminoisobutyric acid (Aib), Gly, Leu, Val, Ile, Lys, Lys(Ac), Lys(Me)₃, D-alanine, or Nle.

Embodiment 44. The peptide of any one of embodiments 1-42, or a pharmaceutically acceptable salt, solvate, or isotope thereof, wherein:
X5 is Ala, α-aminoisobutyric acid (Aib), or D-alanine.

Embodiment 45. The peptide of any one of embodiments 1-42, or a pharmaceutically acceptable salt, solvate, or isotope thereof, wherein:
X5 is Ala.

Embodiment 46. The peptide of any one of embodiments 1-45, or a pharmaceutically acceptable salt, solvate, or isotope thereof, wherein:
X7 is D-alanine, Aib, Gly, Ala, β-alanine, BABA, BAIBA, 3A3MBA, ACPC, ACPA, or ACBA.

Embodiment 47. The peptide of any one of embodiments 1-45, or a pharmaceutically acceptable salt, solvate, or isotope thereof, wherein:
X7 is 3-Ala or BABA.

Embodiment 48. The peptide of any one of embodiments 1-45, or a pharmaceutically acceptable salt, solvate, or isotope thereof, wherein:
X7 is Gly, D-alanine, β-alanine, Ala, Aib, Leu, Val, Ile, Lys, Lys(Ac), Lys(Me)₃, S-β-aminobutanoic acid (BABA), or Nle.

Embodiment 49. The peptide of any one of embodiments 1-45, or a pharmaceutically acceptable salt, solvate, or isotope thereof, wherein:
X7 is D-alanine, β-alanine, α-aminoisobutyric acid (Aib), Gly, or Ala.

Embodiment 50. The peptide of any one of embodiments 1-45, or a pharmaceutically acceptable salt, solvate, or isotope thereof, wherein:
X7 is Gly, D-alanine, or β-alanine.

Embodiment 51. The peptide of any one of embodiments 1-45, or a pharmaceutically acceptable salt, solvate, or isotope thereof, wherein:
X7 is D-alanine or β-alanine.

Embodiment 52. The peptide of any one of embodiments 1-45, or a pharmaceutically acceptable salt, solvate, or isotope thereof, wherein:
X7 is D-alanine.

Embodiment 53. The peptide of any one of embodiments 1-45, or a pharmaceutically acceptable salt, solvate, or isotope thereof, wherein:
X7 is β-alanine.

Embodiment 54. The peptide of any one of embodiments 1-45, or a pharmaceutically acceptable salt, solvate, or isotope thereof, wherein:
X7 is S-β-aminobutanoic acid (BABA).

Embodiment 55. The peptide of any one of embodiments 1-54, or a pharmaceutically acceptable salt, solvate, or isotope thereof, wherein:
X8 is Phe, Trp, Tyr, His, D-histidine, 1-methylhistidine, 3-methylhistidine, N-methylhistidine (NMeHis), 3-(3-pyridyl)alanine (3PAl), 3-(2-pyridyl)alanine (2PAl), 3-(4-pyridyl)alanine (4PAl), 3-(1-pyrazolyl)alanine, 2-amino-3-(pyrazin-2-yl)propanoic acid, 2-amino-3-(furan-2-yl)propanoic acid, 2-amino-3-(naphthalen-2-yl)propanoic acid, 2-methylphenylalanine, 3-methylphenylalanine, 4-methylphenylalanine, 2-fluorophenylalanine, 3-fluorophenylalanine, 4-fluorophenylalanine, 2-methyltryptophan (2MeW), 4-methyltryptophan (4MeW), 5-methyltryptophan (5MeW), 6-methyltryptophan (6MeW), or 7-methyltryptophan (7MeW).

Embodiment 56. The peptide of any one of embodiments 1-54, or a pharmaceutically acceptable salt, solvate, or isotope thereof, wherein:
X8 is His, 3Pal, Phe, D-His, or NMeHis.

Embodiment 57. The peptide of any one of embodiments 1-54, or a pharmaceutically acceptable salt, solvate, or isotope thereof, wherein:
X8 is His or NMeHis.

Embodiment 58. The peptide of any one of embodiments 1-54, or a pharmaceutically acceptable salt, solvate, or isotope thereof, wherein:
X8 is His, D-histidine, 3-(3-pyridyl)alanine (3Pal), or Phe.

Embodiment 59. The peptide of any one of embodiments 1-54, or a pharmaceutically acceptable salt, solvate, or isotope thereof, wherein:
X8 is His.

Embodiment 60. The peptide of any one of embodiments 1-59, or a pharmaceutically acceptable salt, solvate, or isotope thereof, wherein:
X10 is absent, Nle, Met, Lys(Me)₃, Arg, Cha, Hse(Me), or MetO.

Embodiment 61. The peptide of any one of embodiments 1-59, or a pharmaceutically acceptable salt, solvate, or isotope thereof, wherein:
X10 is absent, Met, Arg, norleucine (Nle), 3-(3-pyridyl)alanine (3Pal), Aib, Leu, Val, Lys, Lys(Ac), Lys(Me)₃, or Ile.

Embodiment 62. The peptide of any one of embodiments 1-59, or a pharmaceutically acceptable salt, solvate, or isotope thereof, wherein:
X10 is absent, Met, Arg, norleucine (Nle), 3-(3-pyridyl)alanine (3Pal), cyclohexylalanine (Cha), or Lys(Me)₃.

Embodiment 63. The peptide of any one of embodiments 1-59, or a pharmaceutically acceptable salt, solvate, or isotope thereof, wherein:
X10 is Met or norleucine (Nle).

Embodiment 64. The peptide of any one of embodiments 1-59, or a pharmaceutically acceptable salt, solvate, or isotope thereof, wherein:
X10 is norleucine (Nle).

Embodiment 65. The peptide of any one of embodiments 1-64, or a pharmaceutically acceptable salt, solvate, or isotope thereof, wherein:
X11 is absent, sarcosine (Sar), Gly, Pro, Ala, D-alanine, α-aminoisobutyric acid (Aib), or Arg.

Embodiment 66. The peptide of any one of embodiments 1-64, or a pharmaceutically acceptable salt, solvate, or isotope thereof, wherein:
X11 is absent, sarcosine (Sar), α-aminoisobutyric acid (Aib), or Arg.

Embodiment 67. The peptide of any one of embodiments 1-64, or a pharmaceutically acceptable salt, solvate, or isotope thereof, wherein:
X11 is sarcosine (Sar).

Embodiment 68. The peptide of any one of embodiments 1-64, or a pharmaceutically acceptable salt, solvate, or isotope thereof, wherein:
X11 is absent.

Embodiment 69. The peptide of any one of embodiments 1-68, or a pharmaceutically acceptable salt, solvate, or isotope thereof, wherein:
X12 is a natural or non-natural amino acid or amino acid analog.

Embodiment 70. The peptide of any one of embodiments 1-68, or a pharmaceutically acceptable salt, solvate, or isotope thereof, wherein:
X12 is absent.

Embodiment 71. The peptide of any one of embodiments 1-70, or a pharmaceutically acceptable salt, solvate, or isotope thereof, wherein the peptide comprises an amino acid sequence of Formula (Vb): X0-X1-X2-H-X4-X5-V-X7-X8-L-X10-X11-X12.

Embodiment 72. The peptide of any one of embodiments 1-4, or a pharmaceutically acceptable salt, solvate, or isotope thereof, wherein the peptide comprises an amino acid sequence of Formula (II): X1-X2-Ar-X4-X5-V-X7-X8-L-X10-X11-X12, or a pharmaceutically acceptable salt, solvate, or isotope thereof, wherein:
X1 is absent, α-aminoisobutyric acid (Aib), Arg, Phe, Gly, Ala, N-acetylglycine, or D-alanine;
X2 is Asn, Glu, Gln, Arg, Ala, Asp, Lys, or Lys(Ac);
Ar is histidine, 3-(3-pyridyl)alanine (3PAl), 3-(4-pyridyl)alanine (4Pal), or 3-(2-pyridyl)alanine (2Pal);
X4 is Arg, His, Trp, 7-methyltryptophan (7MeW), 6-methyltryptophan (6MeW), 7-fluorotryptophan (7FW), 6-fluorotryptophan (6FW), or 3-(2-naphthyl)alanine (2Nal), Phe, D-alanine, or Tyr;
X5 is Ala, α-aminoisobutyric acid (Aib), Gly, Leu, Val, Ile, D-alanine, or Nle;
X7 is Gly, D-alanine, β-alanine, Ala, Aib, Leu, Val, Ile, or Nle;
X8 is His, Phe, D-histidine, or 3-(3-pyridyl)alanine (3PAl);
X10 is absent, Met, Arg, norleucine (Nle), Aib, Leu, Val, Ile, 3-(3-pyridyl)alanine (3Pal), cyclohexylalanine (Cha), or Lys(Me)₃;
X11 is absent, sarcosine (Sar), Gly, Pro, Ala, Arg, D-alanine, or α-aminoisobutyric acid (Aib);
X12 is independently a natural or non-natural amino acid or amino acid analog, or absent.

Embodiment 73. The peptide of any one of embodiments 1-71, or a pharmaceutically acceptable salt, solvate, or isotope thereof, wherein:
X1 is Gly, Glu, α-aminoisobutyric acid (Aib), Arg, Phe, D-phenylalanine, or D-serine;
X2 is Asn, Glu, Arg, Ala, Asp, Gln, or Lys(Ac);
Ar is histidine, 3-(3-pyridyl)alanine (3Pal), 3-(4-pyridyl)alanine (4Pal), or 3-(2-pyridyl)alanine (2Pal);
X4 is Arg, His, Trp, 7-methyltryptophan (7MeW), 6-methyltryptophan (6MeW), 7-fluorotryptophan (7FW), 6-fluorotryptophan (6FW), or 3-(2-naphthyl)alanine (2Nal);
X5 is Ala, α-aminoisobutyric acid (Aib), D-alanine;
X7 is D-alanine, β-alanine, α-aminoisobutyric acid (Aib), Gly, Ala, or S-β-aminobutanoic acid (BABA);
X8 is His, D-histidine, 3-(3-pyridyl)alanine (3Pal), or Phe;
X10 is absent, Met, Arg, norleucine (Nle), 3-(3-pyridyl)alanine (3Pal), or Lys(Me)₃;
X11 is absent, sarcosine (Sar), α-aminoisobutyric acid (Aib), or Arg; and
X12 is absent.

Embodiment 74. The peptide of any one of embodiments 1-71, or a pharmaceutically acceptable salt, solvate, or isotope thereof, wherein:
X1 is Gly, α-aminoisobutyric acid (Aib), Arg, Phe, or D-phenylalanine;
X2 is Asn, Glu, Arg, Ala, Asp, Gln, or Lys(Ac);
Ar is histidine, 3-(3-pyridyl)alanine (3Pal), 3-(4-pyridyl)alanine (4Pal), or 3-(2-pyridyl)alanine (2Pal);
X4 is Arg, His, Trp, 7-methyltryptophan (7MeW), 6-methyltryptophan (6MeW), 7-fluorotryptophan (7FW), 6-fluorotryptophan (6FW), or 3-(2-naphthyl)alanine (2Nal);
X5 is Ala, α-aminoisobutyric acid (Aib), or D-alanine;
X7 is D-alanine, β-alanine, α-aminoisobutyric acid (Aib), Gly, or Ala;
X8 is His, D-histidine, 3-(3-pyridyl)alanine (3Pal), or Phe;
X10 is absent, Met, Arg, norleucine (Nle), 3-(3-pyridyl)alanine (3Pal), or Lys(Me)₃;
X11 is absent, sarcosine (Sar), α-aminoisobutyric acid (Aib), or Arg; and
X12 is absent.

Embodiment 75. The peptide of any one of embodiments 1-71, or a pharmaceutically acceptable salt, solvate, or isotope thereof, wherein:
X1 is Gly, Phe, D-serine, or D-phenylalanine;
X2 is Glu, Asn, Arg, or Gln;
Ar is histidine or 3-(3-pyridyl)alanine (3PAl);
X4 is Trp or 7-methyltryptophan;
X5 is Ala;
X7 is D-alanine, S-β-aminobutanoic acid (BABA), or β-alanine;
X8 is His;
X10 is norleucine (Nle);
X11 is absent; and
X12 is absent.

Embodiment 76. The peptide of any one of embodiments 1-71, or a pharmaceutically acceptable salt, solvate, or isotope thereof, wherein:
X1 is Phe or D-phenylalanine;
X2 is Arg;
Ar is histidine or 3-(3-pyridyl)alanine (3PAl);
X4 is 7-methyltryptophan (7MeW) or 7-fluorotryptophan (7FW);
X5 is Ala;
X7 is D-alanine or β-alanine;
X8 is His;
X10 is norleucine (Nle);
X11 is sarcosine (Sar); and
X12 is absent.

Embodiment 77. The peptide of any one of embodiments 1-71, or a pharmaceutically acceptable salt, solvate, or isotope thereof, wherein:
X1 is D-Ser, Ser, or Gly;
X2 is Asn, Gln, Glu, or Arg;
Ar is histidine;
X4 is 7-methyltryptophan (7MeW) or Trp;
X5 is Ala;
X7 is D-alanine, β-alanine, BABA, or Gly;
X8 is H or NMeHis;
X10 is norleucine (Nle) or Met;
X11 is absent; and
X12 is absent.

Embodiment 78. The peptide of any one of embodiments 1-71, or a pharmaceutically acceptable salt, solvate, or isotope thereof, wherein:
X1 is D-Ser, Ser, or Gly;
X2 is Asn, Gln, Arg, Glu, or Asp;
Ar is histidine;
X4 is 7-methyltryptophan (7MeW) or Trp;
X5 is Ala;
X7 is D-alanine, β-alanine or BABA;
X8 is H or NMeHis;
X10 is norleucine (Nle);
X11 is absent; and
X12 is absent.

Embodiment 79. The peptide of any one of embodiments 1-71, or a pharmaceutically acceptable salt, solvate, or isotope thereof, wherein:
X1 is D-Ser, Ser, or Gly;
X2 is Glu or Asp;
Ar is histidine;
X4 is 7-methyltryptophan (7MeW);
X5 is Ala;
X7 is β-alanine or BABA;
X8 is H or NMeHis;
X10 is norleucine (Nle);
X11 is absent; and
X12 is absent.

Embodiment 79a. The peptide of embodiment 77, or a pharmaceutically acceptable salt, solvate, or isotope thereof, wherein:
X0 is Glu or Ser.

Embodiment 79b. The peptide of any one of embodiments 73, 75, 77, 78, or 79, or a pharmaceutically acceptable salt, solvate, or isotope thereof, wherein:
X0 is Glu.

Embodiment 80. The peptide of embodiment 5, or a pharmaceutically acceptable salt, solvate, or isotope thereof, wherein the peptide of Formula (V) is a peptide of Formula (Va):

X(-1)-X0-X1-X2-Ar-X4-X5-V-X7-X8-L-X10-X11-X12,

or a pharmaceutically acceptable salt, solvate, or isotope thereof, wherein:
X(−1), X0, X1, X2, X4, X5, X7, and X8 are each independently a natural or non-natural amino acid or amino acid analog;
Ar is an aromatic amino acid;
X10, X11, and X12 are each independently a natural or non-natural amino acid or amino acid analog, or absent;
wherein the peptide is conjugated to one or more lipid moieties.

Embodiment 81. The peptide of embodiment 80, or a pharmaceutically acceptable salt, solvate, or isotope thereof, wherein:
X1 is Ala, Arg, Asn, Asp, Cys, Glu, Gln, Gly, His, Ile, Lys, Met, Phe, Pro, Ser, Thr, Trp, Tyr, Val, or a non-natural amino acid or amino acid analog.

Embodiment 82. The peptide of embodiment 80, or a pharmaceutically acceptable salt, solvate, or isotope thereof, wherein:
X4 is Ala, Arg, Asn, Asp, Cys, Glu, Gln, Gly, His, Ile, Leu, Lys, Met, Phe, Pro, Ser, Trp, Tyr, Val, or a non-natural amino acid or amino acid analog.

Embodiment 83. The peptide of embodiment 80, or a pharmaceutically acceptable salt, solvate, or isotope thereof, wherein:
X7 is Ala, Arg, Asn, Asp, Cys, Glu, Gln, Gly, His, Ile, Leu, Lys, Met, Pro, Ser, Thr, Trp, Tyr, Val, or a non-natural amino acid or amino acid analog.

Embodiment 84. The peptide of any one of embodiments 80-83, or a pharmaceutically acceptable salt, solvate, or isotope thereof, wherein:
X(−1) is Gly.

Embodiment 85. The peptide of any one of embodiments 1-84, or a pharmaceutically acceptable salt, solvate, or isotope thereof, wherein the peptide comprises the amino acid sequence of any of the peptides in Tables 1, 1A, 2, or 3.

Embodiment 86. The peptide of any one of embodiments 1-84, or a pharmaceutically acceptable salt, solvate, or isotope thereof, wherein the peptide comprises the amino acid sequence of any one of the peptides in Table 1.

Embodiment 87. The peptide of any one of embodiments 1-84, or a pharmaceutically acceptable salt, solvate, or isotope thereof, wherein the peptide comprises the amino acid sequence of any one of the peptides in Table 1A.

Embodiment 88. The peptide of any one of embodiments 1-84, or a pharmaceutically acceptable salt, solvate, or isotope thereof, wherein the peptide comprises an amino acid sequence comprising at least 50%, 55%, 60%, 65%, 70%, 75%, 80%, 85%, 90%, 95%, 96%, 97%, 98%, 99% or 100% sequence identity to any one of SEQ ID NOs 1-5, 7-26, 46-112, 114-126, 128-134, 136-167, 240-245, 247, 248, or 251-278.

Embodiment 89. The peptide of any one of embodiments 1-84, or a pharmaceutically acceptable salt, solvate, or isotope thereof, wherein the peptide comprises the amino acid sequence of any one of SEQ ID NOs: 1-5, 7-26, 46-112, 114-126, 128-134, 136-167, 240-245, 247, 248, or 251-278.

Embodiment 90. The peptide of any one of embodiments 1-84, or a pharmaceutically acceptable salt, solvate, or isotope thereof, wherein the peptide comprises the amino acid sequence of SEQ ID NOs 59, 140, 241, 242, 245, 247, 248, or 251-278.

Embodiment 91. The peptide of any one of embodiments 1-84, or a pharmaceutically acceptable salt, solvate, or isotope thereof, wherein the peptide comprises an amino acid sequence comprising at least 50%, 55%, 60%, 65%, 70%, 75%, 80%, 85%, 90%, 95%, 96%, 97%, 98%, 99% or 100% sequence identity to any one of the peptides in Table 3.

Embodiment 92. The peptide of any one of embodiments 1-84, or a pharmaceutically acceptable salt, solvate, or isotope thereof, wherein the peptide comprises the amino acid sequence of any one of SEQ ID NOs: 6, 27-43, 166-177, 179-212, 214-218, 220-239, 246, 249, 250, or 279-306.

Embodiment 93. The peptide of any one of embodiments 1-84, or a pharmaceutically acceptable salt, solvate, or isotope thereof, wherein the peptide comprises the amino acid sequence of any one of SEQ ID NOs: 33, 223, 237, 238, 246, 249, 250, or 279-306.

Embodiment 94. The peptide of any one of embodiments 88 or 91, or a pharmaceutically acceptable salt, solvate, or isotope thereof, wherein said sequence identity is determined by a BLASTP, CLUSTALW, MUSCLE, MAFFT algorithm, or a CLUSTALW algorithm with the Smith-Waterman homology search algorithm parameters.

Embodiment 95. The peptide of any one of embodiments 1-94, or a pharmaceutically acceptable salt, solvate, or isotope thereof, wherein the one or more lipid moieties are conjugated to the N-terminus of the peptide.

Embodiment 96. The peptide of any one of embodiments 1-94, or a pharmaceutically acceptable salt, solvate, or isotope thereof, wherein the one or more lipid moieties are conjugated to the C-terminus of the peptide.

Embodiment 97. The peptide of any one of embodiments 1-94, or a pharmaceutically acceptable salt, solvate, or isotope thereof, wherein the one or more lipid moieties are conjugated to the C-terminus of the peptide and the N-terminus of the peptide is substituted with an acetyl group.

Embodiment 98. The peptide of any one of embodiments 1-94, or a pharmaceutically acceptable salt, solvate, or isotope thereof, wherein the one or more lipid moieties are conjugated to or any one of the amino acid residues of Formula (I) or (V) having a suitable functional group for attachment to a lipid moiety.

Embodiment 99. The peptide of any one of embodiments 1-94, or a pharmaceutically acceptable salt, solvate, or isotope thereof, wherein the one or more lipid moieties are conjugated to any one of the amino acid residues of Formula (I) or (V) through the amino acid side chain.

Embodiment 100. The peptide of embodiment 99, or a pharmaceutically acceptable salt, solvate, or isotope thereof, wherein the one or more lipid moieties are conjugated to any one of the lysine residues of Formula (I) or (V).

Embodiment 101. The peptide of any one of embodiments 1-94, or a pharmaceutically acceptable salt, solvate, or isotope thereof, wherein the one or more lipid moieties are conjugated to the N-terminus of the peptide and any one of the lysine residues of Formula (I).

Embodiment 102. The peptide of any one of embodiments 1-101, or a pharmaceutically acceptable salt, solvate, or isotope thereof, wherein the lipid moiety or a lipid moiety of the two or more lipid moieties comprises a C₂-C₂₆ fatty acyl group.

Embodiment 103. The peptide of any one of embodiments 1-101, or a pharmaceutically acceptable salt, solvate, or isotope thereof, wherein the lipid moiety or a lipid moiety of the two or more lipid moieties comprises a saturated fatty acyl group.

Embodiment 104. The peptide of embodiment 103, or a pharmaceutically acceptable salt, solvate, or isotope thereof, wherein the fatty acyl group comprises caprylyl, caprinoyl, lauroyl, myristoyl, palmitoyl, stearoyl, arachidonoyl, behenoyl, lignoceroyl, ceritoyl, octadecanedioyl, or eicosanedioyl.

Embodiment 104a. The peptide of embodiment 103, or a pharmaceutically acceptable salt, solvate, or isotope thereof, wherein the fatty acyl group comprises octadecanedioyl or eicosanedioyl.

Embodiment 105. The peptide of embodiment 103, or a pharmaceutically acceptable salt, solvate, or isotope thereof, wherein the fatty acyl group comprises palmitoyl or stearoyl.

Embodiment 106. The peptide of any one of embodiments 1-105, or a pharmaceutically acceptable salt, solvate, or isotope thereof, wherein the lipid moiety or a lipid moiety of the two or more lipid moieties comprises -(L¹)ₚ-(L²)_{q}-(L³)ᵣ, wherein:
each L¹ is independently —((CH₂)ᵥNR¹)_{w}—CO(CH₂)ₓO(PEG)_{y}(CH₂)_{z}NR¹—.
   PEG is —CH₂CH₂O—;
   v is 2-6;
   w is 0-1;
   x is 1-4;
   y is 1-4;
   z is 2-24;
   R¹ is H or —CH₃;
each L² is independently a natural or unnatural amino acid or amino acid analog;
each L³ is independently a substituted or unsubstituted C₂-C₂₆ fatty acyl group;
p is 0-8;
q is 0-6; and
r is 1 or 2.

Embodiment 107. The peptide of embodiment 106, or a pharmaceutically acceptable salt, solvate, or isotope thereof, wherein:
each L¹ is independently —((CH₂)ᵥNR¹)_{w}—CO(CH₂)ₓO(PEG)_{y}(CH₂)_{z}NR¹—;
each L² is independently a natural or unnatural amino acid or amino acid analog;
each L³ is independently a C₂-C₂₆ fatty acyl group, wherein the C₂-C₂₆ fatty acyl group is unsubstituted or substituted with one or more of oxo, —OH, —C(═O)OH, tetrazolyl, benzoic acid, or naphthoic acid;
p is 0-4;
q is 0-2; and
r is 1 or 2.

Embodiment 108. The peptide of embodiment 106, or a pharmaceutically acceptable salt, solvate, or isotope thereof, wherein:
each L¹ is independently —CO(CH₂)O(PEG)(CH₂)₂NH— or —((CH₂)ᵥNH)_{w}O(CH₂)O(PEG)(CH₂)₂NH—;
each L² is independently glutamic acid, proline, aspartic acid, or 2,4-diaminobutyric acid;
each L³ is independently lauroyl, myristoyl, palmitoyl, stearoyl, or arachidonoyl, wherein lauroyl, myristoyl, palmitoyl, stearoyl, and arachidonoyl are unsubstituted or substituted with one or more of oxo, —OH, —C(═O)OH, tetrazolyl, benzoic acid, or naphthoic acid;
p is 0-4;
q is 0-2; and
r is 1 or 2.

Embodiment 109. The peptide of embodiment 106, or a pharmaceutically acceptable salt, solvate, or isotope thereof, wherein:
each L¹ is independently —CO(CH₂)O(PEG)(CH₂)₂NH— or —((CH₂)₃NH)—CO(CH₂)O(PEG)(CH₂)₂NH—;
each L² is independently glutamic acid;
each L³ is independently palmitoyl or stearoyl, wherein palmitoyl and stearoyl are unsubstituted or substituted with one or more of oxo, —OH, or —C(═O)OH;
p is 0-4;
q is 0-2; and
r is 1 or 2.

Embodiment 110. The peptide of any one of embodiments 1-105, or a pharmaceutically acceptable salt, solvate, or isotope thereof, wherein the lipid moiety or a lipid moiety of the two or more lipid moieties comprises -(L¹)ₚ-(L²)_{q}-(L³)ᵣ, wherein:
each L¹ is OEG;
   OEG is —NH-PEG-PEG-CH₂C(═O)—;
   PEG is —CH₂CH₂O—;
each L² is independently a natural or unnatural amino acid or amino acid analog;
each L³ is independently a substituted or unsubstituted C₂-C₂₆ fatty acyl group;
p is 0-8;
q is 0-6; and
r is 1 or 2.

Embodiment 111. The peptide of embodiment 110, or a pharmaceutically acceptable salt, solvate, or isotope thereof, wherein the lipid moiety or a lipid moiety of the two or more lipid moieties comprises -(L¹)ₚ-(L²)_{q}-(L³)ᵣ, wherein:
each L¹ is OEG;
   OEG is —NH-PEG-PEG-CH₂C(═O)—;
   PEG is —CH₂CH₂O—;
each L² is Glu or D-Glu;
each L³ is independently a substituted or unsubstituted C₂-C₂₆ fatty acyl group;
p is 0-8;
q is 0-6; and
r is 1 or 2.

Embodiment 112. The peptide of embodiment 110, or a pharmaceutically acceptable salt, solvate, or isotope thereof, wherein the lipid moiety or a lipid moiety of the two or more lipid moieties comprises -(L¹)ₚ-(L²)_{q}-(L³)ᵣ, wherein:
each L¹ is OEG;
   OEG is —NH-PEG-PEG-CH₂C(═O)—;
   PEG is —CH₂CH₂O—;
each L² is Glu or D-Glu;
each L³ is independently palmitoyl, octadecanedioyl, or eicosanedioyl;
p is 0-4;
q is 0-2; and
r is 1.

Embodiment 113. The peptide of any one of embodiments 106-112, or a pharmaceutically acceptable salt, solvate, or isotope thereof, wherein -(L¹)ₚ-(L²)_{q}-(L³)ᵣ is

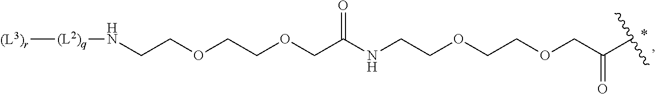

wherein * represents the attachment point to a nitrogen atom of the peptide.

Embodiment 114. The peptide of any one of embodiments 106-112, or a pharmaceutically acceptable salt, solvate, or isotope thereof, wherein -(L¹)ₚ-(L²)_{q}-(L³)ᵣ is

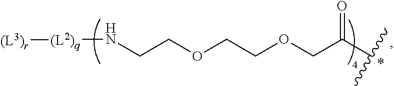

wherein * represents the attachment point to a nitrogen atom of the peptide.

Embodiment 115. The peptide of any one of embodiments 106-112, or a pharmaceutically acceptable salt, solvate, or isotope thereof, wherein -(L¹)ₚ-(L²)_{q}-(L³)ᵣ is

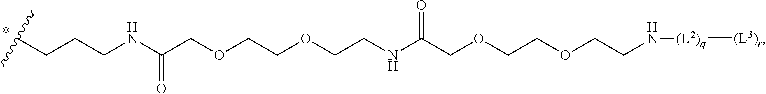

wherein * represents the attachment point to a nitrogen atom of the peptide.

Embodiment 116. The peptide of any one of embodiments 106-115, or a pharmaceutically acceptable salt, solvate, or isotope thereof, wherein -(L¹)ₚ-(L²)_{q}-(L³)ᵣ is

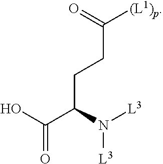

Embodiment 117. The peptide of any one of embodiments 106-115, or a pharmaceutically acceptable salt, solvate, or isotope thereof, wherein -(L¹)ₚ-(L²)_{q}-(L³)ᵣ is

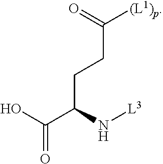

Embodiment 118. The peptide of any one of embodiments 106-117, or a pharmaceutically acceptable salt, solvate, or isotope thereof, wherein L³ is

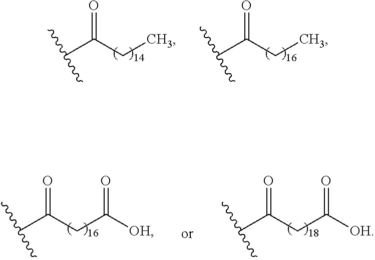

Embodiment 119. The peptide of any one of embodiments 106-117, or a pharmaceutically acceptable salt, solvate, or isotope thereof, wherein L³ is

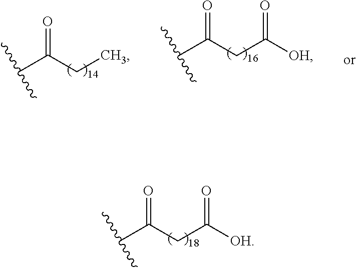

Embodiment 120. The peptide of any one of embodiments 106-117, or a pharmaceutically acceptable salt, solvate, or isotope thereof, wherein -(L¹)ₚ-(L²)_{q}-(L³)ᵣ is -(L¹)₂-(L²)-(L³).

Embodiment 121. The peptide of any one of embodiments 106-120, or a pharmaceutically acceptable salt, solvate, or isotope thereof, wherein -(L¹)ₚ-(L²)_{q}-(L³)ᵣ is

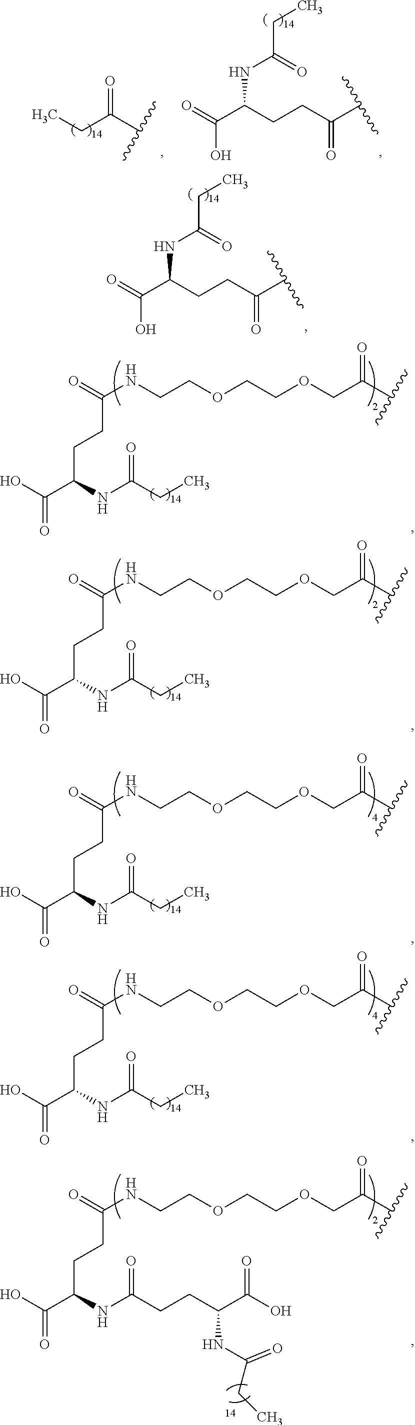

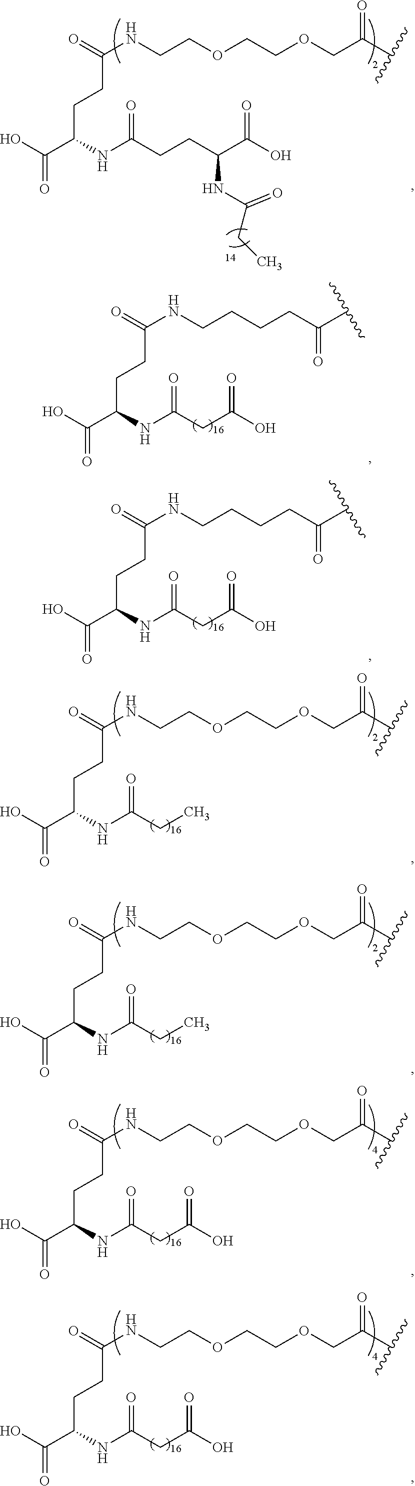

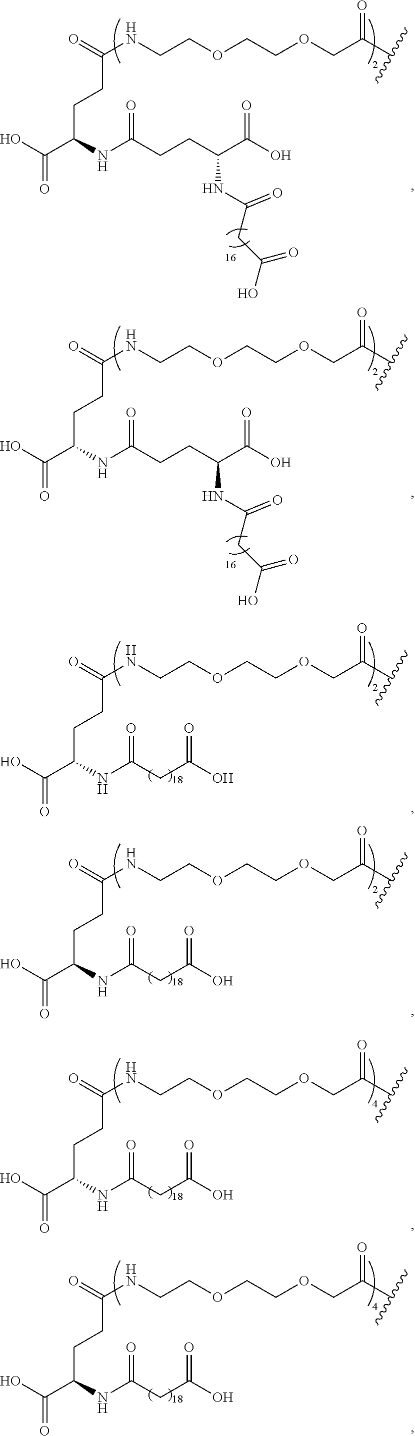

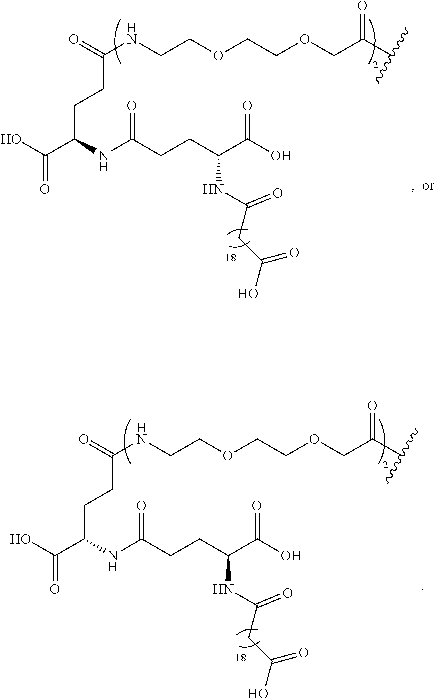

Embodiment 122. The peptide of any one of embodiments 106-120, or a pharmaceutically acceptable salt, solvate, or isotope thereof, wherein -(L¹)ₚ-(L²)_{q}-(L³)ᵣ is

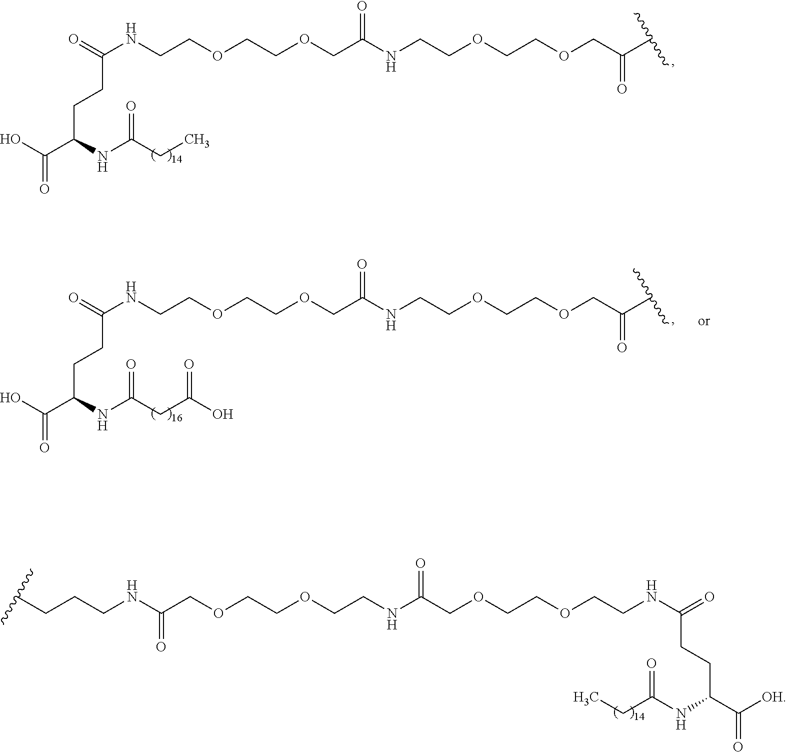

Embodiment 123. The peptide of any one of embodiments 106-120, or a pharmaceutically acceptable salt, solvate, or isotope thereof, wherein -(L¹)ₚ-(L²)_{q}-(L³)ᵣ is

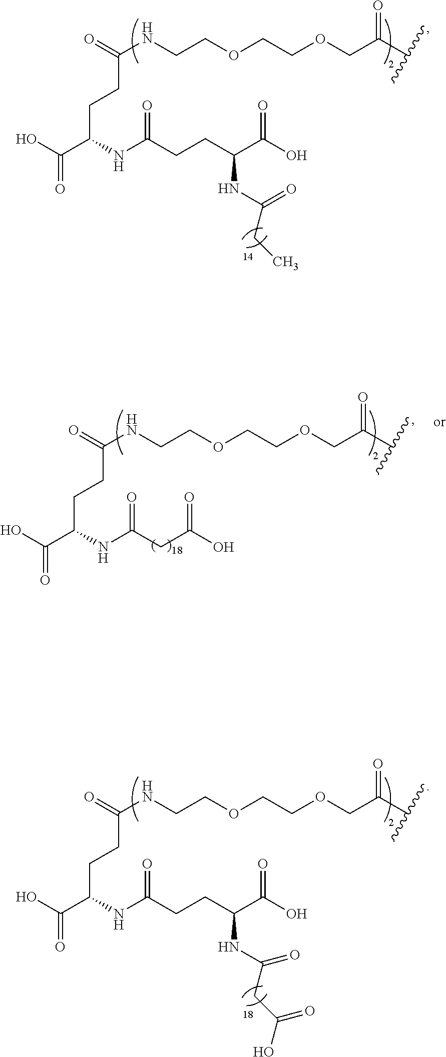

Embodiment 124. The peptide of any one of embodiments 106-120, or a pharmaceutically acceptable salt, solvate, or isotope thereof, wherein -(L¹)ₚ-(L²)_{q}-(L³)ᵣ is °

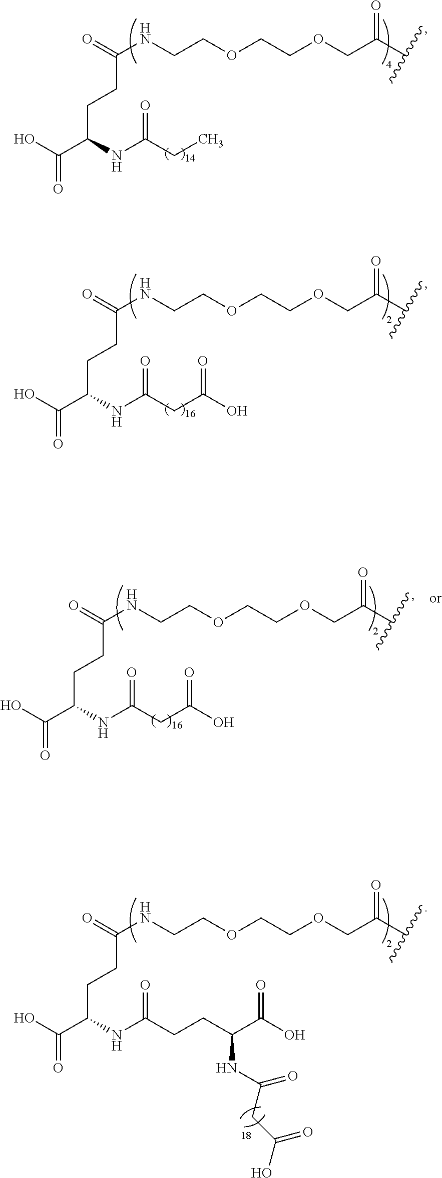

Embodiment 125. The peptide of embodiment 106-124, or a pharmaceutically acceptable salt, solvate, or isotope thereof, wherein the peptide is conjugated to 1 or 2 of -(L¹)ₚ-(L²)_{q}-(L³)ᵣ.

Embodiment 126. The peptide of embodiment 1-125, or a pharmaceutically acceptable salt, solvate, or isotope thereof, wherein the peptide is conjugated to 1 or 2 of

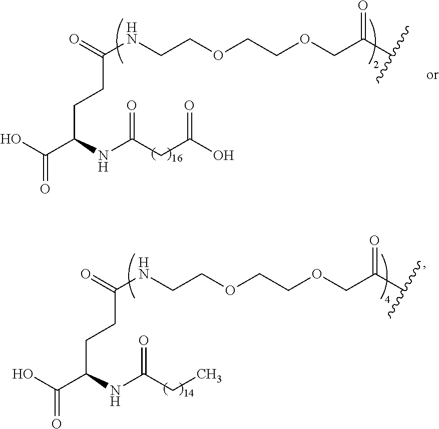

Embodiment 127. The peptide of any one of embodiments 1-125, or a pharmaceutically acceptable salt, solvate, or isotope thereof, wherein the peptide is conjugated to 1 or 2 of

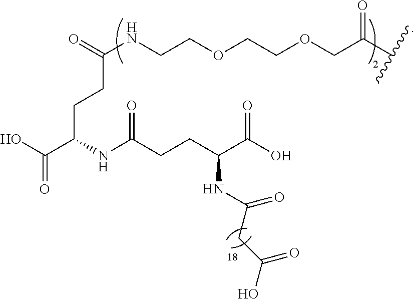

Embodiment 128. The peptide of embodiment 106-120, or a pharmaceutically acceptable salt, solvate, or isotope thereof, wherein the peptide is conjugated to -(L¹)ₚ-(L²)_{q}-(L³)ᵣ through the N-terminus of the peptide and a lysine residue of the peptide.

Embodiment 129. A peptide of Formula (VI):

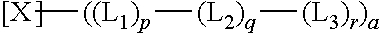

or a pharmaceutically acceptable salt, solvate, or isotope thereof, wherein:
X is a peptide comprising an amino acid sequence of Formula (V):

X0-X1-X2-Ar-X4-X5-V-X7-X8-L-X10-X11-X12,

wherein:
X4, X5, X7, and X8 are each independently a natural or non-natural amino acid or amino acid analog;
Ar is an aromatic amino acid;
X0, X1, X2, X10, X11, and X12 are each independently a natural or non-natural amino acid or amino acid analog, or absent;
each L¹ is independently —((CH₂)ᵥNR¹)_{w}—CO(CH₂)ₓO(PEG)_{y}(CH₂)_{z}NR¹—;
   PEG is —CH₂CH₂O—;
   v is 2-6;
   w is 0-1;
   x is 1-4;
   y is 1-4;
   z is 2-24;
   R¹ is H or —CH₃;
or each L¹ is OEG;
   OEG is —NH-PEG-PEG-CH₂C(═O)—;
   PEG is —CH₂CH₂O—;
each L² is independently a natural or unnatural amino acid or amino acid analog;
each L³ is independently a substituted or unsubstituted C₂-C₂₆ fatty acyl group;
a is 1, 2, 3, or 4;
p is 0-8;
q is 0-6; and
r is 1 or 2.

Embodiment 130. A peptide of Formula (IV):

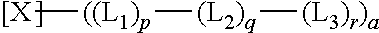

or a pharmaceutically acceptable salt, solvate, or isotope thereof, wherein:
X is a peptide comprising an amino acid sequence of Formula (I):

X1-X2-Ar-X4-X5-V-X7-X8-L-X10-X11-X12,

or a pharmaceutically acceptable salt, solvate, or isotope thereof, wherein:
X4, X5, X7, and X8 are each independently a natural or non-natural amino acid or amino acid analog;
Ar is an aromatic amino acid;
X1, X2, X10, X11, and X12 are each independently a natural or non-natural amino acid or amino acid analog, or absent;
each L¹ is independently —((CH₂)ᵥNR¹)_{w}—CO(CH₂)ₓO(PEG)_{y}(CH₂)_{z}NR¹—;
   PEG is —CH₂CH₂O—;
   v is 2-6;
   w is 0-1;
   x is 1-4;
   y is 1-4;
   z is 2-24;
   R¹ is H or —CH₃;
or each L¹ is OEG;
   OEG is —NH-PEG-PEG-CH₂C(═O)—;
   PEG is —CH₂CH₂O—;
each L² is independently a natural or unnatural amino acid or amino acid analog;
each L³ is independently a substituted or unsubstituted C₂-C₂₆ fatty acyl group;
a is 1, 2, 3, or 4;
p is 0-8;
q is 0-6; and
r is 1 or 2.

Embodiment 131. The peptide of embodiment 129 or 130, or a pharmaceutically acceptable salt, solvate, or isotope thereof, wherein each (-(L¹)ₚ-(L²)_{q}-(L³)ᵣ) is independently attached to the peptide X at the N-terminus of the peptide, the C-terminus of the peptide, or any one of the amino acid residues of Formula (I) or (V) having a suitable functional group for attachment to a lipid moiety.

Embodiment 132. The peptide of embodiment 129 or 130, or a pharmaceutically acceptable salt, solvate, or isotope thereof, wherein a is 2.

Embodiment 133. The peptide of any one of embodiments 129-132, or a pharmaceutically acceptable salt, solvate, or isotope thereof, wherein each (-(L¹)ₚ-(L²)_{q}-(L³)ᵣ) is independently attached to the N-terminus of peptide X.

Embodiment 134. The peptide of any one of embodiments 129-133, or a pharmaceutically acceptable salt, solvate, or isotope thereof, wherein each (-(L¹)ₚ-(L²)_{q}-(L³)ᵣ) is independently attached to any one of the amino acid residues of Formula (I) or (V) having a suitable functional group for attachment to a lipid moiety.

Embodiment 135. The peptide of embodiment 129 or 130, or a pharmaceutically acceptable salt, solvate, or isotope thereof, wherein a is 1.

Embodiment 136. The peptide of any one of embodiments 129-135, or a pharmaceutically acceptable salt, solvate, or isotope thereof, wherein the peptide comprises an amino acid sequence of Formula (II): X1-X2-Ar-X4-X5-V-X7-X8-L-X10-X11-X12, or a pharmaceutically acceptable salt, solvate, or isotope thereof, wherein:
X1 is absent, α-aminoisobutyric acid (Aib), Arg, Phe, Gly, Ala, N-acetylglycine, or D-alanine;
X2 is Asn, Glu, Gln, Arg, Ala, Asp, or Lys(Ac);
Ar is histidine, 3-(3-pyridyl)alanine (3PAl), 3-(4-pyridyl)alanine (4Pal), or 3-(2-pyridyl)alanine (2Pal);
X4 is Arg, His, Trp, 7-methyltryptophan (7MeW), 6-methyltryptophan (6MeW), 7-fluorotryptophan (7FW), 6-fluorotryptophan (6FW), or 3-(2-naphthyl)alanine (2Nal), Phe, D-alanine, or Tyr;
X5 is Ala, α-aminoisobutyric acid (Aib), Gly, Leu, Val, Ile, D-alanine, or Nle;
X7 is Gly, D-alanine, β-alanine, Ala, Aib, Leu, Val, Ile, or Nle;
X8 is His, Phe, D-histidine, or 3-(3-pyridyl)alanine (3PAl);
X10 is absent, Met, Arg, norleucine (Nle), Aib, Leu, Val, Ile, 3-(3-pyridyl)alanine (3Pal), or Lys(Me)₃;
X11 is absent, sarcosine (Sar), Gly, Pro, Ala, Arg, D-alanine, or α-aminoisobutyric acid (Aib); and
X12 is independently a natural or non-natural amino acid or amino acid analog, or absent.

Embodiment 137. The peptide of any one of embodiments 129-135, or a pharmaceutically acceptable salt, solvate, or isotope thereof, wherein:
X1 is absent, Gly, Glu, α-aminoisobutyric acid (Aib), Arg, Phe, D-phenylalanine, or D-serine;
X2 is Asn, Glu, Arg, Ala, Asp, Gln, or Lys(Ac);
Ar is histidine, 3-(3-pyridyl)alanine (3Pal), 3-(4-pyridyl)alanine (4Pal), or 3-(2-pyridyl)alanine (2Pal);
X4 is Arg, His, Trp, 7-methyltryptophan (7MeW), 6-methyltryptophan (6MeW), 7-fluorotryptophan (7FW), 6-fluorotryptophan (6FW), or 3-(2-naphthyl)alanine (2Nal);
X5 is Ala, α-aminoisobutyric acid (Aib), D-alanine;
X7 is D-alanine, β-alanine, α-aminoisobutyric acid (Aib), Gly, Ala, or S-β-aminobutanoic acid (BABA);
X8 is His, D-histidine, 3-(3-pyridyl)alanine (3Pal), or Phe;
X10 is absent, Met, Arg, norleucine (Nle), 3-(3-pyridyl)alanine (3Pal), or Lys(Me)₃;
X11 is absent, sarcosine (Sar), α-aminoisobutyric acid (Aib), or Arg; and
X12 is absent.

Embodiment 138. The peptide of any one of embodiments 129-135, or a pharmaceutically acceptable salt, solvate, or isotope thereof, wherein:
X1 is absent, Gly, α-aminoisobutyric acid (Aib), Arg, Phe, or D-phenylalanine;
X2 is Asn, Glu, Arg, Ala, Asp, Gln, or Lys(Ac);
Ar is histidine, 3-(3-pyridyl)alanine (3Pal), 3-(4-pyridyl)alanine (4Pal), or 3-(2-pyridyl)alanine (2Pal);
X4 is Arg, His, Trp, 7-methyltryptophan (7MeW), 6-methyltryptophan (6MeW), 7-fluorotryptophan (7FW), 6-fluorotryptophan (6FW), or 3-(2-naphthyl)alanine (2Nal);
X5 is Ala, α-aminoisobutyric acid (Aib), or D-alanine;
X7 is D-alanine, β-alanine, α-aminoisobutyric acid (Aib), Gly, or Ala;
X8 is His, D-histidine, 3-(3-pyridyl)alanine (3Pal), or Phe;
X10 is absent, Met, Arg, norleucine (Nle), 3-(3-pyridyl)alanine (3Pal), or Lys(Me)₃;
X11 is absent, sarcosine (Sar), α-aminoisobutyric acid (Aib), or Arg; and
X12 is absent.

Embodiment 139. The peptide of any one of embodiments 129-135, or a pharmaceutically acceptable salt, solvate, or isotope thereof, wherein:
X1 is Gly, Phe, D-serine, or D-phenylalanine;
X2 is Glu, Asn, Arg, or Gln;
Ar is histidine or 3-(3-pyridyl)alanine (3PAl);
X4 is Trp or 7-methyltryptophan;
X5 is Ala;
X7 is D-alanine, S-β-aminobutanoic acid (BABA), or β-alanine;
X8 is His;
X10 is norleucine (Nle);
X11 is absent; and
X12 is absent.

Embodiment 140. The peptide of any one of embodiments 129-135, or a pharmaceutically acceptable salt, solvate, or isotope thereof, wherein:
X1 is Phe or D-phenylalanine;
X2 is Arg;
Ar is histidine or 3-(3-pyridyl)alanine (3PAl);
X4 is 7-methyltryptophan (7MeW) or 7-fluorotryptophan (7FW);
X5 is Ala;
X7 is D-alanine or β-alanine;
X8 is His;
X10 is norleucine (Nle);
X11 is sarcosine (Sar); and
X12 is absent.

Embodiment 141. The peptide of any one of embodiments 129-135, or a pharmaceutically acceptable salt, solvate, or isotope thereof, wherein:
X1 is D-Ser, Ser, or Gly;
X2 is Asn, Gln, Glu, or Arg;
Ar is histidine;
X4 is 7-methyltryptophan (7MeW) or Trp;
X5 is Ala;
X7 is D-alanine, β-alanine, BABA, or Gly;
X8 is H or NMeHis;
X10 is norleucine (Nle) or Met;
X11 is absent; and
X12 is absent.

Embodiment 142. The peptide of any one of embodiments 129-135, or a pharmaceutically acceptable salt, solvate, or isotope thereof, wherein:
X1 is D-Ser, Ser, or Gly;
X2 is Asn, Gln, Arg, Glu, or Asp;
Ar is histidine;
X4 is 7-methyltryptophan (7MeW) or Trp;
X5 is Ala;
X7 is D-alanine, β-alanine or BABA;
X8 is H or NMeHis;
X10 is norleucine (Nle);
X11 is absent; and
X12 is absent.

Embodiment 143. The peptide of any one of embodiments 129-135, or a pharmaceutically acceptable salt, solvate, or isotope thereof, wherein:
X1 is D-Ser, Ser, or Gly;
X2 is Glu or Asp;
Ar is histidine;
X4 is 7-methyltryptophan (7MeW);
X5 is Ala;
X7 is β-alanine or BABA;
X8 is H or NmeHis;
X10 is norleucine (Nle);
X11 is absent; and
X12 is absent.

Embodiment 143a. The peptide of embodiment 141, or a pharmaceutically acceptable salt, solvate, or isotope thereof, wherein:
X0 is Glu or Ser.

Embodiment 143b. The peptide of any one of embodiments 137, 139, or 141-143, or a pharmaceutically acceptable salt, solvate, or isotope thereof, wherein:
X0 is Glu.

Embodiment 144. The peptide of any one of embodiments 129-143, or a pharmaceutically acceptable salt, solvate, or isotope thereof, wherein the peptide comprises an amino acid sequence comprising at least 50%, 55%, 60%, 65%, 70%, 75%, 80%, 85%, 90%, 95%, 96%, 97%, 98%, 99% or 100% sequence identity to any one of SEQ ID NOs: 6, 27-43, 166-177, 179-212, 214-218, 220-239, 246, 249, 250, or 279-306.

Embodiment 145. The peptide of any one of embodiments 129-143, or a pharmaceutically acceptable salt, solvate, or isotope thereof, wherein the peptide comprises the amino acid sequence of any one of SEQ ID NOs: 6, 27-43, 166-177, 179-212, 214-218, 220-239, 246, 249, 250, or 279-306.

Embodiment 146. The peptide of embodiment 129-143, or a pharmaceutically acceptable salt, solvate, or isotope thereof, wherein the peptide comprises the amino acid sequence of any one of SEQ ID NOs: 33, 223, 237, 238, 246, 249, 250, or 279-306.

Embodiment 147. The peptide of any one of embodiments 1-145, or a pharmaceutically acceptable salt, solvate, or isotope thereof, wherein the peptide is no more than 14 amino acids in length.

Embodiment 148. The peptide of any one of embodiments 1-146, or a pharmaceutically acceptable salt, solvate, or isotope thereof, wherein the peptide is no more than 13 amino acids in length.

Embodiment 149. The peptide of any one of embodiments 1-146, or a pharmaceutically acceptable salt, solvate, or isotope thereof, wherein the peptide is no more than 12 amino acids in length.

Embodiment 150. The peptide of any one of embodiments 1-146, or a pharmaceutically acceptable salt, solvate, or isotope thereof, wherein the peptide is no more than 11 amino acids in length.

Embodiment 151. The peptide of any one of embodiments 1-146, or a pharmaceutically acceptable salt, solvate, or isotope thereof, wherein the peptide is no more than 10 amino acids in length.

Embodiment 152. The peptide of any one of embodiments 1-146, or a pharmaceutically acceptable salt, solvate, or isotope thereof, wherein the peptide is no more than 9 amino acids in length.

Embodiment 153. The peptide of any one of embodiments 1-146, or a pharmaceutically acceptable salt, solvate, or isotope thereof, wherein the peptide is no more than 8 amino acids in length.

Embodiment 154. The peptide of any one of embodiments 1-146, or a pharmaceutically acceptable salt, solvate, or isotope thereof, wherein the peptide is no more than 7 amino acids in length.

Embodiment 155. A peptide comprising the structure:

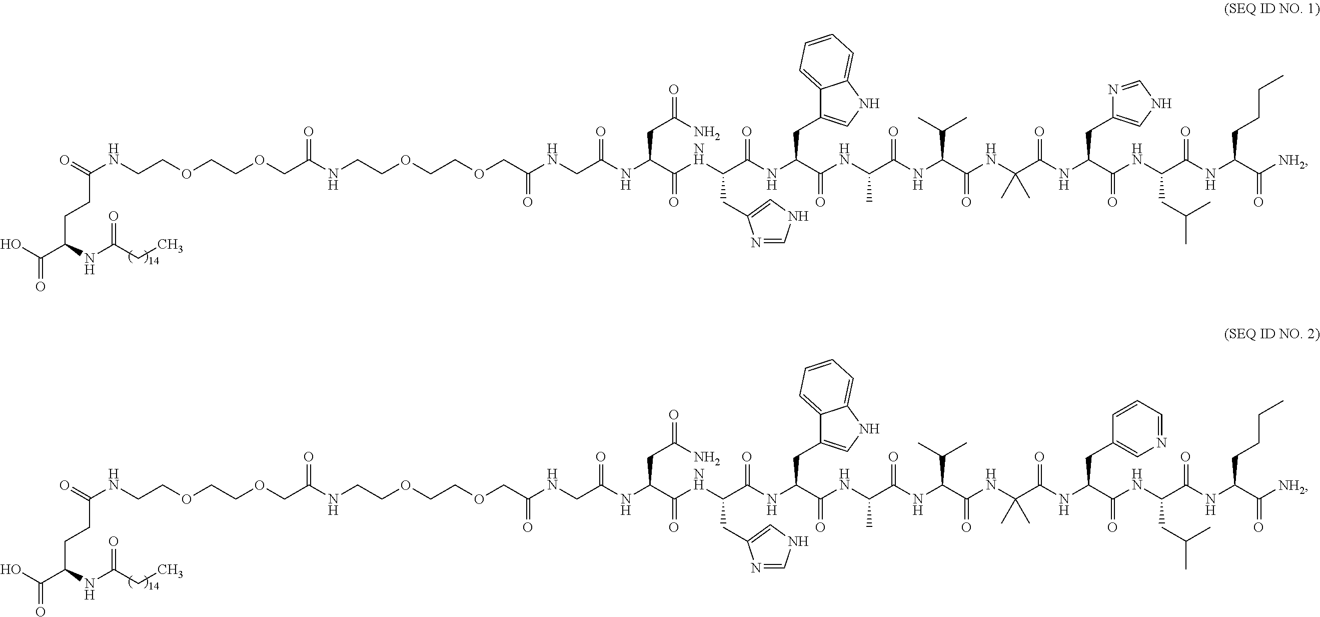

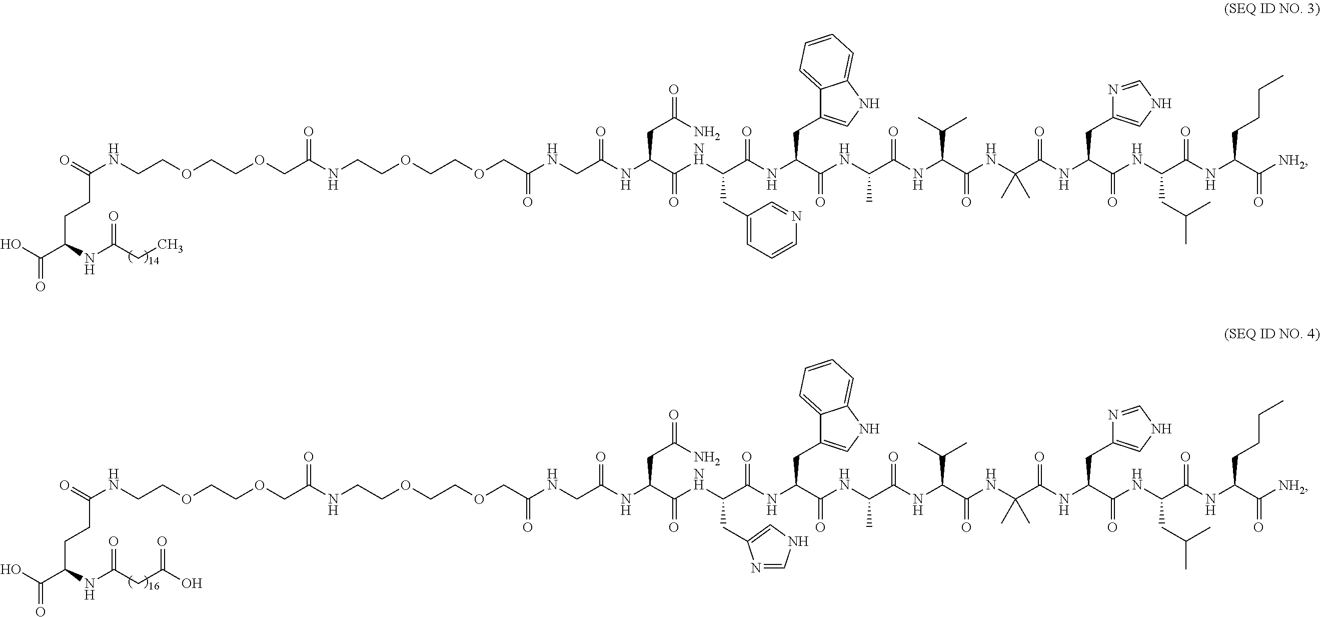

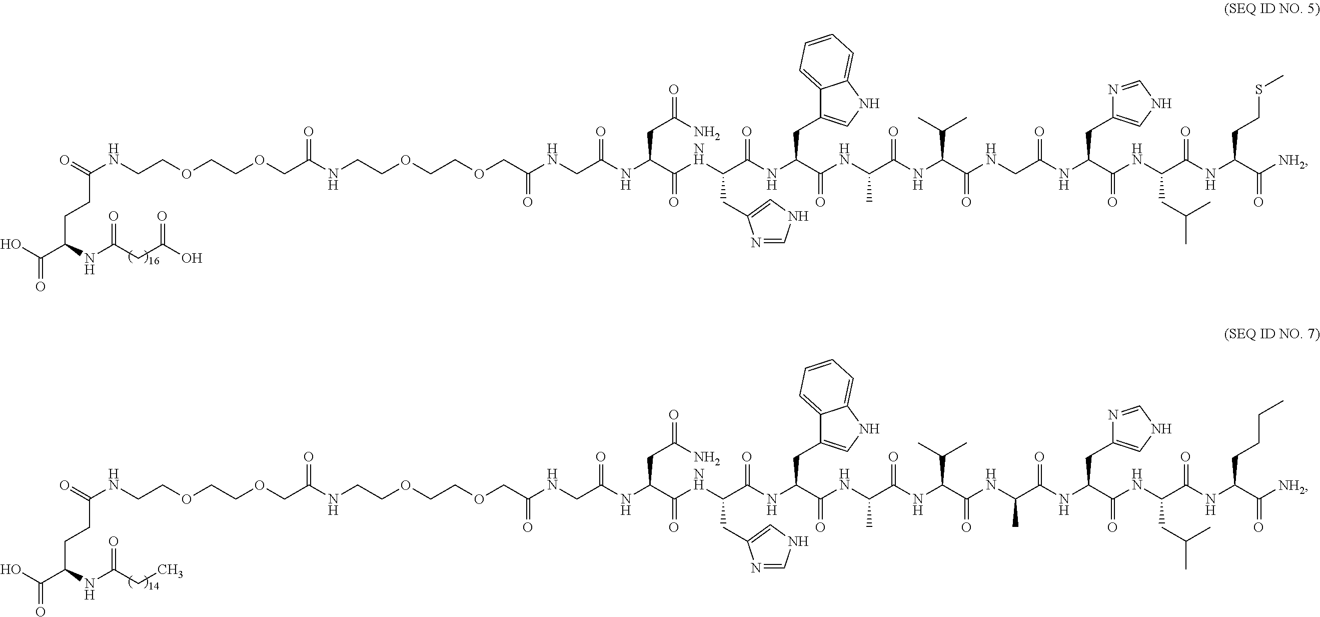

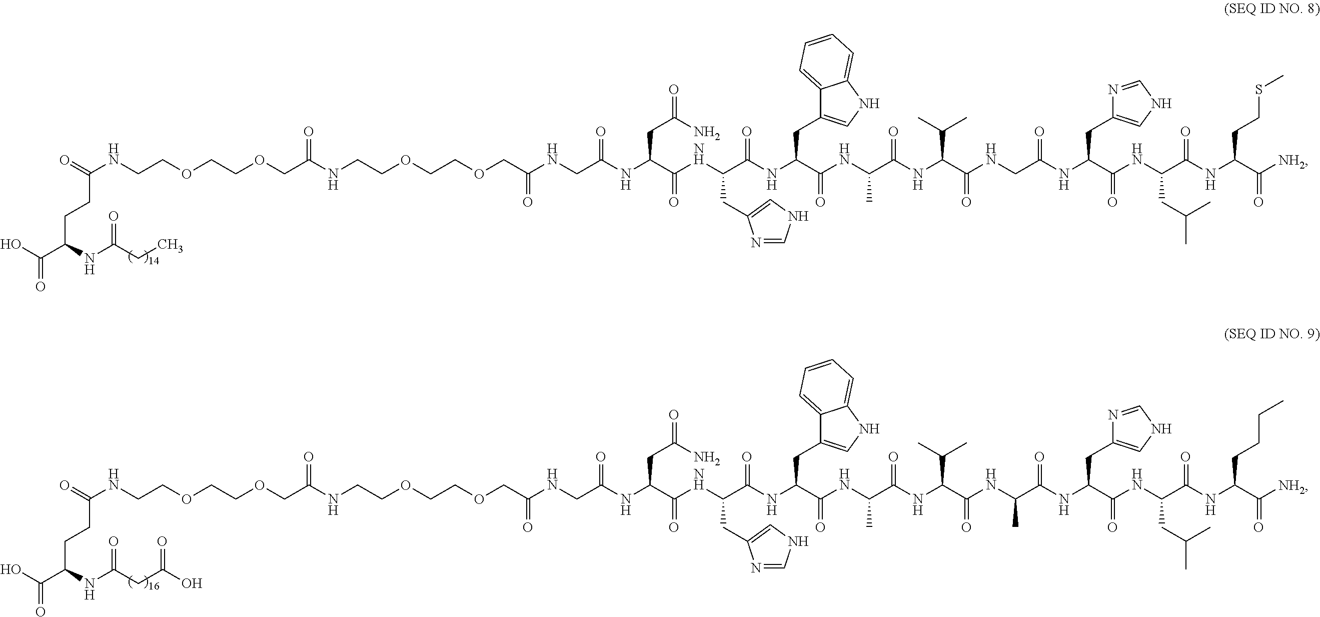

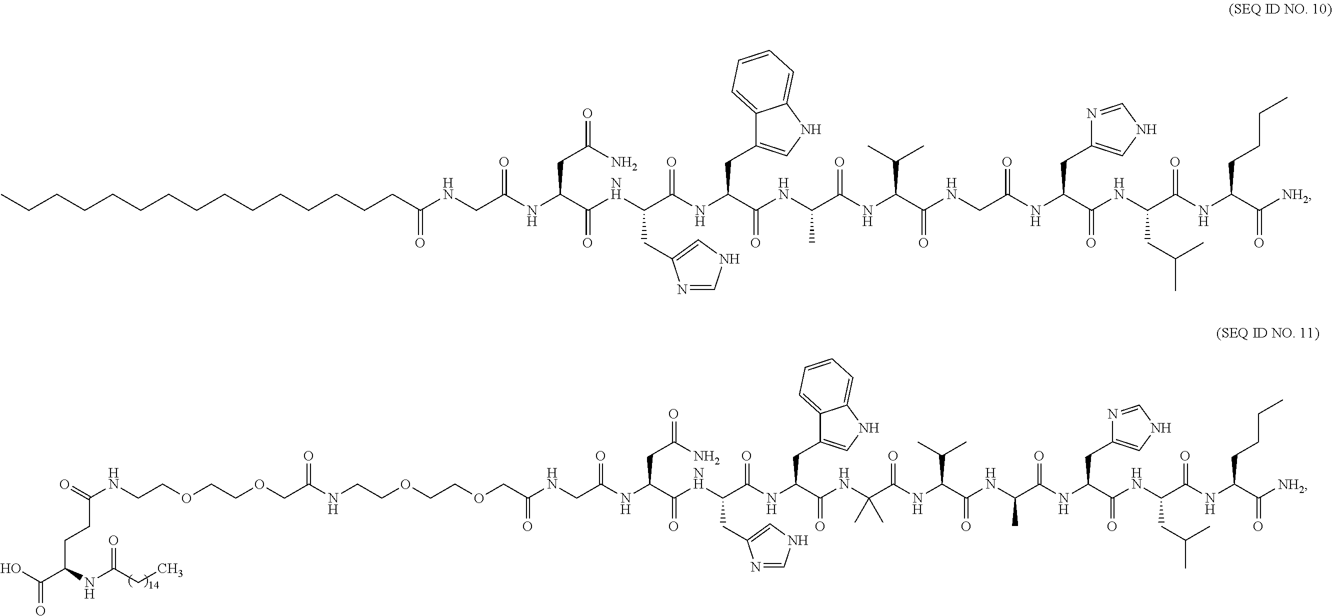

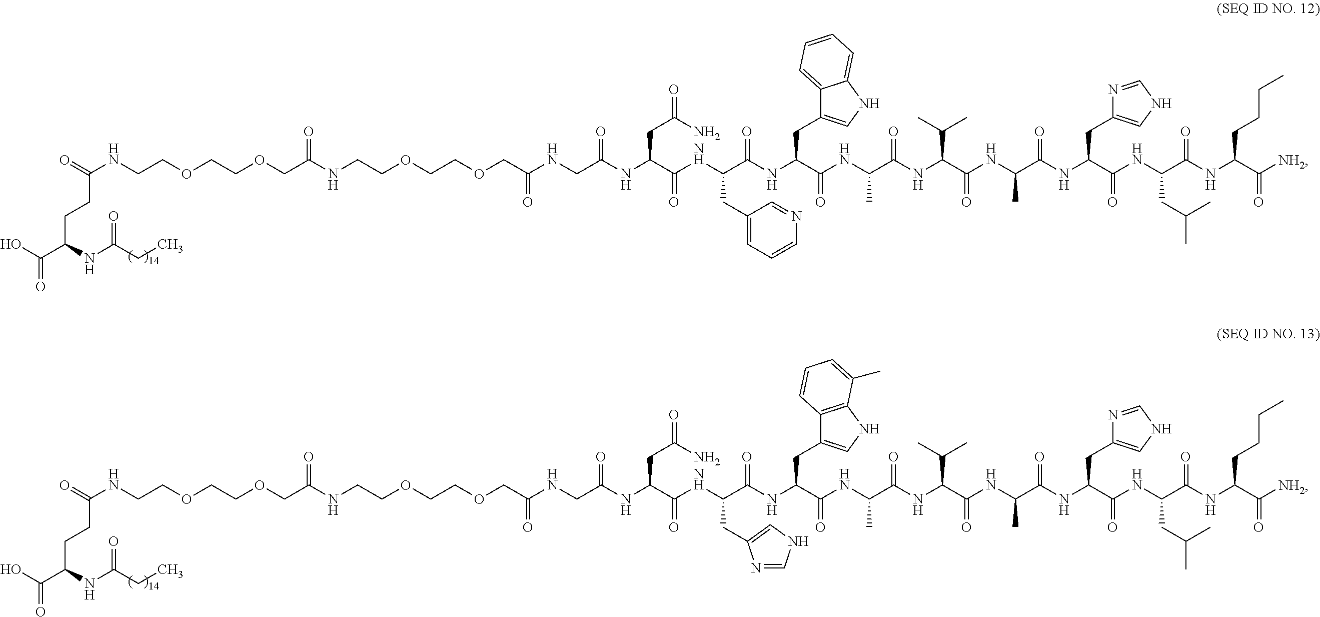

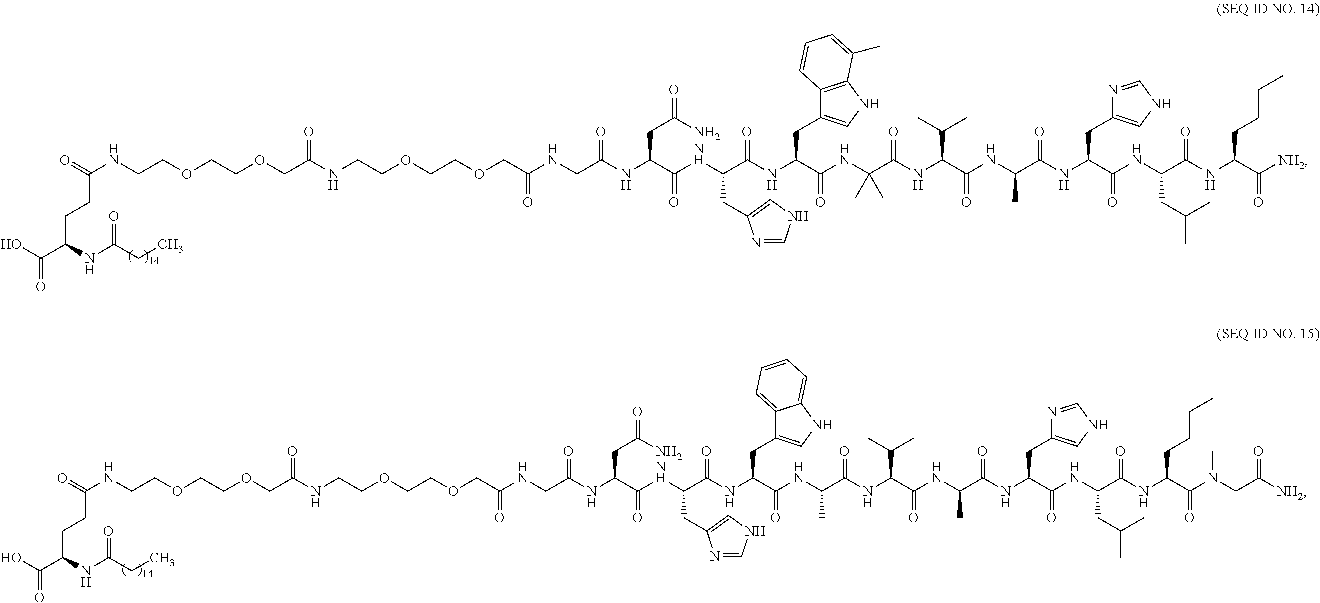

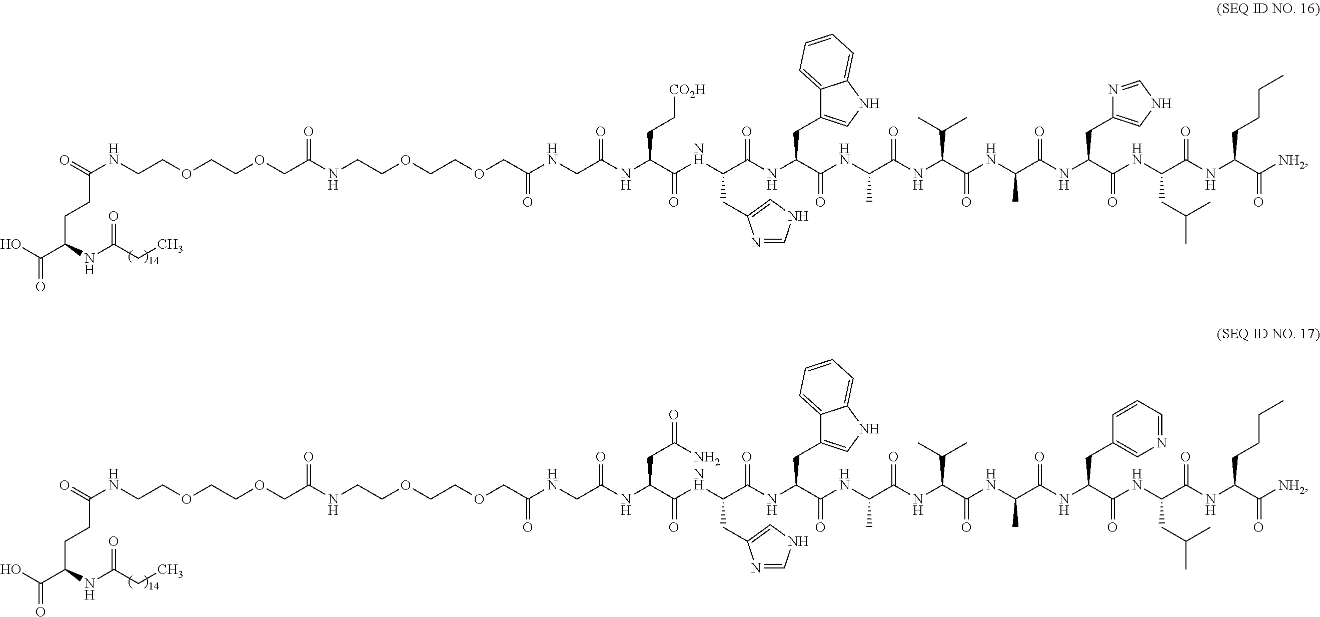

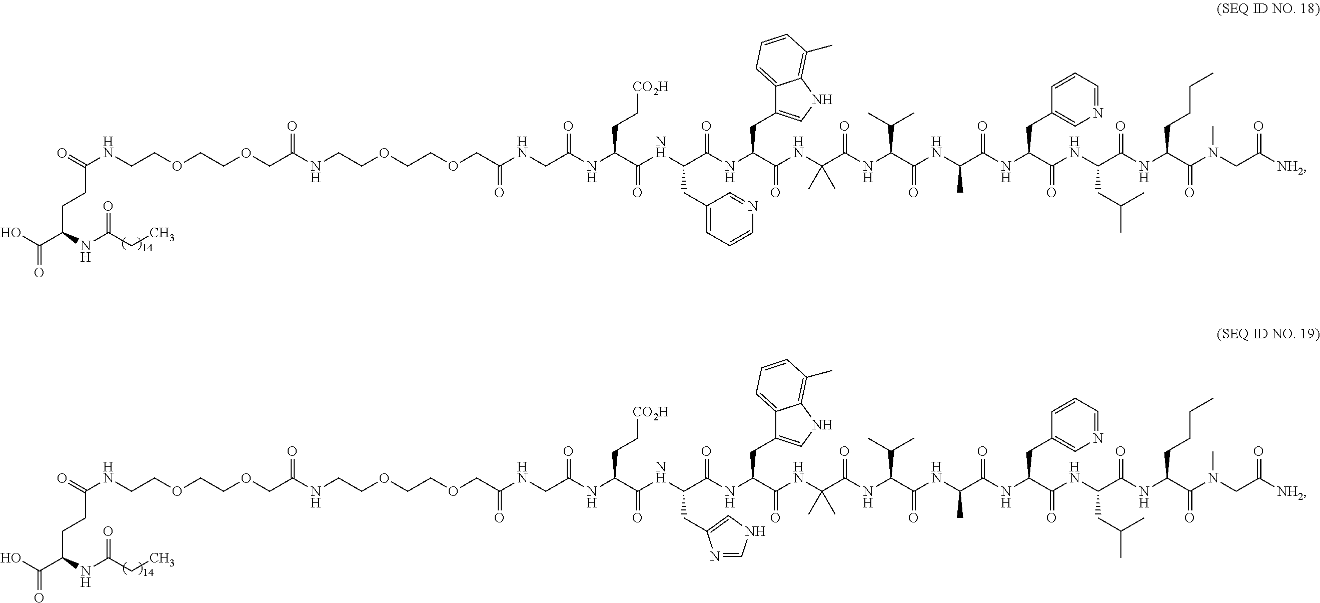

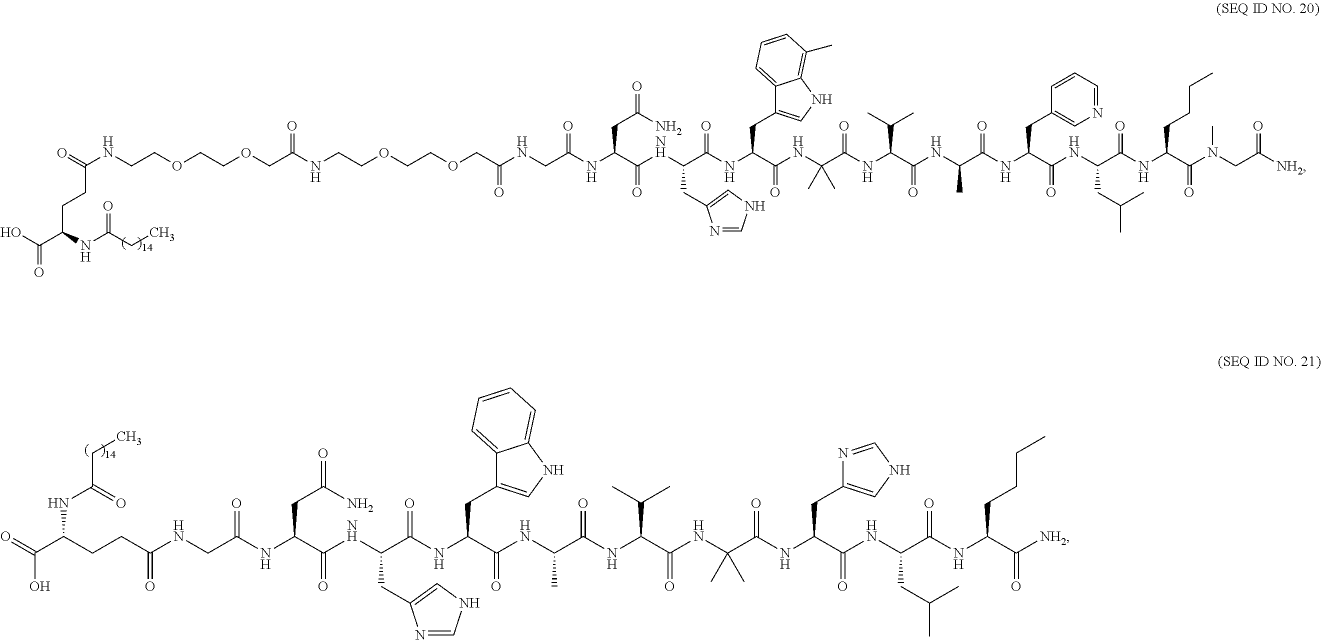

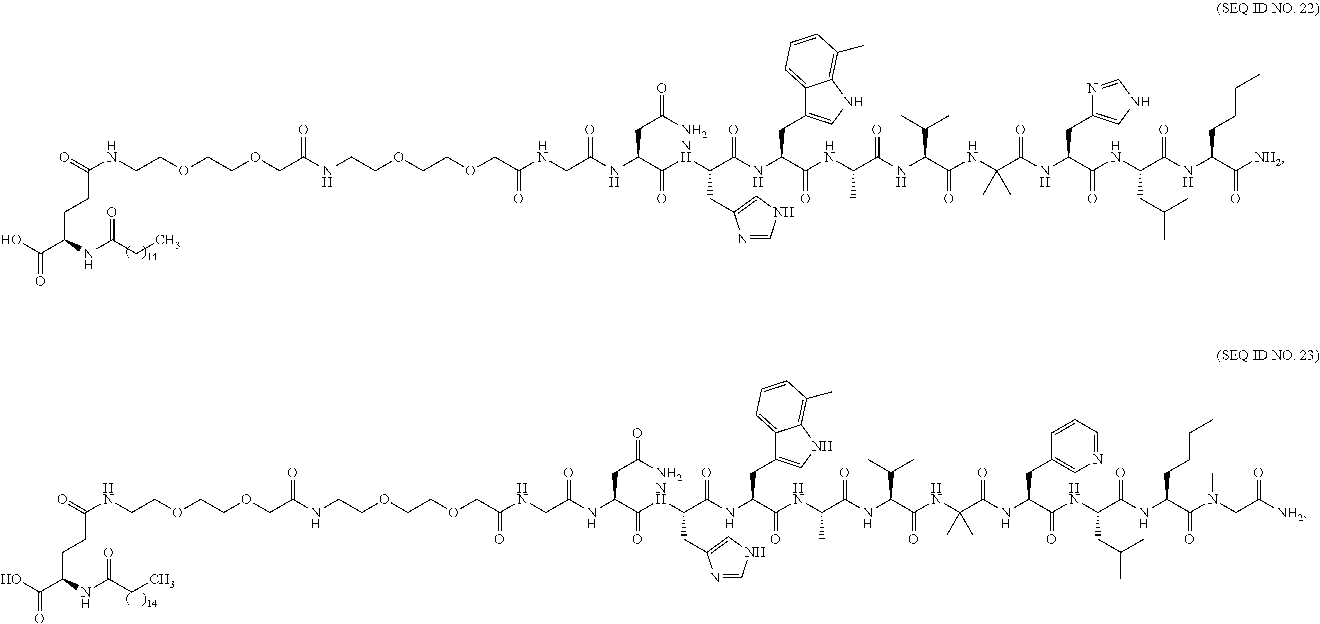

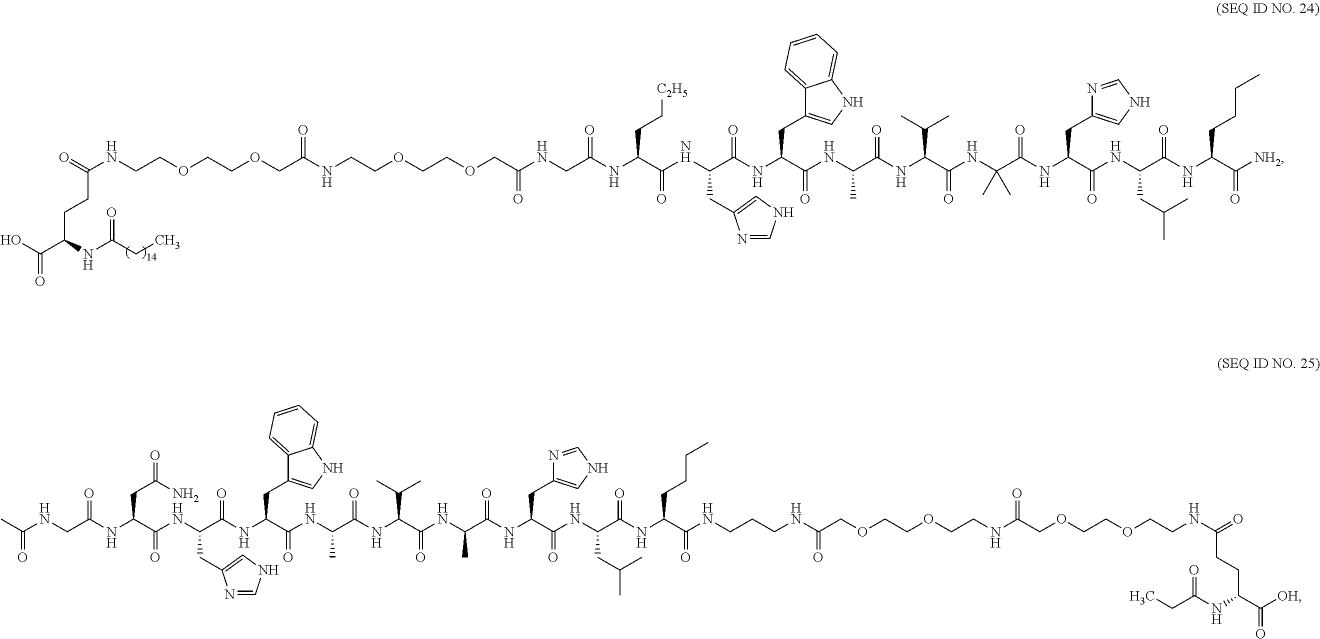

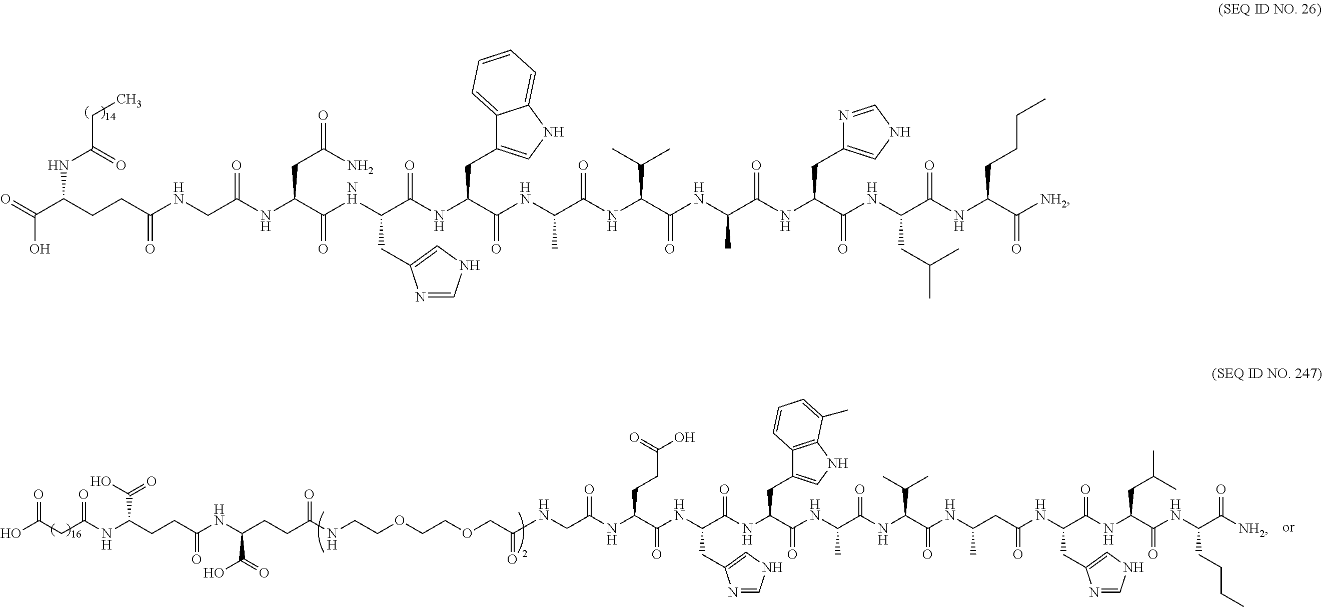

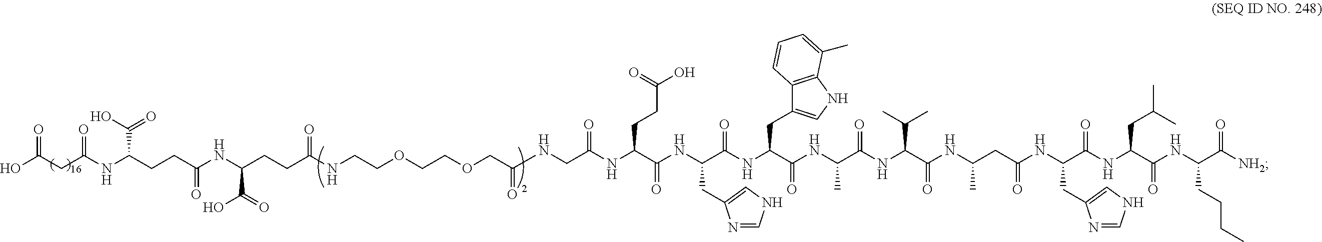

or a pharmaceutically acceptable salt, solvate, or isotope thereof.

Embodiment 156. A peptide comprising the sequence:

(SEQ ID NO. 1)
Palmitoyl-D-γGlu-OEG-OEG-Gly-Asn-His-Trp-Ala-Val-Aib-His-Leu-Nle-NH₂,
(SEQ ID NO. 2)
Palmitoyl-D-γGlu-OEG-OEG-Gly-Asn-His-Trp-Ala-Val-Aib-3Pal-Leu-Nle-NH₂,
(SEQ ID NO. 3)
Palmitoyl-D-γGlu-OEG-OEG-Gly-Asn-3Pal-Trp-Ala-Val-Aib-His-Leu-Nle-NH₂,
(SEQ ID NO. 4)
Octadecanedioyl-D-γGlu-OEG-OEG-Gly-Asn-His-Trp-Ala-Val-Aib-His-Leu-Nle-NH₂,
(SEQ ID NO. 5)
Octadecanedioyl-D-γGlu-OEG-OEG-Gly-Asn-His-Trp-Ala-Val-Gly-His-Leu-Met-NH₂,
(SEQ ID NO. 7)
Palmitoyl-D-γGlu-OEG-OEG-Gly-Asn-His-Trp-Ala-Val-D-Ala-His-Leu-Nle-NH₂,
(SEQ ID NO. 8)
Palmitoyl-D-γGlu-OEG-OEG-Gly-Asn-His-Trp-Ala-Val-Gly-His-Leu-Met-NH₂,
(SEQ ID NO. 9)
Octadecanedioyl-D-γGlu-OEG-OEG-Gly-Asn-His-Trp-Ala-Val-D-Ala-His-Leu-Nle-NH₂,
(SEQ ID NO. 10)
Palmitoyl-Gly-Asn-His-Trp-Ala-Val-D-Ala-His-Leu-Nle-NH₂,
(SEQ ID NO. 11)
Palmitoyl-D-γGlu-OEG-OEG-Gly-Asn-His-Trp-Aib-Val-D-Ala-His-Leu-Nle-NH₂,
(SEQ ID NO. 12)
Palmitoyl-D-γGlu-OEG-OEG-Gly-Asn-3Pal-Trp-Ala-Val-D-Ala-His-Leu-Nle-NH₂,
(SEQ ID NO. 13)
Palmitoyl-D-γGlu-OEG-OEG-Gly-Asn-His-7MethylTrp-Ala-Val-D-Ala-His-Leu-Nle-NH₂,
(SEQ ID NO. 14)
Palmitoyl-D-γGlu-OEG-OEG-Gly-Asn-His-7MethylTrp-Aib-Val-D-Ala-His-Leu-Nle-NH₂,
(SEQ ID NO. 15)
Palmitoyl-D-γGlu-OEG-OEG-Gly-Asn-His-Trp-Ala-Val-D-Ala-His-Leu-Nle-Sar-NH₂,
(SEQ ID NO. 16)
Palmitoyl-D-γGlu-OEG-OEG-Gly-Glu-His-Trp-Ala-Val-D-Ala-His-Leu-Nle-NH₂,
(SEQ ID NO. 17)
Palmitoyl-D-γGlu-OEG-OEG-Gly-Asn-His-Trp-Ala-Val-D-Ala-3Pal-Leu-Nle-NH₂,
(SEQ ID NO. 18)
Palmitoyl-D-γGlu-OEG-OEG-Gly-Glu-3Pal-7MethylTrp-Aib-Val-D-Ala-3Pal-Leu-Nle-Sar-NH₂,
(SEQ ID NO. 19)
Palmitoyl-D-γGlu-OEG-OEG-Gly-Glu-His-7MethylTrp-Aib-Val-D-Ala-3Pal-Leu-Nle-Sar-NH₂,
(SEQ ID NO. 20)
Palmitoyl-D-γGlu-OEG-OEG-Gly-Asn-His-7MethylTrp-Aib-Val-D-Ala-3Pal-Leu-Nle-Sar-NH₂,
(SEQ ID NO. 21)
Palmitoyl-D-γGlu-Gly-Asn-His-Trp-Ala-Val-Aib-His-Leu-Nle-NH₂,
(SEQ ID NO. 22)
Palmitoyl-D-γGlu-OEG-OEG-Gly-Asn-His-7MethylTrp-Ala-Val-Aib-His-Leu-Nle-NH₂,
(SEQ ID NO. 23)
Palmitoyl-D-γGlu-OEG-OEG-Gly-Asn-His-7MethylTrp-Ala-Val-Aib-3Pal-Leu-Nle-Sar-NH₂,
(SEQ ID NO. 24)
Palmitoyl-D-γGlu-OEG-OEG-Gly-Glu-His-Trp-Ala-Val-Aib-His-Leu-Nle-NH₂,
(SEQ ID NO. 25)
Ac-Gly-Asn-His-Trp-Ala-Val-D-Ala-His-Leu-Nle-H₂N(CH₂)₃NH₂-OEG-OEG-(α-Palmitoyl-D-
γGlu),
(SEQ ID NO. 26)
Palmitoyl-D-γGlu-Gly-Asn-His-Trp-Ala-Val-D-Ala-His-Leu-Nle-NH₂,
(SEQ ID NO. 46)
Octadecanedioyl-D-Glu-OEG-OEG-Gly-Asn-His-7MethylTrp-Ala-Val-D-Ala-His-Leu-Nle-NH₂,
(SEQ ID NO. 47)
Octadecanedioyl-D-γGlu-OEG-OEG-Gly-Asn-His-Trp-Ala-Val-D-Ala-His-Leu-Nle-Sar-NH₂,
(SEQ ID NO. 48)
Palmitoyl-D-γGlu-OEG-OEG-Gly-Asn-His-Trp-Ala-Val-D-Ala-His-Leu-Nle-Sar-NH₂,
(SEQ ID NO. 49)
Palmitoyl-D-γGlu-OEG-OEG-Gly-Asn-His-Trp-Ala-Val-D-Ala-His-Leu-Lys(Me)3-NH₂,
(SEQ ID NO. 50)
Ac-Gly-Asn-His-Trp-Ala-Val-D-Ala-His-Leu-ϵK-OEG-OEG-(α-Palmitoyl-D-γGlu)-NH₂,
(SEQ ID NO. 51)
Palmitoyl-D-γGlu-OEG-OEG-Gly-Asn-2Pal-Trp-Ala-Val-^{D}Ala-His-Leu-Nle-NH₂,
(SEQ ID NO. 52)
Palmitoyl-D-γGlu-OEG-OEG-Gly-Asn-4Pal-Trp-Ala-Val-D-Ala-His-Leu-Nle-NH₂,
(SEQ ID NO. 53)
Octadecanedioyl-D-γGlu-OEG-OEG-OEG-OEG-Gly-Asn-His-Trp-Ala-Val-D-Ala-His-Leu-Nle-
NH₂,
(SEQ ID NO. 54)
Palmitoyl-D-γGlu-OEG-OEG-OEG-OEG-Gly-Asn-His-Trp-Ala-Val-D-Ala-His-Leu-Nle-NH₂,
(SEQ ID NO. 55)
Octadecanedioyl-D-γGlu-OEG-OEG-Gly-Lys(OEG-OEG-Octadecanedioyl-D-Glu-His-Trp-Ala-
Val-D-Ala-His-Leu-Nle-NH₂,
(SEQ ID NO. 56)
Palmitoyl-D-γGlu-OEG-OEG-Gly-Asn-His-7FTrp-Ala-Val-D-Ala-His-Leu-Nle-NH₂,
(SEQ ID NO. 57)
Palmitoyl-D-γGlu-OEG-OEG-Gly-Lys(OEG-OEG-α-Palmitoyl-D-γGlu)-His-Trp-Ala-Val-D-
Ala-His-Leu-Nle-NH₂,
(SEQ ID NO. 58)
Palmitoyl-D-γGlu-OEG-OEG-OEG-OEG-Gly-Asn-3Pal-7MethylTrp-Ala-Val-D-Ala-His-Leu-
Nle-NH₂,
(SEQ ID NO. 59)
Palmitoyl-D-γGlu-OEG-OEG-OEG-OEG-Gly-Asn-His-7MethylTrp-Ala-Val-D-Ala-His-Leu-
Nle-NH₂,
(SEQ ID NO. 60)
Octadecanedioyl-D-γGlu-OEG-OEG-Gly-Lys-His-Trp-Ala-Val-D-Ala-His-Leu-Nle-NH₂,
(SEQ ID NO. 61)
Octadecanedioyl-D-γGlu-OEG-OEG-Gly-Arg-His-Trp-Ala-Val-D-Ala-His-Leu-Nle-NH₂,
(SEQ ID NO. 62)
Octadecanedioyl-D-γGlu-OEG-OEG-Gly-Asn-His-Trp-Ala-Val-D-Ala-Phe-Leu-Nle-NH₂,
(SEQ ID NO. 63)
Palmitoyl-D-γGlu-OEG-OEG-Gly-Asn-His-6MethylTrp-Ala-Val-D-Ala-His-Leu-Nle-NH₂,
(SEQ ID NO. 64)
Octadecanedioyl-D-γGlu-OEG-OEG-Gly-Asn-3Pal-Trp-Ala-Val-D-Ala-His-Leu-Nle-NH₂,
(SEQ ID NO. 65)
Octadecanedioyl-D-γGlu-OEG-OEG-Gly-Lys(Ac)-His-Trp-Ala-Val-D-Ala-His-Leu-Nle-NH₂,
(SEQ ID NO. 66)
Octadecanedioyl-D-γGlu-OEG-OEG-Gly-Asn-His-Trp-Ala-Val-D-Ala-3Pal-Leu-Nle-NH₂,
(SEQ ID NO. 67)
Octadecanedioyl-D-γGlu-OEG-OEG-Gly-Glu-His-Trp-Ala-Val-D-Ala-3Pal-Leu-Nle-NH₂,
(SEQ ID NO. 68)
Octadecanedioyl-D-γGlu-OEG-OEG-Gly-Asn-His-His-Ala-Val-D-Ala-His-Leu-Nle-NH₂,
(SEQ ID NO. 69)
Octadecanedioyl-D-γGlu-OEG-OEG-Gly-Asn-His-Trp-Ala-Val-D-Ala-His-Leu-Arg-NH₂,
(SEQ ID NO. 70)
Octadecanedioyl-D-γGlu-OEG-OEG-Gly-Glu-His-Trp-Aib-Val-D-Ala-His-Leu-Nle-NH₂,
(SEQ ID NO. 71)
Octadecanedioyl-D-γGlu-OEG-OEG-Lys(Ac)-His-Trp-Ala-Val-D-Ala-His-Leu-Nle-NH₂,
(SEQ ID NO. 72)
Octadecanedioyl-D-γGlu-OEG-OEG-Gly-Asn-3Pal-Trp-Ala-Val-D-Ala-His-Leu-Nle-Sar-NH₂,
(SEQ ID NO. 73)
Octadecanedioyl-D-γGlu-OEG-OEG-Gly-Asn-3Pal-7MethylTrp-Ala-Val-D-Ala-His-Leu-Nle-
Sar-NH₂,
(SEQ ID NO. 74)
Octadecanedioyl-D-γGlu-OEG-OEG-Aib-Asn-His-Trp-Ala-Val-D-Ala-His-Leu-Nle-NH₂,
(SEQ ID NO. 75)
Palmitoyl-D-γGlu-OEG-OEG-Gly-Asn-His-Trp-Ala-Val-D-Ala-His-Leu-Nle-Aib-NH₂,
(SEQ ID NO. 76)
Octadecanedioyl-D-γGlu-OEG-OEG-Gly-Asn-His-7MethylTrp-Ala-Val-D-Ala-His-Leu-Nle-
Aib-NH₂,
(SEQ ID NO. 77)
Octadecanedioyl-D-γGlu-OEG-OEG-Gly-Lys(Ac)-His-Trp-Ala-Val-D-Ala-His-Leu-Nle-Sar-
NH₂,
(SEQ ID NO. 78)
Octadecanedioyl-D-γGlu-OEG-OEG-OEG-OEG-Gly-Asn-His-7MethyTrp-Ala-Val-D-Ala-His-
Leu-NH₂,
(SEQ ID NO. 79)
Octadecanedioyl-D-γGlu-OEG-OEG-Gly-Asn-His-6MethylTrp-Ala-Val-D-Ala-His-Leu-Nle-
NH₂,
(SEQ ID NO. 80)
Octadecanedioyl-D-γGlu-Ava-Gly-Asn-His-Trp-Ala-Val-D-Ala-His-Leu-Nle-NH₂,
(SEQ ID NO. 81)
Octadecanedioyl-D-γGlu-OEG-OEG-Gly-Asn-His-7MethylTrp-Ala-Val-D-Ala-His-Leu-Nle-
Sar-NH₂,
(SEQ ID NO. 82)
Octadecanedioyl-D-γGlu-OEG-OEG-Gly-Asn-His-Trp-Ala-Val-D-Ala-His-Leu-Nle-Aib-NH₂,
(SEQ ID NO. 83)
Octadecanedioyl-D-γGlu-OEG-OEG-OEG-OEG-Gly-Asn-3Pal-Trp-Ala-Val-D-Ala-His-Leu-
Nle-NH₂,
(SEQ ID NO. 84)
Octadecanedioyl-D-γGlu-OEG-OEG-Gly-Asn-His-6MethylTrp-Ala-Val-D-Ala-His-Leu-Nle-
Sar-NH₂,
(SEQ ID NO. 85)
Octadecanedioyl-γGlu-OEG-OEG-Gly-Asn-His-7MethylTrp-Ala-Val-D-Ala-His-Leu-Nle-NH₂,
(SEQ ID NO. 86)
Octadecanedioyl-γGlu-OEG-OEG-Gly-Asn-His-7FTrp-Ala-Val-D-Ala-His-Leu-Nle-NH₂,
(SEQ ID NO. 87)
Palmitoyl-γGlu-OEG-OEG-Gly-Asn-His-7FTrp-Ala-Val-D-Ala-His-Leu-Nle-NH₂,
(SEQ ID NO. 88)
Octadecanedioyl-γGlu-OEG-OEG-Gly-Arg-His-Trp-Ala-Val-D-Ala-His-Leu-Nle-NH₂,
(SEQ ID NO. 89)
Palmitoyl-γGlu-OEG-OEG-Gly-Arg-His-Trp-Ala-Val-D-Ala-His-Leu-Nle-NH₂,
(SEQ ID NO. 90)
Octadecanedioyl-γGlu-OEG-OEG-OEG-OEG-Gly-Asn-His-7MethylTrp-Ala-Val-D-Ala-His-
Leu-Nle-NH₂,
(SEQ ID NO. 91)
Palmitoyl-γGlu-OEG-OEG-OEG-OEG-Gly-Asn-3Pal-7MethylTrp-Ala-Val-D-Ala-His-Leu-Nle-
NH₂,
(SEQ ID NO. 92)
Palmitoyl-γGlu-OEG-OEG-Gly-Asn-His-Trp-Ala-Val-D-Ala-His-Leu-Nle-Sar-NH₂,
(SEQ ID NO. 93)
Palmitoyl-γGlu-OEG-OEG-Gly-Asn-His-2Nal-Ala-Val-Ala-His-Leu-Nle-NH₂,
(SEQ ID NO. 94)
Palmitoyl-γGlu-OEG-OEG-Gly-Asn-4Pal-Trp-Ala-Val-D-Ala-His-Leu-Nle-NH₂,
(SEQ ID NO. 95)
Octadecanedioyl-γGlu-OEG-OEG-OEG-OEG-Gly-Asn-3Pal-7MeTrp-Ala-Val-D-Ala-His-Leu-
Nle-NH₂,
(SEQ ID NO. 96)
Palmitoyl-γGlu-OEG-OEG-Gly-Asn-His-Trp-Ala-Val-D-Ala-His-Leu-Nle-NH₂,
(SEQ ID NO. 97)
Octadecanedioyl-γGlu-OEG-OEG-Gly-Asn-His-Trp-Ala-Val-D-Ala-His-Leu-Nle-NH₂,
(SEQ ID NO. 98)
Octadecanedioyl-γGlu-OEG-OEG-OEG-OEG-Gly-Asn-His-Trp-Ala-Val-D-Ala-His-Leu-Nle-
NH₂,
(SEQ ID NO. 99)
Octadecanedioyl-γGlu-OEG-OEG-Gly-Asn-His-2Nal-Ala-Val-D-Ala-His-Leu-Nle-Sar-NH₂,
(SEQ ID NO. 100)
Octadecanedioyl-γGlu-OEG-OEG-Arg-Asn-His-Trp-Ala-Val-D-Ala-His-Leu-Nle-Sar-NH₂,
(SEQ ID NO. 101)
Octadecanedioyl-γGlu-OEG-OEG-OEG-OEG-Gly-Asn-His-7FTrp-Ala-Val-D-Ala-His-Leu-Nle-
Sar-NH₂,
(SEQ ID NO. 102)
Octadecanedioyl-γGlu-OEG-OEG-Gly-Asn-His-7FTrp-Ala-Val-D-Ala-His-Leu-Nle-Sar-NH₂,
(SEQ ID NO. 103)
Octadecanedioyl-γGlu-OEG-OEG-OEG-OEG-Gly-Asn-His-7MethylTrp-Ala-Val-D-Ala-His-
Leu-Nle-Sar-NH₂,
(SEQ ID NO. 104)
Octadecanedioyl-γGlu-OEG-OEG-Gly-Asn-His-7MethylTrp-Ala-Val-D-Ala-His-Leu-Nle-Sar-
NH₂,
(SEQ ID NO. 105)
Octadecanedioyl-γGlu-OEG-OEG-Gly-Asn-His-2Nal-Ala-Val-D-Ala-His-Leu-Nle-NH₂,
(SEQ ID NO. 106)
Octadecanedioyl-γGlu-OEG-OEG-Gly-Asn-His-Trp-D-Ala-Val-D-Ala-His-Leu-Nle-NH₂,
(SEQ ID NO. 107)
Octadecanedioyl-γGlu-OEG-OEG-Gly-Asn-His-His-Ala-Val-D-Ala-His-Leu-Nle-NH₂,
(SEQ ID NO. 108)
Octadecanedioyl-γGlu-OEG-OEG-Gly-Asn-His-Arg-Ala-Val-D-Ala-His-Leu-Nle-NH₂,
(SEQ ID NO. 109)
Octadecanedioyl-γGlu-OEG-OEG-OEG-OEG-Gly-Arg-His-7FTrp-Ala-Val-D-Ala-His-Leu-Nle-
NH₂,
(SEQ ID NO. 110)
Octadecanedioyl-γGlu-OEG-OEG-OEG-OEG-Gly-Asn-His-7FTrp-Ala-Val-D-Ala-His-Leu-Nle-
NH₂,
(SEQ ID NO. 111)
Palmitoyl-γGlu-OEG-OEG-Gly-Asn-His-Trp-Ala-Val-D-Ala-His-Leu-Nle-Sar-NH₂,
(SEQ ID NO. 112)
Palmitoyl-γGlu-OEG-OEG-Gly-Asn-His-7MethylTrp-Ala-Val-D-Ala-His-Leu-Nle-Sar-NH₂,
(SEQ ID NO. 114)
Octadecanedioyl-γGlu-OEG-OEG-Gly-Asn-His-Trp-Ala-Val-D-Ala-His-Leu-Nle-Arg-NH₂,
(SEQ ID NO. 115)
Octadecanedioyl-γGlu-OEG-OEG-Gly-Asn-His-Trp-D-Ala-Val-D-Ala-His-Leu-Nle-Sar-NH₂,
(SEQ ID NO. 116)
Octadecanedioyl-γGlu-OEG-OEG-Gly-Arg-His-His-Ala-Val-D-Ala-His-Leu-Nle-NH₂,
(SEQ ID NO. 117)
Octadecanedioyl-γGlu-OEG-OEG-OEG-OEG-Gly-Arg-His-7MethylTrp-Ala-Val-D-Ala-His-
Leu-Nle-NH₂,
(SEQ ID NO. 118)
Octadecanedioyl-γGlu-OEG-OEG-OEG-OEG-Gly-Arg-His-Trp-Ala-Val-D-Ala-His-Leu-Nle-
NH₂,
(SEQ ID NO. 119)
Octadecanedioyl-γGlu-OEG-OEG-Gly-Asn-His-Trp-Ala-Val-D-Ala-His-Leu-Cha-NH₂,
(SEQ ID NO. 120)
Octadecanedioyl-γGlu-OEG-OEG-Gly-Ala-His-Trp-Ala-Val-D-Ala-His-Leu-Cha-NH₂,
(SEQ ID NO. 121)
Octadecanedioyl-γGlu-OEG-OEG-OEG-OEG-Gly-Ala-His-7FTrp-Ala-Val-D-Ala-His-Leu-Nle-
Sar-NH₂,
(SEQ ID NO. 122)
Eicosanedioyl-γGlu-OEG-OEG-OEG-OEG-Gly-Arg-His-7MethylTrp-Ala-Val-D-Ala-His-Leu-
Nle-NH₂,
(SEQ ID NO. 123)
Eicosanedioyl-γGlu-OEG-OEG-Gly-Asn-His-7MethylTrp-Ala-Val-D-Ala-His-Leu-Nle-Sar-NH₂,
(SEQ ID NO. 124)
Octadecanedioyl-γGlu-OEG-OEG-Gly-Ala-His-2Nal-Ala-Val-D-Ala-His-Leu-Nle-Sar-NH₂,
(SEQ ID NO. 125)
Eicosanedioyl-γGlu-OEG-OEG-Gly-Asn-His-7MethylTrp-D-Ala-Val-D-Ala-His-Leu-Nle-Sar-
NH₂,
(SEQ ID NO. 126)
Octadecanedioyl-γGlu-OEG-OEG-Gly-Ala-His-2Nal-Ala-Val-D-Ala-His-Leu-Nle-NH₂,
(SEQ ID NO. 128)
Octadecanedioyl-γGlu-OEG-OEG-Gly-Ala-His-Trp-D-Ala-Val-D-Ala-His-Leu-Nle-Sar-NH₂,
(SEQ ID NO. 129)
Octadecanedioyl-γGlu-OEG-OEG-Gly-Arg-His-7MethyTrp-Ala-Val-D-Ala-His-Leu-Nle-Sar-
NH₂,
(SEQ ID NO. 130)
Octadecanedioyl-γGlu-OEG-OEG-Gly-Asn-His-Arg-Ala-Val-D-Ala-His-Leu-Nle-Sar-NH₂,
(SEQ ID NO. 131)
Eicosanedioyl-γGlu-OEG-OEG-Gly-Asn-His-Trp-Ala-Val-D-Ala-His-Leu-Nle-Sar-NH₂,
(SEQ ID NO. 132)
Eicosanedioyl-γGlu-OEG-OEG-OEG-OEG-Gly-Arg-His-Trp-Ala-Val-D-Ala-His-Leu-Nle-NH₂,
(SEQ ID NO. 133)
Octadecanedioyl-γGlu-OEG-OEG-OEG-OEG-Gly-Asn-His-6FTrp-Ala-Val-D-Ala-His-Leu-Nle-
Sar-NH₂,
(SEQ ID NO. 134)
Octadecanedioyl-γGlu-OEG-OEG-Gly-Asn-His-7MeTrp-Ala-Val-D-Ala-His-Leu-Nle-Arg-NH₂,
(SEQ ID NO. 136)
Octadecanedioyl-γGlu-OEG-OEG-OEG-OEG-Gly-Ala-His-7FTrp-Ala-Val-D-Ala-His-Leu-Nle-
NH₂,
(SEQ ID NO. 137)
Octadecanedioyl-γGlu-OEG-OEG-OEG-OEG-Gly-Arg-His-7FTrp-Ala-Val-D-Ala-His-Leu-Nle-
Sar-NH₂,
(SEQ ID NO. 138)
Eicosanedioyl-γGlu-OEG-OEG-Gly-Asn-His-7FTrp-Ala-Val-D-Ala-His-Leu-Nle-Sar-NH₂,
(SEQ ID NO. 139)
Octadecanedioyl-γGlu-OEG-OEG-Gly-Asp-His-Trp-D-Ala-Val-D-Ala-His-Leu-Nle-NH₂,
(SEQ ID NO. 140)
Octadecanedioyl-γGlu-OEG-OEG-Phe-Asn-His-Trp-Ala-Val-β-Ala-His-Leu-Nle-NH₂,
(SEQ ID NO. 141)
Octadecanedioyl-γGlu-OEG-OEG-Arg-Arg-His-Trp-Ala-Val-D-Ala-His-Leu-Nle-NH₂,
(SEQ ID NO. 142)
Octadecanedioyl-γGlu-OEG-OEG-Gly-Asn-His-Trp-Ala-Val-β-Ala-His-Leu-Nle-NH₂,
(SEQ ID NO. 143)
Octadecanedioyl-γGlu-OEG-OEG-D-Phe-Asn-His-Trp-Ala-Val-D-Ala-His-Leu-Nle-NH₂,
(SEQ ID NO. 144)
Octadecanedioyl-γGlu-OEG-OEG-Phe-Asn-3Pal-Trp-Ala-Val-β-Ala-His-Leu-Nle-NH₂,
(SEQ ID NO. 145)
Octadecanedioyl-γGlu-OEG-OEG-Arg-Asn-His-Trp-Ala-Val-D-Ala-His-Leu-Nle-NH₂,
(SEQ ID NO. 146)
Octadecanedioyl-γGlu-OEG-OEG-Gly-Asn-His-Trp-Ala-Val-D-Ala-His-Leu-Nle,
(SEQ ID NO. 147)
Octadecanedioyl-γGlu-OEG-OEG-Gly-Gln-His-Trp-Ala-Val-D-Ala-His-Leu-Nle-NH₂,
(SEQ ID NO. 148)
Eicosanedioyl-γGlu-OEG-OEG-Gly-Asn-His-Trp-Ala-Val-D-Ala-His-Leu-Nle,
(SEQ ID NO. 149)
Eicosanedioyl-γGlu-OEG-OEG-Gly-Asn-His-Trp-Ala-Val-D-Ala-His-Leu-Nle-NH₂,
(SEQ ID NO. 150)
Eicosanedioyl-γGlu-OEG-OEG-Gly-Arg-His-Trp-Ala-Val-D-Ala-His-Leu-Nle-NH₂,
(SEQ ID NO. 151)
Eicosanedioyl-γGlu-OEG-OEG-Gly-Gln-His-Trp-Ala-Val-D-Ala-His-Leu-Nle-NH₂,
(SEQ ID NO. 152)
Octadecanedioyl-γGlu-OEG-OEG-Gln-His-Trp-Ala-Val-β-Ala-His-Leu-Nle-NH₂,
(SEQ ID NO. 153)
Eicosanedioyl-γGlu-Gly-Arg-His-Trp-Ala-Val-D-Ala-His-Leu-Nle-NH₂,
(SEQ ID NO. 154)
Eicosanedioyl-γGlu-Arg-Asn-His-Trp-Ala-Val-D-Ala-His-Leu-Nle-NH₂,
(SEQ ID NO. 155)
Eicosanedioyl-γGlu-Arg-Asn-His-Trp-Ala-Val-D-Ala-His-Leu-Nle,
(SEQ ID NO. 156)
Octadecanedioyl-γGlu-OEG-OEG-Arg-Asn-His-Trp-Ala-Val-D-Ala-His-Leu-Nle,
(SEQ ID NO. 157)
Octadecanedioyl-γGlu-OEG-OEG-Arg-Arg-His-Trp-Ala-Val-D-Ala-His-Leu-Nle,
(SEQ ID NO. 158)
Eicosanedioyl-γGlu-Arg-Arg-His-Trp-Ala-Val-D-Ala-His-Leu-Nle-NH₂,
(SEQ ID NO. 159)
Octadecanedioyl-γGlu-OEG-OEG-Gly-Asn-His-Trp-Ala-Val-D-Ala-D-His-Leu-Nle-NH₂,
(SEQ ID NO. 160)
Eicosanedioyl-γGlu-Gly-Asn-His-Trp-Ala-Val-D-Ala-D-His-Leu-Nle-NH₂,
(SEQ ID NO. 161)
Octadecanedioyl-γGlu-OEG-OEG-D-Phe-Gln-His-Trp-Ala-Val-D-Ala-His-Leu-Nle-NH₂,
(SEQ ID NO. 162)
Eicosanedioyl-γGlu-Gly-Arg-His-Trp-Ala-Val-D-Ala-His-Leu-Nle,
(SEQ ID NO. 163)
Eicosanedioyl-γGlu-Arg-Arg-His-Trp-Ala-Val-D-Ala-His-Leu-Nle,
(SEQ ID NO. 164)
Eicosanedioy1-γGlu-Gly-Asn-His-Trp-Ala-Val-D-Ala-His-Leu-Nle,
(SEQ ID NO. 165)
Eicosanedioyl-γGlu-Gly-Gln-His-Trp-Ala-Val-D-Ala-His-Leu-Nle-NH₂,
(SEQ ID NO. 166)
Octadecanedioyl-γGlu-OEG-OEG-Gly-Arg-His-Trp-Ala-Val-D-Ala-His-Leu-Nle,
(SEQ ID NO. 167)
Eicosanedioyl-γGlu-OEG-OEG-Gly-Arg-His-Trp-Ala-Val-D-Ala-His-Leu-Nle,
(SEQ ID NO. 240)
Palmitoyl-γGlu-γGlu-OEG-OEG-Gly-Asn-His-Trp-Ala-Val-Gly-His-Leu-Nle-NH₂,
(SEQ ID NO. 241)
Eicosanedioyl-γGlu-OEG-OEG-Gly-Gln-His-Trp-Ala-Val-β-Ala-His-Leu-Nle-NH₂,
(SEQ ID NO. 242)
Eicosanedioyl-γGlu-OEG-OEG-Gly-Arg-His-7MethylTrp-Ala-Val-β-Ala-His-Leu-Nle-NH₂,
(SEQ ID NO. 243)
Octadecanedioyl-γGlu-γGlu-OEG-OEG-Gly-Arg-His-Trp-Ala-Val-β-Ala-His-Leu-Nle-NH₂,
(SEQ ID NO. 244)
Eicosanedioyl-γGlu-γGlu-OEG-OEG-Gly-Arg-His-Trp-Ala-Val-D-Ala-His-Leu-Nle-NH₂,
(SEQ ID NO. 245)
Eicosanedioyl-γGlu-γGlu-OEG-OEG-D-Phe-Asn-3PAl-Trp-Ala-Val-β-Ala-His-Leu-Nle-NH₂,
(SEQ ID NO. 247)
Eicosanedioyl-γGlu-Glu-OEG-OEG-Gly-Glu-His-7MeTrp-Ala-Val-BABA-His-Leu-Nle-NH₂,
(SEQ ID NO. 248)
Eicosanedioyl-γGlu-Glu-OEG-OEG-Glu-D-Ser-Glu-His-7MeTrp-Ala-Val-β-Ala-His-Leu-Nle-
NH₂,
(SEQ ID NO. 251)
Eicosanedioyl-γGlu-Glu-OEG-OEG-Gly-Gln-His-Trp-Ala-Val-β-Ala-His-Leu-Nle-NH₂,
(SEQ ID NO. 252)
Eicosanedioyl-γGlu-Glu-OEG-OEG-Glu-Gly-Arg-His-7MeTrp-Ala-Val-β-Ala-His-Leu-Nle-
NH₂,
(SEQ ID NO. 253)
Eicosanedioyl-γGlu-Glu-OEG-OEG-Arg-Arg-His-7MeTrp-Ala-Val-β-Ala-His-Leu-Nle-NH₂,
(SEQ ID NO. 254)
Eicosanedioyl-γGlu-Glu-OEG-OEG-Arg-Gly-Arg-His-7MeTrp-Ala-Val-β-Ala-His-Leu-Nle-
NH₂,
(SEQ ID NO. 255)
Eicosanedioyl-γGlu-Glu-OEG-OEG-Ser-Gly-Glu-His-7MeTrp-Ala-Val-β-Ala-His-Leu-Nle-
NH₂,
(SEQ ID NO. 256)
Eicosanedioyl-γGlu-Glu-OEG-OEG-Ser-Gly-Arg-His-7MeTrp-Ala-Val-β-Ala-His-Leu-Nle-
NH₂,
(SEQ ID NO. 257)
Eicosanedioyl-γGlu-Glu-OEG-OEG-Glu-Ser-Gln-His-7MeTrp-Ala-Val-β-Ala-His-Leu-Nle-
NH₂,
(SEQ ID NO. 258)
Eicosanedioyl-γGlu-γGlu-OEG-OEG-Gly-Asn-His-7MeTrp-Ala-Val-BABA-His-Leu-Nle-NH₂,
(SEQ ID NO. 259)
Eicosanedioyl-γGlu-γGlu-OEG-OEG-Gly-Gln-His-7MeTrp-Ala-Val-BABA-His-Leu-Nle-NH₂,
(SEQ ID NO. 260)
Eicosanedioyl-γGlu-OEG-OEG-Gly-Dap-His-Trp-Ala-Val-β-Ala-His-Leu-Nle-NH₂,
(SEQ ID NO. 261)
Eicosanedioyl-γGlu-OEG-OEG-Gly-Dap(5CTMPA)-His-Trp-Ala-Val-β-Ala-His-Leu-Nle-NH₂,
(SEQ ID NO. 262)
Eicosanedioyl-γGlu-OEG-OEG-Gly-Dap(2CE-PEG-TMEA)-His-Trp-Ala-Val-β-Ala-His-Leu-
Nle-NH₂,
(SEQ ID NO. 263)
Eicosanedioyl-γGlu-OEG-OEG-Gly-Gln-His-7MeTrp-Ala-Val-BAIBA-His-Leu-Nle-NH₂,
(SEQ ID NO. 264)
Eicosanedioyl-γGlu-OEG-OEG-Gly-Asn-His-7MeTrp-Ala-Val-3A3MBA-His-Leu-Nle-NH₂,
(SEQ ID NO. 265)
Eicosanedioyl-γGlu-OEG-OEG-Gly-Gln-His-Trp-Ala-Val-β-Ala-His-Leu-Hse(Me)-NH₂,
(SEQ ID NO. 266)
Eicosanedioyl-γGlu-OEG-OEG-Gly-Glu-Gly-Glu-His-Trp-Ala-Val-β-Ala-His-Leu-Nle-NH₂,
(SEQ ID NO. 267)
Eicosanedioyl-γGlu-OEG-OEG-Gly-Gln-His-Trp-Ala-Val-β-Ala-NmeHis-Leu-Nle-NH₂,
(SEQ ID NO. 268)
Eicosanedioyl-γGlu-OEG-OEG-Pro-Gln-His-Trp-Ala-Val-β-Ala-His-Leu-Nle-NH₂,
(SEQ ID NO. 269)
Eicosanedioyl-γGlu-OEG-OEG-Hyp-Gln-His-Trp-Ala-Val-β-Ala-His-Leu-Nle-NH₂,
(SEQ ID NO. 270)
Eicosanedioyl-γGlu-Glu-OEG-OEG-Gly-Gln-His-7MeTrp-Ala-Val-BABA-NmeHis-Leu-Nle-
NH₂,
(SEQ ID NO. 271)
Eicosanedioyl-γGlu-γGlu-OEG-OEG-Gly-Gln-His-Trp-Ala-Val-ACPC-His-Leu-Nle-NH₂,
(SEQ ID NO. 272)
Eicosanedioyl-γGlu-γGlu-OEG-OEG-Gly-Gln-His-Trp-Ala-Val-ACPA-His-Leu-Nle-NH₂,
(SEQ ID NO. 273)
Eicosanedioyl-γGlu-Glu-OEG-OEG-Gly-Gln-His-Trp-Ala-Val-ACBA-His-Leu-Nle-NH₂,
(SEQ ID NO. 274)
Eicosanedioyl-γGlu-Glu-OEG-OEG-Gly-Asn-His-αMeTrp-Ala-Val-β-Ala-His-Leu-Nle-NH₂,
(SEQ ID NO. 275)
Eicosanedioyl-γGlu-Glu-PEG5-Gly-Glu-His-7MeTrp-Ala-Val-BABA-His-Leu-Nle-NH₂,
(SEQ ID NO. 276)
Eicosanedioyl-γGlu-OEG-OEG-Gly-Gln-His-Trp-Ala-Val-β-Ala-His-Leu-MetO-NH₂,
(SEQ ID NO. 277)
Eicosanedioyl-γGlu-γGlu-OEG-OEG-Ser-Glu-His-7MeTrp-Ala-Val-BABA-NMeHis-Leu-Nle-
NH₂,
or
(SEQ ID NO. 278)
Eicosanedioyl-γGlu-γGlu-OEG-OEG-Gly-Asp-His-7MeTrp-Ala-Val-BABA-NMeHis-Leu-Nle-
NH₂,

or a pharmaceutically acceptable salt, solvate, or isotope thereof.

Embodiment 157. A peptide comprising the sequence:

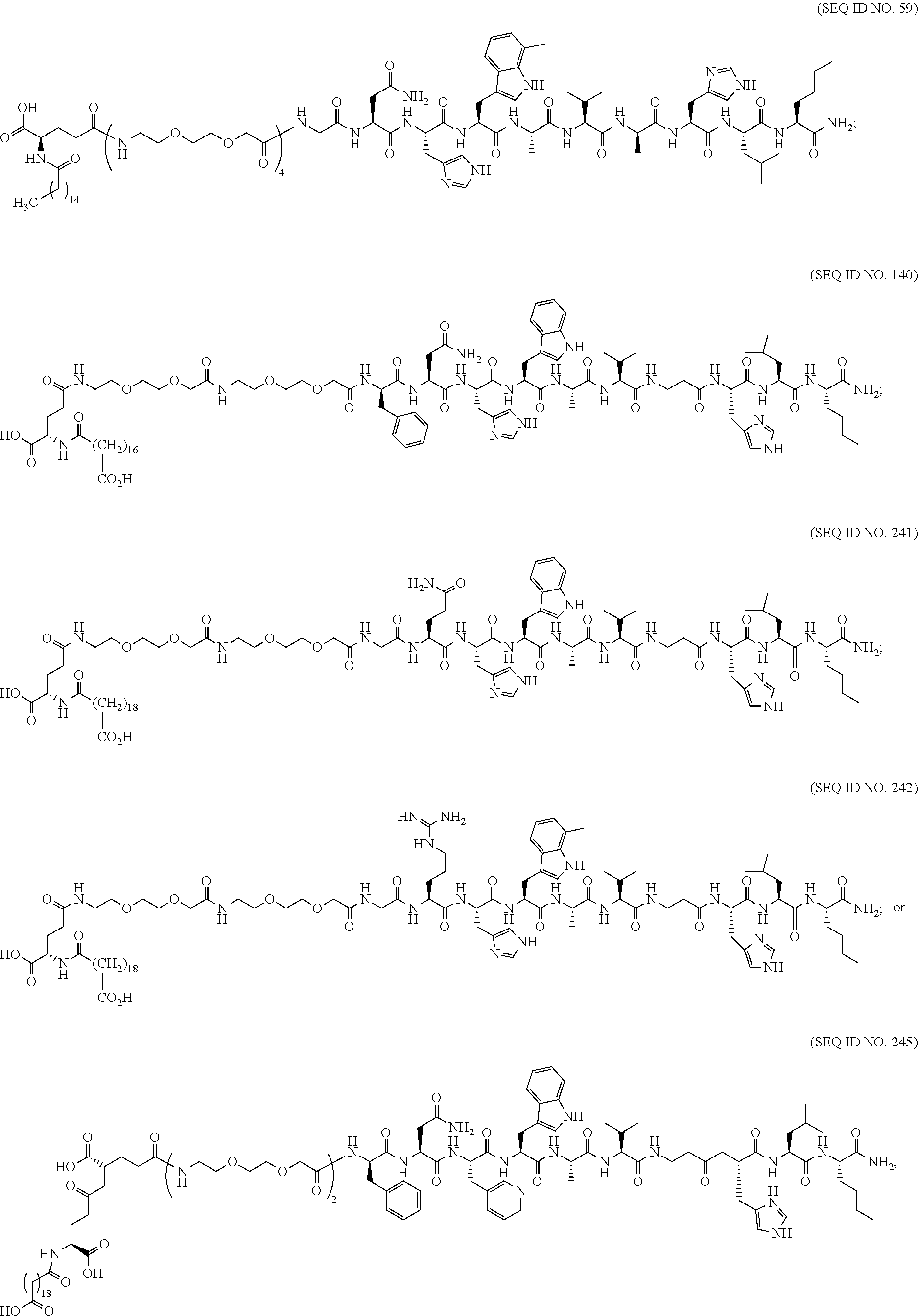

or a pharmaceutically acceptable salt, solvate, or isotope thereof.

Embodiment 158. A peptide comprising the sequence.

(SEQ ID NO. 5)
Octadecanedioyl-D-γGlu-OEG-OEG-Gly-Asn-His-Trp-Ala-Val-Gly-His-Leu-Met-NH₂;
(SEQ ID NO. 7)
Palmitoyl-D-γGlu-OEG-OEG-Gly-Asn-His-Trp-Ala-Val-D-Ala-His-
Leu-Nle-NH₂;
(SEQ ID NO. 8)
Palmitoyl-D-γGlu-OEG-OEG-Gly-Asn-His-Trp-Ala-Val-
Gly-His-Leu-Met-NH₂;
(SEQ ID NO. 9)
Octadecanedioyl-D-γGlu-OEG-OEG-Gly-Asn-His-Trp-Ala-Val-D-Ala-His-Leu-Nle-NH₂;
(SEQ ID NO. 241)
Eicosanedioyl-γGlu-OEG-OEG-Gly-Gln-His-Trp-Ala-Val-β-Ala-His-Leu-Nle-NH₂;
(SEQ ID NO. 247)
Eicosanedioyl-γGlu-Glu-OEG-OEG-Gly-Glu-His-7MeTrp-Ala-Val-BABA-His-Leu-
Nle-NH₂;
(SEQ ID NO. 248)
Eicosanedioyl-γGlu-Glu-OEG-OEG-Glu-D-Ser-Glu-His-
7MeTrp-Ala-Val-β-Ala-His-Leu-Nle-NH₂;
(SEQ ID NO. 252)
Eicosanedioyl-γGlu-Glu-
OEG-OEG-Glu-Gly-Arg-His-7MeTrp-Ala-Val-β-Ala-His-Leu-Nle-NH₂;
(SEQ ID NO. 255)
Eicosanedioyl-γGlu-Glu-OEG-OEG-Ser-Gly-Glu-His-7MeTrp-Ala-Val-β-Ala-
His-Leu-Nle-NH₂;
or
(SEQ ID NO. 257)
Eicosanedioyl-γGlu-Glu-OEG-OEG-Glu-Ser-
Gln-His-7MeTrp-Ala-Val-β-Ala-His-Leu-Nle-NH₂,

or a pharmaceutically acceptable salt, solvate, or isotope thereof.

Embodiment 159. A peptide comprising the sequence:

(SEQ ID NO. 5)
Octadecanedioyl-D-γGlu-OEG-OEG-Gly-Asn-His-Trp-Ala-Val-Gly-His-Leu-Met-NH₂;
(SEQ ID NO. 7)
Palmitoyl-D-γGlu-OEG-OEG-Gly-Asn-His-Trp-Ala-Val-D-Ala-His-
Leu-Nle-NH₂;
(SEQ ID NO. 241)
Eicosanedioyl-γGlu-OEG-OEG-Gly-Gln-His-Trp-Ala-
Val-β-Ala-His-Leu-Nle-NH₂;
(SEQ ID NO. 242)
Eicosanedioyl-γGlu-OEG-OEG-Gly-
Arg-His-7MethylTrp-Ala-Val-β-Ala-His-Leu-Nle-NH₂;
(SEQ ID NO. 247)
Eicosanedioyl-γGlu-γGlu-OEG-OEG-Gly-Glu-His-7MeTrp-Ala-Val-BABA-His-Leu-
Nle-NH₂;
(SEQ ID NO. 248)
Eicosanedioyl-γGlu-γGlu-OEG-OEG-Glu-D-Ser-Glu-His-
7MeTrp-Ala-Val-β-Ala-His-Leu-Nle-NH₂;
(SEQ ID NO. 277)
Eicosanedioyl-γGlu-
YGlu-OEG-OEG-Ser-Glu-His-7MeTrp-Ala-Val-BABA-NMeHis-Leu-Nle-NH₂;
or
Eicosanedioyl-γGlu-γGlu-OEG-OEG-Gly-Asp-His-7MeTrp-Ala-Val-
BABA-NMeHis-Leu-Nle-NH₂

(SEQ ID NO. 278), or a pharmaceutically acceptable salt, solvate, or isotope thereof.

Embodiment 160. A peptide comprising the sequence:
Octadecanedioyl-D-γGlu-OEG-OEG-Gly-Asn-His-Trp-Ala-Val-Gly-His-Leu-Met-NH₂ (SEQ ID NO. 5), or a pharmaceutically acceptable salt, solvate, or isotope thereof.

Embodiment 161. A peptide comprising the sequence:
Palmitoyl-D-γGlu-OEG-OEG-Gly-Asn-His-Trp-Ala-Val-D-Ala-His-Leu-Nle-NH₂(SEQ ID NO. 7), or a pharmaceutically acceptable salt, solvate, or isotope thereof.

Embodiment 162. A peptide comprising the sequence:
Eicosanedioyl-γGlu-OEG-OEG-Gly-Gln-His-Trp-Ala-Val-D-Ala-His-Leu-Nle-NH₂ (SEQ ID NO. 241), or a pharmaceutically acceptable salt, solvate, or isotope thereof.

Embodiment 163. A peptide comprising the sequence:
Eicosanedioyl-γGlu-OEG-OEG-Gly-Arg-His-7MethylTrp-Ala-Val-3-Ala-His-Leu-Nle-NH₂ (SEQ ID NO. 242), or a pharmaceutically acceptable salt, solvate, or isotope thereof.

Embodiment 164. A peptide comprising the sequence:
Eicosanedioyl-γGlu-γGlu-OEG-OEG-Gly-Glu-His-7MeTrp-Ala-Val-BABA-His-Leu-Nle-NH₂ (SEQ ID NO. 247), or a pharmaceutically acceptable salt, solvate, or isotope thereof.

Embodiment 165. A peptide comprising the sequence:
Eicosanedioyl-γGlu-γGlu-OEG-OEG-Glu-D-Ser-Glu-His-7MeTrp-Ala-Val-β-Ala-His-Leu-Nle-NH₂ (SEQ ID NO. 248), or a pharmaceutically acceptable salt, solvate, or isotope thereof.

Embodiment 166. A peptide comprising the sequence:
Eicosanedioyl-γGlu-γGlu-OEG-OEG-Ser-Glu-His-7MeTrp-Ala-Val-BABA-NMeHis-Leu-Nle-NH₂ (SEQ ID NO. 277), or a pharmaceutically acceptable salt, solvate, or isotope thereof.

Embodiment 167. A peptide comprising the sequence:
Eicosanedioyl-γGlu-γGlu-OEG-OEG-Gly-Asp-His-7MeTrp-Ala-Val-BABA-NMeHis-Leu-Nle-NH₂ (SEQ ID NO. 278), or a pharmaceutically acceptable salt, solvate, or isotope thereof.

Embodiment 168. A peptide comprising the sequence:

(SEQ ID NO. 6)
Gly-Asn-His-Trp-Ala-Val-D-Ala-His-Leu-Nle-NH₂;
(SEQ ID NO. 27)
Gly-Asn-His-Trp-Ala-Val-Aib-His-Leu-Nle-NH₂;
(SEQ ID NO. 28)
Gly-Asn-His-Trp-Ala-Val-Aib-3Pal-Leu-Nle-NH₂;
(SEQ ID NO. 29)
Gly-Asn-3Pal-Trp-Ala-Val-Aib-His-Leu-Nle-NH₂;
(SEQ ID NO. 30)
Gly-Asn-His-Trp-Ala-Val-Gly-His-Leu-Met-NH₂;
(SEQ ID NO. 31)
Gly-Asn-His-Trp-Aib-Val-D-Ala-His-Leu-Nle-NH₂;
(SEQ ID NO. 32)
Gly-Asn-3Pal-Trp-Ala-Val-D-Ala-His-Leu-Nle-NH₂;
(SEQ ID NO. 33)
Gly-Asn-His-7MethylTrp-Ala-Val-D-Ala-His-Leu-Nle-NH₂;
(SEQ ID NO. 34)
Gly-Asn-His-7MethylTrp-Aib-Val-D-Ala-His-Leu-Nle-NH₂;
(SEQ ID NO. 35)
Gly-Asn-His-Trp-Ala-Val-D-Ala-His-Leu-Nle-Sar-NH₂;
(SEQ ID NO. 36)
Gly-Glu-His-Trp-Ala-Val-D-Ala-His-Leu-Nle-NH₂;
(SEQ ID NO. 37)
Gly-Asn-His-Trp-Ala-Val-D-Ala-3Pal-Leu-Nle-NH₂;
(SEQ ID NO. 38)
Gly-Glu-3Pal-7MethylTrp-Aib-Val-D-Ala-3Pal-Leu-Nle-Sar-NH₂;
(SEQ ID NO. 39)
Gly-Glu-His-7MethylTrp-Aib-Val-D-Ala-3Pal-Leu-Nle-Sar-NH₂;
(SEQ ID NO. 40)
Gly-Asn-His-7MethylTrp-Aib-Val-D-Ala-3Pal-Leu-Nle-Sar-NH₂;
(SEQ ID NO. 41)
Gly-Asn-His-7MethylTrp-Ala-Val-Aib-His-Leu-Nle-NH₂;
(SEQ ID NO. 42)
Gly-Asn-His-7MethylTrp-Ala-Val-Aib-3Pal-Leu-Nle-Sar-NH₂;
(SEQ ID NO. 43)
Gly-Glu-His-Trp-Ala-Val-Aib-His-Leu-Nle-NH₂;
(SEQ ID NO. 168)
Gly-Asn-3Pal-7MethylTrp-Ala-Val-D-Ala-His-Leu-Nle-NH₂;
(SEQ ID NO. 169)
Gly-Asn-His-Trp-Ala-Val-D-Ala-His-Leu-Lys(Me)₃-NH₂;
(SEQ ID NO. 170)
Gly-Asn-2Pal-Trp-Ala-Val-D-Ala-His-Leu-Nle-NH₂;
(SEQ ID NO. 171)
Gly-Asn-4Pal-Trp-Ala-Val-D-Ala-His-Leu-Nle-NH₂;
(SEQ ID NO. 172)
Gly-Asn-His-7FTrp-Ala-Val-D-Ala-His-Leu-Nle-NH₂;
(SEQ ID NO. 173)
Gly-Lys-His-Trp-Ala-Val-D-Ala-His-Leu-Nle-NH₂;
(SEQ ID NO. 174)
Gly-Arg-His-Trp-Ala-Val-D-Ala-His-Leu-Nle-NH₂;
(SEQ ID NO. 175)
Gly-Asn-His-Trp-Ala-Val-D-Ala-Phe-Leu-Nle-NH₂;
(SEQ ID NO.176)
Gly-Asn-His-6MethylTrp-Ala-Val-D-Ala-His-Leu-Nle-NH₂;
(SEQ ID NO. 177)
Gly-Lys(Ac)-His-Trp-Ala-Val-D-Ala-His-Leu-Nle-NH₂;
(SEQ ID NO.179)
Gly-Glu-His-Trp-Ala-Val-D-Ala-3Pal-Leu-Nle-NH₂;
(SEQ ID NO. 180)
Gly-Asn-His-His-Ala-Val-D-Ala-His-Leu-Nle-NH₂;
(SEQ ID NO. 181)
Gly-Asn-His-Trp-Ala-Val-D-Ala-His-Leu-Arg-NH₂;
(SEQ ID NO. 182)
Gly-Glu-His-Trp-Aib-Val-D-Ala-His-Leu-Nle-NH₂;
(SEQ ID NO. 183)
Lys(Ac)-His-Trp-Ala-Val-D-Ala-His-Leu-Nle-NH₂;
(SEQ ID NO. 184)
Gly-Asn-3Pal-Trp-Ala-Val-D-Ala-His-Leu-Nle-Sar-NH₂;
(SEQ ID NO. 185)
Gly-Asn-3Pal-7MethylTrp-Ala-Val-D-Ala-His-Leu-Nle-Sar-NH₂;
(SEQ ID NO. 186)
Aib-Asn-His-Trp-Ala-Val-D-Ala-His-Leu-Nle-NH₂;
(SEQ ID NO. 187)
Gly-Asn-His-Trp-Ala-Val-D-Ala-His-Leu-Nle-Aib-NH₂;
(SEQ ID NO. 188)
Gly-Asn-His-7MethylTrp-Ala-Val-D-Ala-His-Leu-Nle-Aib-NH₂;
(SEQ ID NO. 189)
Gly-Lys(Ac)-His-Trp-Ala-Val-D-Ala-His-Leu-Nle-Sar-NH₂;
(SEQ ID NO. 190)
Gly-Asn-His-7MethylTrp-Ala-Val-D-Ala-His-Leu-NH₂;
(SEQ ID NO. 191)
Gly-Asn-His-7MethylTrp-Ala-Val-D-Ala-His-Leu-Nle-Sar-NH₂;
(SEQ ID NO. 192)
Gly-Asn-His-6MethylTrp-Ala-Val-D-Ala-His-Leu-Nle-Sar-NH₂;
(SEQ ID NO. 193)
Gly-Arg-His-Trp-Ala-Val-D-Ala-His-Leu-Nle-NH₂;
(SEQ ID NO. 194)
Gly-Asn-His-2Nal-Ala-Val-Ala-His-Leu-Nle-NH₂;
(SEQ ID NO. 195)
Gly-Asn-3Pal-7MeTrp-Ala-Val-D-Ala-His-Leu-Nle-NH₂;
(SEQ ID NO. 196)
Gly-Asn-His-2Nal-Ala-Val-D-Ala-His-Leu-Nle-Sar-NH₂;
(SEQ ID NO. 197)
Arg-Asn-His-Trp-Ala-Val-D-Ala-His-Leu-Nle-Sar-NH₂;
(SEQ ID NO. 198)
Gly-Asn-His-7FTrp-Ala-Val-D-Ala-His-Leu-Nle-Sar-NH₂;
(SEQ ID NO. 199)
Gly-Asn-His-2Nal-Ala-Val-D-Ala-His-Leu-Nle-NH₂;
(SEQ ID NO. 200)
Gly-Asn-His-Trp-D-Ala-Val-D-Ala-His-Leu-Nle-NH₂;
(SEQ ID NO. 201)
Gly-Asn-His-Arg-Ala-Val-D-Ala-His-Leu-Nle-NH₂;
(SEQ ID NO. 202)
Gly-Arg-His-7FTrp-Ala-Val-D-Ala-His-Leu-Nle-NH₂;
(SEQ ID NO. 203)
Gly-Asn-His-Trp-Ala-Val-D-Ala-His-Leu-Nle-Arg-NH₂;
(SEQ ID NO. 204)
Gly-Asn-His-Trp-D-Ala-Val-D-Ala-His-Leu-Nle-Sar-NH₂;
(SEQ ID NO. 205)
Gly-Arg-His-His-Ala-Val-D-Ala-His-Leu-Nle-NH₂;
(SEQ ID NO. 206)
Gly-Arg-His-7MethylTrp-Ala-Val-D-Ala-His-Leu-Nle-NH₂;
(SEQ ID NO. 207)
Gly-Asn-His-Trp-Ala-Val-D-Ala-His-Leu-Cha-NH₂;
(SEQ ID NO. 208)
Gly-Ala-His-Trp-Ala-Val-D-Ala-His-Leu-Cha-NH₂;
(SEQ ID NO. 209)
Gly-Ala-His-7FTrp-Ala-Val-D-Ala-His-Leu-Nle-Sar-NH₂;
(SEQ ID NO. 210)
Gly-Ala-His-2Nal-Ala-Val-D-Ala-His-Leu-Nle-Sar-NH₂;
(SEQ ID NO. 211)
Gly-Asn-His-7MethylTrp-D-Ala-Val-D-Ala-His-Leu-Nle-Sar-NH₂;
(SEQ ID NO. 212)
Gly-Ala-His-2Nal-Ala-Val-D-Ala-His-Leu-Nle-NH₂;
(SEQ ID NO. 214)
Gly-Ala-His-Trp-D-Ala-Val-D-Ala-His-Leu-Nle-Sar-NH₂;
(SEQ ID NO. 215)
Gly-Arg-His-7MethylTrp-Ala-Val-D-Ala-His-Leu-Nle-Sar-NH₂;
(SEQ ID NO. 216)
Gly-Asn-His-Arg-Ala-Val-D-Ala-His-Leu-Nle-Sar-NH₂;
(SEQ ID NO. 217)
Gly-Asn-His-6FTrp-Ala-Val-D-Ala-His-Leu-Nle-Sar-NH₂;
(SEQ ID NO. 218)
Gly-Asn-His-7MethylTrp-Ala-Val-D-Ala-His-Leu-Nle-Arg-NH₂;
(SEQ ID NO. 220)
Gly-Ala-His-7FTrp-Ala-Val-D-Ala-His-Leu-Nle-NH₂;
(SEQ ID NO. 221)
Gly-Arg-His-7FTrp-Ala-Val-D-Ala-His-Leu-Nle-Sar-NH₂;
(SEQ ID NO. 222)
Gly-Asp-His-Trp-D-Ala-Val-D-Ala-His-Leu-Nle-NH₂;
(SEQ ID NO. 223)
Phe-Asn-His-Trp-Ala-Val-β-Ala-His-Leu-Nle-NH₂;
(SEQ ID NO. 224)
Arg-Arg-His-Trp-Ala-Val-D-Ala-His-Leu-Nle-NH₂;
(SEQ ID NO. 225)
Gly-Asn-His-Trp-Ala-Val-β-Ala-His-Leu-Nle-NH₂;
(SEQ ID NO. 226)
D-Phe-Asn-His-Trp-Ala-Val-D-Ala-His-Leu-Nle-NH₂;
(SEQ ID NO. 227)
Phe-Asn-3Pal-Trp-Ala-Val-β-Ala-His-Leu-Nle-NH₂;
(SEQ ID NO. 228)
Arg-Asn-His-Trp-Ala-Val-D-Ala-His-Leu-Nle-NH₂;
(SEQ ID NO. 229)
Gly-Asn-His-Trp-Ala-Val-D-Ala-His-Leu-Nle;
(SEQ ID NO. 230)
Gly-Gln-His-Trp-Ala-Val-D-Ala-His-Leu-Nle-NH₂;
(SEQ ID NO. 231)
Arg-Asn-His-Trp-Ala-Val-D-Ala-His-Leu-Nle;
(SEQ ID NO. 232)
Arg-Arg-His-Trp-Ala-Val-D-Ala-His-Leu-Nle;
(SEQ ID NO. 233)
Gly-Asn-His-Trp-Ala-Val-D-Ala-D-His-Leu-Nle-NH₂;
(SEQ ID NO. 234)
D-Phe-Gln-His-Trp-Ala-Val-D-Ala-His-Leu-Nle-NH₂;
(SEQ ID NO. 235)
Gly-Arg-His-Trp-Ala-Val-D-Ala-His-Leu-Nle;
(SEQ ID NO. 236)
Gly-Asn-His-Trp-Ala-Val-Gly-His-Leu-Nle-NH₂;
(SEQ ID NO. 237)
Gly-Gln-His-Trp-Ala-Val-β-Ala-His-Leu-Nle-NH₂;
(SEQ ID NO. 238)
Gly-Arg-His-7MethylTrp-Ala-Val-β-Ala-His-Leu-Nle-NH₂;
(SEQ ID NO. 239)
Gly-Arg-His-Trp-Ala-Val-β-Ala-His-Leu-Nle-NH₂;
(SEQ ID NO. 246)
D-Phe-Asn-3Pal-Trp-Ala-Val-β-Ala-His-Leu-Nle-NH₂;
(SEQ ID NO. 249)
Gly-Glu-His-7MeTrp-Ala-Val-BABA-His-Leu-Nle-NH₂;
(SEQ ID NO. 250)
Glu-D-Ser-Glu-His-7MeTrp-Ala-Val-β-Ala-His-Leu-Nle-NH₂;
(SEQ ID NO. 279)
Gly-Asn-His-Trp-Ala-Val-D-Ala-His-Leu-Nle;
(SEQ ID NO. 280)
Arg-Arg-His-7MeTrp-Ala-Val-β-Ala-His-Leu-Nle-NH₂;
(SEQ ID NO. 281)
Arg-Gly-Arg-His-7MeTrp-Ala-Val-β-Ala-His-Leu-Nle-NH₂;
(SEQ ID NO. 282)
Ser-Gly-Glu-His-7MeTrp-Ala-Val-β-Ala-His-Leu-Nle-NH₂;
(SEQ ID NO. 283)
Ser-Gly-Arg-His-7MeTrp-Ala-Val-β-Ala-His-Leu-Nle-NH₂;
(SEQ ID NO. 284)
Glu-Ser-Gln-His-7MeTrp-Ala-Val-β-Ala-His-Leu-Nle-NH₂;
(SEQ ID NO. 285)
Gly-Asn-His-7MeTrp-Ala-Val-BABA-His-Leu-Nle-NH₂;
(SEQ ID NO. 286)
Gly-Gln-His-7MeTrp-Ala-Val-BABA-His-Leu-Nle-NH₂;
(SEQ ID NO. 287)
Gly-Dap-His-Trp-Ala-Val-β-Ala-His-Leu-Nle-NH₂;
(SEQ ID NO. 288)
Gly-Dap(5CTMPA)-His-Trp-Ala-Val-β-Ala-His-Leu-Nle-NH₂;
(SEQ ID NO. 289)
Gly-Dap(2CE-PEG-TMEA)-His-Trp-Ala-Val-β-Ala-His-Leu-Nle-NH₂;
(SEQ ID NO. 290)
Gly-Gln-His-7MeTrp-Ala-Val-BAIBA-His-Leu-Nle-NH₂;
(SEQ ID NO. 291)
Gly-Asn-His-7MeTrp-Ala-Val-3A3MBA-His-Leu-Nle-NH₂;
(SEQ ID NO. 292)
Gly-Gln-His-Trp-Ala-Val-β-Ala-His-Leu-Hse(Me)-NH₂;
(SEQ ID NO. 293)
Gly-Glu-Gly-Glu-His-Trp-Ala-Val-β-Ala-His-Leu-Nle-NH₂;
(SEQ ID NO. 294)
Gly-Gln-His-Trp-Ala-Val-β-Ala-NMeHis-Leu-Nle-NH₂;
(SEQ ID NO. 295)
Pro-Gln-His-Trp-Ala-Val-β-Ala-His-Leu-Nle-NH₂;
(SEQ ID NO. 296)
Hyp-Gln-His-Trp-Ala-Val-β-Ala-His-Leu-Nle-NH₂;
(SEQ ID NO. 297)
Gly-Gln-His-7MeTrp-Ala-Val-BABA-NMeHis-Leu-Nle-NH₂;
(SEQ ID NO. 298)
Gly-Gln-His-Trp-Ala-Val-ACPC-His-Leu-Nle-NH₂;
(SEQ ID NO. 299)
Gly-Gln-His-Trp-Ala-Val-ACPA-His-Leu-Nle-NH₂;
(SEQ ID NO. 300)
Gly-Gln-His-Trp-Ala-Val-ACBA-His-Leu-Nle-NH₂;
(SEQ ID NO. 301)
Gly-Asn-His-αMeTrp-Ala-Val-β-Ala-His-Leu-Nle-NH₂;
(SEQ ID NO. 302)
Gly-Glu-His-7MeTrp-Ala-Val-BABA-His-Leu-Nle-NH₂;
(SEQ ID NO. 303)
Gly-Gln-His-Trp-Ala-Val-β-Ala-His-Leu-MetO-NH₂;
(SEQ ID NO. 304)
Ser-Glu-His-7MeTrp-Ala-Val-BABA-NMeHis-Leu-Nle-NH₂;
(SEQ ID NO. 305)
Gly-Asp-His-7MeTrp-Ala-Val-BABA-NMeHis-Leu-Nle-NH₂;
or
(SEQ ID NO. 306)
Glu-Gly-Arg-His-7MeTrp-Ala-Val-β-Ala-His-Leu-Nle-NH₂,

or a pharmaceutically acceptable salt, solvate, or isotope thereof.

Embodiment 169. A peptide comprising the sequence:
Gly-Asn-His-Trp-Ala-Val-Gly-His-Leu-Met-NH₂ (SEQ ID NO. 30);
Gly-Asn-His-Trp-Ala-Val-D-Ala-His-Leu-Nle-NH₂ (SEQ ID NO. 6);
Gly-Gln-His-Trp-Ala-Val-β-Ala-His-Leu-Nle-NH₂(SEQ ID NO. 237);
Gly-Glu-His-7MeTrp-Ala-Val-BABA-His-Leu-Nle-NH₂(SEQ ID NO. 249);
Glu-D-Ser-Glu-His-7MeTrp-Ala-Val-β-Ala-His-Leu-Nle-NH₂ (SEQ ID NO. 250);
Glu-Gly-Arg-His-7MeTrp-Ala-Val-β-Ala-His-Leu-Nle (SEQ ID NO. 306);
Ser-Gly-Glu-His-7MeTrp-Ala-Val-β-Ala-His-Leu-Nle-NH₂(SEQ ID NO. 282);
Glu-Ser-Gln-His-7MeTrp-Ala-Val-β-Ala-His-Leu-Nle-NH₂ (SEQ ID NO. 284), or a pharmaceutically acceptable salt, solvate, or isotope thereof.

Embodiment 170. A peptide comprising the sequence:

(SEQ ID NO. 30)
Gly-Asn-His-Trp-Ala-Val-Gly-His-Leu-Met-NH₂;
(SEQ ID NO. 6)
Gly-Asn-His-Trp-Ala-Val-D-Ala-His-Leu-Nle-NH₂;
(SEQ ID NO. 237)
Gly-Gln-His-Trp-Ala-Val-β-Ala-His-Leu-Nle-NH₂;
(SEQ ID NO. 249)
Gly-Glu-His-7MeTrp-Ala-Val-BABA-His-Leu-Nle-NH₂;
(SEQ ID NO. 250)
Glu-D-Ser-Glu-His-7MeTrp-Ala-Val-β-Ala-His-Leu-
Nle-NH₂;,
(SEQ ID NO. 304)
Ser-Glu-His-7MeTrp-Ala-Val-BABA-NMeHis-Leu-Nle-
NH₂;
(SEQ ID NO. 305)
Gly-Asp-His-7MeTrp-Ala-Val-BABA-NMeHis-Leu-Nle-
NH₂,

or a pharmaceutically acceptable salt, solvate, or isotope thereof.

Embodiment 171. A peptide comprising the sequence:
Gly-Asn-His-Trp-Ala-Val-Gly-His-Leu-Met-NH₂ (SEQ ID NO. 30);

(SEQ ID NO. 30)
Gly-Asn-His-Trp-Ala-Val-Gly-His-Leu-Met-NH₂;

or a pharmaceutically acceptable salt, solvate, or isotope thereof.

Embodiment 172. A peptide comprising the sequence:
Gly-Asn-His-Trp-Ala-Val-D-Ala-His-Leu-Nle-NH₂ (SEQ ID NO. 6);

(SEQ ID NO. 6)
Gly-Asn-His-Trp-Ala-Val-D-Ala-His-Leu-Nle-NH₂;

or a pharmaceutically acceptable salt, solvate, or isotope thereof.

Embodiment 173. A peptide comprising the sequence:

(SEQ ID NO. 237)
Gly-Gln-His-Trp-Ala-Val-β-Ala-His-Leu-Nle-NH₂;

or a pharmaceutically acceptable salt, solvate, or isotope thereof.

Embodiment 174. A peptide comprising the sequence:
Gly-Glu-His-7MeTrp-Ala-Val-BABA-His-Leu-Nle-NH₂(SEQ ID NO. 249);

(SEQ ID NO. 249)
Gly-Glu-His-7MeTrp-Ala-Val-BABA-His-Leu-Nle-NH₂;

or a pharmaceutically acceptable salt, solvate, or isotope thereof.

Embodiment 175. A peptide comprising the sequence:

(SEQ ID NO. 250)
Glu-D-Ser-Glu-His-7MeTrp-Ala-Val-β-Ala-His-Leu-
Nle-NH₂;

or a pharmaceutically acceptable salt, solvate, or isotope thereof.

Embodiment 176. A peptide comprising the sequence:

(SEQ ID NO. 304)
Ser-Glu-His-7MeTrp-Ala-Val-BABA-NMeHis-Leu-Nle-NH₂;

or a pharmaceutically acceptable salt, solvate, or isotope thereof.

Embodiment 177. A peptide comprising the sequence:
Gly-Asp-His-7MeTrp-Ala-Val-BABA-NMeHis-Leu-Nle-NH₂ (SEQ ID NO. 305);

(SEQ ID NO. 305)
Gly-Asp-His-7MeTrp-Ala-Val-BABA-NMeHis-Leu-Nle-NH₂;

or a pharmaceutically acceptable salt, solvate, or isotope thereof.

Embodiment 178. A peptide comprising the sequence:
Glu-Gly-Arg-His-7MeTrp-Ala-Val-β-Ala-His-Leu-Nle-NH₂ (SEQ ID NO. 306);

(SEQ ID NO. 306)
Glu-Gly-Arg-His-7MeTrp-Ala-Val-β-Ala-His-Leu-Nle-
NH₂;

or a pharmaceutically acceptable salt, solvate, or isotope thereof.

Embodiment 179. A peptide comprising the sequence:

(SEQ ID NO. 284)
Glu-Ser-Gln-His-7MeTrp-Ala-Val-β-Ala-His-Leu-Nle-
NH₂;

or a pharmaceutically acceptable salt, solvate, or isotope thereof.

Embodiment 180. A peptide comprising the sequence:

(SEQ ID NO. 282)
Ser-Gly-Glu-His-7MeTrp-Ala-Val-β-Ala-His-Leu-Nle-
NH₂;

or a pharmaceutically acceptable salt, solvate, or isotope thereof.

Embodiment 181. A long-acting GRP peptide analog comprising;
an amino acid sequence of GRP10 (SEQ ID NO. 44); and
a lipid moiety of any one of embodiments 95-128.

Embodiment 182. A pharmaceutical composition comprising:
(i) the peptide of any one of embodiments 1-180, or a pharmaceutically acceptable salt or solvate thereof, and
(ii) a pharmaceutically acceptable carrier, diluent, or excipient.

Embodiment 183. The pharmaceutical composition of embodiment 182, wherein the pharmaceutical composition further comprises an absorption enhancer.

Embodiment 184. The pharmaceutical composition of embodiment 182, wherein the absorption enhancer is for use in an oral formulation.

Embodiment 185. The pharmaceutical composition of embodiment 182, wherein the absorption enhancer is selected from the group consisting of sodium caprate, sodium caprylate, labrasol, and sodium N-(8-[2-hydroxylbenzoyl]amino)caprylate.

Embodiment 186. Use of the peptide of any one of embodiments 1-180, or the pharmaceutical composition of any one of embodiments 182-185, for the preparation of a medicament for the treatment of a disorder.

Embodiment 187. The use of embodiment 186, wherein the disorder is a gastrointestinal disease.

Embodiment 188. The use of embodiment 186, wherein the disorder is obesity.

Embodiment 189. A method for weight management in a subject in need thereof, the method comprising: administering a therapeutically effective amount of a lipidated gastrin releasing peptide (GRP), or a pharmaceutically acceptable salt, solvate, or isotope thereof.

Embodiment 190. A method for reducing body weight of a subject in need thereof, the method comprising: administering a therapeutically effective amount of a lipidated gastrin releasing peptide (GRP), or a pharmaceutically acceptable salt, solvate, or isotope thereof.

Embodiment 191. A method for treating obesity in a subject in need thereof, the method comprising: administering a therapeutically effective amount of a lipidated gastrin releasing peptide (GRP), or a pharmaceutically acceptable salt, solvate, or isotope thereof.

Embodiment 192. The method of any one of embodiments 189-191, wherein the lipidated GRP comprises an amino acid sequence of SEQ ID NO. 44, or a pharmaceutically acceptable salt, solvate, or isotope thereof.

Embodiment 193. The method of any one of embodiments 189-191, wherein the lipidated GRP has a longer half-life than non-lipidated SEQ ID NO. 44, or a pharmaceutically acceptable salt, solvate, or isotope thereof.

Embodiment 194. A method for weight management in a subject in need thereof, the method comprising: administering a therapeutically effective amount of a lipidated gastrin releasing peptide (GRP) analog, or a pharmaceutically acceptable salt, solvate, or isotope thereof.

Embodiment 195. A method for reducing body weight of a subject in need thereof, the method comprising: administering a therapeutically effective amount of a lipidated gastrin releasing peptide (GRP) analog, or a pharmaceutically acceptable salt, solvate, or isotope thereof.

Embodiment 196. A method for treating obesity in a subject in need thereof, the method comprising: administering a therapeutically effective amount of a lipidated gastrin releasing peptide (GRP) analog, or a pharmaceutically acceptable salt, solvate, or isotope thereof.

Embodiment 197. The method of any one of embodiments 194-196, wherein the lipidated GRP analog comprises a peptide of any one of embodiments 1-180, or a pharmaceutically acceptable salt, solvate, or isotope thereof.

Embodiment 198. The method of any one of embodiments 194-196, wherein the lipidated GRP analog comprises a peptide of any one of SEQ ID NO. 1-5, 7-26, 46-112, 114-126, 128-134, 136-167, 240-245, 247, 248, or 251-278, or a pharmaceutically acceptable salt, solvate, or isotope thereof.

Embodiment 199. The method of any one of embodiments 194-196, wherein the lipidated GRP analog has a longer half-life than non-lipidated SEQ ID NO. 44, or a pharmaceutically acceptable salt, solvate, or isotope thereof.

Embodiment 200. A method for weight management in a subject in need thereof, the method comprising: administering a therapeutically effective amount of a peptide of any one of embodiments 1-180, or a pharmaceutically acceptable salt, solvate, or isotope thereof, or the pharmaceutical composition of any one of embodiments 182-185.

Embodiment 201. A method for reducing body weight of a subject in need thereof, the method comprising: administering a therapeutically effective amount of a peptide of any one of embodiments 1-180, or a pharmaceutically acceptable salt, solvate, or isotope thereof, or the pharmaceutical composition of any one of embodiments 182-185.

Embodiment 202. A method for treating obesity in a subject in need thereof, the method comprising: administering a therapeutically effective amount of a peptide of any one of embodiments 1-180, or a pharmaceutically acceptable salt, solvate, or isotope thereof, or the pharmaceutical composition of any one of embodiments 182-185.

Embodiment 203. The method of any one of embodiments 189-202, wherein the administering occurs weekly.

Embodiment 204. The method of any one of embodiments 189-202, wherein the administering occurs every 7-14 days, semimonthly, or monthly.

Embodiment 205. The method of any one of embodiments 187-203, wherein the administering occurs semimonthly or monthly.

Embodiment 206. The method of any one of embodiments 187-203, wherein the administering occurs monthly.

Embodiment 207. The method of any one of embodiments 187-206, wherein the subject is a human.

Embodiment 208. The method of any one of embodiments 187-207, wherein the subject is overweight.

Embodiment 209. The method of any one of embodiments 187-208, wherein the subject is obese.

Embodiment 210. The method of any one of embodiments 187-209, wherein the subject has at least one weight-related comorbid condition selected from the group consisting of:
hypertension, type 2 diabetes mellitus, dyslipidemia, metabolic-associated steatohepatitis, sleep apnea, and urinary incontinence.

Embodiment 211. The method of any one of embodiments 187-210, wherein the subject has received at least one previous treatment of a weight management therapy.

Embodiment 212. The method of any one of embodiments 187-210, wherein administering the peptide reduces a body weight of the subject by at least 5% compared to a body weight of an otherwise identical subject not administered the peptide.

Embodiment 213. The method of any one of embodiments 187-212, wherein the peptide is administered intravenously.

Embodiment 214. The method of any one of embodiments 187-212, wherein the peptide is administered subcutaneously.

Embodiment 215. The method of any one of embodiments 187-212, wherein the peptide is administered orally.

Embodiment 216. The method of any one of embodiments 187-215, further comprising administering with an absorption enhancer.

Embodiment 217. The method of embodiment 216, wherein the absorption enhancer is for use in an oral formulation.

Embodiment 218. The method of embodiment 216, wherein the absorption enhancer is selected from the group consisting of: sodium caprate, sodium caprylate, labrasol, and sodium N-(8-[2-hydroxylbenzoyl]amino) caprylate.

Embodiment 219. The method of any one of embodiments 187-218, further comprising administering a therapeutically effective amount of another weight loss agent

### EXAMPLES

The following examples are provided to further illustrate some embodiments of the present disclosure but are not intended to limit the scope of the disclosure.

As used above, and throughout the description of the invention, the following abbreviations, unless otherwise indicated, shall be understood to have the following meanings:

### Abbreviations

ACN or MeCN or CH₃CN: acetonitrile;
Ac₂O: acetic anhydride;
brine: saturated aqueous NaCl solution;
DEPBT: 3-(diethoxyphosphoryloxy)-1,2,3-benzotriazin-4(3H)-one
DCM: dichloromethane;
DEPBT: 3-(diethoxyphosphoryloxy)-1,2,3-benzotriazin-4(3H)-one
DIEA or DIPEA: N,N-diisopropylethylamine;
DMF: dimethylformamide;
DMSO: dimethyl sulfoxide;
EtOAc or EA: ethyl acetate;
Fmoc: fluorenylmethoxycarbonyl;
HATU: 1-[bis(dimethylamino)methylene]-1H-1,2,3-triazolo[4,5-b]pyridinium 3-oxide hexafluorophosphate;
HBTU: hexafluorophosphate benzotriazole tetramethyl uronium
HCl: hydrochloric acid or hydrochloride;
Hex: hexanes;
H₂O: water;
HBTU: N,N,N′,N′-tetramethyl-O-(1H-benzotriazol-1-yl)uronium hexafluorophosphate;
HPLC: high-performance liquid chromatography;
H₂O: water
LCMS: liquid chromatography-mass spectrometry;
MBHA resin: methylbenzhydryl amine resin;
MeOH: methanol;
MS: mass spectrometry;
MTBE: methyl tert-butyl ether;
NMP: N-methyl-2-pyrrolidone;
N₂: nitrogen;
PBS: phosphate-buffered saline;
PE: petroleum ether;
PEG: —CH₂CH₂O—;
Prep-HPLC: preparative high-performance liquid chromatography;
RP-HPLC: reversed-phase high-performance liquid chromatography;
SPPS: solid-phase peptide synthesis;
TFA: trifluoroacetic acid;
THF: tetrahydrofuran;
t-Bu: tert-butyl;
rt: room temperature;
min: minute;
h or hr: hour;
mg: milligrams; kg: kilograms;
mL or ml: milliliter;
eq: equivalents;
mol: mole;
mmol: millimole;
UV: ultraviolet;
v/v: volume/volume.

### Example 1: Synthesis of Peptide 7

Peptide 7 was synthesized by standard Solid-phase Peptide Synthesis (SPPS) using Fmoc/t-Bu chemistry. The assembly was performed on a Rink-amide MBHA resin (100-200 mesh; loading 0.651 mmol/g) in a peptide solid phase synthesis tube under a nitrogen atmosphere. The constructed peptide was isolated from the resin and protecting groups removed by cleavage with strong acid followed by precipitation. The peptide purification was performed by prep-HPLC. Lyophilization of pure fractions gave the final product.

Swell Resin: 1.55 g of Rink Amide MBHA solid phase resin (0.651 mmol/g loading) was transferred to a 50-L peptide vessel with filter frit, ground glass joint and vacuum side arm. The resin was washed with DMF (100 mL×3).

Step 1: Coupling of Fmoc-Nle-OH: A solution of 9H-fluoren-9-ylmethyl carbamate (1.5 g, 0.651 mmol/g loading) and piperidine (100 mL 20% purity in DMF) was agitated with the resin under N₂ for 30 min, and then filtered. The resin (filter cake) was washed with DMF (100 ml×3). To a mixture of Fmoc-Nle-OH (6.9 g, 19.5 mmol, 3 eq) and DIPEA (5.1 g, 39 mmol, 6 eq) in DMF (100 mL) was added HBTU (7.0 g, 18 mmol, 2.85 eq) at 25° C. The mixture was stirred at 25° C. for 20 min. And then the mixture was added to the resin. The resulting mixture was agitated under N₂ for 40 min. After the reaction was judged complete, the resin was filtered and washed with DMF (100 mL×3), and then diluted with DIEA (6.8 mL, 3 eq) and Ac₂O (1.83 mL, 3 eq) in DMF (100 mL). The resulting mixture was agitated under N₂ at 25° C. for 30 min, and then filtered and washed with DMF (100 ml×3), DCM (100 ml×3) and DMF (100 ml×3).

Step 2: Coupling of Fmoc-L-Leu-OH: A solution of the resin and piperidine (100 mL, 20% purity in DMF) was agitated under N₂ for 30 min, and then filtered. The resin (filter cake) was washed with DMF (100 ml×3). To a mixture of Fmoc-L-Leu-OH (6.9 g, 19 mmol, 3 eq) and DIPEA (5.0 g, 39 mmol, 6 eq) in DMF (100 mL) was added HBTU (7.0 g, 18 mmol, 2.85 eq) at 25° C. The mixture was stirred at 25° C. for 20 min. And then the mixture was added to the deprotected Nle-amide resin. The resulting mixture was agitated under N₂ for 40 min. After the reaction was judged complete, the resin was filtered and washed with DMF (100 mL×3).

Step 3: Coupling of Fmoc-HIS(TRT)-OH: A solution of the resin and piperidine (100 mL, 20% purity in DMF) was agitated under N₂ for 30 min, and then filtered. The resin (filter cake) was washed with DMF (100 ml×3). To a mixture of FMOC-HIS(TRT)-OH (12 g, 19 mmol, 3 eq) and DIPEA (5.0 g, 39 mmol, 6 eq) in DMF (100 mL) was added HBTU (7.0 g, 18 mmol, 2.85 eq) at 25° C. The mixture was stirred at 25° C. for 20 min. And then the mixture was added to the deprotected the Leu-Nle-amide resin. The resulting mixture was agitated under N₂ for 40 min. After the reaction was judged complete, the resin was filtered and washed with DMF (100 mL×3).

Step 4: Coupling of Fmoc-D-ALA-OH: A solution of the resin and piperidine (100 mL 20% purity in DMF) was agitated under N₂ for 30 min, and then filtered. The resin (filter cake) was washed with DMF (100 ml×3). A mixture of Fmoc-D-ALA-OH (6 g, 19 mmol, 3 eq) and DIPEA (5.0 g, 39 mmol, 6 eq) in DMF (100 mL) was added HBTU (7.0 g, 18 mmol, 2.85 eq) at 25° C. The mixture was stirred at 25° C. for 20 min. And then to the mixture was added the deprotected His(Trt)-Leu-Nle-amide resin. The resulting mixture was agitated under N₂ for 40 min. After the reaction was judged complete, the resin was filtered and washed with DMF (100 mL×3).

Step 5: Coupling of Fmoc-L-Val-OH: A solution of the resin and piperidine (100 mL, 20% purity in DMF) was agitated under N₂ for 30 min, and then filtered. The resin (filter cake) was washed with DMF (100 ml×3). To a mixture of Fmoc-L-Val-OH (6.6 g, 19 mmol, 3 eq) and DIPEA (5.0 g, 39 mmol, 6 eq) in DMF (100 mL) was added HBTU (7.0 g, 18 mmol, 2.85 eq) at 25° C. The mixture was stirred at 25° C. for 20 min. And then the mixture was added to the deprotected the Ala-His(Trt)-Leu-Nle-amide resin. The resulting mixture was agitated under N₂ for 40 min. After the reaction was judged complete, the resin was filtered and washed with DMF (100 mL×3).

Step 6: Coupling of Fmoc-Ala-OH: A solution of the resin and piperidine (100 mL, 20% purity in DMF) was agitated under N₂ for 30 min, and then filtered. The resin (filter cake) was washed with DMF (100 ml×3). To a mixture of Fmoc-Ala-OH (6.1 g, 19 mmol, 3 eq) and DIPEA (5.0 g, 39 mmol, 6 eq) in DMF (100 mL) was added HBTU (7.0 g, 18 mmol, 2.85 eq) at 25° C. The mixture was stirred at 25° C. for 20 min. And then the mixture was added to the deprotected the Val-Ala-His(Trt)-Leu-Nle-amide resin (SEQ ID NO: 307). The resulting mixture was agitated under N₂ for 40 min. After the reaction was judged complete, the resin was filtered and washed with DMF (100 mL×3).

Step 7: Coupling of Fmoc-Trp(Boc)-OH: A solution of the resin and piperidine (100 mL, 20% purity in DMF) was agitated under N₂ for 30 min, and then filtered. The resin (filter cake) was washed with DMF (100 ml×3). To a mixture of Fmoc-Trp(Boc)-OH (10.3 g, 19 mmol, 3 eq) and DIPEA (5.0 g, 39 mmol, 6 eq) in DMF (100 mL) was added HBTU (7.0 g, 18 mmol, 2.85 eq) at 25° C. The mixture was stirred at 25° C. for 20 min. And then the mixture was added to the deprotected the Ala-Val-Ala-His(Trt)-Leu-Nle-amide resin (SEQ ID NO: 308). The resulting mixture was agitated under N₂ for 40 min. After the reaction was judged complete, the resin was filtered and washed with DMF (100 mL×3).

Step 8: Coupling of Fmoc-His(Trt)-OH: A solution of the resin and piperidine (100 mL, 20% purity in DMF) was agitated under N₂ for 30 min, and then filtered. The resin (filter cake) was washed with DMF (100 ml×3). To a mixture of Fmoc-His(Trt)-OH (12 g, 19 mmol, 3 eq) and DIPEA (5.0 g, 39 mmol, 6 eq) in DMF (100 mL) was added HBTU (7.0 g, 18 mmol, 2.85 eq) at 25° C. The mixture was stirred at 25° C. for 20 min. And then the mixture was added to the deprotected Trp(Boc)-Ala-Val-Ala-His(Trt)-Leu-Nle-amide resin (SEQ ID NO: 309). The resulting mixture was agitated under N₂ for 40 min. After the reaction was judged complete, the resin was filtered and washed with DMF (100 mL×3).

Step 9: Coupling of Fmoc-Asn(Trt)-OH: A solution of the resin and piperidine (100 mL, 20% purity in DMF) was agitated under N₂ for 30 min, and then filtered. The resin (filter cake) was washed with DMF (100 ml×3). To a mixture of Fmoc-Asn(Trt)-OH (12 g, 19 mmol, 3 eq) and DIPEA (5.0 g, 39 mmol, 6 eq) in DMF (100 mL) was added HBTU (7.0 g, 18 mmol, 2.85 eq) at 25° C. The mixture was stirred at 25° C. for 20 min. And then the mixture was added to the deprotected His(Trt)-Trp(Boc)-Ala-Val-Ala-His(Trt)-Leu-Nle-amide resin (SEQ ID NO: 310). The resulting mixture was agitated under N₂ for 40 min. After the reaction was judged complete, the resin was filtered and washed with DMF (100 mL×3).

Step 10: Coupling of Fmoc-Gly-OH: A solution of the resin and piperidine (100 mL, 20% purity in DMF) was agitated under N₂ for 30 min, and then filtered. The resin (filter cake) was washed with DMF (100 ml×3). To a mixture of Fmoc-Gly-OH (5.8 g, 19 mmol, 3 eq) and DIPEA (5.0 g, 39 mmol, 6 eq) in DMF (100 mL) was added HBTU (7.0 g, 18 mmol, 2.85 eq) at 25° C. The mixture was stirred at 25° C. for 20 min. And then the mixture was added to the deprotected Asn(Trt)-His(Trt)-Trp(Boc)-Ala-Val-Ala-His(Trt)-Leu-Nle-amide resin (SEQ ID NO: 311). The resulting mixture was agitated under N₂ for 40 min. After the reaction was judged complete, the resin was filtered and washed with DMF (100 mL×3).

Step 11: Coupling of Fmoc-NH-PEG₂-CH₂COOH: A solution of the resin and piperidine (100 mL, 20% purity in DMF) was agitated under N₂ for 30 min, and then filtered. The resin (filter cake) was washed with DMF (100 ml×3). To a mixture of Fmoc-NH-PEG₂-CH₂COOH (2.8 g, 19 mmol, 3 eq) and DIPEA (5.0 g, 39 mmol, 6 eq) in DMF (100 mL) was added HBTU (7.0 g, 18 mmol, 2.85 eq) at 25° C. The mixture was stirred at 25° C. for 20 min. And then the mixture was added to the deprotected Gly-Asn(Trt)-HIS(Trt)-Trp(Boc)-Ala-Val-Ala-His(Trt)-Leu-Nle-amide resin (SEQ ID NO: 312). The resulting mixture was agitated under N₂ for 40 min. After the reaction was judged complete, the resin was filtered and washed with DMF (100 mL×3).

Step 12: Coupling of Fmoc-NH-PEG₂-CH₂COOH: A solution of the resin and piperidine (100 mL, 20% purity in DMF) was agitated under N₂ for 30 min, and then filtered. The resin (filter cake) was washed with DMF (100 ml×3). To a mixture of Fmoc-NH-PEG₂-CH₂COOH (2.8 g, 19 mmol, 3 eq) and DIPEA (5.0 g, 39 mmol, 6 eq) in DMF (100 mL) was added HBTU (7.0 g, 18 mmol, 2.85 eq) at 25° C. The mixture was stirred at 25° C. for 20 min. And then the mixture was added to the deprotected H₂N-PEG₂-Gly-Asn(Trt)-His(Trt)-Trp(Boc)-Ala-Val-Ala-His(Trt)-Leu-Nle-amide resin (SEQ ID NO: 313). The resulting mixture was agitated under N₂ for 40 min. After the reaction was judged complete, the resin was filtered and washed with DMF (100 mL×3).

Step 13: Coupling of Fmoc-D-Glu-OtBu: A solution of the resin and piperidine (100 mL, 20% purity in DMF) was agitated under N₂ for 30 min, and then filtered. The resin (filter cake) was washed with DMF (100 ml×3). To a mixture of Fmoc-D-Glu-OtBu (3.1 g, 19 mmol, 3 eq) and DIPEA (5.0 g, 39 mmol, 6 eq) in DMF (100 mL) was added HBTU (7.0 g, 18 mmol, 2.85 eq) at 25° C. The mixture was stirred at 25° C. for 20 min. And then the mixture was added to the deprotected H₂N-OEG-OEG-Gly-Asn(Trt)-His(Trt)-Trp(Boc)-Ala-Val-Ala-His(Trt)-Leu-Nle-amide resin (SEQ ID NO: 314). The resulting mixture was agitated under N₂ for 40 min. After the reaction was judged complete, the resin was filtered and washed with DMF (100 mL×3).

Step 14: Coupling of Palmitic Acid: A solution of the resin and piperidine (100 mL, 20% purity in DMF) was agitated under N₂ for 30 min, and then filtered. The resin (filter cake) was washed with DMF (100 ml×3). To a mixture of palmitic acid (1.8 g, 19 mmol, 3 eq) and DIPEA (5.0 g, 39 mmol, 6 eq) in DMF (100 mL) was added HBTU (7.0 g, 18 mmol, 2.85 eq) at 25° C. The mixture was stirred at 25° C. for 20 min. And then the mixture was added to the deprotected Glu(OtBu)-NH-OEG-OEG-Gly-Asn(Trt)-His(Trt)-Trp(Boc)-Ala-Val-Ala-His(Trt)-Leu-Nle-amide resin (SEQ ID NO: 315). The resulting mixture was agitated under N₂ for 40 min. After the reaction was judged complete, the resin was filtered and washed with DMF (100 mL×3).

Step 15: TFA Cleavage and Ether Precipitation: A solution of thioanisole (4.1 g, 33 mmol, 13.5 eq), phenol (1.9 g, 20 mmol, 8.4 eq) and ethane-1,2-dithiol (2.3 g, 25 mmol, 10.2 eq) in TFA (63 mL) and H₂O (1.8 mL) was stirred at 0° C. for 15 min in round-bottom flask, and then the palmitic acid-Glu(OtBu)-NH-OEG-OEG-Gly-Asn(Trt)-His(Trt)-Trp(Boc)-Ala-Val-Ala-His(Trt)-Leu-Nle-amide resin (SEQ ID NO: 316) (6.48 g, 2.44 mmol, 1 eq) was added to the mixture at 0° C. The resulting mixture was stirred at 25° C. for 4 hr. The reaction mixture was filtered, and the filtrate was added to cold MTBE (180 mL) to form a white precipitate. The white precipitate was filtered to give the crude peptide. The crude peptide was purified by prep-HPLC (column: CD05-Phenomenex luna C18 150×40×10 um; mobile phase: [A: water (0.1% TFA), B: ACN]; B %: 27%-57%, 10 min) to give Peptide 7 as a white solid. LCMS: (ES+) m/z (M+H)⁺=1774.

### Example 2: Synthesis of Peptide 25 (SEQ ID NO. 25) and SEQ ID NO. 317

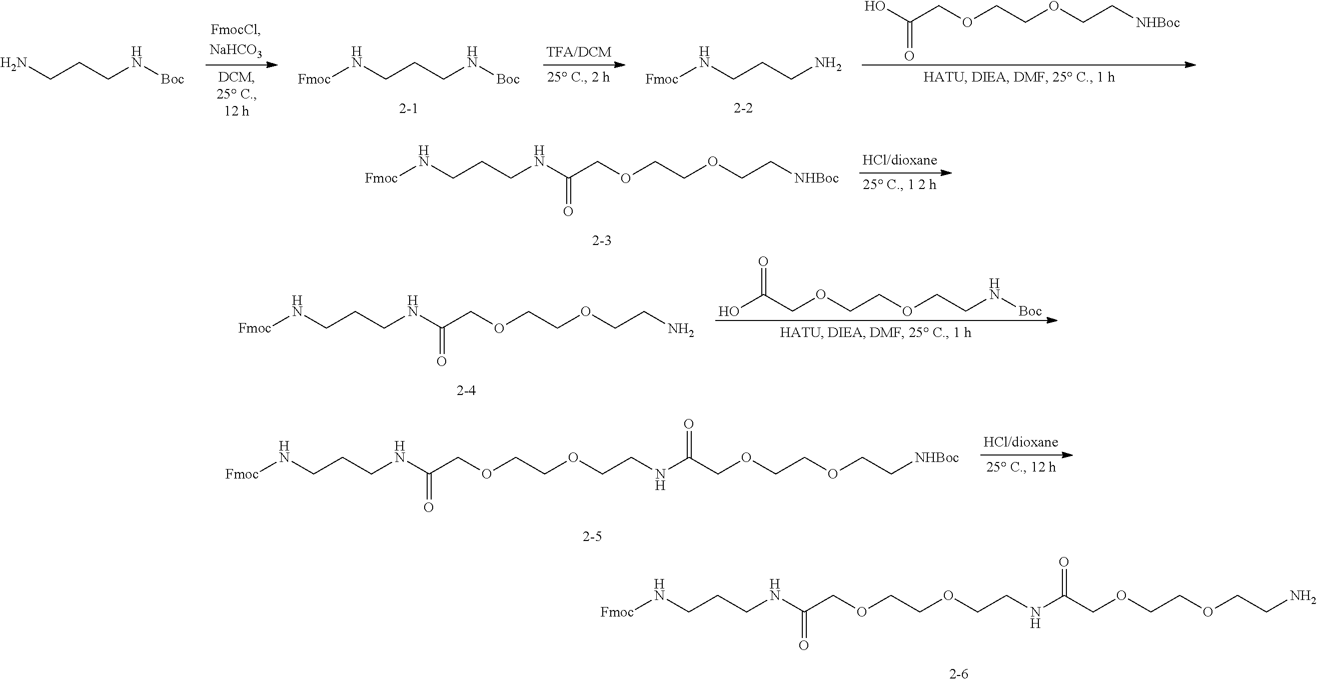

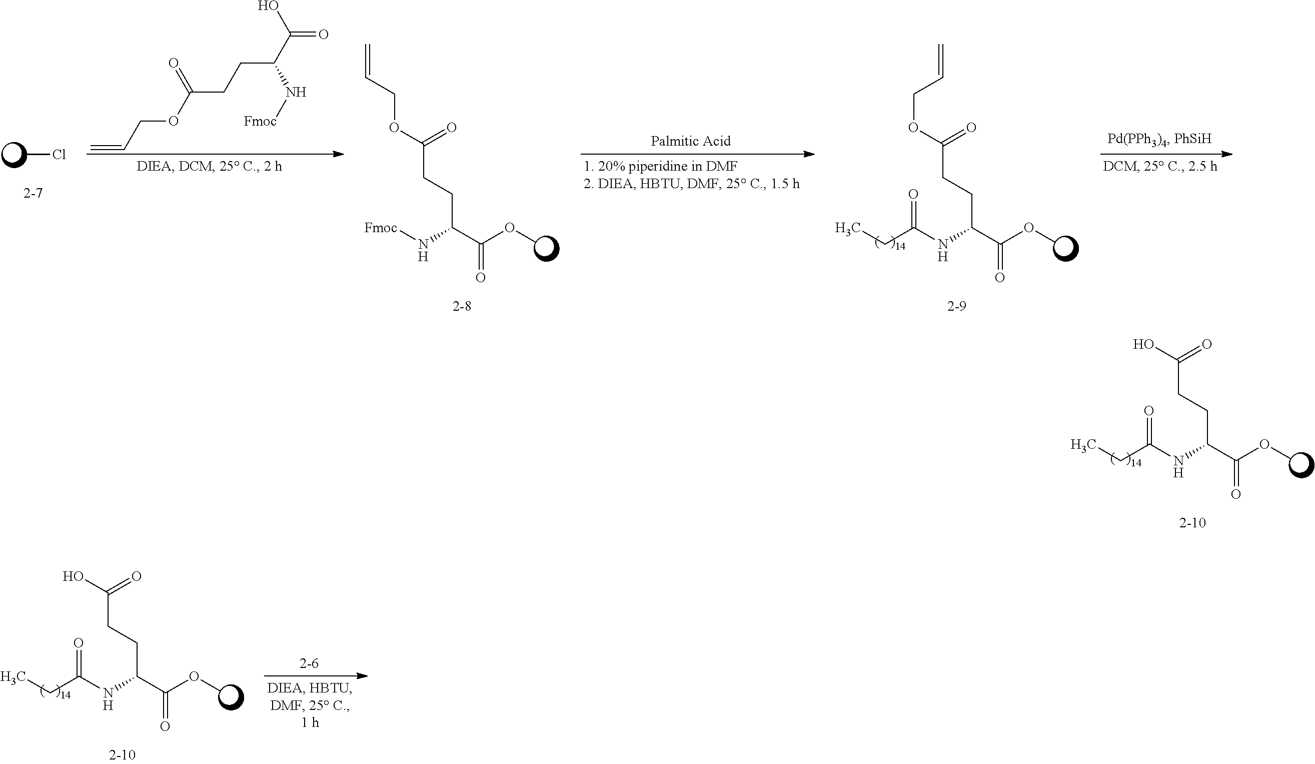

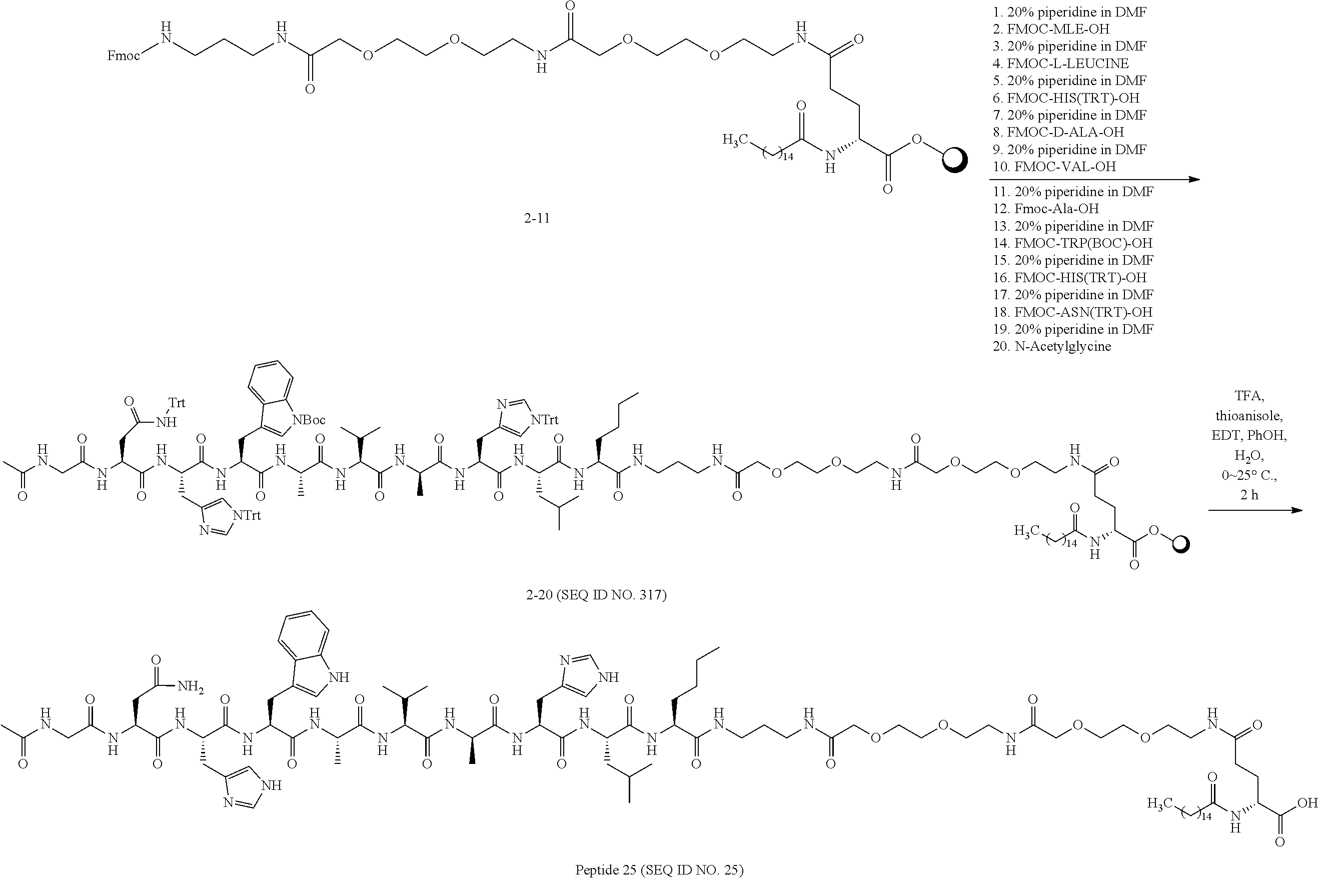

Step 1: (9H-fluoren-9-yl)methyl tert-butyl propane-1,3-diyldicarbamate (2-1): Tert-butyl (3-aminopropyl)carbamate (5.0 g, 29 mmol, 5 mL, 1.0 q was added to a mixture of DCM (90 mL) and saturated aqueous NaHCO₃ (90 mL) solution. And then Fmoc-Cl (8.0 g, 30 mmol, 1.1 eq) was added to the reaction mixture. The mixture was stirred at 25° C. for 12 hrs. The water layer was removed, and the organic layer was washed with water (50 mL×3), then dried over Na₂SO₄, filtered and concentrated under reduced pressure to give a residue. The residue was purified by flash silica gel chromatography (ISCO®; 80 g SepaFlash® Silica Flash Column, Eluent of 20˜100% Ethyl acetate/Petroleum ether gradient @100 mL/min) to give 2-1 (11 g, 92% yield) as a white solid.

¹H NMR (400 MHz, CD₃OD) δ=7.85-7.77 (m, 2H), 7.66 (d, J=7.6 Hz, 2H), 7.44-7.38 (in, 2H), 7.36-7.28 (in, 2H), 4.37 (d, J=6.8 Hz, 2H), 4.24-4.18 (m, 1H), 3.15 (t, J=6.8 Hz, 2H), 3.07 (t, J=6.4 Hz, 2H), 1.70-1.58 (m, 2H), 1.45 (s, 9H).

Step 2: (9H-fluoren-9-yl)methyl (3-aminopropyl)carbamate (2-2): To a solution of (9H-fluoren-9-yl)methyl tert-butyl propane-1,3-diyldicarbamate (2-1) (11 g, 26 mmol, 1.0 eq) in DCM (100 mL) was added TFA (0.27 mol, 20 mL, 10 eq). The mixture was stirred at 25° C. for 2 hrs. The reaction mixture was concentrated under reduced pressure to give a residue. The crude product was purified by reverse phase HPLC (column: Santai Technologies SepaFlash® cartridge 184.4×26.7 mm×40-60 μm; mobile phase: [A: water (0.1% HCl), B: ACN]; B %: 20%-40%) to give 2-2 (8.8 g, 99% yield, HCl) as a white solid.

¹H NMR (400 MHz, DMSO-d6) δ=7.90 (d, J=7.6 Hz, 2H), 7.84 (s, 2H), 7.68 (d, J=7.6 Hz, 2H), 7.46-7.39 (m, 3H), 7.37-7.30 (m, 2H), 4.33 (d, J=6.8 Hz, 2H), 4.25-4.18 (m, 1H), 3.05 (q, J=6.4 Hz, 2H), 2.82-2.70 (m, 2H), 1.74-1.63 (m, 2H).

Step 3: (9H-fluoren-9-yl)methyl (2,2-dimethyl-4,13-dioxo-3,8,11-trioxa-5,14-diazaheptadecan-17-yl)carbamate (2-3): To a solution of 2,2-dimethyl-4-oxo-3,8,11-trioxa-5-azatridecan-13-oic acid (12 g, 47 mmol, 2.0 eq) in DMF (80 mL) was added DIEA (0.12 mol, 20 mL, 5.0 eq) and HATU (18 g, 47 mmol, 2.0 eq). The mixture was stirred at 25° C. for 30 min, and then (9H-fluoren-9-yl)methyl (3-aminopropyl)carbamate (2-2) (7.8 g, 23 mmol, 1.0 eq, HCl salt) was added dropwise at 25° C. The resulting mixture was stirred at 25° C. for 30 min. The reaction mixture was diluted with H₂O (500 mL) and extracted with EtOAc (200 mL×3). The combined organic layers were washed with saturated brine (200 mL×3), dried over Na₂SO₄, filtered and concentrated under reduced pressure to give a residue. The residue was purified by flash silica gel chromatography (ISCO®; 120 g SepaFlash® Silica Flash Column, Eluent of 0˜10% Methanol/Ethyl acetate gradient @100 mL/min) to give 2-3 (12 g, 94% yield) as a yellow gum.

¹H NMR (400 MHz, DMSO-d6) δ=7.89 (d, J=7.6 Hz, 2H), 7.68 (d, J=7.2 Hz, 2H), 7.44-7.38 (m, 2H), 7.36-7.30 (m, 2H), 7.26 (t, J=5.6 Hz, 1H), 6.78 (bt, J=5.6 Hz, 1H), 4.31 (d, J=6.8 Hz, 2H), 4.25-4.17 (m, 1H), 3.86 (s, 2H), 3.61-3.49 (m, 4H), 3.42-3.36 (m, 2H), 3.14-3.02 (m, 4H), 2.98 (q, J=6.8 Hz, 2H), 1.62-1.45 (m, 2H), 1.36 (s, 9H).

Step 4: (9H-fluoren-9-yl)methyl (3-(2-(2-(2-aminoethoxy)ethoxy)acetamido)propyl)carbamate (2-4): A mixture of (9H-fluoren-9-yl)methyl (2,2-dimethyl-4,13-dioxo-3,8,11-trioxa-5,14-diazaheptadecan-17-yl)carbamate (2-3) (2.0 g, 3.7 mmol, 1.0 eq) in HCl/dioxane (2.0 M, 20 mL) was degassed and purged with N₂ 3 times. And then the mixture was stirred at 25° C. for 12 hrs. The reaction mixture was concentrated under reduced pressure to give 2-4 (1.76 g, crude, HCl salt) as a yellow gum.

Step 5: (9H-fluoren-9-yl)methyl tert-butyl (8,17-dioxo-3,6,12,15-tetraoxa-9,18-diazahenicosane-1,21-diyl)dicarbamate (2-5): To a solution of 2,2-dimethyl-4-oxo-3,8,11-trioxa-5-azatridecan-13-oic acid (1.1 g, 4.1 mmol, 1.1 eq) in ACN (20 mL) was added DIEA (18 mmol, 3.2 mL, 5.0 eq) and HATU (2.8 g, 7.4 mmol, 2.0 eq). The mixture was stirred at 25° C. for 30 min, and then (9H-fluoren-9-yl)methyl (3-(2-(2-(2-aminoethoxy)ethoxy)acetamido)propyl)carbamate (2-4) (1.76 g, 3.7 mmol, 1.0 eq, HCl salt) was added at 25° C. The resulting mixture was stirred at 25° C. for 30 min. The reaction mixture was diluted with H₂O (150 mL) and extracted with EtOAc (50 mL×3). The combined organic layers were washed with saturated brine (150 mL), dried over Na₂SO₄, filtered and concentrated under reduced pressure to give a residue. The residue was purified by flash silica gel chromatography (ISCO®; 40 g SepaFlash® Silica Flash Column, Eluent of 0˜10% Methanol/Ethyl acetate gradient @100 mL/min) to give (2-5) (2.3 g, 88% yield) as a yellow gum.

LCMS: (ES⁺) m/z (M+H)⁺=687.4.

¹H NMR (400 MHz, DMSO-d6) δ=8.16 (d, J=2.4 Hz, 1H), 7.89 (d, J=7.6 Hz, 2H), 7.68 (d, J=7.6 Hz, 2H), 7.66-7.61 (m, 1H), 7.45-7.37 (m, 2H), 7.36-7.29 (m, 2H), 7.25 (t, J=6.0 Hz, 1H), 6.82-6.69 (m, 1H), 4.31 (d, J=6.8 Hz, 2H), 4.24-4.18 (m, 1H), 3.86 (d, J=1.6 Hz, 4H), 3.67-3.58 (m, 4H), 3.48-3.43 (m, 2H), 3.38 (t, J=6.0 Hz, 2H), 3.30-3.26 (m, 2H), 3.18-3.02 (m, 8H), 2.98 (q, J=6.4 Hz, 2H), 1.54 (quin, J=6.8 Hz, 2H), 1.36 (s, 9H).

Step 6: (9H-fluoren-9-yl)methyl (1-amino-8,17-dioxo-3,6,12,15-tetraoxa-9,18-diazahenicosan-21-yl)carbamate (2-6): A mixture of (9H-fluoren-9-yl)methyl tert-butyl (8,17-dioxo-3,6,12,15-tetraoxa-9,18-diazahenicosane-1,21-diyl)dicarbamate (2-5) (2.3 g, 3.2 mmol, 1.0 eq) in HCl/dioxane (2.0 M, 20 mL) was stirred at 25° C. for 12 hrs. The reaction mixture was concentrated under reduced pressure to give (2-6) (2 g, crude, HCl salt) as a yellow gum.

Step 7: Peptide 25 was synthesized by standard Solid-phase Peptide Synthesis (SPPS) using Fmoc/t-Bu chemistry. The assembly was performed on a 2-chlorotrityl chloride resin (100-200 mesh; loading 0.565 mmol/g) on the peptide solid phase synthesis tube under a nitrogen atmosphere. The constructed peptide was isolated from the resin and protecting groups cleavaged with strong acid followed by precipitation. Peptide purification was performed by prep-HPLC. Lyophilization of pure fractions gave the final product.

Swell Resin: 3.0 g of 2-chlorotrityl chloride solid phase resin (0.565 mmol/g loading) was transferred to a 100-mL peptide vessel with filter frit, ground glass joint and vacuum side arm. The resin was washed with DMF (30 mL×3).

Step 8: Coupling of Fmoc-D-Glu(OAll)-OH: To a solution of 2-chlorotrityl chloride resin (3.0 g, 0.565 mmol/g loading) in DCM (50 mL) was added (R)-2-((((9H-fluoren-9-yl)methoxy)carbonyl)amino)-5-(allyloxy)-5-oxopentanoic acid (0.90 g, 2.2 mmol, 1.3 eq) and DIEA (0.66 g, 5.1 mmol, 0.89 mL, 3.0 eq). The mixture was agitated under N₂ for 2 hrs, and then MeOH (0.25 mol, 10 mL) was added at 25° C. The mixture was stirred at 25° C. for 30 min. The resin was filtered. The resin was washed with DMF (30 mL×2), MeOH (30 mL), DCM (30 mL), MeOH (30 mL), DCM (30 mL) and DMF (30 mL×2) prior to starting the next deprotection or coupling cycle.

Step 9: Coupling of Palmitic Acid: A solution of the resin and piperidine (30 mL 20% purity in DMF) was agitated under N₂ for 30 min, and then filtered. The resin (filter cake) was washed with DMF (30 ml×5). To a mixture of palmitic acid (1.3 g, 5.1 mmol, 3.0 eq) and DIPEA (1.77 mL, 10 mmol, 6.0 eq) in DMF (30 mL) was added HBTU (1.8 g, 4.8 mmol, 2.85 eq) at 25° C. The mixture was stirred at 25° C. for 20 min. And then the mixture was added to the deprotected the resin. The resulting mixture was agitated under N₂ for 40 min. After the reaction was judged complete, the resin was filtered and washed with DMF (30 mL×5) prior to starting the next deprotection or coupling cycle.

Step 10: Removing of Allyl protecting group: To a stirred suspension of resin (0.72 g, 1.0 mmol, 1.0 eq) in DCM (30 mL) was added dropwise PhSiH (0.42 g, 3.89 mmol, 0.48 mL, 3.8 eq). And then Pd(PPh₃)₄ (0.12 g, 0.1 mmol, 0.1 eq) was added and the reaction mixture was stirred for 2.5 h at 25° C. After the reaction was judged complete, the resin was filtered and washed with DMF (30 mL×5) prior to starting the next deprotection or coupling cycle.

Step 11: Coupling of 2-6: To a solution of the resin (0.67 g, 1.0 mmol, 1.0 eq) in DMF (30 mL) was added DIEA (1.07 ml, 6.1 mmol, 6.0 eq) and HBTU (1.1 g, 2.9 mmol, 2.85 eq). The mixture was agitated under N₂ for 20 min, and then (9H-fluoren-9-yl)methyl (1-amino-8,17-dioxo-3,6,12,15-tetraoxa-9,18-diazahenicosan-21-yl)carbamate (2-6) (0.95 g, 1.5 mmol, 1.5 eq, HCl) was added to the resin at 25° C. The resulting mixture was agitated under N₂ for 40 min. After the reaction was judged complete, the resin was filtered and washed with DMF (30 mL×3) prior to starting the next deprotection or coupling cycle.

Step 12: Coupling of Fmoc-Nle-OH: A solution of the resin (1.25 g, 1.0 mmol) and piperidine (30 mL 20% purity in DMF) was agitated under N₂ for 30 min, and then filtered. The resin (filter cake) was washed with DMF (30 ml×5). To a mixture of Fmoc-Nle-OH (1.1 g, 3.0 mmol, 3.0 eq) and DIPEA (1.06 mL, 6.0 mmol, 6 eq) in DMF (30 mL) was added HBTU (1.1 g, 2.9 mmol, 2.85 eq) at 25° C. The mixture was stirred at 25° C. for 20 min. And then the mixture was added to the deprotected resin. The resulting mixture was agitated under N₂ for 40 min. After the reaction was judged complete, the resin was filtered and washed with DMF (100 mL×3) prior to starting the next deprotection or coupling cycle.

Steps 13˜21: Step 12 was repeated sequentially using the amino acid derivatives and coupling reagents in Table D.

TABLE D
Amino acid derivatives and corresponding reagents used in SPPS.

| Step | Amino Acid Derivative | Coupling Reagents |
|---|---|---|
| 13 | Fmoc-Leu-OH (3.0 eq) | HBTU (2.85 eq) and DIEA (6.0 eq), 40 min |
| 14 | Fmoc-His(Trt)-OH (3.0 eq) | HBTU (2.85 eq) and DIEA (6.0 eq), 40 min |
| 15 | Fmoc-D-Ala-OH (3.0 eq) | HBTU (2.85 eq) and DIEA (6.0 eq), 40 min |
| 16 | Fmoc-Val-OH (3.0 eq) | HBTU (2.85 eq) and DIEA (6.0 eq), 40 min |
| 17 | Fmoc-Ala-OH (3.0 eq) | HBTU (2.85 eq) and DIEA (6.0 eq), 40 min |
| 18 | Fmoc-Trp-OH (3.0 eq) | HBTU (2.85 eq) and DIEA (6.0 eq), 40 min |
| 19 | Fmoc-His(Trt)-OH (3.0 eq) | DEPBT (2.85 eq) and DIEA (6.0 eq), 40 min |
| 20 | Fmoc-Asn-OH (3.0 eq) | HBTU (2.85 eq) and DIEA (6.0 eq), 40 min |
| 21 | N-Acetylglycine (3.0 eq) | HBTU (2.85 eq) and DIEA (6.0 eq), 40 min |

Step 22: TFA Cleavage and Ether precipitation: A solution of thioanisole (0.8 mL), phenol (0.4 mL) and ethane-1,2-dithiol (0.4 mL) in TFA (14 mL) and H₂O (0.4 mL) (TFA/thioanisole/phenol/ethane-1,2-dithiol/H₂O v/v/v/v/v, 87.5%/5%/2.5%/2.5%/2.5%, 8 mL/g resin) was stirred at 0° C. for 15 min in round-bottom flask, and then the N-Acetylglycine-Asn(Trt)-His(Trt)-Trp(Boc)-Ala-Val-D-Ala His(Trt)-Leu-Nle-H₂N(CH₂)₃NH₂—OEG-OEG-α-palmitoyl-D-γGlu(tBu)-chloride resin (SEQ ID NO: 317) (2.0 g resin) was added to the mixture at 0° C. The resulting mixture was stirred at 25° C. for 2 hrs. The reaction mixture was filtered and the filtrate was added to cold MTBE (96 mL) to form a white precipitate. The white precipitate was filtered to give the crude peptide. The crude peptide was purified by prep-HPLC (column: Phenomenex luna C18 150×40×10 um; mobile phase: [A: water (0.1% TFA), B: ACN]; B %: 35%-55%, 14 min) to give Peptide 25 as a white solid. LCMS: (ES+) m/z (M/2+H)⁺=937.4; (M/3+H)⁺=625.3; (M/4+H)⁺=469.2.

### Example 3: Synthesis of Peptide 55 (SEQ ID NO. 55)

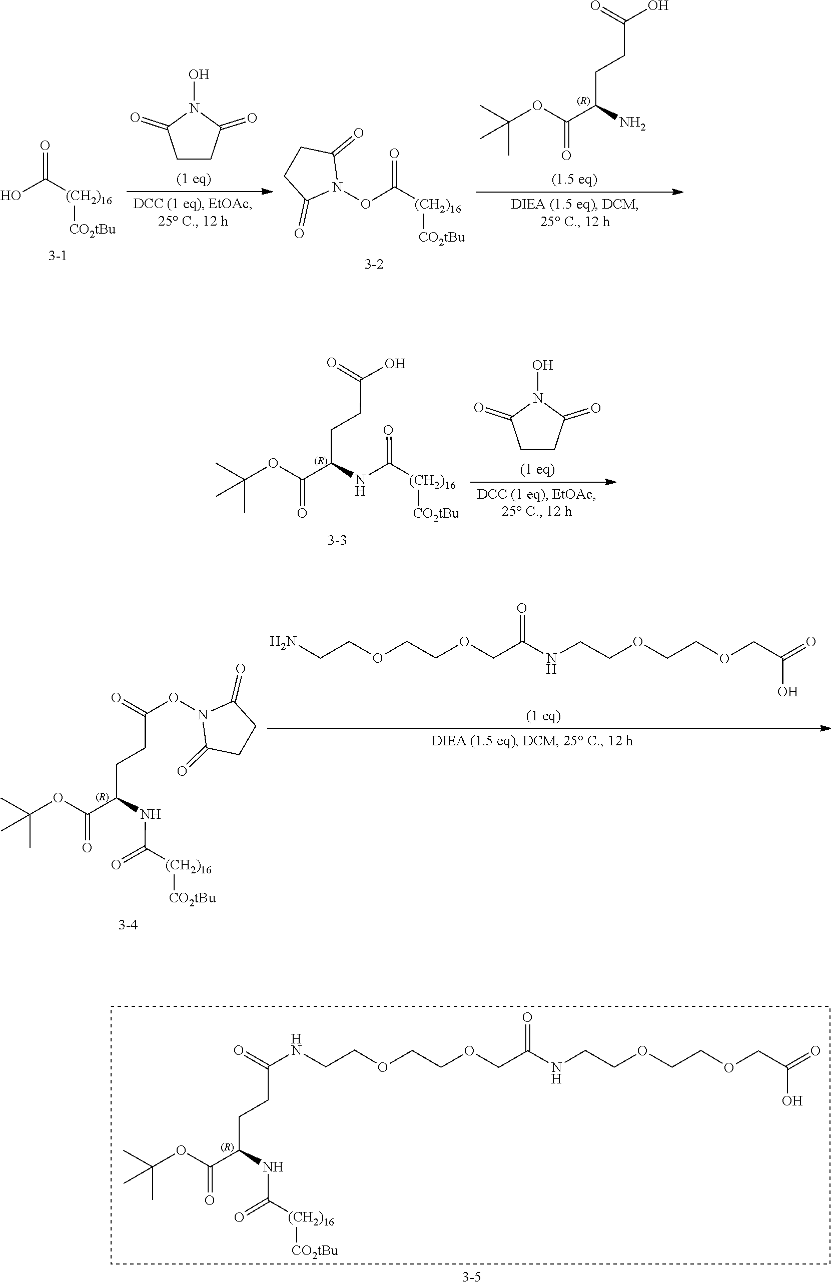

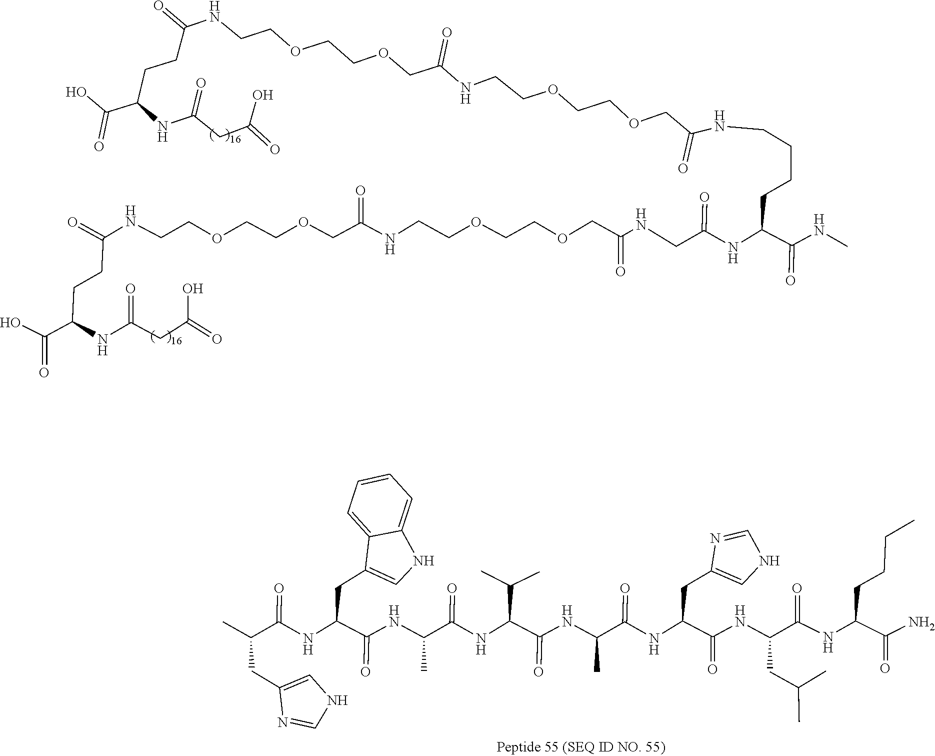

Step 1: 1-(tert-butyl) 18-(2,5-dioxopyrrolidin-1-yl) octadecanedioate (3-2): To a solution of 1-18-(tert-butoxy)-18-oxooctadecanoic acid (30 g, 81 mmol, 1.0 eq) in EtOAc (300 mL) was added 1-hydroxypyrrolidine-2,5-dione (11 g, 97 mmol, 1.2 eq) followed by the addition of DCC (20 mL, 97 mmol, 1.2 eq) in EtOAc (100 mL). The reaction mixture was stirred at 25° C. for 12 hrs. The reaction mixture was filtered and the filtrate was concentrated under reduced pressure to remove solvent. The residue was purified by Flash chromatography (ISCO®; 330 g SepaFlash® Silica Flash Column, Eluent of 0˜20% Ethylacetate/Petroleum ether gradient @100 mL/min) to give 3-2 (26 g, 69% yield) as a white solid.

¹H NMR (400 MHz, CDCl₃) δ=2.84 (s, 4H), 2.62-2.59 (t, J=7.6 Hz, 2H), 2.22-2.18 (t, J=7.6 Hz, 2H), 1.77-1.73 (m, 2H), 1.60-1.58 (m, 2H), 1.56-1.44 (m, 11H), 1.28-1.26 (m, 22H).

Step 2: (R)-5-(tert-butoxy)-4-(18-(tert-butoxy)-18-oxooctadecanamido)-5-oxopentanoic acid (3-3): To a solution of 1-(tert-butyl)18-(2,5-dioxopyrrolidin-1-yl) octadecanedioate 3-2 (26 g, 56 mmol, 1.0 eq) in DCM (250 mL) was added (R)-4-amino-5-(tert-butoxy)-5-oxopentanoic acid (17 g, 83 mmol, 1.5 eq) and DIEA (14 mL, 83 mmol, 1.5 eq). The mixture was stirred at 25° C. for 12 hrs. The pH of the reaction mixture was adjusted to 3˜4 by addition of 2N aqueous HCl and extracted with EA (100 mL×3). The combined organic layers were washed with saturated aqueous sodium chloride solution (100 mL×2), dried over anhydrous Na₂SO₄, filtered and concentrated under reduced pressure to give 3-3 (29 g, crude) as a white solid. LCMS: (ES⁺) m/z (M+H)⁺=556.4

¹H NMR (400 MHz, CDCl₃) δ=6.25-6.23 (d, J=8.0 Hz, 1H), 4.55-4.52 (m, 1H), 2.43-2.42 (m, 2H), 2.27-2.19 (m, 5H), 2.20-2.18 (m, 1H), 1.64-1.58 (m, 5H), 1.48 (s, 9H), 1.45 (s, 9H), 1.30-1.26 (m, 23H).

Step 3: 1-(tert-butyl) 5-(2,5-dioxopyrrolidin-1-yl) (18-(tert-butoxy)-18-oxooctadecanoyl)-D-glutamate (3-4): To a solution of (R)-5-(tert-butoxy)-4-(18-(tert-butoxy)-18-oxooctadecanamido)-5-oxopentanoic acid 3-3 (29 g, 52 mmol, 1.0 eq) in EtOAc (300 mL) was added 1-hydroxypyrrolidine-2,5-dione (7.2 g, 63 mmol, 1.2 eq) followed by the addition of DCC (13 mL, 63 mmol, 1.2 eq) in EtOAc (100 mL), and the reaction mixture was stirred at 25° C. for 12 hrs. The reaction mixture was filtered and the filtrate was concentrated under reduced pressure to remove solvent. The residue was purified by Flash chromatography (ISCO®; 330 g SepaFlash® Silica Flash Column, Eluent of 0˜50% Ethylacetate/Petroleum ether gradient @ 100 mL/min) to give 3-4 (25 g, 70% yield) a white solid.

¹H NMR (400 MHz, CDCl₃) δ=6.20-6.18 (d, J=7.6 Hz, 1H), 4.63-4.58 (m, 1H), 2.84 (s, 4H), 2.76-2.73 (m, 2H), 2.36-2.24 (m, 1H), 2.22-2.18 (m, 4H), 1.64-1.58 (m, 6H), 1.49 (s, 9H), 1.45 (s, 9H), 1.29-1.25 (m, 23H).

Step 4: Octadecanedioyl(OtBu)-γGlu(OtBu)-OEG-OEG-OH (3-5): To a solution of 1-(tert-butyl) 5-(2,5-dioxopyrrolidin-1-yl) (18-(tert-butoxy)-18-oxooctadecanoyl)-D-glutamate 3-4 (25 g, 38.29 mmol, 1 eq) in DCM (250 mL) was added NH₂-OEG-OEG-OH (18 g, 57 mmol, 1.5 eq) and DIEA (10 mL, 57 mmol, 1.5 eq). The mixture was stirred at 25° C. for 2 hrs. The pH of the reaction mixture was adjusted to 3˜4 by addition of 2N aqueous HCl and extracted with EA (100 mL×3). The combined organic layers were washed with saturated aqueous sodium chloride solution (100 mL×2), dried over anhydrous Na₂SO₄, filtered and concentrated under reduced pressure to give a residue. The residue was purified by prep-HPLC (column: Welch Xtimate C18 150×25 mm×5 μm; mobile phase: [A: water (0.1% FA), B: ACN]; B %: 100%-100%, 30 min) to give 3-5 (19 g, 64% yield) as a white solid. LCMS: (ES⁺) m/z (M+H)⁺=846.7

¹H NMR (400 MHz, CDCl₃) δ=7.36-7.27 (m, 1H), 7.00-6.98 (t, J=5.6 Hz, 1H), 6.51-6.49 (d, J=7.6 Hz, 1H), 4.46-4.45 (m, 1H), 4.17 (s, 2H), 4.02-4.01 (m, 2H), 3.76-3.74 (m, 2H), 3.68-3.61 (m, 6H), 3.60-3.59 (m, 4H), 3.53-3.48 (m, 4H), 2.31-2.26 (m, 2H), 2.24-2.19 (m, 5H), 1.99-1.85 (m, 1H), 1.60-1.58 (m, 5H), 1.48 (s, 9H), 1.45 (s, 9H), 1.28-1.25 (m, 23H).

Step 5: Peptide 55 was synthesized by standard Solid-phase Peptide Synthesis (SPPS) using Fmoc/t-Bu chemistry. The assembly was performed on a Rink-amide MBHA resin (100-200 Mesh; loading 0.787 mmol/g) on the peptide solid phase synthesis tube under a nitrogen atmosphere. The constructed peptide was isolated from the resin and protecting groups removed by cleavage with strong acid followed by precipitation. Peptide purification was performed prep-HPLC. Lyophilization of pure fractions gave the final product.

Swell Resin: 2 g of Rink Amide MBHA solid phase resin (0.787 mmol/g loading) was transferred to a 20-mL peptide vessel with filter frit, ground glass joint and vacuum side arm. The resin was washed with DMF (50 mL×3).

Step 6: Coupling of Fmoc-Nle-OH: A solution of 9H-fluoren-9-ylmethyl carbamate (0.38 g, 0.787 mmol/g loading), piperidine (20 mL, 20% purity in DMF) was agitated under N₂ for 30 min, and then filtered. The resin (filter cake) was washed with DMF (50 ml×6). To a mixture of Fmoc-Nle-OH (1.7 g, 4.7 mmol, 3 eq) and DIEA (1.6 mL, 9.44 mmol, 6 eq) in DMF (20 mL) was added HBTU (1.7 g, 4.5 mmol, 2.85 eq) at 25° C. The mixture was stirred at 25° C. for 20 min. And then the mixture was added to the deprotected resin. The resulting mixture was agitated under N₂ for 40 min. After the reaction was judged complete, the resin was filtered and washed with DMF (50 mL×6), and then diluted with DIEA (0.8 mL, 3 eq) and Ac₂O (0.4 mL, 3 eq) in DMF (100 mL). The resulting mixture was agitated under N₂ at 25° C. for 30 min, and then filtered and washed with DMF (50 ml×6) prior to starting the next deprotection or coupling cycle.

Steps 7˜20: Step 6 was repeated sequentially using the amino acid derivatives and coupling reagents in Table E.

TABLE E
Amino acid derivatives and coupling reagents used in SPPS.

| Step | Amino Acid Derivative | Coupling Reagents |
|---|---|---|
| 7 | Fmoc-Leu-OH (3.00 equiv.) | HBTU (2.85 equiv.) and DIEA |
| | | (6.00 equiv.), 40 min |
| 8 | Fmoc-His(Trt)-OH (3.00 | HBTU (2.85 equiv.) and DIEA |
| | equiv.) | (6.00 equiv.), 40 min |
| 9 | Fmoc-D-Ala-OH (6.00 equiv.) | HBTU (5.7 equiv.) and DIEA |
| | | (12.00 equiv.), 40 min |
| 10 | Fmoc-Val-OH (3.00 equiv.) | HBTU (2.85 equiv.) and DIEA |
| | | (6.00 equiv.), 40 min |
| 11 | Fmoc-Ala-OH (6.00 equiv.) | HBTU (5.7 equiv.) and DIEA |
| | | (12.00 equiv.), 40 min |
| 12 | Fmoc-Trp(Boc)-OH (3.00 | HBTU (2.85 equiv.) and DIEA |
| | equiv.) | (6.00 equiv.), 40 min |
| 13 | Fmoc-His(Trt)-OH (3.00 | HBTU (2.85 equiv.) and DIEA |
| | equiv.) | (6.00 equiv.), 40 min |
| 14 | Fmoc-Asn-OH (3.00 equiv.) | HBTU (2.85 equiv.) and DIEA |
| | | (6.00 equiv.), 40 min |
| 15 | Fmoc-Lys(Alloc)-OH (3.00 | HBTU (5.7 equiv.) and DIEA |
| | equiv.) | (12.00 equiv.), 40 min |
| 16 | Fmoc-Gly-OH (3.00 equiv.) | HBTU (2.85 equiv.) and DIEA |
| | | (6.00 equiv.), 40 min |
| 17 | Fmoc-OEG-OH (3.00 equiv.) | HBTU (2.85 equiv.) and DIEA |
| | | (6.00 equiv.), 40 min |
| 18 | Fmoc-OEG-OH (3.00 equiv.) | HBTU (2.85 equiv.) and DIEA |
| | | (6.00 equiv.), 40 min |
| 19 | Fmoc-Glu(OtBu)-OH (3.00 | HBTU (2.85 equiv.) and DIEA |
| | equiv.) | (6.00 equiv.), 40 min |
| 20 | Octadecanedioic(OtBu) | HBTU (2.85 equiv.) and DIEA |
| | Acid (3.00 equiv.) | (6.00 equiv.), 40 min |

Step 21: To a stirred suspension of Octadecanedioyl(OtBu)-γGlu(OtBu)-OEG-OEG-Gly-Lys(Alloc)-Asn-His(Trt)-Trp(Boc)-Ala-Val-D-Ala-His(Trt)-Leu-Nle-N₁₁₂ (SEQ ID NO: 318) (3.3 g, 1.3 mmol, 1.0 eq) (on Rink-amide Resin, 100-200 Mesh, loading 0.787 mmol/g) in DCM (50 mL) was added dropwise phenylsilane (0.5 g, 4.8 mmol, 0.6 mL, 3.8 eq). Pd(PPh₃)₄ (0.15 g, 0.1 mmol, 0.1 eq) was added the mixture and the resulting mixture was agitated under N₂ for 2.5 hrs. After the reaction was judged complete, the resin was filtered and washed with DMF (50 mL×6) to give octadecanedioic(OtBu) Acid-Glu(OtBu)-OEG-OEG-Gly-Lys-Asn-His(Trt)-Trp(Boc)-Ala-Val-DAla-His(Trt)-Leu-Nle-resin (SEQ ID NO: 319) (3-21).

Step 22: Coupling of Octadecanedioyl(OtBu)-γGlu(OtBu)-OEG-OEG-OH (3-5): To a solution of octadecanedioyl(OtBu)-γGlu(OtBu)-OEG-OEG-OH (3-5) (2.6 g, 3.0 mmol, 4.0 eq) in DMF (30 mL) was added HBTU (0.8 g, 4.5 mmol, 2.85 eq) and DIEA (0.8 mL, 4.6 mmol, 6.0 eq) at 25° C. The mixture was stirred at 25° C. for 20 min. And then the mixture was added to octadecanedioyl(OtBu)-γGlu(OtBu)-OEG-OEG-Gly-Lys-Asn-His(Trt)-Trp(Boc)-Ala-Val-D-Ala-His(Trt)-Leu-Nle-resin (SEQ ID NO: 320) (3-21). The resulting mixture was agitated under N₂ for 40 min. After the reaction was judged complete, the resin was filtered and washed with DMF (50 mL×6), and then diluted with DIEA (0.8 mL, 3 eq) and Ac₂O (0.4 mL, 3 eq) in DMF (100 mL). The resulting mixture was agitated under N₂ at 25° C. for 30 min, and then filtered and washed with DMF (50 ml×6) to give octadecanedioyl(OtBu)-γGlu(OtBu)-OEG-OEG-Gly-εLys(Octadecanedioyl(OtBu)-γGlu(OtBu)-OEG-OEG)-Asn-His(Trt)-Trp(Boc)-Ala-Val-D-Ala-His(Trt)-Leu-Nle-resin (SEQ ID NO: 321) (3-22).

Step 23: TFA Cleavage and Ether precipitation: A solution of thioanisole (1.2 mL), phenol (0.6 mL) and ethane-1,2-dithiol (0.6 mL) in TFA (21 mL) and H₂O (0.6 mL) (TFA/thioanisole/phenol/ethane-1,2-dithiol/H₂O v/v/v/v/v, 87.5%/5%/2.5%/2.5%/2.5%, 24 mL, 8 mL/g) was stirred at 0° C. for 15 min in a round-bottom flask, and then the octadecanedioyl(OtBu)-γGlu(OtBu)-OEG-OEG-Gly-εLys(Octadecanedioyl(OtBu)-γGlu(OtBu)-OEG-OEG)-Asn-His(Trt)-Trp(Boc)-Ala-Val-D-Ala-His(Trt)-Leu-Nle-resin (SEQ ID NO: 322) (3-22) (3.0 g) was added to the mixture at 0° C. The resulting mixture was stirred at 25° C. for 1.75 hrs. The reaction mixture was filtered and the filtrate was added to cold MTBE (120 mL) to form a white precipitate. The white precipitate was filtered to give the crude peptide. The crude peptide was purified by prep-HPLC (column: CD05-Phenomenex luna C18 150×40×10 um; mobile phase: [A: water (0.1% TFA), B: ACN]; B %: 25%-55%, 20 min) to give Peptide 55 as a white solid. LCMS: (ES+) m/z (M/2+H)⁺=1281.9; (M/3+H)⁺=854.9; (M/4+H)⁺=641.5.

### Example 4: Synthesis of Peptide 166 (SEQ ID NO. 166)

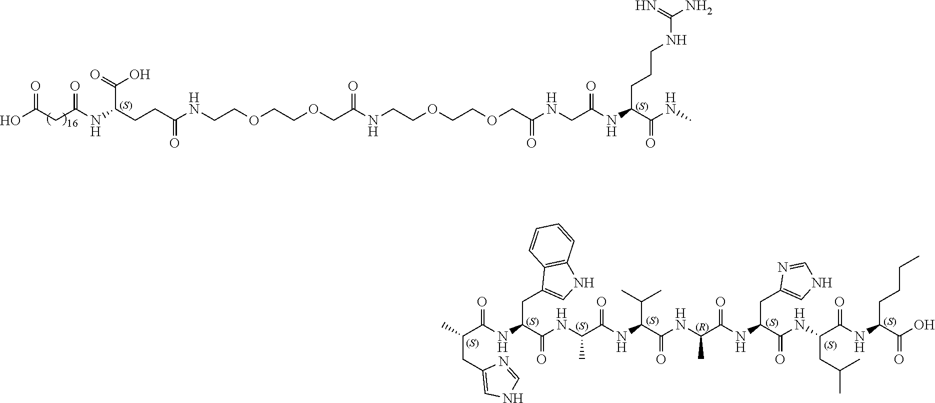

Peptide 166 was synthesized by standard Solid-phase Peptide Synthesis (SPPS) using Fmoc chemistry. The assembly was performed on a 2-Chlorotrityl Chloride Resin (100-200 Mesh; loading 1.365 mmol/g) in the peptide solid phase synthesis tube under a nitrogen atmosphere. The constructed peptide was isolated from the resin and protecting groups cleaved by treatment with strong acid followed by precipitation. Peptide purification was performed by pr-HPLC. Lyophilization of pure fractions gives the final product.

Swell Resin: 2.0 g of 2-Chlorotrityl Chloride Resin (1.365 mmol/g loading) was transferred to a 100-mL peptide vessel with filter frit, ground glass joint and vacuum side arm. The resin was washed with DMF (100 mL×3).

Step 1: Coupling of Fmoc-Nle-OH: To a solution of 2-chlorotrityl chloride (2.0 g, 1.365 mmol/g loading) in DCM (50 ml) was added Fmoc-Nle-OH (4.81 g, 13.60 mmol, 3 eq) and DIPEA (2.64 g, 20.40 mmol, 3 eq) at 25° C. The mixture was agitated under N₂ for 2 hrs. After the reaction was judged complete, the resin was filtered and washed with DMF (100 mL×3), and then diluted in MeOH (30 mL). The resulting mixture was agitated under N₂ at 25° C. for 30 min, and then filtered and washed with DMF (100 ml×3), DCM (100 ml×3) and DMF (100 ml×3) prior to starting the next deprotection or coupling cycle.

Steps 2-14: Step 1 was repeated sequentially using amino acid derivatives and coupling reagents in Table F.

TABLE F
Amino acid derivatives and corresponding reagents used in SPPS.

| Step | Amino Acid Derivative | Coupling Reagents |
|---|---|---|
| 2 | Fmoc-Leu-OH (3.00 equiv.) | DEPBT (2.85 equiv.) and DIEA (6.00 equiv.), 40 min |
| 3 | Fmoc- His (Trt)-OH (3.00 equiv.) | DEPBT (2.85 equiv.) and DIEA (6.00 equiv.), 40 min |
| 4 | Fmoc-D-Ala-OH (6.00 equiv.) | DEPBT (5.85 equiv.) and DIEA (12.00 equiv.), 80 min |
| 5 | Fmoc-Val-OH (3.00 equiv.) | DEPBT (2.85 equiv.) and DIEA (6.00 equiv.), 40 min |
| 6 | Fmoc-Ala-OH (6.00 equiv.) | DEPBT (5.85 equiv.) and DIEA (12.00 equiv.), 80 min |
| 7 | Fmoc-Trp (Boc)-OH (3.00 equiv.) | DEPBT (2.85 equiv.) and DIEA (6.00 equiv.), 40 min |
| 8 | Fmoc-His(Trt)-OH (3.00 equiv.) | DEPBT (2.85 equiv.) and DIEA (6.00 equiv.), 40 min |
| 9 | Fmoc-Arg(Pbf)-OH (3.00 equiv.) | DEPBT (2.85 equiv.) and DIEA (6.00 equiv.), 40 min |
| 10 | Fmoc-Gly-OH (3.00 equiv.) | DEPBT (2.85 equiv.) and DIEA (6.00 equiv.), 40 min |
| 11 | Fmoc-OEG-OH (3.00 equiv.) | DEPBT (2.85 equiv.) and DIEA (6.00 equiv.), 40 min |
| 12 | Fmoc-OEG-OH (3.00 equiv.) | DEPBT (2.85 equiv.) and DIEA (6.00 equiv.), 40 min |
| 13 | Fmoc-Glu(OtBu)-OH (3.00 equiv.) | DEPBT (2.85 equiv.) and DIEA (6.00 equiv.), 40 min |
| 14 | Octadecanedioic Acid(OtBu) | DEPBT (2.85 equiv.) and DIEA (6.00 equiv.), 120 min |
| | (3.00 equiv.) | |

Step 15: TFA Cleavage and Ether precipitation: A solution of methyl(phenyl)sulfane (1.6 mL), phenol (0.8 mL) and ethane-1,2-dithiol (0.8 mL) in TFA (28 mL) and H₂O (0.8 mL) (TFA/thioanisole/phenol/ethane-1,2-dithiol/H₂O v/v/v/v/v, 87.5%/5%/2.5%/2.5%/2.5%) was stirred at 0° C. for 30 min in a round-bottom flask, and then the octadecanedioyl(OtBu)-Glu(OtBu)-OEG-OEG-Gly-Arg(Pbf)-His(Trt)-Trp(Boc)-Ala-Val-D-Ala-His(Trt)-Leu-Nle resin (SEQ ID NO: 323) (1.83 g, 0.6 mmol, 1 eq) was added to the mixture at 0° C. The resulting mixture was stirred at 25° C. for 4 hrs. The reaction mixture was filtered and the filtrate was added to cold MTBE (192 mL) to form a white precipitate. The white precipitate was filtered to give the crude peptide. The crude peptide was purified by prep-HPLC (column: CD24-WePure Biotech XPT C18 150×25×7 um; mobile phase: [A: H₂O (0.1% TFA), B: ACN]; % B: 26%-56%) to give Peptide 166 as a white solid. LCMS: (ES+) m/z (M/2+H)⁺=938.4.

### Example 4a: Synthesis of Peptide 247 (SEQ ID NO. 247)

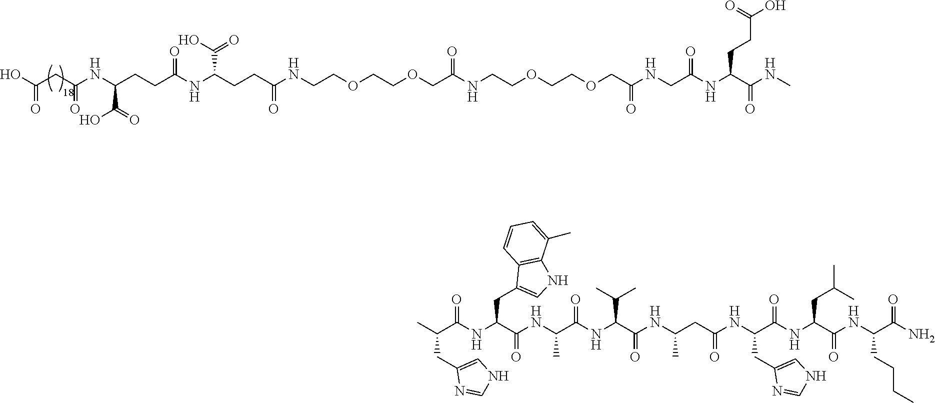

Peptide 247 was synthesized by standard Solid-phase Peptide Synthesis (SPPS) using Fmoc/t-Bu chemistry. The assembly was performed on a Rink-amide MBHA resin (100-200 Mesh; loading 0.51 mmol/g) in a peptide solid phase synthesis tube under a nitrogen atmosphere. The constructed peptide was isolated from the resin and protecting groups removed by cleavage with strong acid followed by precipitation. Peptide purification was performed prep-HPLC. Lyophilization of pure fractions gave the final product.

Swell Resin: 3 g of Rink Amide MBHA solid phase resin (0.51 mmol/g loading) was transferred to a 20-mL peptide vessel with filter frit, ground glass joint and vacuum side arm. The resin was washed with DMF (50 mL×3).

Step 1: Coupling of Fmoc-Nle-OH: A solution of 9H-fluoren-9-ylmethyl carbamate (0.36 g, 0.51 mmol/g loading) and piperidine (30 mL, 20 purity in DIF) was agitated under N₂ for 30 mmi, and then filtered. The resin (filter cake) was washed with DMF (60 mL×6). To a mixture of Fmoc-Nle-OH (2.12 g, 6.00 mmol, 4.0 eq) and DIEA (1.57 mL, 9.00 mmol, 6 eq) in DMF (20 mL) was added DEPBT (1.35 g, 4.50 mmol, 3.00 eq) at 25 (C. The mixture was stirred at 25° C. for 20 min. And then the mixture was added to the deprotected resin. The resulting mixture was agitated under N₂ for 40 min. After the reaction was judged complete, the resin was filtered and washed with DMF (50 mL×6), and then diluted with DIEA (0.78 mL, 3 eq) and Ac₂O (0.42 mL, 3 eq) in DMF (100 mL). The resulting mixture was agitated under N₂ at 25° C. for 30 mi, and then filtered and washed with DMF (60 mL×6) prior to starting the next deprotection or coupling cycle.

Steps 2˜15: Step 1 was repeated sequentially using the acid derivatives and coupling reagents in Table E.

TABLE F
Acid derivatives and coupling reagents used in SPPS.

| Step | Acid Derivative | Coupling Reagents |
|---|---|---|
| 2 | Fmoc-Leu-OH (3.00 equiv.) | DEPBT (2.85 equiv.) and DIEA (6.00 equiv.), 40 min |
| 3 | Fmoc-His(Trt)-OH (3.00 equiv.) | DEPBT (2.85 equiv.) and DIEA (6.00 equiv.), 40 min |
| 4 | Fmoc-BABA-OH (6.00 equiv.) | DEPBT (5.7 equiv.) and DIEA (12.00 equiv.), 40 min |
| 5 | Fmoc-Val-OH (3.00 equiv.) | DEPBT (2.85 equiv.) and DIEA (6.00 equiv.), 40 min |
| 6 | Fmoc-Ala-OH (6.00 equiv.) | DEPBT (5.7 equiv.) and DIEA (12.00 equiv.), 40 min |
| 7 | Fmoc-7MeTrp-OH (3.00 equiv.) | DEPBT (2.85 equiv.) and DIEA (6.00 equiv.), 40 min |
| 8 | Fmoc-His(Trt)-OH (3.00 equiv.) | DEPBT (2.85 equiv.) and DIEA (6.00 equiv.), 40 min |
| 9 | Fmoc-Glu(OtBu)-OH (3.00 equiv.) | DEPBT (2.85 equiv.) and DIEA (6.00 equiv.), 40 min |
| 10 | Fmoc-Gly-OH (3.00 equiv.) | DEPBT (5.7 equiv.) and DIEA (12.00 equiv.), 40 min |
| 11 | Fmoc-OEG-OH (3.00 equiv.) | DEPBT (2.85 equiv.) and DIEA (6.00 equiv.), 40 min |
| 12 | Fmoc-OEG-OH (3.00 equiv.) | DEPBT (2.85 equiv.) and DIEA (6.00 equiv.), 40 min |
| 13 | Fmoc-γGlu(OtBu)-OH (3.00 equiv.) | DEPBT (2.85 equiv.) and DIEA (6.00 equiv.), 40 min |
| 14 | Fmoc-γGlu(OtBu)-OH (3.00 equiv.) | DEPBT (2.85 equiv.) and DIEA (6.00 equiv.), 40 min |
| 15 | Eicosanedioyl(OtBu) Acid (3.00 equiv.) | DEPBT (2.85 equiv.) and DIEA (6.00 equiv.), 40 min |

Step 16: TFA Cleavage and Ether precipitation: A solution of thioanisole (2.8 mL), phenol (1.4 mL) and ethane-1,2-dithiol (1.4 mL) in TFA (49 mL) and H₂O (1.4 mL) (TFA/thioanisole/phenol/ethane-1,2-dithiol/H₂O v/v/v/v/v, 87.5%/5%/2.5%/2.5%/2.5%, 56 mL, 8 mL/g) was stirred at 0° C. for 15 min in a round-bottom flask, and then the Eicosanedioyl(OtBu)-γGlu(OtBu)-γGlu(OtBu)-OEG-OEG-Gly-Glu(OtBu)-His(Trt)-7MeTrp-Ala-Val-BABA-His(Trt)-Leu-Nle-resin (SEQ ID NO: 324) (7 g) was added to the mixture at 0° C. The resulting mixture was stirred at 25° C. for 1.75 hrs. The reaction mixture was filtered and the filtrate was added to cold MTBE (144 mL) to form a white precipitate. The white precipitate was filtered to give the crude peptide. The crude peptide was purified by prep-HPLC (column: CD18-Welch Ultimate C18 150×40×7 um; mobile phase: [A: water (0.1% TFA), B: ACN]; B %: 15%-45%, over 15.0 min) to give Peptide 247 as a white solid. LCMS: (ES+) m/z (M/2+H)⁺=1017.1; (M/3+H)⁺=678.4.

### Example 5: Synthesis of Peptide 248 (SEQ ID NO. 248)

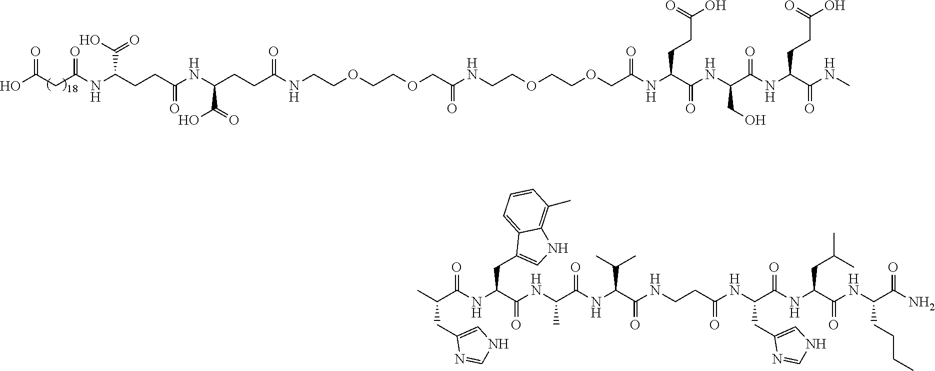

Peptide 248 was synthesized by standard Solid-phase Peptide Synthesis (SPPS) using Fmoc/t-Bu chemistry. The assembly was performed on a Rink-amide MBHA resin (100-200 Mesh; loading 0.60 mmol/g) in a peptide solid phase synthesis tube under a nitrogen atmosphere. The constructed peptide was isolated from the resin and protecting groups removed by cleavage with strong acid followed by precipitation. Peptide purification was performed prep-HPLC. Lyophilization of pure fractions gave the final product.

Swell Resin: 5 g of Rink Amide MBHA solid phase resin (0.60 mmol/g loading) was transferred to a 50-mL peptide vessel with filter frit, ground glass joint and vacuum side arm. The resin was washed with DMF (100 mL×3).

Step 1: Coupling of Fmoc-Nle-OH: A solution of 9H-fluoren-9-ylmethyl carbamate (0.71 g, 0.60 mmol/g loading) and piperidine (50 mL, 20% purity in DMF) was agitated under N₂ for 30 min, and then filtered. The resin (filter cake) was washed with DMF (100 mL×6). To a mixture of Fmoc-Nle-OH (3.18 g, 9.0 mmol, 3 eq) and DIEA (3.14 mL, 18 mmol, 6 eq) in DMF (30 mL) was added DEPBT (2.56 g, 8.6 mmol, 2.85 eq) at 25° C. The mixture was stirred at 25° C. for 20 min. And then the mixture was added to the deprotected resin. The resulting mixture was agitated under N₂ for 40 mi. After the reaction was judged complete, the resin was filtered and washed with DMF (100 mL×6), and then diluted with DIEA (3.14 mL, 3 eq) and AC₂O (1.6 mL, 3 eq) in DF (50 mL). The resulting mixture was agitated under N_{q} at 25° C. for 30 m, and then filtered and washed with DMF (100 mL×6) prior to starting the next deprotection or coupling cycle.

Steps 2˜16: Step 1 was repeated sequentially using the acid derivatives and coupling reagents in Table E.

TABLE G
Acid derivatives and coupling reagents used in SPPS.

| Step | Acid Derivative | Coupling Reagents |
|---|---|---|
| 2 | Fmoc-Leu-OH (3.00 equiv.) | DEPBT (2.85 equiv.) and DIEA (6.00 equiv.), 1 h |
| 3 | Fmoc-His(Trt)-OH (3.00 equiv.) | DEPBT (2.85 equiv.) and DIEA (6.00 equiv.), 1 h |
| 4 | Fmoc-β-Ala-OH (3.00 equiv.) | DEPBT (2.85 equiv.) and DIEA (6.00 equiv.), 1 h |
| 5 | Fmoc-Val-OH (3.00 equiv.) | DEPBT (2.85 equiv.) and DIEA (6.00 equiv.), 1 h |
| 6 | Fmoc-Ala-OH (6.00 equiv.) | DEPBT (5.7 equiv.) and DIEA (12.00 equiv.), 1 h |
| 7 | Fmoc-7MeTrp-OH (3.00 equiv.) | DEPBT (2.85 equiv.) and DIEA (6.00 equiv.), 1 h |
| 8 | Fmoc-His(Trt)-OH (3.00 equiv.) | DEPBT (2.85 equiv.) and DIEA (6.00 equiv.), 1 h |
| 9 | Fmoc-Glu(OtBu)-OH (3.00 equiv.) | DEPBT (2.85 equiv.) and DIEA (6.00 equiv.), 1 h |
| 10 | Fmoc-D-Ser(OtBu)-OH (6.00 equiv.) | DEPBT (5.7 equiv.) and DIEA (12.00 equiv.), 1 h |
| 11 | Fmoc-Glu(OtBu)-OH (3.00 equiv.) | DEPBT (2.85 equiv.) and DIEA (6.00 equiv.), 1 h |
| 12 | Fmoc-OEG-OH (3.00 equiv.) | DEPBT (2.85 equiv.) and DIEA (6.00 equiv.), 1 h |
| 13 | Fmoc-OEG-OH (3.00 equiv.) | DEPBT (2.85 equiv.) and DIEA (6.00 equiv.), 1 h |
| 14 | Fmoc-γGlu(OtBu)-OH (3.00 equiv.) | DEPBT (2.85 equiv.) and DIEA (6.00 equiv.), 1 h |
| 15 | Fmoc-γGlu(OtBu)-OH (3.00 equiv.) | DEPBT (2.85 equiv.) and DIEA (6.00 equiv.), 1 h |
| 16 | Eicosanedioyl(OtBu) Acid (3.00 equiv.) | DEPBT (2.85 equiv.) and DIEA (6.00 equiv.), 1 h |

Step 17: TFA Cleavage and Ether precipitation: A solution of thioanisole (5.6 mL), phenol (2.8 mL) and ethane-1,2-dithiol (2.8 mL) in TFA (98 mL) and H₂O (2.8 mL) (TFA/thioanisole/phenol/ethane-1,2-dithiol/H₂O v/v/v/v/v, 87.5%/5%/2.5%/2.5%/2.5%, 112 mL, 8 mL/g) was stirred at 0° C. for 15 min in a round-bottom flask, and then the Eicosanedioyl(OtBu)-γGlu(OtBu)-γGlu(OtBu)-OEG-OEG-Glu(OtBu)-D-Ser-Glu(OtBu)-His(Trt)-7MeTrp-Ala-Val-β-Ala-His(Trt)-Leu-Nle-resin (SEQ ID NO: 325) (14.0 g) was added to the mixture at 0° C. The resulting mixture was stirred at 25° C. for 3 hrs. The reaction mixture was filtered and the filtrate was added to cold MTBE (672 mL) to form a white precipitate. The white precipitate was filtered to give the crude peptide. The crude peptide was purified by prep-HPLC (column: CD31—WePure Biotech XPT C18 250×70×7 um; mobile phase: [A: water (0.1% TFA), B: ACN]; B %: 30%-60%, 30 min) to give Peptide 248 as a white solid. LCMS: (ES+) m/z (M/2+H)⁺=1089.5; (M/3+H)⁺=726.7.

The peptides below were prepared in a similar manner to those described in Examples 1-above.

| Peptide | Observed Mass |
|---|---|
| 1 | (M/2 + H)⁺ = 894.5 |
| 2 | (M + H)⁺ = 1800 |
| 3 | (M + H)⁺ = 1800 |
| 4 | (M/2 + H)⁺ = 923.9 |
| 5 | (M + H)⁺ = 1837.0 |
| 6 | (M + H)⁺ = 1116.3 |
| 7 | (M + H)⁺ = 1773.9 |
| 8 | (M + H)⁺ = 1779.0 |
| 9 | (M + H)⁺ = 1731.7 |
| 10 | (M + H)⁺ = 1354.8 |
| 11 | (M/2 + H)⁺ = 894.5 |
| 12 | (M/2 + H)⁺ = 893.3 |
| 13 | (M + H)⁺ = 1787.9 |
| 14 | (M + H)⁺ = 1802.8 |
| 15 | (M/2 + H)⁺ = 923.3 |
| 16 | (M/2 + H)⁺ = 895.3 |
| 17 | (M/2 + H)⁺ = 893.4 |
| 18 | (M/2 + H)⁺ = 955.9 |
| 19 | (M/2 + H)⁺ = 950.4 |
| 20 | (M/2 + H)⁺ = 943.0 |
| 21 | (M/2 + H)⁺ = 749.9 |
| 22 | (M + H)⁺ = 1803 |
| 23 | (M/2 + H)⁺ = 942.9 |
| 24 | (M/2 + H)⁺ = 902.4 |
| 25 | (M/2 + H)⁺ = 937.4 |
| 26 | (M/2 + H)⁺ = 742.8 |
| 46 | (M/2 + H)⁺ = 923.9 |
| 47 | (M/2 + H)⁺ = 952.4 |
| 48 | (M/2 + H)⁺ = 894.9 |
| 49 | (M/2 + H)⁺ = 916.4 |
| 50 | (M/2 + H)⁺ = 916.5 |
| 51 | (M + H)⁺ = 1786.1 |
| 52 | (M/2 + H)⁺ = 893.4 |
| 53 | (M/2 + H)⁺ = 1062.1 |
| 54 | (M/2 + H)⁺ = 1033.0 |
| 55 | (M/2 + H)⁺ = 1281.9 |
| 56 | (M/2 + H)⁺ = 896.9 |
| 57 | (M/2 + H)⁺ = 1223.9 |
| 58 | (M/2 + H)⁺ = 1045.5 |
| 59 | (M/2 + H)⁺ = 1039.6 |
| 60 | (M/2 + H)⁺ = 923.7 |
| | (M/3 + H)⁺ = 616.2 |
| 61 | (M/2 + H)⁺ = 937.9 |
| | (M/3 + H)⁺ = 625.7 |
| | (M/4 + H)⁺ = 469.4 |
| 62 | (M + H)⁺ = 1842.9 |
| 63 | (M/2 + H)⁺ = 894.9 |
| 64 | (M/2 + H)⁺ = 922.4 |
| | (M/3 + H)⁺ = 615.3 |
| 65 | (M/2 + H)⁺ = 945.2 |
| | (M/3 + H)⁺ = 630.5 |
| 66 | (M/2 + H)⁺ = 922.4. |
| 67 | (M/2 + H)⁺ = 929.8 |
| | (M/3 + H)⁺ = 620.3 |
| 68 | (M/2 + H)⁺ = 892.3 |
| 69 | (M/2 + H)⁺ = 938.4 |
| 70 | (M/2 + H)⁺ = 931.5 |
| | (M/3 + H)⁺ = 621.3 |
| 71 | (M/2 + H)⁺ = 916.3 |
| 72 | (M/2 + H)⁺ = 957.9 |
| 73 | (M/2 + H)⁺ = 964.9 |
| | (M/3 + H)⁺ = 643.6 |
| 74 | (M/2 + H)⁺ = 930.9 |
| | (M/3 + H)⁺ = 621.0 |
| 75 | (M/2 + H)⁺ = 930.4 |
| | (M/3 + H)⁺ = 620.6 |
| 76 | (M/2 + H)⁺ = 966.4 |
| 77 | (M/2 + H)⁺ = 980.4 |
| 78 | (M/2 + H)⁺ = 1068.1 |
| | (M/3 + H)⁺ = 712.7 |
| | (M/4 + H)⁺ = 534.8 |
| 79 | (M + H)⁺ = 1848.9 |
| | (M/2 + H)⁺ = 923.8 |
| | (M/3 + H)⁺ = 616.3 |
| 80 | (M/2 + H)⁺ = 821.3 |
| 81 | (M/2 + H)⁺ = 959.4 |
| 82 | (M + H)⁺ = 1918.0 |
| | (M/2 + H)⁺ = 959.4 |
| | (M/3 + H)⁺ = 640.0 |
| 83 | (M/2 + H)⁺ = 1067.5 |
| | (M/3 + H)⁺ = 712.1 |
| | (M/4 + H)⁺ = 534.3 |
| 84 | (M/2 + H)⁺ = 959.3 |
| | (M/3 + H)⁺ = 639.9 |
| 85 | (M/2 + H)⁺ = 923.8 |
| | (M/3 + H)⁺ = 616.3 |
| 86 | (M/2 + H)⁺ = 925.9 |
| | (M/3 + H)⁺ = 617.5 |
| 87 | (M/2 + H)⁺ = 896.8 |
| | (M/3 + H)⁺ = 598.2 |
| 88 | (M/2 + H)⁺ = 937.9 |
| | (M/3 + H)⁺ = 625.6 |
| 89 | (M/2 + H)⁺ = 908.9 |
| | (M/3 + H)⁺ = 606.2 |
| 90 | (M/2 + H)⁺ = 1069.0 |
| | (M/3 + H)⁺ = 713.0 |
| | (M/4 + H)⁺ = 535.0 |
| 91 | (M/2 + H)⁺ = 1045.7 |
| | (M/3 + H)⁺ = 697.4 |
| | (M/4 + H)⁺ = 523.3 |
| 92 | (M/2 + H)⁺ = 923.4 |
| 93 | (M/2 + H)⁺ = 893.1 |
| 94 | (M + H)⁺ = 1785.8 |
| 95 | (M/2 + H)⁺ = 1074.5 |
| | (M/3 + H)⁺ = 716.7 |
| | (M/4 + H)⁺ = 537.6 |
| 96 | (M/2 + H)⁺ = 887.8 |
| | (M/3 + H)⁺ = 592.2 |
| 97 | (M/2 + H)⁺ = 916.7 |
| | (M/3 + H)⁺ = 611.5 |
| 98 | (M/2 + H)⁺ = 1062.0 |
| | (M/3 + H)⁺ = 708.4 |
| | (M/4 + H)⁺ = 531.5 |
| 99 | (M/2 + H)⁺ = 957.5 |
| | (M/3 + H)⁺ = 638.7 |
| 100 | (M/2 + H)⁺ = 1001.9 |
| | (M/3 + H)⁺ = 668.3 |
| | (M/4 + H)⁺ = 501.3 |
| 101 | (M/2 + H)⁺ = 1106.5 |
| | (M/3 + H)⁺ = 738.1 |
| | (M/4 + H)⁺ = 553.8 |
| 102 | (M/2 + H)⁺ = 961.4 |
| | (M/3 + H)⁺ = 641.2 |
| 103 | (M/2 + H)⁺ = 1104.5 |
| | (M/3 + H)⁺ = 736.7 |
| | (M/4 + H)⁺ = 552.8 |
| 104 | (M/2 + H)⁺ = 959.4 |
| | (M/3 + H)⁺ = 639.9 |
| 105 | (M/2 + H)⁺ = 922.3 |
| | (M/3 + H)⁺ = 615.3 |
| 106 | (M/2 + H)⁺ = 916.5 |
| | (M/3 + H)⁺ = 611.3 |
| 107 | (M/2 + H)⁺ = 892.2 |
| | (M/3 + H)⁺ = 595.2 |
| 108 | (M/2 + H)⁺ = 901.8 |
| | (M/3 + H)⁺ = 601.6 |
| 109 | (M/2 + H)⁺ = 1092.0 |
| | (M/3 + H)⁺ = 728.4 |
| | (M/4 + H)⁺ = 546.6 |
| 110 | (M/2 + H)⁺ = 1070.9 |
| | (M/3 + H)⁺ = 714.3 |
| | (M/4 + H)⁺ = 536.0 |
| 111 | (M + H)⁺ = 1904.0 |
| 112 | (M/2 + H)⁺ = 959.3 |
| 114 | (M/2 + H)⁺ = 995.0. |
| 115 | (M/2 + H)⁺ = 952.5. |
| 116 | (M/2 + H)⁺ = 913.0 |
| | (M/3 + H)⁺ = 609.0 |
| | (M/4 + H)⁺ = 457.0 |
| 117 | (M/2 + H)⁺ = 1090.0 |
| | (M/3 + H)⁺ = 727.0 |
| | (M/4 + H)⁺ = 545.6 |
| 118 | (M/2 + H)⁺ = 1083.1 |
| | (M/3 + H)⁺ = 722.4 |
| | (M/4 + H)⁺ = 542.1 |
| 119 | (M/2 + H)⁺ = 937.0 |
| | (M/3 + H)⁺ = 625.0 |
| 120 | (M/2 + H)⁺ = 915.4 |
| | (M/3 + H)⁺ = 610.6 |
| 121 | (M/2 + H)⁺ = 1085.0 |
| | (M/3 + H)⁺ = 727.3 |
| | (M/4 + H)⁺ = 543.1 |
| 122 | (M/2 + H)⁺ = 1104.1 |
| | (M/3 + H)⁺ = 736.4 |
| | (M/4 + H)⁺ = 552.6 |
| 123 | (M/2 + H)⁺ = 973.4 |
| | (M/3 + H)⁺ = 649.3 |
| 124 | (M/2 + H)⁺ = 936.5 |
| | (M/3 + H)⁺ = 624.7 |
| 125 | (M/2 + H)⁺ = 973.5 |
| | (M/3 + H)⁺ = 649.4 |
| 126 | (M/2 + H)⁺ = 900.9 |
| | (M/3 + H)⁺ = 601.0 |
| 128 | (M/2 + H)⁺ = 930.9 |
| | (M/3 + H)⁺ = 620.9 |
| 129 | (M/2 + H)⁺ = 1008.9 |
| | (M/3 + H)⁺ = 673.0 |
| 130 | (M/2 + H)⁺ = 937.04 |
| | (M/3 + H)⁺ = 625.02 |
| | (M/4 + H)⁺ = 469.02 |
| 131 | (M/2 + H)⁺ = 966.04 |
| | (M/3 + H)⁺ = 644.36 |
| 132 | (M/2 + H)⁺ = 1096.63 |
| | (M/3 + H)⁺ = 731.42 |
| | (M/4 + H)⁺ = 548.82 |
| 133 | (M/2 + H)⁺ = 1106.1 |
| | (M/3 + H)⁺ = 738.1 |
| | (M/4 + H)⁺ = 553.8 |
| 134 | (M/2 + H)⁺ = 1002.0 |
| | (M/3 + H)⁺ = 668.4 |
| | (M/4 + H)⁺ = 501.5 |
| 136 | (M/3 + H)⁺ = 699.6 |
| | (M/4 + H)⁺ = 525.1 |
| 137 | (M/2 + H)⁺ = 1127.7 |
| | (M/3 + H)⁺ = 752.1 |
| | (M/4 + H)⁺ = 564.3 |
| 138 | (M/2 + H)⁺ = 975.04 |
| | (M/3 + H)⁺ = 650.36 |
| 139 | (M + H)⁺ = 1834.0 |
| | (M/2 + H)⁺ = 917.4 |
| | (M/3 + H)⁺ = 611.9 |
| 140 | (M/2 + H)⁺ = 962.0 |
| | (M/3 + H)⁺ = 641.7 |
| 141 | (M/2 + H)⁺ = 987.6 |
| | (M/3 + H)⁺ = 658.7 |
| | (M/4 + H)⁺ = 494.3 |
| 142 | (M/2 + H)⁺ = 916.9 |
| | (M/3 + H)⁺ = 611.6 |
| 143 | (M/2 + H)⁺ = 961.9 |
| | (M/3 + H)⁺ = 641.7 |
| 144 | (M/2 + H)⁺ = 967.4 |
| | (M/3 + H)⁺ = 645.3 |
| 145 | (M/2 + H)⁺ = 966.5 |
| | (M/3 + H)⁺ = 644.7 |
| 146 | (M/2 + H)⁺ = 917.4 |
| | (M/3 + H)⁺ = 611.9 |
| 147 | (M/2 + H)⁺ = 923.9 |
| | (M/3 + H)⁺ = 616.3 |
| 148 | (M/2 + H)⁺ = 931.5 |
| | (M/3 + H)⁺ = 621.3 |
| 149 | (M/2 + H)⁺ = 931.0 |
| | (M/3 + H)⁺ = 621.0 |
| | [(M + Na)/2]⁺ = 941.9 |
| 150 | (M/2 + H)⁺ = 951.9 |
| | (M/3 + H)⁺ = 635.0 |
| 151 | (M/2 + H)⁺ = 937.9 |
| | (M/3 + H)⁺ = 625.6 |
| 152 | (M + H)⁺ = 1789.9 |
| | M/2 + H)⁺ = 895.5 |
| | (M/3 + H)⁺ = 597.3 |
| 153 | (M/2 + H)⁺ = 806.8 |
| | M/3 + H)⁺ = 538.2 |
| 154 | (M/2 + H)⁺ = 835.4 |
| | (M/3 + H)⁺ = 557.2 |
| 155 | (M/2 + H)⁺ = 835.8 |
| | (M/2 + H)⁺ = 557.6 |
| 156 | (M/2 + H)⁺ = 966.9 |
| | (M/3 + H)⁺ = 645.0 |
| | (M/4 + H)⁺ = 484.0 |
| 157 | (M/2 + H)⁺ = 988.0 |
| | (M/3 + H)⁺ = 659.0 |
| | (M/4 + H)⁺ = 494.5 |
| 158 | (M + H)⁺ = 1712.1 |
| | (M/2 + H)⁺ = 856.4 |
| | (M/3 + H)⁺ = 571.2 |
| 159 | (M/2 + H)⁺ = 916.8 |
| | (M/3 + H)⁺ = 611.6 |
| 160 | (M/2 + H)⁺ = 785.8 |
| 161 | (M/2 + H)⁺ = 968.9 |
| | M/3 + H)⁺ = 646.4 |
| 162 | (M/2 + H)⁺ = 807.3 |
| | (M/3 + H)⁺ = 538.6 |
| 163 | (M/2 + H)⁺ = 856.9 |
| | (M/3 + H)⁺ = 571.7 |
| 164 | (M/2 + H)⁺ = 786.3 |
| 165 | (M/2 + H)⁺ = 792.7 |
| 166 | (M/2 + H)⁺ = 938.4 |
| | (M/3 + H)⁺ = 626.0 |
| 167 | (M/2 + H)⁺ = 952.4 |
| | (M/3 + H)⁺ = 635.4 |
| 240 | (M/2 + H)⁺ = 945.1 |
| | (M/3 + H)⁺ = 630.4 |
| 241 | (M/2 + H)⁺ = 937.9 |
| | (M/3 + H)⁺ = 625.6 |
| 242 | (M/2 + H)⁺ = 959.0 |
| | (M/3 + H)⁺ = 639.7 |
| | (M/4 + H)⁺ = 479.7 |
| 243 | (M/2 + H)⁺ = 1002.5 |
| | (M/3 + H)⁺ = 668.6 |
| 244 | (M/2 + H)⁺ = 1016.5 |
| | (M/3 + H)⁺ = 678.1 |
| | (M/4 + H)⁺ = 508.8 |
| 245 | (M/2 + H)⁺ = 1046.0 |
| | (M/3 + H)⁺ = 697.7 |
| 251 | [M + 2H]²⁺ = 1002.5 |
| 252 | [M + 2H]²⁺ = 1088.1 |
| | [M + 3H]³⁺ = 725.7 |
| | [M + 4H]⁴⁺ = 544.6 |
| 253 | [M + 2H]²⁺ = 1073.1 |
| | [M + 3H]³⁺ = 715.7 |
| | [M + 4H]⁴⁺ = 537.1 |
| 254 | [M + 2H]²⁺ = 1101.6 |
| | [M + 3H]³⁺ = 734.8 |
| | [M + 4H]⁴⁺ = 551.4 |
| 255 | [M + 2H]²⁺ = 1053.5 |
| | [M + 3H]³⁺ = 702.7 |
| 256 | [M + 2H]²⁺ = 1067.1 |
| | [M + 3H]³⁺ = 711.7 |
| 257 | [M + 2H]²⁺ = 1089.0 |
| | [M + 3H]³⁺ = 726.4 |
| 258 | [M + 2H]²⁺ = 1009.5 |
| | [M + 3H]³⁺ = 673.3 |
| 259 | [M + 2H]²⁺ = 1016.5 |
| | [M + 3H]³⁺ = 678.0 |
| 260 | [M + H]⁺ = 1832.9 |
| | [M + 2H]²⁺ = 916.8 |
| 261 | [M + 2H]²⁺ = 994.5 |
| | [M + 3H]³⁺ = 663.4 |
| 262 | [M + 2H]²⁺ = 1017.5 |
| | [M + 3H]³⁺ = 678.7 |
| 263 | [M + 2H]²⁺ = 952.0 |
| | [M + 3H]³⁺ = 635.0 |
| 264 | [M + 2H]²⁺ = 951.9 |
| | [M + 3H]³⁺ = 635.0 |
| 265 | [M + 2H]²⁺ = 938.9 |
| | [M + 3H]³⁺ = 626.3 |
| 266 | [M + 2H]²⁺ = 1031.6 |
| | [M + 3H]³⁺ = 688.1 |
| 267 | [M + 2H]²⁺ = 945.0 |
| | [M + 3H]³⁺ = 630.3 |
| 268 | [M + 2H]²⁺ = 958.0 |
| | [M + 3H]³⁺ = 639.0 |
| 269 | [M + 2H]²⁺ = 966.0 |
| | [M + 3H]³⁺ = 644.3 |
| 270 | [M + 2H]²⁺ = 1023.5 |
| | [M + 3H]³⁺ = 682.7 |
| 271 | [M + 2H]²⁺ = 1022.6 |
| | [M + 3H]³⁺ = 682.1 |
| 272 | [M + 2H]²⁺ = 1015.4 |
| | [M + 3H]³⁺ = 677.3 |
| 273 | [M + 2H]²⁺ = 1022.5 |
| | [M + 3H]³⁺ = 682.1 |
| 274 | [M + 2H]²⁺ = 1002.6 |
| | [M + 3H]³⁺ = 668.7 |
| 275 | [M + 2H]²⁺ = 1010.5 |
| | [M + 3H]³⁺ = 674.1 |
| 276 | [M + 2H]²⁺ = 955.0 |
| | [M + 3H]³⁺ = 637.0 |
| 277 | [M + 2H]²⁺ = 1039.0 |
| | [M + 3H]³⁺ = 693.0 |
| 278 | [M + 2H]²⁺ = 1017.1 |
| | [M + 3H]³⁺ = 678.4 |

### Example A-1: In Vitro Testing of Peptides for IP1 GRPR Activity

U2OS cells stably expressing human GRP receptors (DiscoverX, #93-0229C3) were cultured according to manufacturer's instructions. Cells were cultured to 8500 confluency, then collected using AccuTase (Innovative Cell Technologies, #AT104). Cells were washed and plated on a poly-D-lysine-coated 384-well plate (ThermoFisher, #A3890401) at a density of 2,500 cells/well in 15 mL assay buffer (AssayComplete Cell Plating 5 Reagent, DiscoverX #93-0563R5), then incubated overnight at 37° C. and 5% C₀₂.

The following morning, a homogenous time-resolved fluorescence (HTRF) assay was performed in a 384-well plate format using the IP1-Gq assay kit (Cisbio, 62IPAPEB). Test compounds were made as a stock in DMSO, then serially diluted to generate 10-point dose response curves in IP1 stimulation buffer. Five microliters of the prepared compounds were then added to the plate containing cells and assay buffer in duplicate, and the cells were incubated at 37° C. and 5% CO₂ for 90 minutes. Following compound incubation, 8 mL of IP1 lysis/detection buffer (prepared according to manufacturer's instructions) was added to each well, and the plates were incubated at room temperature for 1 hour.

HTRF signals were measured using a plate reader (ClarioSTAR), calculating the ratio between emissions at 665 nm and 620 nm (HTRF ratio). HTRF ratio for positive (Max, 1 M GRP; SEQ ID NO. 45) and negative (Min, DMSO) controls were used to normalize HTRF data and generate values for % activity. EC₅₀ and Max activity values were determined using a standard 4-parameter fit using GraphPad Prism.

TABLE 4
IP1 GRPR Activity Data

| Peptide | hIP1 GRPR EC₅₀ (nM) |
|---|---|
| GRP10 | 15 |
| 1 | 6.9 |
| 2 | 822 |
| 3 | 8.7 |
| 4 | 1420 |
| 5 | 7.5 |
| 6 | 26.8 |
| 7 | 0.6 |
| 8 | 1.7 |
| 9 | 9.4 |
| 10 | 3.8 |
| 11 | 3.2 |
| 12 | 1.8 |
| 13 | 1.08 |
| 14 | 10.8 |
| 15 | 1.4 |
| 16 | 5.8 |
| 17 | 3 |
| 18 | ~1000 |
| 19 | >1000 |
| 20 | >1000 |
| 21 | 120 |
| 22 | 10.3 |
| 23 | 1090 |
| 24 | 183 |
| 25 | 10000 |
| 26 | 1.8 |
| 46 | 9.9 |
| 47 | 216 |
| 48 | 1.1 |
| 49 | 4040 |
| 50 | >10,000 |
| 51 | 0.95 |
| 52 | 1.75 |
| 53 | 42 |
| 54 | 3.12 |
| 55 | 82.7 |
| 56 | 1.97 |
| 57 | 72.8 |
| 58 | 4.22 |
| 59 | 2.01 |
| 60 | 7.27 |
| 61 | 8.25 |
| 62 | 83.7 |
| 63 | 4.75 |
| 64 | 49.4 |
| 65 | 6.05 |
| 66 | 308 |
| 67 | 1050 |
| 68 | >10000 |
| 69 | >10000 |
| 70 | 972 |
| 71 | 6.2 |
| 72 | 1050 |
| 73 | 1190 |
| 74 | 25.5 |
| 75 | 18.2 |
| 76 | 358 |
| 77 | 184 |
| 78 | 14 |
| 79 | 529 |
| 80 | 5.11 |
| 81 | 505 |
| 82 | 1230 |
| 83 | 109 |
| 84 | 2550 |
| 85 | 14.7 |
| 86 | 17.6 |
| 87 | 1.73 |
| 88 | 5.51 |
| 89 | 1.57 |
| 90 | 12.6 |
| 91 | 3.72 |
| 92 | 1.64 |
| 93 | 6.95 |
| 94 | 1.45 |
| 95 | 110 |
| 96 | 1.58 |
| 97 | 8.91 |
| 98 | 11.2 |
| 99 | >10000 |
| 100 | 99.9 |
| 101 | 352 |
| 102 | 181 |
| 103 | 345 |
| 104 | 221 |
| 105 | 232 |
| 106 | 467 |
| 107 | >10000 |
| 108 | >10000 |
| 109 | 18.7 |
| 110 | 12.4 |
| 111 | 197 |
| 112 | >10000 |
| 114 | >10000 |
| 115 | >10000 |
| 116 | >10000 |
| 117 | 16.5 |
| 118 | 8.02 |
| 119 | 660 |
| 120 | 443 |
| 121 | 356 |
| 122 | 3.88 |
| 123 | 60.1 |
| 124 | 2380 |
| 125 | >10000 |
| 126 | 92.5 |
| 128 | 1060 |
| 129 | 334 |
| 130 | >10000 |
| 131 | 54.2 |
| 132 | 4.18 |
| 133 | 600 |
| 134 | >10000 |
| 136 | 40.5 |
| 137 | 488 |
| 138 | 81.8 |
| 139 | >10000 |
| 140 | 4.8 |
| 141 | 7.72 |
| 142 | 3.55 |
| 143 | 10.7 |
| 144 | 5.99 |
| 145 | 5.2 |
| 146 | 279 |
| 147 | 5.72 |
| 148 | 126 |
| 149 | 1.89 |
| 150 | 4.09 |
| 151 | 2.92 |
| 152 | 3.9 |
| 153 | 2.87 |
| 154 | 7.09 |
| 155 | 132 |
| 156 | 358 |
| 157 | 161 |
| 158 | 1.68 |
| 159 | 101 |
| 160 | 31.5 |
| 161 | 21.4 |
| 162 | 19.6 |
| 163 | 88.5 |
| 164 | 125 |
| 165 | 1.55 |
| 166 | 248 |
| 167 | 114 |
| 240 | 11.9 |
| 241 | 2.0 |
| 242 | 3.9 |
| 243 | 3.6 |
| 244 | 2.8 |
| 245 | 11.9 |
| 247 | 1.8 |
| 248 | 10.5 |
| 251 | 4.4 |
| 252 | 7.5 |
| 253 | 3.7 |
| 254 | 3.7 |
| 255 | 6.9 |
| 256 | 3.7 |
| 257 | 12.5 |
| 258 | 6.0 |
| 259 | 8.2 |
| 260 | 0.99 |
| 261 | 4 |
| 262 | 4 |
| 263 | 2.7 |
| 264 | 8.6 |
| 265 | 7.7 |
| 266 | 11 |
| 267 | 7.4 |
| 268 | 4.3 |
| 269 | 2.4 |
| 270 | 3.8 |
| 271 | 10.2 |
| 272 | 6.0 |
| 273 | 12.3 |
| 274 | 4.9 |
| 275 | 12.3 |
| 276 | 63.8 |
| 277 | 13.1 |
| 278 | 10.8 |

### Example A-2a: In Vivo Testing of Peptides

Male C57BL/6J mice ordered from Jackson Laboratory at age of 13 weeks old. They were singly housed in a 12 h reverse light cycle room (9:00 am lights out to 9:00 pm light on) for minimal 2 weeks before the study. Animals were provided standard rodent chow (PicoLab Rodent Diet, 5053) ad libitum. During the period prior to the study day, mice were exposed to one pellet of high fat diet (Research Diets D12492i) to avoid any effects of novelty in the study. On three separate days during this run-in period, mice were given one dose of phosphate-buffered saline (PBS; 5 mL/kg) by subcutaneous injection to acclimate them to the procedure. For baseline measures, food was removed from cages 1 hour prior to the dark cycle onset. Pre-weighed meals of standard rodent chow were provided to each animal following acclimation dosing at 1 hour following the onset of the dark cycle, and a baseline measurement began. A second dose of PBS was administered 5 hours following the provision of food. Food intake and bodyweight measurements were recorded at 5 and 24 hours following the presentation of the pre-weighed meal. These measurements and the resulting bodyweight changes were then used to assign mice to balanced cohorts.

On the day of the study, food was removed from cages 1 hour prior to the dark cycle onset. One hour following onset of the dark cycle, mice were given a subcutaneous dose of either vehicle (PBS, 5 mL/kg; pH 5.0), liraglutide (37 μg/kg) or a peptide of the disclosure (500 nmol/kg; pH 5.5). Following dosing, pre-weighed high fat diet was provided. A second administration of peptide or saline was given 6 hours following the first dose. Food intake measurements were taken at 2 and 6 hours following the provision of the pre-weighed high fat diet. Food intake and body weight measurements were additionally taken 24 hours following the provision of the pre-weighed high fat diet.

Mice administered a peptide of the disclosure demonstrated a reduction in food intake compared to the amount consumed by mice administered vehicle and also showed a reduction in body weight relative to mice administered vehicle.

Table 5 describes the percent reduction in food intake and the percent body weight change with peptides and control, as measured relative to the vehicle group.

TABLE 5
Reduction in Food Intake and Body Weight
After Subcutaneous Administration in Mice

| | % Reduction in Cumulative | % Body Weight |
|---|---|---|
| | Food Intake over 24 hours | Change at 24 hours |
| Peptide | (compared with vehicle) | (compared with vehicle) |
| Liraglutide | 63 | −10.3 |
| 5 | 44 | −6 |
| 7 | 61 | −7.7 |
| 8 | 46 | −5.2 |

### Example A-2b: In Vivo Testing of Peptides

Male C57BL/6J mice, approximately 10 weeks old (Jackson Laboratory), were group housed in a 12-h light cycle room (6:00 AM on, 6:00 PM off) upon arrival and acclimated to the facility for a minimum of 1 week prior to the study. Animals were provided standard rodent chow (PicoLab Rodent Diet, 5053) ad libitum. Animals were single caged for a minimum of 3 days prior to the study. During the period prior to the study day, mice were exposed to one pellet of high fat diet (Research Diets D12492i) to avoid any effects of novelty in the study. On three separate days during this run-in period, mice were given one dose of phosphate-buffered saline (PBS; 5 mL/kg) by subcutaneous injection to acclimate them to the procedure. For baseline measures, pre-weighed meals of standard rodent chow were provided to each animal following acclimation dosing at 3:30 PM, and body weight was recorded. The next morning at 9:30 AM, food intake and body weight measurements were recorded at 16 hours following the presentation of the pre-weighed meal. These measurements and the resulting body weight changes were then used to assign mice to balanced cohorts.

On the day of the study, mice were weighed and given a subcutaneous dose of either vehicle (PBS, 5 mL/kg; pH 5.0-8.5) or a peptide of the disclosure (500 nmol/kg; pH 5.0-8.5). Following dosing, a pre-weighed high fat diet was provided. Food intake and body weight measurements were taken at 16 hours following the provision of the pre-weighed high fat diet.

Mice administered a peptide of the disclosure demonstrated a reduction in food intake compared to the amount consumed by mice administered vehicle and also showed a reduction in body weight relative to mice administered vehicle.

Table 6 below describes the percent reduction in food intake and the percent body weight (BW) change with peptides as measured relative to the vehicle group.

TABLE 6
Reduction in Food Intake and Body Weight
After Subcutaneous Administration in Mice

| | % Reduction in | |
|---|---|---|
| | Cumulative Food | % Body Weight |
| | Intake over 16 hours | Change at 16 hours |
| Peptide | (compared with vehicle) | (compared with vehicle) |
| Liraglutide | 43 | −4 |
| 5 | 33 | −4 |
| 7 | 39 | −6 |
| 241 | 74 | −10 |
| 242 | 55 | −9 |
| 247 | 65 | −11 |
| 248 | 54 | −9 |
| 277 | 69 | −12 |
| 278 | 63 | −12 |

### Example A-3. Pharmacokinetics after Intravenous Peptide Administration

Table 6 describes the pharmacokinetics for peptides of the disclosure in C57BL/6 mice. GPR-10 and peptides were dosed IV at 2 mg/kg as a formulation of 0.4 mg/mL in 500 DMSO+3000 PEG400+65% water (pH 6-7). As shown in Table 6, following IV administration, lipid-conjugated peptides of the disclosure had a significantly longer half-life (Ti/2) and reduced clearance (CL) relative to GRP10 and the non-lipidated peptide.

TABLE 7
Pharmacokinetics of Peptides After Administration in Mice

| Peptide | T_{1/2} (h) | CL (mg/min/kg) |
|---|---|---|
| GRP10 | 0.3 | 4536 |
| 5 | 4.9 | 0.4 |
| 6 | <0.1 | * |
| 7 | 2.1 | 1.2 |
| 8 | 2 | 1.2 |
| 9 | 5.7 | 0.5 |
| 241 | 7.0 | 0.23 |
| 247 | 7.5 | 0.18 |
| 248 | 9.7 | 0.12 |
| 252 | 6.6 | 0.12 |
| 255 | 9.4 | 0.09 |
| 257 | 9.4 | 0.09 |

* Clearance of Peptide 6 occurred too rapidly to be measured.

### Example A-4: Solubility in Buffer

Solubility was measured in an appropriate buffer as follows. A known amount of compound, e.g., 20 mg, was placed in a vessel equipped with a stirring bar. A measured amount of buffer (e.g., 200-400 μL of 200 mM phosphate buffer) was added, and in some cases, additional surfactants, e.g., polysorbate 80 (PS80), polyethylene glycol (15)-hydroxystearate (Solutol® HS15), etc., were added. A measured amount of purified water (e.g., 1.6 mL) was added, and the pH was adjusted to the desired pH, e.g., pH 8, by addition of 0.5 N aqueous NaOH or 0.5 N aqueous HCl. The volume of added acid or base was recorded. The mixture was stirred at 300 rpm for 10 min. Additional purified water was added to reach a given volume, e.g., 4 mL, of solution. After stirring the sample at 300 rpm at room temperature for 1 h or 24 h, the appearance was observed and the pH measured. A portion of the sample was taken to filter or centrifuge for testing the 1- or 24-hour solubility by HPLC.

Following the above procedure, the solubility of Peptide 247 in 20 mM pH 8.0 phosphate buffer with 0.3% PS80+10% HS15 was found to be 4.20 mg/mL at 24 h.

What is claimed is:

1. A peptide of Formula (VI): 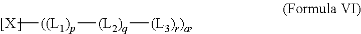
or a pharmaceutically acceptable salt thereof, wherein:
X is a peptide comprising an amino acid sequence of Formula (V):
X0-X1-X2-Ar-X4-X5-V-X7-X8-L-X10-X11-X12,
wherein:
X0 is absent or Glu;
X1 is D-Ser, Ser, or Gly;
X2 is Asn, Glu, Arg, Ala, Asp, Gln, or Lys(Ac);
Ar is His, 3-(3-pyridyl)alanine (3Pal), 3-(4-pyridyl)alanine (4Pal), or 3-(2-pyridyl)alanine (2Pal);
X4 is Arg, His, Trp, 7-methyltryptophan (7MeTrp), 6-methyltryptophan (6MeTrp), 7-fluorotryptophan (7FTrp), 6-fluorotryptophan (6FTrp), or 3-(2-naphthyl)alanine (2Nal);
X5 is Ala, α-aminoisobutyric acid (Aib), or D-Ala;
X7 is D-Ala, 3-Ala, α-aminoisobutyric acid (Aib), Gly, Ala, or S-β-aminobutanoic acid (BABA);
X8 is His or NMeHis;
X10 is absent, Met, Arg, norleucine (Nle), 3-(3-pyridyl)alanine (3Pal), or Lys(Me)₃;
X11 is absent, sarcosine (Sar), α-aminoisobutyric acid (Aib), or Arg; and
X12 is absent;
each L¹ is independently —((CH₂)ᵥNR¹)_{w}—CO(CH₂)ₓO(PEG)_{y}(CH₂)_{z}NR¹—;
PEG is —CH₂CH₂O—;
v is 2-6; w is 0-1; x is 1-4; y is 1-4; z is 2-24;
R¹ is H or —CH₃;
or each L¹ is OEG;
OEG is —NH-PEG-PEG-CH₂C(═O)—;
PEG is —CH₂CH₂O—;
each L² is independently a natural or unnatural amino acid or amino acid analog;
each L³ is independently a substituted or unsubstituted C₂-C₂₆ fatty acyl group;
a is 1, 2, 3, or 4;
p is 0-8;
q is 0-6; and
r is 1 or 2.

2. The peptide of claim 1, or a pharmaceutically acceptable salt thereof, wherein:
X0 is absent or Glu;
X1 is D-Ser, Ser, or Gly;
X2 is Asn, Gln, Glu, or Arg;
Ar is His;
X4 is 7-methyltryptophan (7MeTrp) or Trp;
X5 is Ala;
X7 is D-Ala, 3-Ala, BABA, or Gly;
X8 is His or NMeHis;
X10 is norleucine (Nle) or Met;
X11 is absent; and
X12 is absent.

3. The peptide of claim 1, or a pharmaceutically acceptable salt thereof, wherein:
X0 is absent.

4. The peptide of claim 1, or a pharmaceutically acceptable salt thereof, wherein:
X0 is Glu.

5. The peptide of claim 1, or a pharmaceutically acceptable salt thereof, wherein:
Ar is His.

6. The peptide of claim 1, or a pharmaceutically acceptable salt thereof, wherein:
X4 is Trp or 7MeTrp.

7. The peptide of claim 1, or a pharmaceutically acceptable salt thereof, wherein:
X10 is Nle.

8. The peptide of claim 1, or a pharmaceutically acceptable salt thereof, wherein:
a is 1.

9. The peptide of claim 1, or a pharmaceutically acceptable salt thereof, wherein:
(-(L¹)ₚ-(L²)_{q}-(L³)ᵣ) is attached to the N-terminus of peptide X.

10. The peptide of claim 1, or a pharmaceutically acceptable salt thereof, wherein:
each L¹ is OEG; and
each L² is Glu or D-Glu.

11. The peptide of claim 1, or a pharmaceutically acceptable salt thereof, wherein:
L³ is

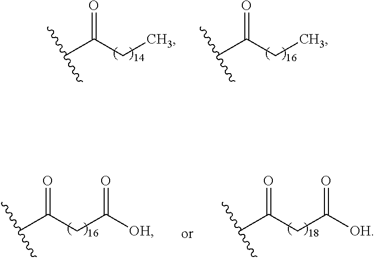

12. The peptide of claim 1, or a pharmaceutically acceptable salt thereof, wherein:
-(L¹)ₚ-(L²)_{g}-(L³)ᵣ is

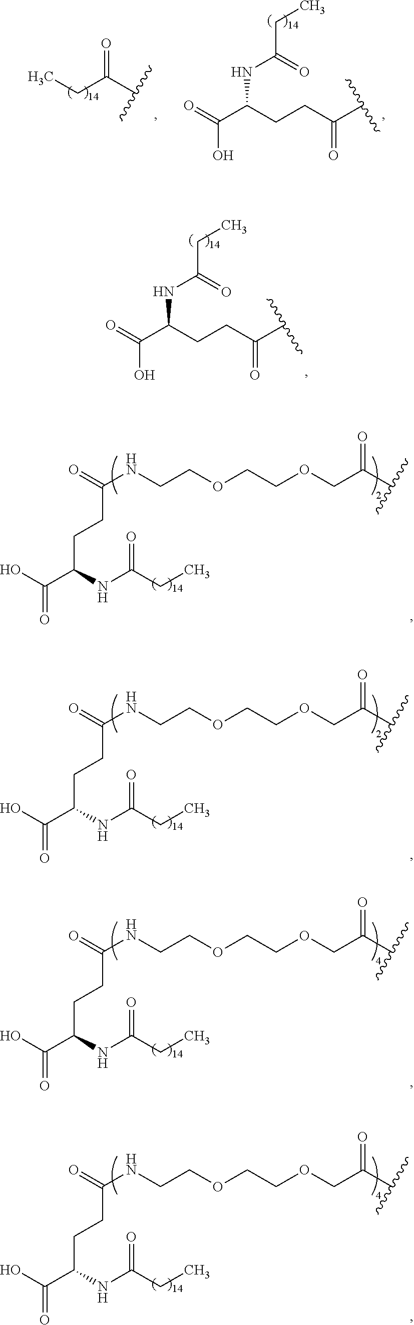

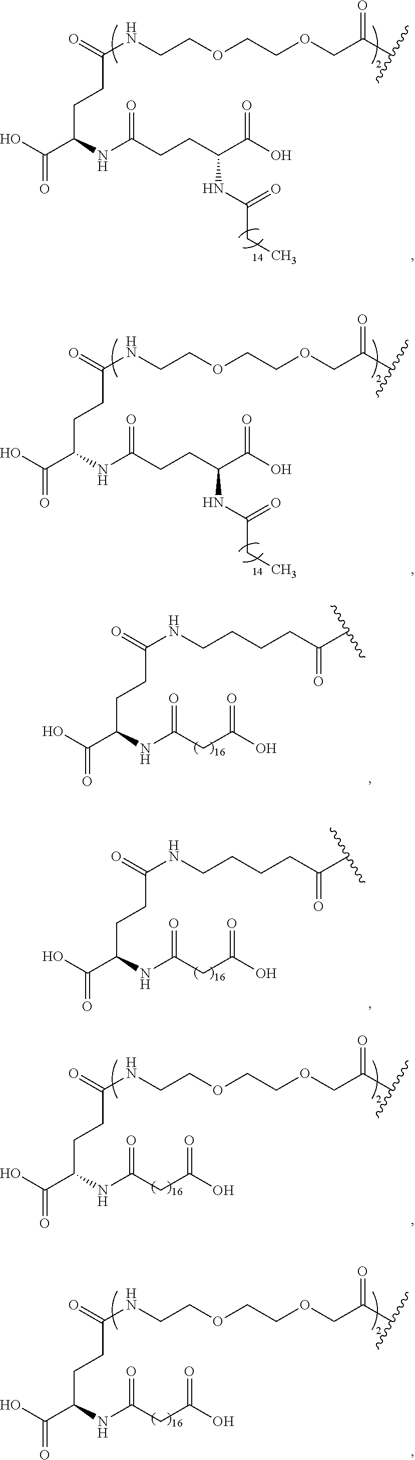

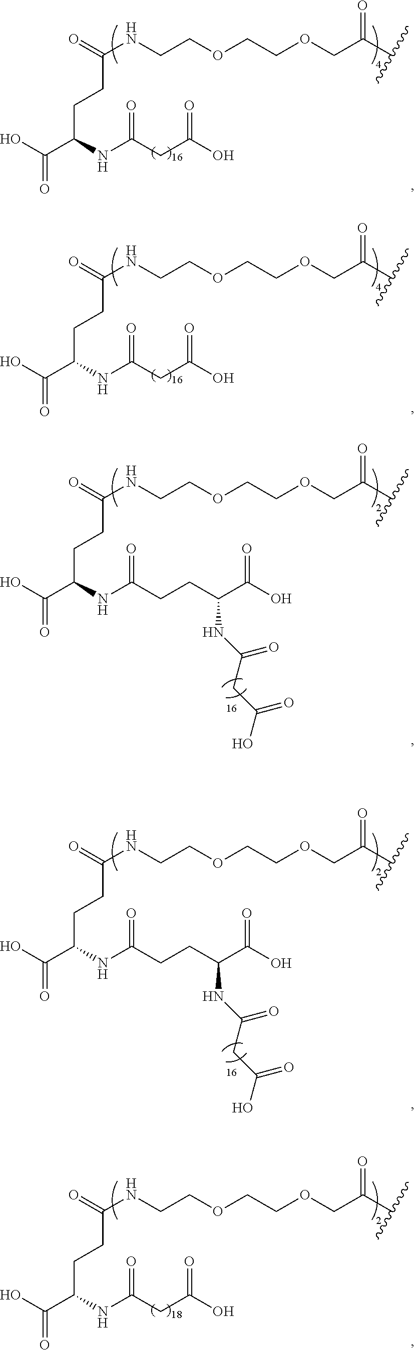

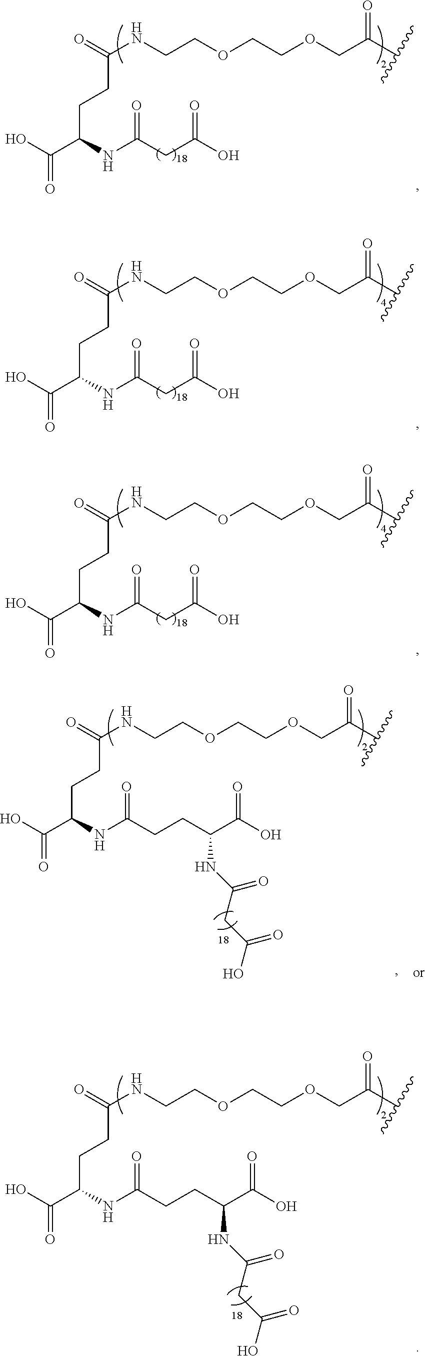

13. The peptide of claim 1, or a pharmaceutically acceptable salt thereof, wherein:
-(L¹)ₚ-(L²)_{g}-(L³)ᵣ is

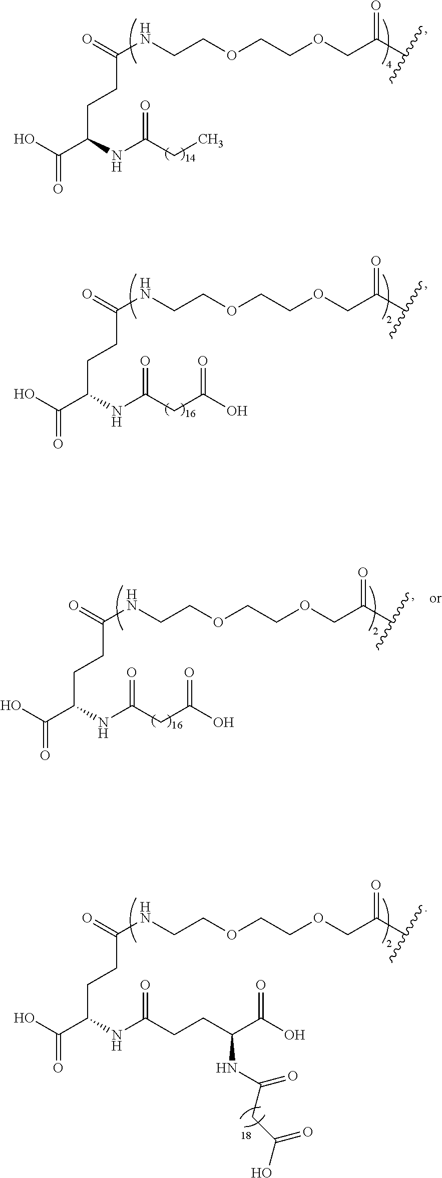

14. The peptide of claim 1, or a pharmaceutically acceptable salt thereof, wherein the peptide comprises the sequence:
(SEQ ID NO. 1)
Palmitoyl-D-γGlu-OEG-OEG-Gly-Asn-His-Trp-Ala-Val-Aib-His-Leu-Nle-NH₂,
(SEQ ID NO. 2)
Palmitoyl-D-γGlu-OEG-OEG-Gly-Asn-His-Trp-Ala-Val-Aib-3Pal-Leu-Nle-NH₂,
(SEQ ID NO. 3)
Palmitoyl-D-γGlu-OEG-OEG-Gly-Asn-3Pal-Trp-Ala-Val-Aib-His-Leu-Nle-NH₂,
(SEQ ID NO. 4)
Octadecanedioyl-D-γGlu-OEG-OEG-Gly-Asn-His-Trp-Ala-Val-Aib-His-Leu-Nle-NH₂,
(SEQ ID NO. 5)
Octadecanedioyl-D-γGlu-OEG-OEG-Gly-Asn-His-Trp-Ala-Val-Gly-His-Leu-Met-NH₂,
(SEQ ID NO. 7)
Palmitoyl-D-γGlu-OEG-OEG-Gly-Asn-His-Trp-Ala-Val-D-Ala-His-Leu-Nle-NH₂,
(SEQ ID NO. 8)
Palmitoyl-D-γGlu-OEG-OEG-Gly-Asn-His-Trp-Ala-Val-Gly-His-Leu-Met-NH₂,
(SEQ ID NO. 9)
Octadecanedioyl-D-γGlu-OEG-OEG-Gly-Asn-His-Trp-Ala-Val-D-Ala-His-Leu-Nle-NH₂,
(SEQ ID NO. 10)
Palmitoyl-Gly-Asn-His-Trp-Ala-Val-D-Ala-His-Leu-Nle-NH₂,
(SEQ ID NO. 11)
Palmitoyl-D-γGlu-OEG-OEG-Gly-Asn-His-Trp-Aib-Val-D-Ala-His-Leu-Nle-NH₂,
(SEQ ID NO. 12)
Palmitoyl-D-γGlu-OEG-OEG-Gly-Asn-3Pal-Trp-Ala-Val-D-Ala-His-Leu-Nle-NH₂,
(SEQ ID NO. 13)
Palmitoyl-D-γGlu-OEG-OEG-Gly-Asn-His-7MethylTrp-Ala-Val-D-Ala-His-Leu-Nle-NH₂,
(SEQ ID NO. 14)
Palmitoyl-D-γGlu-OEG-OEG-Gly-Asn-His-7MethylTrp-Aib-Val-D-Ala-His-Leu-Nle-NH₂,
(SEQ ID NO. 15)
Palmitoyl-D-γGlu-OEG-OEG-Gly-Asn-His-Trp-Ala-Val-D-Ala-His-Leu-Nle-Sar-NH₂,
(SEQ ID NO. 16)
Palmitoyl-D-γGlu-OEG-OEG-Gly-Glu-His-Trp-Ala-Val-D-Ala-His-Leu-Nle-NH₂,
(SEQ ID NO. 17)
Palmitoyl-D-γGlu-OEG-OEG-Gly-Asn-His-Trp-Ala-Val-D-Ala-3Pal-Leu-Nle-NH₂,
(SEQ ID NO. 18)
Palmitoyl-D-γGlu-OEG-OEG-Gly-Glu-3Pal-7MethylTrp-Aib-Val-D-Ala-3Pal-Leu-Nle-Sar-NH₂,
(SEQ ID NO. 19)
Palmitoyl-D-γGlu-OEG-OEG-Gly-Glu-His-7MethylTrp-Aib-Val-D-Ala-3Pal-Leu-Nle-Sar-NH₂,
(SEQ ID NO. 20)
Palmitoyl-D-γGlu-OEG-OEG-Gly-Asn-His-7MethylTrp-Aib-Val-D-Ala-3Pal-Leu-Nle-Sar-NH₂,
(SEQ ID NO. 21)
Palmitoyl-D-γGlu-Gly-Asn-His-Trp-Ala-Val-Aib-His-Leu-Nle-NH₂,
(SEQ ID NO. 22)
Palmitoyl-D-γGlu-OEG-OEG-Gly-Asn-His-7MethylTrp-Ala-Val-Aib-His-Leu-Nle-NH₂,
(SEQ ID NO. 23)
Palmitoyl-D-γGlu-OEG-OEG-Gly-Asn-His-7MethylTrp-Ala-Val-Aib-3Pal-Leu-Nle-Sar-NH₂,
(SEQ ID NO. 24)
Palmitoyl-D-γGlu-OEG-OEG-Gly-Glu-His-Trp-Ala-Val-Aib-His-Leu-Nle-NH₂,
(SEQ ID NO. 25)
Ac-Gly-Asn-His-Trp-Ala-Val-D-Ala-His-Leu-Nle-H₂N(CH₂)₃NH₂-OEG-OEG-(α-Palmitoyl-D-
γGlu),
(SEQ ID NO. 26)
Palmitoyl-D-γGlu-Gly-Asn-His-Trp-Ala-Val-D-Ala-His-Leu-Nle-NH₂,
(SEQ ID NO. 46)
Octadecanedioyl-D-Glu-OEG-OEG-Gly-Asn-His-7MethylTrp-Ala-Val-D-Ala-His-Leu-Nle-NH₂,
(SEQ ID NO. 47)
Octadecanedioyl-D-γGlu-OEG-OEG-Gly-Asn-His-Trp-Ala-Val-D-Ala-His-Leu-Nle-Sar-NH₂,
(SEQ ID NO. 48)
Palmitoyl-D-γGlu-OEG-OEG-Gly-Asn-His-Trp-Ala-Val-D-Ala-His-Leu-Nle-Sar-NH₂,
(SEQ ID NO. 49)
Palmitoyl-D-γGlu-OEG-OEG-Gly-Asn-His-Trp-Ala-Val-D-Ala-His-Leu-Lys(Me)3-NH₂,
(SEQ ID NO. 50)
Ac-Gly-Asn-His-Trp-Ala-Val-D-Ala-His-Leu-ϵK-OEG-OEG-(α-Palmitoyl-D-γGlu)-NH₂,
(SEQ ID NO. 51)
Palmitoyl-D-γGlu-OEG-OEG-Gly-Asn-2Pal-Trp-Ala-Val-^{D}Ala-His-Leu-Nle-NH₂,
(SEQ ID NO. 52)
Palmitoyl-D-γGlu-OEG-OEG-Gly-Asn-4Pal-Trp-Ala-Val-D-Ala-His-Leu-Nle-NH₂,
(SEQ ID NO. 53)
Octadecanedioyl-D-γGlu-OEG-OEG-OEG-OEG-Gly-Asn-His-Trp-Ala-Val-D-Ala-His-Leu-Nle-
NH₂,
(SEQ ID NO. 54)
Palmitoyl-D-γGlu-OEG-OEG-OEG-OEG-Gly-Asn-His-Trp-Ala-Val-D-Ala-His-Leu-Nle-NH₂,
(SEQ ID NO. 55)
Octadecanedioyl-D-γGlu-OEG-OEG-Gly-Lys(OEG-OEG-Octadecanedioyl-D-Glu-His-Trp-Ala-
Val-D-Ala-His-Leu-Nle-NH₂,
(SEQ ID NO. 56)
Palmitoyl-D-γGlu-OEG-OEG-Gly-Asn-His-7FTrp-Ala-Val-D-Ala-His-Leu-Nle-NH₂,
(SEQ ID NO. 57)
Palmitoyl-D-γGlu-OEG-OEG-Gly-Lys(OEG-OEG-α-Palmitoyl-D-γGlu)-His-Trp-Ala-Val-D-
Ala-His-Leu-Nle-NH₂,
(SEQ ID NO. 58)
Palmitoyl-D-γGlu-OEG-OEG-OEG-OEG-Gly-Asn-3Pal-7MethylTrp-Ala-Val-D-Ala-His-Leu-
Nle-NH₂,
(SEQ ID NO. 59)
Palmitoyl-D-γGlu-OEG-OEG-OEG-OEG-Gly-Asn-His-7MethylTrp-Ala-Val-D-Ala-His-Leu-
Nle-NH₂,
(SEQ ID NO. 60)
Octadecanedioyl-D-γGlu-OEG-OEG-Gly-Lys-His-Trp-Ala-Val-D-Ala-His-Leu-Nle-NH₂,
(SEQ ID NO. 61)
Octadecanedioyl-D-γGlu-OEG-OEG-Gly-Arg-His-Trp-Ala-Val-D-Ala-His-Leu-Nle-NH₂,
(SEQ ID NO. 62)
Octadecanedioyl-D-γGlu-OEG-OEG-Gly-Asn-His-Trp-Ala-Val-D-Ala-Phe-Leu-Nle-NH₂,
(SEQ ID NO. 63)
Palmitoyl-D-γGlu-OEG-OEG-Gly-Asn-His-6MethylTrp-Ala-Val-D-Ala-His-Leu-Nle-NH₂,
(SEQ ID NO. 64)
Octadecanedioyl-D-γGlu-OEG-OEG-Gly-Asn-3Pal-Trp-Ala-Val-D-Ala-His-Leu-Nle-NH₂,
(SEQ ID NO. 65)
Octadecanedioyl-D-γGlu-OEG-OEG-Gly-Lys(Ac)-His-Trp-Ala-Val-D-Ala-His-Leu-Nle-NH₂,
(SEQ ID NO. 66)
Octadecanedioyl-D-γGlu-OEG-OEG-Gly-Asn-His-Trp-Ala-Val-D-Ala-3Pal-Leu-Nle-NH₂,
(SEQ ID NO. 67)
Octadecanedioyl-D-γGlu-OEG-OEG-Gly-Glu-His-Trp-Ala-Val-D-Ala-3Pal-Leu-Nle-NH₂,
(SEQ ID NO. 68)
Octadecanedioyl-D-γGlu-OEG-OEG-Gly-Asn-His-His-Ala-Val-D-Ala-His-Leu-Nle-NH₂,
(SEQ ID NO. 69)
Octadecanedioyl-D-γGlu-OEG-OEG-Gly-Asn-His-Trp-Ala-Val-D-Ala-His-Leu-Arg-NH₂,
(SEQ ID NO. 70)
Octadecanedioyl-D-γGlu-OEG-OEG-Gly-Glu-His-Trp-Aib-Val-D-Ala-His-Leu-Nle-NH₂,
(SEQ ID NO. 71)
Octadecanedioyl-D-γGlu-OEG-OEG-Lys(Ac)-His-Trp-Ala-Val-D-Ala-His-Leu-Nle-NH₂,
(SEQ ID NO. 72)
Octadecanedioyl-D-γGlu-OEG-OEG-Gly-Asn-3Pal-Trp-Ala-Val-D-Ala-His-Leu-Nle-Sar-NH₂,
(SEQ ID NO. 73)
Octadecanedioyl-D-γGlu-OEG-OEG-Gly-Asn-3Pal-7MethylTrp-Ala-Val-D-Ala-His-Leu-Nle-
Sar-NH₂,
(SEQ ID NO. 74)
Octadecanedioyl-D-γGlu-OEG-OEG-Aib-Asn-His-Trp-Ala-Val-D-Ala-His-Leu-Nle-NH₂,
(SEQ ID NO. 75)
Palmitoyl-D-γGlu-OEG-OEG-Gly-Asn-His-Trp-Ala-Val-D-Ala-His-Leu-Nle-Aib-NH₂,
(SEQ ID NO. 76)
Octadecanedioyl-D-γGlu-OEG-OEG-Gly-Asn-His-7MethylTrp-Ala-Val-D-Ala-His-Leu-Nle-
Aib-NH₂,
(SEQ ID NO. 77)
Octadecanedioyl-D-γGlu-OEG-OEG-Gly-Lys(Ac)-His-Trp-Ala-Val-D-Ala-His-Leu-Nle-Sar-
NH₂,
(SEQ ID NO. 78)
Octadecanedioyl-D-γGlu-OEG-OEG-OEG-OEG-Gly-Asn-His-7MethyTrp-Ala-Val-D-Ala-His-
Leu-NH₂,
(SEQ ID NO. 79)
Octadecanedioyl-D-γGlu-OEG-OEG-Gly-Asn-His-6MethylTrp-Ala-Val-D-Ala-His-Leu-Nle-
NH₂,
(SEQ ID NO. 80)
Octadecanedioyl-D-γGlu-Ava-Gly-Asn-His-Trp-Ala-Val-D-Ala-His-Leu-Nle-NH₂,
(SEQ ID NO. 81)
Octadecanedioyl-D-γGlu-OEG-OEG-Gly-Asn-His-7MethylTrp-Ala-Val-D-Ala-His-Leu-Nle-
Sar-NH₂,
(SEQ ID NO. 82)
Octadecanedioyl-D-γGlu-OEG-OEG-Gly-Asn-His-Trp-Ala-Val-D-Ala-His-Leu-Nle-Aib-NH₂,
(SEQ ID NO. 83)
Octadecanedioyl-D-γGlu-OEG-OEG-OEG-OEG-Gly-Asn-3Pal-Trp-Ala-Val-D-Ala-His-Leu-
Nle-NH₂,
(SEQ ID NO. 84)
Octadecanedioyl-D-γGlu-OEG-OEG-Gly-Asn-His-6MethylTrp-Ala-Val-D-Ala-His-Leu-Nle-
Sar-NH₂,
(SEQ ID NO. 85)
Octadecanedioyl-γGlu-OEG-OEG-Gly-Asn-His-7MethylTrp-Ala-Val-D-Ala-His-Leu-Nle-NH₂,
(SEQ ID NO. 86)
Octadecanedioyl-γGlu-OEG-OEG-Gly-Asn-His-7FTrp-Ala-Val-D-Ala-His-Leu-Nle-NH₂,
(SEQ ID NO. 87)
Palmitoyl-γGlu-OEG-OEG-Gly-Asn-His-7FTrp-Ala-Val-D-Ala-His-Leu-Nle-NH₂,
(SEQ ID NO. 88)
Octadecanedioyl-γGlu-OEG-OEG-Gly-Arg-His-Trp-Ala-Val-D-Ala-His-Leu-Nle-NH₂,
(SEQ ID NO. 89)
Palmitoyl-γGlu-OEG-OEG-Gly-Arg-His-Trp-Ala-Val-D-Ala-His-Leu-Nle-NH₂,
(SEQ ID NO. 90)
Octadecanedioyl-γGlu-OEG-OEG-OEG-OEG-Gly-Asn-His-7MethylTrp-Ala-Val-D-Ala-His-
Leu-Nle-NH₂,
(SEQ ID NO. 91)
Palmitoyl-γGlu-OEG-OEG-OEG-OEG-Gly-Asn-3Pal-7MethylTrp-Ala-Val-D-Ala-His-Leu-Nle-
NH₂,
(SEQ ID NO. 92)
Palmitoyl-γGlu-OEG-OEG-Gly-Asn-His-Trp-Ala-Val-D-Ala-His-Leu-Nle-Sar-NH₂,
(SEQ ID NO. 93)
Palmitoyl-γGlu-OEG-OEG-Gly-Asn-His-2Nal-Ala-Val-Ala-His-Leu-Nle-NH₂,
(SEQ ID NO. 94)
Palmitoyl-γGlu-OEG-OEG-Gly-Asn-4Pal-Trp-Ala-Val-D-Ala-His-Leu-Nle-NH₂,
(SEQ ID NO. 95)
Octadecanedioyl-γGlu-OEG-OEG-OEG-OEG-Gly-Asn-3Pal-7MeTrp-Ala-Val-D-Ala-His-Leu-
Nle-NH₂,
(SEQ ID NO. 96)
Palmitoyl-γGlu-OEG-OEG-Gly-Asn-His-Trp-Ala-Val-D-Ala-His-Leu-Nle-NH₂,
(SEQ ID NO. 97)
Octadecanedioyl-γGlu-OEG-OEG-Gly-Asn-His-Trp-Ala-Val-D-Ala-His-Leu-Nle-NH₂,
(SEQ ID NO. 98)
Octadecanedioyl-γGlu-OEG-OEG-OEG-OEG-Gly-Asn-His-Trp-Ala-Val-D-Ala-His-Leu-Nle-
NH₂,
(SEQ ID NO. 99)
Octadecanedioyl-γGlu-OEG-OEG-Gly-Asn-His-2Nal-Ala-Val-D-Ala-His-Leu-Nle-Sar-NH₂,
(SEQ ID NO. 100)
Octadecanedioyl-γGlu-OEG-OEG-Arg-Asn-His-Trp-Ala-Val-D-Ala-His-Leu-Nle-Sar-NH₂,
(SEQ ID NO. 101)
Octadecanedioyl-γGlu-OEG-OEG-OEG-OEG-Gly-Asn-His-7FTrp-Ala-Val-D-Ala-His-Leu-Nle-
Sar-NH₂,
(SEQ ID NO. 102)
Octadecanedioyl-γGlu-OEG-OEG-Gly-Asn-His-7FTrp-Ala-Val-D-Ala-His-Leu-Nle-Sar-NH₂,
(SEQ ID NO. 103)
Octadecanedioyl-γGlu-OEG-OEG-OEG-OEG-Gly-Asn-His-7MethylTrp-Ala-Val-D-Ala-His-
Leu-Nle-Sar-NH₂,
(SEQ ID NO. 104)
Octadecanedioyl-γGlu-OEG-OEG-Gly-Asn-His-7MethylTrp-Ala-Val-D-Ala-His-Leu-Nle-Sar-
NH₂,
(SEQ ID NO. 105)
Octadecanedioyl-γGlu-OEG-OEG-Gly-Asn-His-2Nal-Ala-Val-D-Ala-His-Leu-Nle-NH₂,
(SEQ ID NO. 106)
Octadecanedioyl-γGlu-OEG-OEG-Gly-Asn-His-Trp-D-Ala-Val-D-Ala-His-Leu-Nle-NH₂,
(SEQ ID NO. 107)
Octadecanedioyl-γGlu-OEG-OEG-Gly-Asn-His-His-Ala-Val-D-Ala-His-Leu-Nle-NH₂,
(SEQ ID NO. 108)
Octadecanedioyl-γGlu-OEG-OEG-Gly-Asn-His-Arg-Ala-Val-D-Ala-His-Leu-Nle-NH₂,
(SEQ ID NO. 109)
Octadecanedioyl-γGlu-OEG-OEG-OEG-OEG-Gly-Arg-His-7FTrp-Ala-Val-D-Ala-His-Leu-Nle-
NH₂,
(SEQ ID NO. 110)
Octadecanedioyl-γGlu-OEG-OEG-OEG-OEG-Gly-Asn-His-7FTrp-Ala-Val-D-Ala-His-Leu-Nle-
NH₂,
(SEQ ID NO. 111)
Palmitoyl-γGlu-OEG-OEG-Gly-Asn-His-Trp-Ala-Val-D-Ala-His-Leu-Nle-Sar-NH₂,
(SEQ ID NO. 112)
Palmitoyl-γGlu-OEG-OEG-Gly-Asn-His-7MethylTrp-Ala-Val-D-Ala-His-Leu-Nle-Sar-NH₂,
(SEQ ID NO. 114)
Octadecanedioyl-γGlu-OEG-OEG-Gly-Asn-His-Trp-Ala-Val-D-Ala-His-Leu-Nle-Arg-NH₂,
(SEQ ID NO. 115)
Octadecanedioyl-γGlu-OEG-OEG-Gly-Asn-His-Trp-D-Ala-Val-D-Ala-His-Leu-Nle-Sar-NH₂,
(SEQ ID NO. 116)
Octadecanedioyl-γGlu-OEG-OEG-Gly-Arg-His-His-Ala-Val-D-Ala-His-Leu-Nle-NH₂,
(SEQ ID NO. 117)
Octadecanedioyl-γGlu-OEG-OEG-OEG-OEG-Gly-Arg-His-7MethylTrp-Ala-Val-D-Ala-His-
Leu-Nle-NH₂,
(SEQ ID NO. 118)
Octadecanedioyl-γGlu-OEG-OEG-OEG-OEG-Gly-Arg-His-Trp-Ala-Val-D-Ala-His-Leu-Nle-
NH₂,
(SEQ ID NO. 119)
Octadecanedioyl-γGlu-OEG-OEG-Gly-Asn-His-Trp-Ala-Val-D-Ala-His-Leu-Cha-NH₂,
(SEQ ID NO. 120)
Octadecanedioyl-γGlu-OEG-OEG-Gly-Ala-His-Trp-Ala-Val-D-Ala-His-Leu-Cha-NH₂,
(SEQ ID NO. 121)
Octadecanedioyl-γGlu-OEG-OEG-OEG-OEG-Gly-Ala-His-7FTrp-Ala-Val-D-Ala-His-Leu-Nle-
Sar-NH₂,
(SEQ ID NO. 122)
Eicosanedioyl-γGlu-OEG-OEG-OEG-OEG-Gly-Arg-His-7MethylTrp-Ala-Val-D-Ala-His-Leu-
Nle-NH₂,
(SEQ ID NO. 123)
Eicosanedioyl-γGlu-OEG-OEG-Gly-Asn-His-7MethylTrp-Ala-Val-D-Ala-His-Leu-Nle-Sar-NH₂,
(SEQ ID NO. 124)
Octadecanedioyl-γGlu-OEG-OEG-Gly-Ala-His-2Nal-Ala-Val-D-Ala-His-Leu-Nle-Sar-NH₂,
(SEQ ID NO. 125)
Eicosanedioyl-γGlu-OEG-OEG-Gly-Asn-His-7MethylTrp-D-Ala-Val-D-Ala-His-Leu-Nle-Sar-
NH₂,
(SEQ ID NO. 126)
Octadecanedioyl-γGlu-OEG-OEG-Gly-Ala-His-2Nal-Ala-Val-D-Ala-His-Leu-Nle-NH₂,
(SEQ ID NO. 128)
Octadecanedioyl-γGlu-OEG-OEG-Gly-Ala-His-Trp-D-Ala-Val-D-Ala-His-Leu-Nle-Sar-NH₂,
(SEQ ID NO. 129)
Octadecanedioyl-γGlu-OEG-OEG-Gly-Arg-His-7MethyTrp-Ala-Val-D-Ala-His-Leu-Nle-Sar-
NH₂,
(SEQ ID NO. 130)
Octadecanedioyl-γGlu-OEG-OEG-Gly-Asn-His-Arg-Ala-Val-D-Ala-His-Leu-Nle-Sar-NH₂,
(SEQ ID NO. 131)
Eicosanedioyl-γGlu-OEG-OEG-Gly-Asn-His-Trp-Ala-Val-D-Ala-His-Leu-Nle-Sar-NH₂,
(SEQ ID NO. 132)
Eicosanedioyl-γGlu-OEG-OEG-OEG-OEG-Gly-Arg-His-Trp-Ala-Val-D-Ala-His-Leu-Nle-NH₂,
(SEQ ID NO. 133)
Octadecanedioyl-γGlu-OEG-OEG-OEG-OEG-Gly-Asn-His-6FTrp-Ala-Val-D-Ala-His-Leu-Nle-
Sar-NH₂,
(SEQ ID NO. 134)
Octadecanedioyl-γGlu-OEG-OEG-Gly-Asn-His-7MeTrp-Ala-Val-D-Ala-His-Leu-Nle-Arg-NH₂,
(SEQ ID NO. 136)
Octadecanedioyl-γGlu-OEG-OEG-OEG-OEG-Gly-Ala-His-7FTrp-Ala-Val-D-Ala-His-Leu-Nle-
NH₂,
(SEQ ID NO. 137)
Octadecanedioyl-γGlu-OEG-OEG-OEG-OEG-Gly-Arg-His-7FTrp-Ala-Val-D-Ala-His-Leu-Nle-
Sar-NH₂,
(SEQ ID NO. 138)
Eicosanedioyl-γGlu-OEG-OEG-Gly-Asn-His-7FTrp-Ala-Val-D-Ala-His-Leu-Nle-Sar-NH₂,
(SEQ ID NO. 139)
Octadecanedioyl-γGlu-OEG-OEG-Gly-Asp-His-Trp-D-Ala-Val-D-Ala-His-Leu-Nle-NH₂,
(SEQ ID NO. 140)
Octadecanedioyl-γGlu-OEG-OEG-Phe-Asn-His-Trp-Ala-Val-β-Ala-His-Leu-Nle-NH₂,
(SEQ ID NO. 141)
Octadecanedioyl-γGlu-OEG-OEG-Arg-Arg-His-Trp-Ala-Val-D-Ala-His-Leu-Nle-NH₂,
(SEQ ID NO. 142)
Octadecanedioyl-γGlu-OEG-OEG-Gly-Asn-His-Trp-Ala-Val-β-Ala-His-Leu-Nle-NH₂,
(SEQ ID NO. 143)
Octadecanedioyl-γGlu-OEG-OEG-D-Phe-Asn-His-Trp-Ala-Val-D-Ala-His-Leu-Nle-NH₂,
(SEQ ID NO. 144)
Octadecanedioyl-γGlu-OEG-OEG-Phe-Asn-3Pal-Trp-Ala-Val-β-Ala-His-Leu-Nle-NH₂,
(SEQ ID NO. 145)
Octadecanedioyl-γGlu-OEG-OEG-Arg-Asn-His-Trp-Ala-Val-D-Ala-His-Leu-Nle-NH₂,
(SEQ ID NO. 146)
Octadecanedioyl-γGlu-OEG-OEG-Gly-Asn-His-Trp-Ala-Val-D-Ala-His-Leu-Nle,
(SEQ ID NO. 147)
Octadecanedioyl-γGlu-OEG-OEG-Gly-Gln-His-Trp-Ala-Val-D-Ala-His-Leu-Nle-NH₂,
(SEQ ID NO. 148)
Eicosanedioyl-γGlu-OEG-OEG-Gly-Asn-His-Trp-Ala-Val-D-Ala-His-Leu-Nle,
(SEQ ID NO. 149)
Eicosanedioyl-γGlu-OEG-OEG-Gly-Asn-His-Trp-Ala-Val-D-Ala-His-Leu-Nle-NH₂,
(SEQ ID NO. 150)
Eicosanedioyl-γGlu-OEG-OEG-Gly-Arg-His-Trp-Ala-Val-D-Ala-His-Leu-Nle-NH₂,
(SEQ ID NO. 151)
Eicosanedioyl-γGlu-OEG-OEG-Gly-Gln-His-Trp-Ala-Val-D-Ala-His-Leu-Nle-NH₂,
(SEQ ID NO. 152)
Octadecanedioyl-γGlu-OEG-OEG-Gln-His-Trp-Ala-Val-β-Ala-His-Leu-Nle-NH₂,
(SEQ ID NO. 153)
Eicosanedioyl-γGlu-Gly-Arg-His-Trp-Ala-Val-D-Ala-His-Leu-Nle-NH₂,
(SEQ ID NO. 154)
Eicosanedioyl-γGlu-Arg-Asn-His-Trp-Ala-Val-D-Ala-His-Leu-Nle-NH₂,
(SEQ ID NO. 155)
Eicosanedioyl-γGlu-Arg-Asn-His-Trp-Ala-Val-D-Ala-His-Leu-Nle,
(SEQ ID NO. 156)
Octadecanedioyl-γGlu-OEG-OEG-Arg-Asn-His-Trp-Ala-Val-D-Ala-His-Leu-Nle,
(SEQ ID NO. 157)
Octadecanedioyl-γGlu-OEG-OEG-Arg-Arg-His-Trp-Ala-Val-D-Ala-His-Leu-Nle,
(SEQ ID NO. 158)
Eicosanedioyl-γGlu-Arg-Arg-His-Trp-Ala-Val-D-Ala-His-Leu-Nle-NH₂,
(SEQ ID NO. 159)
Octadecanedioyl-γGlu-OEG-OEG-Gly-Asn-His-Trp-Ala-Val-D-Ala-D-His-Leu-Nle-NH₂,
(SEQ ID NO. 160)
Eicosanedioyl-γGlu-Gly-Asn-His-Trp-Ala-Val-D-Ala-D-His-Leu-Nle-NH₂,
(SEQ ID NO. 161)
Octadecanedioyl-γGlu-OEG-OEG-D-Phe-Gln-His-Trp-Ala-Val-D-Ala-His-Leu-Nle-NH₂,
(SEQ ID NO. 162)
Eicosanedioyl-γGlu-Gly-Arg-His-Trp-Ala-Val-D-Ala-His-Leu-Nle,
(SEQ ID NO. 163)
Eicosanedioyl-γGlu-Arg-Arg-His-Trp-Ala-Val-D-Ala-His-Leu-Nle,
(SEQ ID NO. 164)
Eicosanedioy1-γGlu-Gly-Asn-His-Trp-Ala-Val-D-Ala-His-Leu-Nle,
(SEQ ID NO. 165)
Eicosanedioyl-γGlu-Gly-Gln-His-Trp-Ala-Val-D-Ala-His-Leu-Nle-NH₂,
(SEQ ID NO. 166)
Octadecanedioyl-γGlu-OEG-OEG-Gly-Arg-His-Trp-Ala-Val-D-Ala-His-Leu-Nle,
(SEQ ID NO. 167)
Eicosanedioyl-γGlu-OEG-OEG-Gly-Arg-His-Trp-Ala-Val-D-Ala-His-Leu-Nle,
(SEQ ID NO. 240)
Palmitoyl-γGlu-γGlu-OEG-OEG-Gly-Asn-His-Trp-Ala-Val-Gly-His-Leu-Nle-NH₂,
(SEQ ID NO. 241)
Eicosanedioyl-γGlu-OEG-OEG-Gly-Gln-His-Trp-Ala-Val-β-Ala-His-Leu-Nle-NH₂,
(SEQ ID NO. 242)
Eicosanedioyl-γGlu-OEG-OEG-Gly-Arg-His-7MethylTrp-Ala-Val-β-Ala-His-Leu-Nle-NH₂,
(SEQ ID NO. 243)
Octadecanedioyl-γGlu-γGlu-OEG-OEG-Gly-Arg-His-Trp-Ala-Val-β-Ala-His-Leu-Nle-NH₂,
(SEQ ID NO. 244)
Eicosanedioyl-γGlu-γGlu-OEG-OEG-Gly-Arg-His-Trp-Ala-Val-D-Ala-His-Leu-Nle-NH₂,
(SEQ ID NO. 245)
Eicosanedioyl-γGlu-γGlu-OEG-OEG-D-Phe-Asn-3PAl-Trp-Ala-Val-β-Ala-His-Leu-Nle-NH₂,
(SEQ ID NO. 247)
Eicosanedioyl-γGlu-Glu-OEG-OEG-Gly-Glu-His-7MeTrp-Ala-Val-BABA-His-Leu-Nle-NH₂,
(SEQ ID NO. 248)
Eicosanedioyl-γGlu-Glu-OEG-OEG-Glu-D-Ser-Glu-His-7MeTrp-Ala-Val-β-Ala-His-Leu-Nle-
NH₂,
(SEQ ID NO. 251)
Eicosanedioyl-γGlu-Glu-OEG-OEG-Gly-Gln-His-Trp-Ala-Val-β-Ala-His-Leu-Nle-NH₂,
(SEQ ID NO. 252)
Eicosanedioyl-γGlu-Glu-OEG-OEG-Glu-Gly-Arg-His-7MeTrp-Ala-Val-β-Ala-His-Leu-Nle-
NH₂,
(SEQ ID NO. 253)
Eicosanedioyl-γGlu-Glu-OEG-OEG-Arg-Arg-His-7MeTrp-Ala-Val-β-Ala-His-Leu-Nle-NH₂,
(SEQ ID NO. 254)
Eicosanedioyl-γGlu-Glu-OEG-OEG-Arg-Gly-Arg-His-7MeTrp-Ala-Val-β-Ala-His-Leu-Nle-
NH₂,
(SEQ ID NO. 255)
Eicosanedioyl-γGlu-Glu-OEG-OEG-Ser-Gly-Glu-His-7MeTrp-Ala-Val-β-Ala-His-Leu-Nle-
NH₂,
(SEQ ID NO. 256)
Eicosanedioyl-γGlu-Glu-OEG-OEG-Ser-Gly-Arg-His-7MeTrp-Ala-Val-β-Ala-His-Leu-Nle-
NH₂,
(SEQ ID NO. 257)
Eicosanedioyl-γGlu-Glu-OEG-OEG-Glu-Ser-Gln-His-7MeTrp-Ala-Val-β-Ala-His-Leu-Nle-
NH₂,
(SEQ ID NO. 258)
Eicosanedioyl-γGlu-γGlu-OEG-OEG-Gly-Asn-His-7MeTrp-Ala-Val-BABA-His-Leu-Nle-NH₂,
(SEQ ID NO. 259)
Eicosanedioyl-γGlu-γGlu-OEG-OEG-Gly-Gln-His-7MeTrp-Ala-Val-BABA-His-Leu-Nle-NH₂,
(SEQ ID NO. 260)
Eicosanedioyl-γGlu-OEG-OEG-Gly-Dap-His-Trp-Ala-Val-β-Ala-His-Leu-Nle-NH₂,
(SEQ ID NO. 261)
Eicosanedioyl-γGlu-OEG-OEG-Gly-Dap(5CTMPA)-His-Trp-Ala-Val-β-Ala-His-Leu-Nle-NH₂,
(SEQ ID NO. 262)
Eicosanedioyl-γGlu-OEG-OEG-Gly-Dap(2CE-PEG-TMEA)-His-Trp-Ala-Val-β-Ala-His-Leu-
Nle-NH₂,
(SEQ ID NO. 263)
Eicosanedioyl-γGlu-OEG-OEG-Gly-Gln-His-7MeTrp-Ala-Val-BAIBA-His-Leu-Nle-NH₂,
(SEQ ID NO. 264)
Eicosanedioyl-γGlu-OEG-OEG-Gly-Asn-His-7MeTrp-Ala-Val-3A3MBA-His-Leu-Nle-NH₂,
(SEQ ID NO. 265)
Eicosanedioyl-γGlu-OEG-OEG-Gly-Gln-His-Trp-Ala-Val-β-Ala-His-Leu-Hse(Me)-NH₂,
(SEQ ID NO. 266)
Eicosanedioyl-γGlu-OEG-OEG-Gly-Glu-Gly-Glu-His-Trp-Ala-Val-β-Ala-His-Leu-Nle-NH₂,
(SEQ ID NO. 267)
Eicosanedioyl-γGlu-OEG-OEG-Gly-Gln-His-Trp-Ala-Val-β-Ala-NmeHis-Leu-Nle-NH₂,
(SEQ ID NO. 268)
Eicosanedioyl-γGlu-OEG-OEG-Pro-Gln-His-Trp-Ala-Val-β-Ala-His-Leu-Nle-NH₂,
(SEQ ID NO. 269)
Eicosanedioyl-γGlu-OEG-OEG-Hyp-Gln-His-Trp-Ala-Val-β-Ala-His-Leu-Nle-NH₂,
(SEQ ID NO. 270)
Eicosanedioyl-γGlu-Glu-OEG-OEG-Gly-Gln-His-7MeTrp-Ala-Val-BABA-NmeHis-Leu-Nle-
NH₂,
(SEQ ID NO. 271)
Eicosanedioyl-γGlu-γGlu-OEG-OEG-Gly-Gln-His-Trp-Ala-Val-ACPC-His-Leu-Nle-NH₂,
(SEQ ID NO. 272)
Eicosanedioyl-γGlu-γGlu-OEG-OEG-Gly-Gln-His-Trp-Ala-Val-ACPA-His-Leu-Nle-NH₂,
(SEQ ID NO. 273)
Eicosanedioyl-γGlu-Glu-OEG-OEG-Gly-Gln-His-Trp-Ala-Val-ACBA-His-Leu-Nle-NH₂,
(SEQ ID NO. 274)
Eicosanedioyl-γGlu-Glu-OEG-OEG-Gly-Asn-His-αMeTrp-Ala-Val-β-Ala-His-Leu-Nle-NH₂,
(SEQ ID NO. 275)
Eicosanedioyl-γGlu-Glu-PEG5-Gly-Glu-His-7MeTrp-Ala-Val-BABA-His-Leu-Nle-NH₂,
(SEQ ID NO. 276)
Eicosanedioyl-γGlu-OEG-OEG-Gly-Gln-His-Trp-Ala-Val-β-Ala-His-Leu-MetO-NH₂,
(SEQ ID NO. 277)
Eicosanedioyl-γGlu-γGlu-OEG-OEG-Ser-Glu-His-7MeTrp-Ala-Val-BABA-NMeHis-Leu-Nle-
NH₂,
or
(SEQ ID NO. 278)
Eicosanedioyl-γGlu-γGlu-OEG-OEG-Gly-Asp-His-7MeTrp-Ala-Val-BABA-NMeHis-Leu-Nle-
NH₂,
or a pharmaceutically acceptable salt thereof.

15. The peptide of claim 1, or a pharmaceutically acceptable salt thereof, wherein the peptide comprises the sequence:
Octadecanedioyl-D-γGlu-OEG-OEG-Gly-Asn-His-Trp-Ala-Val-Gly-His-Leu-Met-NH₂ (SEQ ID NO. 5); or a pharmaceutically acceptable salt thereof.

16. The peptide of claim 1, or a pharmaceutically acceptable salt thereof, wherein the peptide comprises the sequence:
Palmitoyl-D-γGlu-OEG-OEG-Gly-Asn-His-Trp-Ala-Val-D-Ala-His-Leu-Nle-NH₂(SEQ ID NO. 7); or a pharmaceutically acceptable salt thereof.

17. The peptide of claim 1, or a pharmaceutically acceptable salt thereof, wherein the peptide comprises the sequence:
Eicosanedioyl-γGlu-OEG-OEG-Gly-Gln-His-Trp-Ala-Val-β-Ala-His-Leu-Nle-NH₂ (SEQ ID NO. 241); or a pharmaceutically acceptable salt thereof.

18. The peptide of claim 1, or a pharmaceutically acceptable salt thereof, wherein the peptide comprises the sequence:
Eicosanedioyl-γGlu-OEG-OEG-Gly-Arg-His-7MethylTrp-Ala-Val-β-Ala-His-Leu-Nle-NH₂ (SEQ ID NO. 242); or a pharmaceutically acceptable salt thereof.

19. The peptide of claim 1, or a pharmaceutically acceptable salt thereof, wherein the peptide comprises the sequence:
Eicosanedioyl-γGlu-γGlu-OEG-OEG-Gly-Glu-His-7MeTrp-Ala-Val-BABA-His-Leu-Nle-NH₂ (SEQ ID NO. 247); or a pharmaceutically acceptable salt thereof.

20. The peptide of claim 1, or a pharmaceutically acceptable salt thereof, wherein the peptide comprises the sequence:
Eicosanedioyl-γGlu-γGlu-OEG-OEG-Glu-D-Ser-Glu-His-7MeTrp-Ala-Val-3-Ala-His-Leu-Nle-NH₂ (SEQ ID NO. 248); or a pharmaceutically acceptable salt thereof.

21. The peptide of claim 1, or a pharmaceutically acceptable salt thereof, wherein the peptide comprises the sequence:
Eicosanedioyl-γGlu-γGlu-OEG-OEG-Glu-Gly-Arg-His-7MeTrp-Ala-Val-D-Ala-His-Leu-Nle-NH₂ (SEQ ID NO. 255); or a pharmaceutically acceptable salt thereof.

22. The peptide of claim 1, or a pharmaceutically acceptable salt thereof, wherein the peptide comprises the sequence:
Eicosanedioyl-γGlu-7Glu-OEG-OEG-Glu-Ser-Gln-His-7MeTrp-Ala-Val-β-Ala-His-Leu-Nle-NH₂ (SEQ ID NO. 257); or a pharmaceutically acceptable salt thereof.

23. The peptide of claim 1, or a pharmaceutically acceptable salt thereof, wherein the peptide comprises the sequence:
Eicosanedioyl-γGlu-γGlu-OEG-OEG-Ser-Glu-His-7MeTrp-Ala-Val-BABA-NMeHis-Leu-Nle-NH₂ (SEQ ID NO. 277); or a pharmaceutically acceptable salt thereof.

24. The peptide of claim 1, or a pharmaceutically acceptable salt thereof, wherein the peptide comprises the sequence:
Eicosanedioyl-γGlu-γGlu-OEG-OEG-Gly-Asp-His-7MeTrp-Ala-Val-BABA-NMeHis-Leu-Nle-NH₂ (SEQ ID NO. 278); or a pharmaceutically acceptable salt thereof.

25. A pharmaceutical composition comprising the peptide of claim 1, or a pharmaceutically acceptable salt thereof, and a pharmaceutically acceptable carrier, diluent, or excipient.

26. A method for reducing body weight of a subject in need thereof, the method comprising administering a therapeutically effective amount of a peptide of claim 1, or a pharmaceutically acceptable salt thereof.

27. A method for treating obesity in a subject in need thereof, the method comprising administering a therapeutically effective amount of a peptide of claim 1, or a pharmaceutically acceptable salt thereof.

28. A peptide comprising the sequence:
(SEQ ID NO. 6)
Gly-Asn-His-Trp-Ala-Val-D-Ala-His-Leu-Nle-NH₂;
(SEQ ID NO. 237)
Gly-Gln-His-Trp-Ala-Val-β-Ala-His-Leu-Nle-NH₂;
(SEQ ID NO. 238)
Gly-Arg-His-7MeTrp-Ala-Val-β-Ala-His-Leu-Nle-NH₂
(SEQ ID NO. 249)
Gly-Glu-His-7MeTrp-Ala-Val-BABA-His-Leu-Nle-NH₂;
(SEQ ID NO. 250)
Glu-D-Ser-Glu-His-7MeTrp-Ala-Val-β-Ala-His-Leu-
Nle-NH₂;
(SEQ ID NO. 304)
Ser-Glu-His-7MeTrp-Ala-Val-BABA-NMeHis-Leu-Nle-
NH₂;
(SEQ ID NO. 305)
Gly-Asp-His-7MeTrp-Ala-Val-BABA-NMeHis-Leu-Nle-
NH₂,
or a pharmaceutically acceptable salt thereof.

29. A pharmaceutical composition comprising the peptide of claim 28, or a pharmaceutically acceptable salt thereof, and a pharmaceutically acceptable carrier, diluent, or excipient.

30. A method for treating obesity in a subject in need thereof, the method comprising administering a therapeutically effective amount of a peptide of claim 28, or a pharmaceutically acceptable salt thereof.


SEQUENCE LISTING (325 sequences)

SEQ ID NO: 1 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = alpha-aminoisobutyric acid
  SITE 13: note = amidated norleucine
EXXGNHWAVXHLX

SEQ ID NO: 2 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = alpha-aminoisobutyric acid
  SITE 11: note = 3-(3-pyridyl)alanine
  SITE 13: note = amidated norleucine
EXXGNHWAVXXLX

SEQ ID NO: 3 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 6: note = 3-(3-pyridyl)alanine
  SITE 10: note = alpha-aminoisobutyric acid
  SITE 13: note = amidated norleucine
EXXGNXWAVXHLX

SEQ ID NO: 4 - protein, 13 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = alpha-aminoisobutyric acid
  SITE 13: note = amidated norleucine
EXXGNHWAVXHLX

SEQ ID NO: 5 - protein, 13 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = Amidated Methionine
EXXGNHWAVGHLM

SEQ ID NO: 6 - protein, 10 aa, synthetic construct
  SITE 7: note = D-Alanine
  SITE 10: note = amidated norleucine
GNHWAVXHLX

SEQ ID NO: 7 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGNHWAVXHLX

SEQ ID NO: 8 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 13: note = Amidated Methionine
EXXGNHWAVGHLM

SEQ ID NO: 9 - protein, 13 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGNHWAVXHLX

SEQ ID NO: 10 - protein, 10 aa, synthetic construct
  SITE 1: note = Palmitoyl-Glycine
  SITE 7: note = D-Alanine
  SITE 10: note = amidated norleucine
GNHWAVXHLX

SEQ ID NO: 11 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 8: note = alpha-aminoisobutyric acid
  SITE 10: note = D-Alanine
  SITE 13: note = amidated norleucine
EXXGNHWXVXHLX

SEQ ID NO: 12 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 6: note = 3-(3-pyridyl)alanine
  SITE 10: note = D-Alanine
  SITE 13: note = amidated norleucine
EXXGNXWAVXHLX

SEQ ID NO: 13 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7MethylTrp
  SITE 10: note = D-Alanine
  SITE 13: note = amidated norleucine
EXXGNHWAVXHLX

SEQ ID NO: 14 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7Methyl-Tryptophan
  SITE 8: note = alpha-aminoisobutyric acid
  SITE 10: note = D-Alanine
  SITE 13: note = amidated norleucine
EXXGNHWXVXHLX

SEQ ID NO: 15 - protein, 14 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = norleucine
  SITE 14: note = Amidated Sarcosine
EXXGNHWAVXHLXX

SEQ ID NO: 16 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGEHWAVXHLX

SEQ ID NO: 17 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 11: note = 3-(3-pyridyl)alanine
  SITE 13: note = amidated norleucine
EXXGNHWAVXXLX

SEQ ID NO: 18 - protein, 14 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 6: note = 3-(3-pyridyl)alanine
  SITE 7: note = 7Methyl-Tryptophan
  SITE 8: note = alpha-aminoisobutyric acid
  SITE 10: note = D-Alanine
  SITE 11: note = 3-(3-pyridyl)alanine
  SITE 13: note = norleucine
  SITE 14: note = Amidated Sarcosine
EXXGEXWXVXXLXX

SEQ ID NO: 19 - protein, 14 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7Methyl-Tryptophan
  SITE 8: note = alpha-aminoisobutyric acid
  SITE 10: note = D-Alanine
  SITE 11: note = 3-(3-pyridyl)alanine
  SITE 13: note = norleucine
  SITE 14: note = Amidated Sarcosine
EXXGEHWXVXXLXX

SEQ ID NO: 20 - protein, 14 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7Methyl-Tryptophan
  SITE 8: note = alpha-aminoisobutyric acid
  SITE 10: note = D-Alanine
  SITE 11: note = 3-(3-pyridyl)alanine
  SITE 13: note = norleucine
  SITE 14: note = Amidated Sarcosine
EXXGNHWXVXXLXX

SEQ ID NO: 21 - protein, 11 aa, synthetic construct
  SITE 1: note = Palmitoyl-D-gamma-Glutamic Acid
  SITE 8: note = alpha-aminoisobutyric acid
  SITE 11: note = amidated norleucine
EGNHWAVXHLX

SEQ ID NO: 22 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7Methyl-Tryptophan
  SITE 10: note = alpha-aminoisobutyric acid
  SITE 13: note = amidated norleucine
EXXGNHWAVXHLX

SEQ ID NO: 23 - protein, 14 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7Methyl-Tryptophan
  SITE 10: note = alpha-aminoisobutyric acid
  SITE 11: note = 3-(3-pyridyl)alanine
  SITE 13: note = norleucine
  SITE 14: note = Amidated Sarcosine
EXXGNHWAVXXLXX

SEQ ID NO: 24 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = alpha-aminoisobutyric acid
  SITE 13: note = amidated norleucine
EXXGEHWAVXHLX

SEQ ID NO: 25 - protein, 10 aa, synthetic construct
  SITE 1: note = Acetylated-Glycine
  SITE 7: note = D-Alanine
  SITE 10: note = norleucine-H2N(CH2)3NH2-NH-CH2CH2O-CH2CH2O-CH2C(=O) -NH-CH2CH2O-CH2CH2O-CH2C(=O)-(alpha-Palmitoyl-D-gamma-Glut amic Acid)
GNHWAVXHLX

SEQ ID NO: 26 - protein, 11 aa, synthetic construct
  SITE 1: note = Palmitoyl-D-gamma-Glutamic Acid
  SITE 8: note = D-Alanine
  SITE 11: note = amidated norleucine
EGNHWAVXHLX

SEQ ID NO: 27 - protein, 10 aa, synthetic construct
  SITE 7: note = alpha-aminoisobutyric acid
  SITE 10: note = amidated norleucine
GNHWAVXHLX

SEQ ID NO: 28 - protein, 10 aa, synthetic construct
  SITE 7: note = alpha-aminoisobutyric acid
  SITE 8: note = 3-(3-pyridyl)alanine
  SITE 10: note = amidated norleucine
GNHWAVXXLX

SEQ ID NO: 29 - protein, 10 aa, synthetic construct
  SITE 3: note = 3-(3-pyridyl)alanine
  SITE 7: note = alpha-aminoisobutyric acid
  SITE 10: note = amidated norleucine
GNXWAVXHLX

SEQ ID NO: 30 - protein, 10 aa, synthetic construct
  SITE 10: note = Amidated Methionine
GNHWAVGHLM

SEQ ID NO: 31 - protein, 10 aa, synthetic construct
  SITE 5: note = alpha-aminoisobutyric acid
  SITE 7: note = D-Alanine
  SITE 10: note = amidated norleucine
GNHWXVXHLX

SEQ ID NO: 32 - protein, 10 aa, synthetic construct
  SITE 3: note = 3-(3-pyridyl)alanine
  SITE 7: note = D-Alanine
  SITE 10: note = amidated norleucine
GNXWAVXHLX

SEQ ID NO: 33 - protein, 10 aa, synthetic construct
  SITE 4: note = 7Methyl-Tryptophan
  SITE 7: note = D-Alanine
  SITE 10: note = amidated norleucine
GNHWAVXHLX

SEQ ID NO: 34 - protein, 10 aa, synthetic construct
  SITE 4: note = 7Methyl-Tryptophan
  SITE 5: note = alpha-aminoisobutyric acid
  SITE 7: note = D-Alanine
  SITE 10: note = amidated norleucine
GNHWXVXHLX

SEQ ID NO: 35 - protein, 11 aa, synthetic construct
  SITE 7: note = D-Alanine
  SITE 10: note = norleucine
  SITE 11: note = Amidated Sarcosine
GNHWAVXHLXX

SEQ ID NO: 36 - protein, 10 aa, synthetic construct
  SITE 7: note = D-Alanine
  SITE 10: note = amidated norleucine
GEHWAVXHLX

SEQ ID NO: 37 - protein, 10 aa, synthetic construct
  SITE 7: note = D-Alanine
  SITE 8: note = 3-(3-pyridyl)alanine
  SITE 10: note = amidated norleucine
GNHWAVXXLX

SEQ ID NO: 38 - protein, 11 aa, synthetic construct
  SITE 3: note = 3-(3-pyridyl)alanine
  SITE 4: note = 7Methyl-Tryptophan
  SITE 5: note = alpha-aminoisobutyric acid
  SITE 7: note = D-Alanine
  SITE 8: note = 3-(3-pyridyl)alanine
  SITE 10: note = norleucine
  SITE 11: note = Amidated Sarcosine
GEXWXVXXLXX

SEQ ID NO: 39 - protein, 11 aa, synthetic construct
  SITE 4: note = 7Methyl-Tryptophan
  SITE 5: note = alpha-aminoisobutyric acid
  SITE 7: note = D-Alanine
  SITE 8: note = 3-(3-pyridyl)alanine
  SITE 10: note = norleucine
  SITE 11: note = Amidated Sarcosine
GEHWXVXXLXX

SEQ ID NO: 40 - protein, 11 aa, synthetic construct
  SITE 4: note = 7Methyl-Tryptophan
  SITE 5: note = alpha-aminoisobutyric acid
  SITE 7: note = D-Alanine
  SITE 8: note = 3-(3-pyridyl)alanine
  SITE 10: note = norleucine
  SITE 11: note = Amidated Sarcosine
GNHWXVXXLXX

SEQ ID NO: 41 - protein, 10 aa, synthetic construct
  SITE 4: note = 7Methyl-Tryptophan
  SITE 7: note = alpha-aminoisobutyric acid
  SITE 10: note = amidated norleucine
GNHWAVXHLX

SEQ ID NO: 42 - protein, 11 aa, synthetic construct
  SITE 4: note = 7Methyl-Tryptophan
  SITE 7: note = alpha-aminoisobutyric acid
  SITE 8: note = 3-(3-pyridyl)alanine
  SITE 10: note = norleucine
  SITE 11: note = Amidated Sarcosine
GNHWAVXXLXX

SEQ ID NO: 43 - protein, 10 aa, synthetic construct
  SITE 7: note = alpha-aminoisobutyric acid
  SITE 10: note = amidated norleucine
GEHWAVXHLX

SEQ ID NO: 44 - protein, 10 aa, synthetic construct
  SITE 10: note = Amidated Methionine
GNHWAVGHLM

SEQ ID NO: 45 - protein, 27 aa, synthetic construct
  SITE 25: note = Amidated Methionine
VPLPAGGGTVLTKMYPRGNHWAVGHLM

SEQ ID NO: 46 - protein, 13 aa, synthetic construct
  SITE 1: note = Octadecanedioyl-D-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7Methyl-Tryptophan
  SITE 10: note = D-Alanine
  SITE 13: note = amidated norleucine
EXXGNHWAVXHLX

SEQ ID NO: 47 - protein, 14 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = norleucine
  SITE 14: note = Amidated Sarcosine
EXXGNHWAVXHLXX

SEQ ID NO: 48 - protein, 14 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = norleucine
  SITE 14: note = Amidated Sarcosine
EXXGNHWAVXHLXX

SEQ ID NO: 49 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = Lys(Me)3-NH2
EXXGNHWAVXHLK

SEQ ID NO: 50 - protein, 12 aa, synthetic construct
  SITE 1: note = Acetylated-Glycine
  SITE 7: note = epsilon-Lysine
  SITE 10: note = -NH-CH2CH2O-CH2CH2O-CH2C(=O)-
  SITE 11: note = -NH-CH2CH2O-CH2CH2O-CH2C(=O)-
  SITE 12: note = (alpha-Palmitoyl-D-gamma-Glu)-NH2
GNHWAVXHLKXX

SEQ ID NO: 51 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 6: note = 3-(2-pyridyl)alanine
  SITE 10: note = D-Alanine
  SITE 13: note = amidated norleucine
EXXGNXWAVAHLX

SEQ ID NO: 52 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 6: note = 3-(4-pyridyl)alanine
  SITE 10: note = D-Alanine
  SITE 13: note = amidated norleucine
EXXGNXWAVXHLX

SEQ ID NO: 53 - protein, 15 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 12: note = D-Alanine
  SITE 15: note = amidated norleucine
EXXXXGNHWAVXHLX

SEQ ID NO: 54 - protein, 15 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 12: note = D-Alanine
  SITE 15: note = amidated norleucine
EXXXXGNHWAVXHLX

SEQ ID NO: 55 - protein, 13 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 5: note = Lys(-NH-CH2CH2O-CH2CH2O-CH2C(=O)-NH-CH2CH2O-CH2CH2O -CH2C(=O)-Octadecanedioyl-D-Glu)
  SITE 10: note = D-Alanine
  SITE 13: note = amidated norleucine
EXXGKHWAVXHLX

SEQ ID NO: 56 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7F-Tryptophan
  SITE 10: note = D-Alanine
  SITE 13: note = amidated norleucine
EXXGNHWAVXHLX

SEQ ID NO: 57 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 5: note = Lys(-NH-CH2CH2O-CH2CH2O-CH2C(=O)-NH-CH2CH2O-CH2CH2O -CH2C(=O)-gamma-Palmitoyl-D-gamma-Glutamic Acid)
  SITE 10: note = D-Alanine
  SITE 13: note = amidated norleucine
EXXGKHWAVXHLX

SEQ ID NO: 58 - protein, 15 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 8: note = 3-(3-pyridyl)alanine
  SITE 9: note = 7Methyl-Tryptophan
  SITE 12: note = D-Alanine
  SITE 15: note = amidated norleucine
EXXXXGNXWAVXHLX

SEQ ID NO: 59 - protein, 15 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 9: note = 7Methyl-Tryptophan
  SITE 12: note = D-Alanine
  SITE 15: note = amidated norleucine
EXXXXGNHWAVXHLX

SEQ ID NO: 60 - protein, 13 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGKHWAVXHLX

SEQ ID NO: 61 - protein, 13 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGRHWAVXHLX

SEQ ID NO: 62 - protein, 13 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGNHWAVXFLX

SEQ ID NO: 63 - protein, 13 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 6Methyl-Tryptophan
  SITE 10: note = D-Alanine
  SITE 13: note = amidated norleucine
EXXGNHWAVXHLX

SEQ ID NO: 64 - protein, 13 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 6: note = 3-(3-pyridyl)alanine
  SITE 10: note = D-Alanine
  SITE 13: note = amidated norleucine
EXXGNXWAVXHLX

SEQ ID NO: 65 - protein, 13 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 5: note = Acetylated Lysine
  SITE 10: note = D-Alanine
  SITE 13: note = amidated norleucine
EXXGKHWAVXHLX

SEQ ID NO: 66 - protein, 13 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 11: note = 3-(3-pyridyl)alanine
  SITE 13: note = amidated norleucine
EXXGNHWAVXXLX

SEQ ID NO: 67 - protein, 13 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 11: note = 3-(3-pyridyl)alanine
  SITE 13: note = amidated norleucine
EXXGEHWAVXXLX

SEQ ID NO: 68 - protein, 13 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGNHHAVXHLX

SEQ ID NO: 69 - protein, 13 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = Amidated Arginine
EXXGNHWAVXHLR

SEQ ID NO: 70 - protein, 13 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 8: note = alpha-aminoisobutyric acid
  SITE 10: note = D-Alanine
  SITE 13: note = amidated norleucine
EXXGEHWXVXHLX

SEQ ID NO: 71 - protein, 12 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = Acetylated Lysine
  SITE 9: note = D-Alanine
  SITE 12: note = amidated norleucine
EXXKHWAVXHLX

SEQ ID NO: 72 - protein, 14 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 6: note = 3-(3-pyridyl)alanine
  SITE 10: note = D-Alanine
  SITE 13: note = norleucine
  SITE 14: note = Amidated Sarcosine
EXXGNXWAVXHLXX

SEQ ID NO: 73 - protein, 14 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 6: note = 3-(3-pyridyl)alanine
  SITE 7: note = 7Methyl-Tryptophan
  SITE 10: note = D-Alanine
  SITE 13: note = norleucine
  SITE 14: note = Amidated Sarcosine
EXXGNXWAVXHLXX

SEQ ID NO: 74 - protein, 13 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = alpha-aminoisobutyric acid
  SITE 10: note = D-Alanine
  SITE 13: note = amidated norleucine
EXXXNHWAVXHLX

SEQ ID NO: 75 - protein, 14 aa, synthetic construct
  SITE 1: note = Palmitoyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = norleucine
  SITE 14: note = Amidated alpha-aminoisobutyric acid
EXXGNHWAVXHLXX

SEQ ID NO: 76 - protein, 14 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7Methyl-Tryptophan
  SITE 10: note = D-Alanine
  SITE 13: note = norleucine
  SITE 14: note = Amidated alpha-aminoisobutyric acid
EXXGNHWAVXHLXX

SEQ ID NO: 77 - protein, 14 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 5: note = Acetylated Lysine
  SITE 10: note = D-Alanine
  SITE 13: note = norleucine
  SITE 14: note = Amidated Sarcosine
EXXGKHWAVXHLXX

SEQ ID NO: 78 - protein, 14 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 9: note = 7MethyTrp
  SITE 12: note = D-Alanine
  SITE 13: note = Amidated Leucine
EXXXXGNHWAVXHL

SEQ ID NO: 79 - protein, 13 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 6Methyl-Tryptophan
  SITE 10: note = D-Alanine
  SITE 13: note = amidated norleucine
EXXGNHWAVXHLX

SEQ ID NO: 80 - protein, 12 aa, synthetic construct
  SITE 1: note = Octadecanedioyl-D-gamma-Glutamic Acid
  SITE 2: note = Aminovaleric acid
  SITE 9: note = D-Alanine
  SITE 12: note = amidated norleucine
EXGNHWAVXHLX

SEQ ID NO: 81 - protein, 14 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7Methyl-Tryptophan
  SITE 10: note = D-Alanine
  SITE 13: note = norleucine
  SITE 14: note = Amidated Sarcosine
EXXGNHWAVXHLXX

SEQ ID NO: 82 - protein, 14 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = norleucine
  SITE 14: note = Amidated alpha-aminoisobutyric acid
EXXGNHWAVXHLXX

SEQ ID NO: 83 - protein, 15 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 8: note = 3-(3-pyridyl)alanine
  SITE 12: note = D-Alanine
  SITE 15: note = amidated norleucine
EXXXXGNXWAVXHLX

SEQ ID NO: 84 - protein, 14 aa, synthetic construct
  SITE 1: note = Octadecanedioyl-D-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 6Methyl-Tryptophan
  SITE 10: note = D-Alanine
  SITE 13: note = norleucine
  SITE 14: note = Amidated Sarcosine
EXXGNHWAVXHLXX

SEQ ID NO: 85 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7Methyl-Tryptophan
  SITE 10: note = D-Alanine
  SITE 13: note = amidated norleucine
EXXGNHWAVXHLX

SEQ ID NO: 86 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7F-Tryptophan
  SITE 10: note = D-Alanine
  SITE 13: note = amidated norleucine
EXXGNHWAVXHLX

SEQ ID NO: 87 - protein, 13 aa, synthetic construct
  SITE 1: note = Palmitoyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7F-Tryptophan
  SITE 10: note = D-Alanine
  SITE 13: note = amidated norleucine
EXXGNHWAVXHLX

SEQ ID NO: 88 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGRHWAVXHLX

SEQ ID NO: 89 - protein, 13 aa, synthetic construct
  SITE 1: note = Palmitoyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGRHWAVXHLX

SEQ ID NO: 90 - protein, 15 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 9: note = 7Methyl-Tryptophan
  SITE 12: note = D-Alanine
  SITE 15: note = amidated norleucine
EXXXXGNHWAVXHLX

SEQ ID NO: 91 - protein, 15 aa, synthetic construct
  SITE 1: note = Palmitoyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 8: note = 3-(3-pyridyl)alanine
  SITE 9: note = 7Methyl-Tryptophan
  SITE 12: note = D-Alanine
  SITE 15: note = amidated norleucine
EXXXXGNXWAVXHLX

SEQ ID NO: 92 - protein, 14 aa, synthetic construct
  SITE 1: note = Palmitoyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = norleucine
  SITE 14: note = Amidated Sarcosine
EXXGNHWAVXHLXX

SEQ ID NO: 93 - protein, 13 aa, synthetic construct
  SITE 1: note = Palmitoyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 3-(2-naphthyl)-alanine
  SITE 13: note = amidated norleucine
EXXGNHXAVAHLX

SEQ ID NO: 94 - protein, 13 aa, synthetic construct
  SITE 1: note = Palmitoyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 6: note = 3-(4-pyridyl)alanine
  SITE 10: note = D-Alanine
  SITE 13: note = amidated norleucine
EXXGNXWAVXHLX

SEQ ID NO: 95 - protein, 15 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 8: note = 3-(3-pyridyl)alanine
  SITE 9: note = 7-methyltryptophan
  SITE 12: note = D-Alanine
  SITE 15: note = amidated norleucine
EXXXXGNXWAVXHLX

SEQ ID NO: 96 - protein, 13 aa, synthetic construct
  SITE 1: note = Palmitoyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGNHWAVXHLX

SEQ ID NO: 97 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGNHWAVXHLX

SEQ ID NO: 98 - protein, 15 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 12: note = D-Alanine
  SITE 15: note = amidated norleucine
EXXXXGNHWAVXHLX

SEQ ID NO: 99 - protein, 14 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 3-(2-naphthyl)alanine
  SITE 10: note = D-Alanine
  SITE 13: note = norleucine
  SITE 14: note = Amidated Sarcosine
EXXGNHXAVXHLXX

SEQ ID NO: 100 - protein, 14 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = norleucine
  SITE 14: note = Amidated Sarcosine
EXXRNHWAVXHLXX

SEQ ID NO: 101 - protein, 16 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 9: note = 7F-Tryptophan
  SITE 12: note = D-Alanine
  SITE 15: note = norleucine
  SITE 16: note = Amidated Sarcosine
EXXXXGNHWAVXHLXX

SEQ ID NO: 102 - protein, 14 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7F-Tryptophan
  SITE 10: note = D-Alanine
  SITE 13: note = norleucine
  SITE 14: note = Amidated Sarcosine
EXXGNHWAVXHLXX

SEQ ID NO: 103 - protein, 16 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 9: note = 7Methyl-Tryptophan
  SITE 12: note = D-Alanine
  SITE 15: note = norleucine
  SITE 16: note = Amidated Sarcosine
EXXXXGNHWAVXHLXX

SEQ ID NO: 104 - protein, 14 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7Methyl-Tryptophan
  SITE 10: note = D-Alanine
  SITE 13: note = norleucine
  SITE 14: note = Amidated Sarcosine
EXXGNHWAVXHLXX

SEQ ID NO: 105 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 3-(2-naphthyl)alanine
  SITE 10: note = D-Alanine
  SITE 13: note = amidated norleucine
EXXGNHXAVXHLX

SEQ ID NO: 106 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 8: note = D-Alanine
  SITE 10: note = D-Alanine
  SITE 13: note = amidated norleucine
EXXGNHWXVXHLX

SEQ ID NO: 107 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGNHHAVXHLX

SEQ ID NO: 108 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGNHRAVXHLX

SEQ ID NO: 109 - protein, 15 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 9: note = 7F-Tryptophan
  SITE 12: note = D-Alanine
  SITE 15: note = amidated norleucine
EXXXXGRHWAVXHLX

SEQ ID NO: 110 - protein, 15 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 9: note = 7F-Tryptophan
  SITE 12: note = D-Alanine
  SITE 15: note = amidated norleucine
EXXXXGNHWAVXHLX

SEQ ID NO: 111 - protein, 14 aa, synthetic construct
  SITE 1: note = Palmitoyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = norleucine
  SITE 14: note = Amidated Sarcosine
EXXGNHWAVXHLXX

SEQ ID NO: 112 - protein, 14 aa, synthetic construct
  SITE 1: note = Palmitoyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7Methyl-Tryptophan
  SITE 10: note = D-Alanine
  SITE 13: note = norleucine
  SITE 14: note = Amidated Sarcosine
EXXGNHWAVXHLXX

SEQ ID NO: 113 - skipped

SEQ ID NO: 114 - protein, 14 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = norleucine
  SITE 14: note = Amidated Arginine
EXXGNHWAVXHLXR

SEQ ID NO: 115 - protein, 14 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 8: note = D-Alanine
  SITE 10: note = D-Alanine
  SITE 13: note = norleucine
  SITE 14: note = Amidated Sarcosine
EXXGNHWXVXHLXX

SEQ ID NO: 116 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGRHHAVXHLX

SEQ ID NO: 117 - protein, 15 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 9: note = 7Methyl-Tryptophan
  SITE 12: note = D-Alanine
  SITE 15: note = amidated norleucine
EXXXXGRHWAVXHLX

SEQ ID NO: 118 - protein, 15 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 12: note = D-Alanine
  SITE 15: note = amidated norleucine
EXXXXGRHWAVXHLX

SEQ ID NO: 119 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = Cha-NH2
EXXGNHWAVXHLX

SEQ ID NO: 120 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = Cha-NH2
EXXGAHWAVXHLX

SEQ ID NO: 121 - protein, 16 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 9: note = 7F-Tryptophan
  SITE 12: note = D-Alanine
  SITE 15: note = norleucine
  SITE 16: note = Amidated Sarcosine
EXXXXGAHWAVXHLXX

SEQ ID NO: 122 - protein, 15 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 9: note = 7Methyl-Tryptophan
  SITE 12: note = D-Alanine
  SITE 15: note = amidated norleucine
EXXXXGRHWAVXHLX

SEQ ID NO: 123 - protein, 14 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7Methyl-Tryptophan
  SITE 10: note = D-Alanine
  SITE 13: note = norleucine
  SITE 14: note = Amidated Sarcosine
EXXGNHWAVXHLXX

SEQ ID NO: 124 - protein, 14 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 3-(2-naphthyl)alanine
  SITE 10: note = D-Alanine
  SITE 13: note = norleucine
  SITE 14: note = Amidated Sarcosine
EXXGAHXAVXHLXX

SEQ ID NO: 125 - protein, 14 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7Methyl-Tryptophan
  SITE 8: note = D-Alanine
  SITE 10: note = D-Alanine
  SITE 13: note = norleucine
  SITE 14: note = Amidated Sarcosine
EXXGNHWXVXHLXX

SEQ ID NO: 126 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 3-(2-naphthyl)alanine
  SITE 10: note = D-Alanine
  SITE 13: note = amidated norleucine
EXXGAHXAVXHLX

SEQ ID NO: 127 - skipped

SEQ ID NO: 128 - protein, 14 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 8: note = D-Alanine
  SITE 10: note = D-Alanine
  SITE 13: note = norleucine
  SITE 14: note = Amidated Sarcosine
EXXGAHWXVXHLXX

SEQ ID NO: 129 - protein, 14 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7-methyltryptophan
  SITE 10: note = D-Alanine
  SITE 13: note = norleucine
  SITE 14: note = Amidated Sarcosine
EXXGRHWAVXHLXX

SEQ ID NO: 130 - protein, 14 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = norleucine
  SITE 14: note = Amidated Sarcosine
EXXGNHRAVXHLXX

SEQ ID NO: 131 - protein, 14 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = norleucine
  SITE 14: note = Amidated Sarcosine
EXXGNHWAVXHLXX

SEQ ID NO: 132 - protein, 15 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 12: note = D-Alanine
  SITE 15: note = amidated norleucine
EXXXXGRHWAVXHLX

SEQ ID NO: 133 - protein, 16 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 9: note = 6-fluorotryptophan
  SITE 12: note = D-Alanine
  SITE 15: note = norleucine
  SITE 16: note = Amidated Sarcosine
EXXXXGNHWAVXHLXX

SEQ ID NO: 134 - protein, 14 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7-methyltryptophan
  SITE 10: note = D-Alanine
  SITE 13: note = norleucine
  SITE 14: note = Arg-NH2
EXXGNHWAVXHLXR

SEQ ID NO: 135 - skipped

SEQ ID NO: 136 - protein, 15 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 9: note = 7F-Tryptophan
  SITE 12: note = D-Alanine
  SITE 15: note = amidated norleucine
EXXXXGAHWAVXHLX

SEQ ID NO: 137 - protein, 16 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = -NH-CH2CH2O-CH2CH2O-CH2C(=O)-
  SITE 5: note = -NH-CH2CH2O-CH2CH2O-CH2C(=O)-
  SITE 9: note = 7F-Tryptophan
  SITE 12: note = D-Alanine
  SITE 15: note = norleucine
  SITE 16: note = Amidated Sarcosine
EXXXXGRHWAVXHLXX

SEQ ID NO: 138 - protein, 14 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7F-Tryptophan
  SITE 10: note = D-Alanine
  SITE 13: note = norleucine
  SITE 14: note = Amidated Sarcosine
EXXGNHWAVXHLXX

SEQ ID NO: 139 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 8: note = D-Alanine
  SITE 10: note = D-Alanine
  SITE 13: note = Amidated norleucine
EXXGDHWXVXHLX

SEQ ID NO: 140 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = Beta-Alanine
  SITE 13: note = amidated norleucine
EXXFNHWAVAHLX

SEQ ID NO: 141 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXRRHWAVXHLX

SEQ ID NO: 142 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = Beta-Alanine
  SITE 13: note = amidated norleucine
EXXGNHWAVAHLX

SEQ ID NO: 143 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = D-phenylalanine
  SITE 10: note = D-Alanine
  SITE 13: note = amidated norleucine
EXXFNHWAVXHLX

SEQ ID NO: 144 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 6: note = 3-(3-pyridyl)alanine
  SITE 10: note = Beta-Alanine
  SITE 13: note = amidated norleucine
EXXFNXWAVAHLX

SEQ ID NO: 145 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXRNHWAVXHLX

SEQ ID NO: 146 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = norleucine
EXXGNHWAVXHLX

SEQ ID NO: 147 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGQHWAVXHLX

SEQ ID NO: 148 - protein, 13 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = norleucine
EXXGNHWAVXHLX

SEQ ID NO: 149 - protein, 13 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGNHWAVXHLX

SEQ ID NO: 150 - protein, 13 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGRHWAVXHLX

SEQ ID NO: 151 - protein, 13 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = amidated norleucine
EXXGQHWAVXHLX

SEQ ID NO: 152 - protein, 12 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 9: note = Beta-Alanine
  SITE 12: note = amidated norleucine
EXXQHWAVAHLX

SEQ ID NO: 153 - protein, 11 aa, synthetic construct
  SITE 1: note = Eicosanedioyl-gamma-Glutamic Acid
  SITE 8: note = D-Alanine
  SITE 11: note = amidated norleucine
EGRHWAVXHLX

SEQ ID NO: 154 - protein, 11 aa, synthetic construct
  SITE 1: note = Eicosanedioyl-gamma-Glutamic Acid
  SITE 8: note = D-Alanine
  SITE 11: note = amidated norleucine
ERNHWAVXHLX

SEQ ID NO: 155 - protein, 11 aa, synthetic construct
  SITE 1: note = Eicosanedioyl-gamma-Glutamic Acid
  SITE 8: note = D-Alanine
  SITE 11: note = norleucine
ERNHWAVXHLX

SEQ ID NO: 156 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = norleucine
EXXRNHWAVXHLX

SEQ ID NO: 157 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = norleucine
EXXRRHWAVXHLX

SEQ ID NO: 158 - protein, 11 aa, synthetic construct
  SITE 1: note = Eicosanedioyl-gamma-Glutamic Acid
  SITE 8: note = D-Alanine
  SITE 11: note = amidated norleucine
ERRHWAVXHLX

SEQ ID NO: 159 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 11: note = D-histidine
  SITE 13: note = amidated norleucine
EXXGNHWAVXHLX

SEQ ID NO: 160 - protein, 11 aa, synthetic construct
  SITE 1: note = Eicosanedioyl-gamma-Glutamic Acid
  SITE 8: note = D-Alanine
  SITE 9: note = D-histidine
  SITE 11: note = amidated norleucine
EGNHWAVXHLX

SEQ ID NO: 161 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = D-phenylalanine
  SITE 10: note = D-Alanine
  SITE 13: note = amidated norleucine
EXXFQHWAVXHLX

SEQ ID NO: 162 - protein, 11 aa, synthetic construct
  SITE 1: note = Eicosanedioyl-gamma-Glutamic Acid
  SITE 8: note = D-Alanine
  SITE 11: note = norleucine
EGRHWAVXHLX

SEQ ID NO: 163 - protein, 11 aa, synthetic construct
  SITE 1: note = Eicosanedioyl-gamma-Glutamic Acid
  SITE 8: note = D-Alanine
  SITE 11: note = norleucine
ERRHWAVXHLX

SEQ ID NO: 164 - protein, 11 aa, synthetic construct
  SITE 1: note = Eicosanedioyl-gamma-Glutamic Acid
  SITE 8: note = D-Alanine
  SITE 11: note = norleucine
EGNHWAVXHLX

SEQ ID NO: 165 - protein, 11 aa, synthetic construct
  SITE 1: note = Eicosanedioyl-gamma-Glutamic Acid
  SITE 8: note = D-Alanine
  SITE 11: note = amidated norleucine
EGQHWAVXHLX

SEQ ID NO: 166 - protein, 13 aa, synthetic construct
  SITE 1: note = Octadecanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = norleucine
EXXGRHWAVXHLX

SEQ ID NO: 167 - protein, 13 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = D-Alanine
  SITE 13: note = norleucine
EXXGRHWAVXHLX

SEQ ID NO: 168 - protein, 10 aa, synthetic construct
  SITE 3: note = 3-(3-pyridyl)alanine
  SITE 4: note = 7Methyl-Tryptophan
  SITE 7: note = D-Alanine
  SITE 10: note = amidated norleucine
GNXWAVXHLX

SEQ ID NO: 169 - protein, 10 aa, synthetic construct
  SITE 7: note = D-Alanine
  SITE 10: note = Amidated N-epsilon-trimethyllysine
GNHWAVXHLK

SEQ ID NO: 170 - protein, 10 aa, synthetic construct
  SITE 3: note = 3-(2-pyridyl)alanine
  SITE 7: note = D-Alanine
  SITE 10: note = amidated norleucine
GNXWAVXHLX

SEQ ID NO: 171 - protein, 10 aa, synthetic construct
  SITE 3: note = 3-(4-pyridyl)alanine
  SITE 7: note = D-Alanine
  SITE 10: note = amidated norleucine
GNXWAVXHLX

SEQ ID NO: 172 - protein, 10 aa, synthetic construct
  SITE 4: note = 7F-Tryptophan
  SITE 7: note = D-Alanine
  SITE 10: note = amidated norleucine
GNHWAVXHLX

SEQ ID NO: 173 - protein, 10 aa, synthetic construct
  SITE 7: note = D-Alanine
  SITE 10: note = amidated norleucine
GKHWAVXHLX

SEQ ID NO: 174 - protein, 10 aa, synthetic construct
  SITE 7: note = D-Alanine
  SITE 10: note = amidated norleucine
GRHWAVXHLX

SEQ ID NO: 175 - protein, 10 aa, synthetic construct
  SITE 7: note = D-Alanine
  SITE 10: note = amidated norleucine
GNHWAVXFLX

SEQ ID NO: 176 - protein, 10 aa, synthetic construct
  SITE 4: note = 6-methyltryptophan
  SITE 7: note = D-Alanine
  SITE 10: note = amidated norleucine
GNHWAVXHLX

SEQ ID NO: 177 - protein, 10 aa, synthetic construct
  SITE 2: note = Acetylated Lysine
  SITE 7: note = D-Alanine
  SITE 10: note = amidated norleucine
GKHWAVXHLX

SEQ ID NO: 178 - skipped

SEQ ID NO: 179 - protein, 10 aa, synthetic construct
  SITE 7: note = D-Alanine
  SITE 8: note = 3-(3-pyridyl)alanine
  SITE 10: note = amidated norleucine
GEHWAVXXLX

SEQ ID NO: 180 - protein, 10 aa, synthetic construct
  SITE 7: note = D-Alanine
  SITE 10: note = amidated norleucine
GNHHAVXHLX

SEQ ID NO: 181 - protein, 10 aa, synthetic construct
  SITE 7: note = D-Alanine
  SITE 10: note = Amidated Arginine
GNHWAVXHLR

SEQ ID NO: 182 - protein, 10 aa, synthetic construct
  SITE 5: note = alpha-aminoisobutyric acid
  SITE 7: note = D-Alanine
  SITE 10: note = amidated norleucine
GEHWXVXHLX

SEQ ID NO: 183 - protein, 9 aa, synthetic construct
  SITE 1: note = Acetylated Lysine
  SITE 6: note = D-Alanine
  SITE 9: note = amidated norleucine
KHWAVXHLX

SEQ ID NO: 184 - protein, 11 aa, synthetic construct
  SITE 3: note = 3-(3-pyridyl)alanine
  SITE 7: note = D-Alanine
  SITE 10: note = norleucine
  SITE 11: note = Amidated Sarcosine
GNXWAVXHLXX

SEQ ID NO: 185 - protein, 11 aa, synthetic construct
  SITE 3: note = 3-(3-pyridyl)alanine
  SITE 4: note = 7Methyl-Tryptophan
  SITE 7: note = D-Alanine
  SITE 10: note = norleucine
  SITE 11: note = Amidated Sarcosine
GNXWAVXHLXX

SEQ ID NO: 186 - protein, 10 aa, synthetic construct
  SITE 1: note = alpha-aminoisobutyric acid
  SITE 7: note = D-Alanine
  SITE 10: note = amidated norleucine
XNHWAVXHLX

SEQ ID NO: 187 - protein, 11 aa, synthetic construct
  SITE 7: note = D-Alanine
  SITE 10: note = norleucine
  SITE 11: note = Amidated alpha-aminoisobutyric acid
GNHWAVXHLXX

SEQ ID NO: 188 - protein, 11 aa, synthetic construct
  SITE 4: note = 7Methyl-Tryptophan
  SITE 7: note = D-Alanine
  SITE 10: note = norleucine
  SITE 11: note = Amidated alpha-aminoisobutyric acid
GNHWAVXHLXX

SEQ ID NO: 189 - protein, 11 aa, synthetic construct
  SITE 2: note = Acetylated Lysine
  SITE 7: note = D-Alanine
  SITE 10: note = norleucine
  SITE 11: note = Amidated Sarcosine
GKHWAVXHLXX

SEQ ID NO: 190 - protein, 9 aa, synthetic construct
  SITE 4: note = 7Methyl-Tryptophan
  SITE 7: note = D-Alanine
  SITE 9: note = Amidated Leucine
GNHWAVXHL

SEQ ID NO: 191 - protein, 11 aa, synthetic construct
  SITE 4: note = 7Methyl-Tryptophan
  SITE 7: note = D-Alanine
  SITE 10: note = norleucine
  SITE 11: note = Amidated Sarcosine
GNHWAVXHLXX

SEQ ID NO: 192 - protein, 11 aa, synthetic construct
  SITE 4: note = 6-methyltryptophan
  SITE 7: note = D-Alanine
  SITE 10: note = norleucine
  SITE 11: note = Amidated Sarcosine
GNHWAVXHLXX

SEQ ID NO: 193 - protein, 10 aa, synthetic construct
  SITE 7: note = D-Alanine
  SITE 10: note = amidated norleucine
GRHWAVXHLX

SEQ ID NO: 194 - protein, 10 aa, synthetic construct
  SITE 4: note = 3-(2-naphthyl)alanine
  SITE 10: note = amidated norleucine
GNHXAVAHLX

SEQ ID NO: 195 - protein, 10 aa, synthetic construct
  SITE 3: note = 3-(3-pyridyl)alanine
  SITE 4: note = 7-methyltryptophan
  SITE 7: note = D-Alanine
  SITE 10: note = amidated norleucine
GNXWAVXHLX

SEQ ID NO: 196 - protein, 11 aa, synthetic construct
  SITE 4: note = 3-(2-naphthyl)alanine
  SITE 7: note = D-Alanine
  SITE 10: note = norleucine
  SITE 11: note = Amidated Sarcosine
GNHXAVXHLXX

SEQ ID NO: 197 - protein, 11 aa, synthetic construct
  SITE 7: note = D-Alanine
  SITE 10: note = norleucine
  SITE 11: note = Amidated Sarcosine
RNHWAVXHLXX

SEQ ID NO: 198 - protein, 11 aa, synthetic construct
  SITE 4: note = 7F-Tryptophan
  SITE 7: note = D-Alanine
  SITE 10: note = norleucine
  SITE 11: note = Amidated Sarcosine
GNHWAVXHLXX

SEQ ID NO: 199 - protein, 10 aa, synthetic construct
  SITE 4: note = 3-(2-naphthyl)alanine
  SITE 7: note = D-Alanine
  SITE 10: note = amidated norleucine
GNHXAVXHLX

SEQ ID NO: 200 - protein, 10 aa, synthetic construct
  SITE 5: note = D-Alanine
  SITE 7: note = D-Alanine
  SITE 10: note = amidated norleucine
GNHWXVXHLX

SEQ ID NO: 201 - protein, 10 aa, synthetic construct
  SITE 7: note = D-Alanine
  SITE 10: note = amidated norleucine
GNHRAVXHLX

SEQ ID NO: 202 - protein, 10 aa, synthetic construct
  SITE 4: note = 7F-Tryptophan
  SITE 7: note = D-Alanine
  SITE 10: note = amidated norleucine
GRHWAVXHLX

SEQ ID NO: 203 - protein, 11 aa, synthetic construct
  SITE 7: note = D-Alanine
  SITE 10: note = norleucine
  SITE 11: note = Amidated Arginine
GNHWAVXHLXR

SEQ ID NO: 204 - protein, 11 aa, synthetic construct
  SITE 5: note = D-Alanine
  SITE 7: note = D-Alanine
  SITE 10: note = norleucine
  SITE 11: note = Amidated Sarcosine
GNHWXVXHLXX

SEQ ID NO: 205 - protein, 10 aa, synthetic construct
  SITE 7: note = D-Alanine
  SITE 10: note = amidated norleucine
GRHHAVXHLX

SEQ ID NO: 206 - protein, 10 aa, synthetic construct
  SITE 4: note = 7Methyl-Tryptophan
  SITE 7: note = D-Alanine
  SITE 10: note = amidated norleucine
GRHWAVXHLX

SEQ ID NO: 207 - protein, 10 aa, synthetic construct
  SITE 7: note = D-Alanine
  SITE 10: note = Amidated cyclohexylalanine
GNHWAVXHLX

SEQ ID NO: 208 - protein, 10 aa, synthetic construct
  SITE 7: note = D-Alanine
  SITE 10: note = Amidated cyclohexylalanine
GAHWAVXHLX

SEQ ID NO: 209 - protein, 11 aa, synthetic construct
  SITE 4: note = 7F-Tryptophan
  SITE 7: note = D-Alanine
  SITE 10: note = norleucine
  SITE 11: note = Amidated Sarcosine
GAHWAVXHLXX

SEQ ID NO: 210 - protein, 11 aa, synthetic construct
  SITE 4: note = 3-(2-naphthyl)alanine
  SITE 7: note = D-Alanine
  SITE 10: note = norleucine
  SITE 11: note = Amidated Sarcosine
GAHXAVXHLXX

SEQ ID NO: 211 - protein, 11 aa, synthetic construct
  SITE 4: note = 7Methyl-Tryptophan
  SITE 5: note = D-Alanine
  SITE 7: note = D-Alanine
  SITE 10: note = norleucine
  SITE 11: note = Amidated Sarcosine
GNHWXVXHLXX

SEQ ID NO: 212 - protein, 10 aa, synthetic construct
  SITE 4: note = 3-(2-naphthyl)alanine
  SITE 7: note = D-Alanine
  SITE 10: note = amidated norleucine
GAHXAVXHLX

SEQ ID NO: 213 - skipped

SEQ ID NO: 214 - protein, 11 aa, synthetic construct
  SITE 5: note = D-Alanine
  SITE 7: note = D-Alanine
  SITE 10: note = norleucine
  SITE 11: note = Amidated Sarcosine
GAHWXVXHLXX

SEQ ID NO: 215 - protein, 11 aa, synthetic construct
  SITE 4: note = 7Methyl-Tryptophan
  SITE 7: note = D-Alanine
  SITE 10: note = norleucine
  SITE 11: note = Amidated Sarcosine
GRHWAVXHLXX

SEQ ID NO: 216 - protein, 11 aa, synthetic construct
  SITE 7: note = D-Alanine
  SITE 10: note = norleucine
  SITE 11: note = Amidated Sarcosine
GNHRAVXHLXX

SEQ ID NO: 217 - protein, 11 aa, synthetic construct
  SITE 4: note = 6-fluorotryptophan
  SITE 7: note = D-Alanine
  SITE 10: note = norleucine
  SITE 11: note = Amidated Sarcosine
GNHWAVXHLXX

SEQ ID NO: 218 - protein, 11 aa, synthetic construct
  SITE 4: note = 7Methyl-Tryptophan
  SITE 7: note = D-Alanine
  SITE 10: note = norleucine
  SITE 11: note = Amidated Arginine
GNHWAVXHLXR

SEQ ID NO: 219 - skipped

SEQ ID NO: 220 - protein, 10 aa, synthetic construct
  SITE 4: note = 7F-Tryptophan
  SITE 7: note = D-Alanine
  SITE 10: note = amidated norleucine
GAHWAVXHLX

SEQ ID NO: 221 - protein, 11 aa, synthetic construct
  SITE 4: note = 7F-Tryptophan
  SITE 7: note = D-Alanine
  SITE 10: note = norleucine
  SITE 11: note = Amidated Sarcosine
GRHWAVXHLXX

SEQ ID NO: 222 - protein, 10 aa, synthetic construct
  SITE 5: note = D-Alanine
  SITE 7: note = D-Alanine
  SITE 10: note = amidated norleucine
GDHWXVXHLX

SEQ ID NO: 223 - protein, 10 aa, synthetic construct
  SITE 7: note = Beta-Alanine
  SITE 10: note = amidated norleucine
FNHWAVAHLX

SEQ ID NO: 224 - protein, 10 aa, synthetic construct
  SITE 7: note = D-Alanine
  SITE 10: note = amidated norleucine
RRHWAVXHLX

SEQ ID NO: 225 - protein, 10 aa, synthetic construct
  SITE 7: note = Beta-Alanine
  SITE 10: note = amidated norleucine
GNHWAVAHLX

SEQ ID NO: 226 - protein, 11 aa, synthetic construct
  SITE 1: note = D-phenylalanine
  SITE 7: note = D-Alanine
  SITE 10: note = amidated norleucine
DFNHWAVXHLX

SEQ ID NO: 227 - protein, 10 aa, synthetic construct
  SITE 3: note = 3-(3-pyridyl)alanine
  SITE 7: note = Beta-Alanine
  SITE 10: note = amidated norleucine
FNXWAVAHLX

SEQ ID NO: 228 - protein, 10 aa, synthetic construct
  SITE 7: note = D-Alanine
  SITE 10: note = amidated norleucine
RNHWAVXHLX

SEQ ID NO: 229 - protein, 10 aa, synthetic construct
  SITE 7: note = D-Alanine
  SITE 10: note = norleucine
GNHWAVXHLX

SEQ ID NO: 230 - protein, 10 aa, synthetic construct
  SITE 7: note = D-Alanine
  SITE 10: note = amidated norleucine
GQHWAVXHLX

SEQ ID NO: 231 - protein, 10 aa, synthetic construct
  SITE 7: note = D-Alanine
  SITE 10: note = norleucine
RNHWAVXHLX

SEQ ID NO: 232 - protein, 10 aa, synthetic construct
  SITE 7: note = D-Alanine
  SITE 10: note = norleucine
RRHWAVXHLX

SEQ ID NO: 233 - protein, 10 aa, synthetic construct
  SITE 7: note = D-Alanine
  SITE 8: note = D-histidine
  SITE 10: note = amidated norleucine
GNHWAVXHLX

SEQ ID NO: 234 - protein, 10 aa, synthetic construct
  SITE 1: note = D-phenylalanine
  SITE 7: note = D-Alanine
  SITE 10: note = amidated norleucine
FQHWAVXHLX

SEQ ID NO: 235 - protein, 10 aa, synthetic construct
  SITE 7: note = D-Alanine
  SITE 10: note = amidated norleucine
GRHWAVXHLX

SEQ ID NO: 236 - protein, 10 aa, synthetic construct
  SITE 10: note = amidated norleucine
GNHWAVGHLX

SEQ ID NO: 237 - protein, 10 aa, synthetic construct
  SITE 7: note = Beta-Alanine
  SITE 10: note = amidated norleucine
GQHWAVAHLX

SEQ ID NO: 238 - protein, 10 aa, synthetic construct
  SITE 4: note = 7Methyl-Tryptophan
  SITE 7: note = Beta-Alanine
  SITE 10: note = amidated norleucine
GRHWAVAHLX

SEQ ID NO: 239 - protein, 10 aa, synthetic construct
  SITE 7: note = Beta-Alanine
  SITE 10: note = amidated norleucine
GRHWAVAHLX

SEQ ID NO: 240 - protein, 14 aa, synthetic construct
  SITE 1: note = Palmitoyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 14: note = amidated norleucine
EEXXGNHWAVGHLX

SEQ ID NO: 241 - protein, 13 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = Beta-Alanine
  SITE 13: note = amidated norleucine
EXXGQHWAVAHLX

SEQ ID NO: 242 - protein, 13 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7Methyl-Tryptophan
  SITE 10: note = Beta-Alanine
  SITE 13: note = amidated norleucine
EXXGRHWAVAHLX

SEQ ID NO: 243 - protein, 14 aa, synthetic construct
  SITE 1: note = Octadecanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 11: note = Beta-Alanine
  SITE 14: note = amidated norleucine
EEXXGRHWAVAHLX

SEQ ID NO: 244 - protein, 14 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 11: note = D-Alanine
  SITE 14: note = amidated norleucine
EEXXGRHWAVXHLX

SEQ ID NO: 245 - protein, 14 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 5: note = D-phenylalanine
  SITE 7: note = 3-(3-pyridyl)alanine
  SITE 11: note = Beta-Alanine
  SITE 14: note = amidated norleucine
EEXXFNXWAVAHLX

SEQ ID NO: 246 - protein, 10 aa, synthetic construct
  SITE 1: note = D-phenylalanine
  SITE 3: note = 3-(3-pyridyl)alanine
  SITE 7: note = Beta-Alanine
  SITE 10: note = amidated norleucine
FNXWAVAHLX

SEQ ID NO: 247 - protein, 14 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 8: note = 7-methyltryptophan
  SITE 11: note = S-beta-aminobutanoic acid
  SITE 14: note = amidated norleucine
EEXXGEHWAVXHLX

SEQ ID NO: 248 - protein, 15 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 6: note = D-Serine
  SITE 9: note = 7-methyltryptophan
  SITE 12: note = Beta-Alanine
  SITE 15: note = amidated norleucine
EEXXESEHWAVAHLX

SEQ ID NO: 249 - protein, 10 aa, synthetic construct
  SITE 4: note = 7-methyltryptophan
  SITE 7: note = S-beta-aminobutanoic acid
  SITE 10: note = amidated norleucine
GEHWAVXHLX

SEQ ID NO: 250 - protein, 11 aa, synthetic construct
  SITE 2: note = D-Serine
  SITE 5: note = 7-methyltryptophan
  SITE 8: note = Beta-Alanine
  SITE 11: note = amidated norleucine
ESEHWAVAHLX

SEQ ID NO: 251 - protein, 14 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 11: note = Beta-Alanine
  SITE 14: note = amidated norleucine
EEXXGQHWAVAHLX

SEQ ID NO: 252 - protein, 15 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 9: note = 7-methyltryptophan
  SITE 12: note = Beta-Alanine
  SITE 15: note = amidated norleucine
EEXXEGRHWAVAHLX

SEQ ID NO: 253 - protein, 14 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 8: note = 7-methyltryptophan
  SITE 11: note = Beta-Alanine
  SITE 14: note = amidated norleucine
EEXXRRHWAVAHLX

SEQ ID NO: 254 - protein, 15 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 9: note = 7-methyltryptophan
  SITE 12: note = Beta-Alanine
  SITE 15: note = amidated norleucine
EEXXRGRHWAVAHLX

SEQ ID NO: 255 - protein, 15 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 9: note = 7-methyltryptophan
  SITE 12: note = Beta-Alanine
  SITE 15: note = amidated norleucine
EEXXSGEHWAVAHLX

SEQ ID NO: 256 - protein, 15 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 9: note = 7-methyltryptophan
  SITE 12: note = Beta-Alanine
  SITE 15: note = amidated norleucine
EEXXSGRHWAVAHLX

SEQ ID NO: 257 - protein, 15 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 9: note = 7-methyltryptophan
  SITE 12: note = Beta-Alanine
  SITE 15: note = amidated norleucine
EEXXESQHWAVAHLX

SEQ ID NO: 258 - protein, 14 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 8: note = 7-methyltryptophan
  SITE 11: note = S-beta-aminobutanoic acid
  SITE 14: note = amidated norleucine
EEXXGNHWAVXHLX

SEQ ID NO: 259 - protein, 14 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 8: note = 7-methyltryptophan
  SITE 11: note = S-beta-aminobutanoic acid
  SITE 14: note = amidated norleucine
EEXXGQHWAVXHLX

SEQ ID NO: 260 - protein, 14 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 6: note = (S)-2,3-diaminopropanoic acid
  SITE 11: note = Beta-Alanine
  SITE 14: note = amidated norleucine
EXXGSXHWAVAHLX

SEQ ID NO: 261 - protein, 13 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 5: note = (S)-6-((2-amino-2-carboxyethyl)amino)-N,N,N-trimeth yl-6-oxohexan-1-aminium
  SITE 10: note = Beta-Alanine
  SITE 13: note = amidated norleucine
EXXGXHWAVAHLX

SEQ ID NO: 262 - protein, 13 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 5: note = (S)-2-(2-(3-((2-amino-2-carboxyethyl)amino)-3-oxopr opoxy)ethoxy)-N,N,N-trimethylethan-1-aminium
  SITE 10: note = Beta-Alanine
  SITE 13: note = amidated norleucine
EXXGXHWAVAHLX

SEQ ID NO: 263 - protein, 13 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7-methyltryptophan
  SITE 10: note = (S)-beta-aminoisobutyric acid
  SITE 13: note = amidated norleucine
EXXGQHWAVXHLX

SEQ ID NO: 264 - protein, 13 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 7: note = 7-methyltryptophan
  SITE 10: note = 3-amino-3-methylbutanoic acid
  SITE 13: note = amidated norleucine
EXXGNHWAVXHLX

SEQ ID NO: 265 - protein, 13 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = Beta-Alanine
  SITE 13: note = Amidated O-methyl-L-homoserine
EXXGQHWAVAHLS

SEQ ID NO: 266 - protein, 15 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 12: note = Beta-Alanine
  SITE 15: note = amidated norleucine
EXXGEGEHWAVAHLX

SEQ ID NO: 267 - protein, 13 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = Beta-Alanine
  SITE 11: note = N-methyl-L-histidine
  SITE 13: note = amidated norleucine
EXXGQHWAVAHLX

SEQ ID NO: 268 - protein, 13 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = Beta-Alanine
  SITE 13: note = amidated norleucine
EXXPQHWAVAHLX

SEQ ID NO: 269 - protein, 13 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 4: note = (2S,4R)-4-hydroxyproline
  SITE 10: note = Beta-Alanine
  SITE 13: note = amidated norleucine
EXXPQHWAVAHLX

SEQ ID NO: 270 - protein, 14 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 8: note = 7-methyltryptophan
  SITE 11: note = S-beta-aminobutanoic acid
  SITE 12: note = N-methyl-L-histidine
  SITE 14: note = amidated norleucine
EEXXGQHWAVXHLX

SEQ ID NO: 271 - protein, 14 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 11: note = (1S,2S)-2-aminocyclopentane-1-carboxylic acid
  SITE 14: note = amidated norleucine
EEXXGQHWAVXHLX

SEQ ID NO: 272 - protein, 14 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 11: note = 2-(1-aminocyclopropyl)acetic acid
  SITE 14: note = amidated norleucine
EEXXGQHWAVXHLX

SEQ ID NO: 273 - protein, 14 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 11: note = 2-(1-aminocyclobutyl)acetic acid
  SITE 14: note = amidated norleucine
EEXXGQHWAVXHLX

SEQ ID NO: 274 - protein, 14 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 8: note = alpha-MeTrp
  SITE 11: note = Beta-Alanine
  SITE 14: note = amidated norleucine
EEXXGNHWAVAHLX

SEQ ID NO: 275 - protein, 13 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2CH2O-CH2CH2O-CH2CH2O-CH2-C(= O)-
  SITE 7: note = 7-methyltryptophan
  SITE 10: note = S-beta-aminobutanoic acid
  SITE 13: note = amidated norleucine
EEXGEHWAVXHLX

SEQ ID NO: 276 - protein, 13 aa, synthetic construct
  SITE 1: note = Eicosanedioyl-gamma-Glutamic Acid
  SITE 2: note = -NH-CH2CH2O-CH2CH2O-CH2C(=O)-
  SITE 3: note = -NH-CH2CH2O-CH2CH2O-CH2C(=O)-
  SITE 10: note = Beta-Alanine
  SITE 13: note = MetO-NH2
EXXGQHWAVAHLM

SEQ ID NO: 277 - protein, 14 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 8: note = 7-methyltryptophan
  SITE 11: note = S-beta-aminobutanoic acid
  SITE 12: note = N-methyl-L-histidine
  SITE 14: note = amidated norleucine
EEXXSEHWAVXHLX

SEQ ID NO: 278 - protein, 14 aa, synthetic construct
  SITE 1: note = Eicosanedioyl-gamma-Glutamic Acid
  SITE 2: note = gamma-Glutamic Acid
  SITE 3: note = -NH-CH2CH2O-CH2CH2O-CH2C(=O)-
  SITE 4: note = -NH-CH2CH2O-CH2CH2O-CH2C(=O)-
  SITE 8: note = 7-methyltryptophan
  SITE 11: note = S-beta-aminobutanoic acid
  SITE 12: note = N-methyl-L-histidine
  SITE 14: note = amidated norleucine
EEXXGDHWAVXHLX

SEQ ID NO: 279 - protein, 10 aa, synthetic construct
  SITE 7: note = D-Alanine
  SITE 10: note = norleucine
GNHWAVXHLX

SEQ ID NO: 280 - protein, 10 aa, synthetic construct
  SITE 4: note = 7-methyltryptophan
  SITE 7: note = Beta-Alanine
  SITE 10: note = amidated norleucine
RRHWAVAHLX

SEQ ID NO: 281 - protein, 11 aa, synthetic construct
  SITE 5: note = 7-methyltryptophan
  SITE 8: note = Beta-Alanine
  SITE 11: note = amidated norleucine
RGRHWAVAHLX

SEQ ID NO: 282 - protein, 11 aa, synthetic construct
  SITE 5: note = 7-methyltryptophan
  SITE 8: note = Beta-Alanine
  SITE 11: note = amidated norleucine
SGEHWAVAHLX

SEQ ID NO: 283 - protein, 11 aa, synthetic construct
  SITE 5: note = 7-methyltryptophan
  SITE 8: note = Beta-Alanine
  SITE 11: note = amidated norleucine
SGRHWAVAHLX

SEQ ID NO: 284 - protein, 11 aa, synthetic construct
  SITE 5: note = 7-methyltryptophan
  SITE 8: note = Beta-Alanine
  SITE 11: note = amidated norleucine
ESQHWAVAHLX

SEQ ID NO: 285 - protein, 10 aa, synthetic construct
  SITE 4: note = 7-methyltryptophan
  SITE 7: note = S-beta-aminobutanoic acid
  SITE 10: note = amidated norleucine
GNHWAVXHLX

SEQ ID NO: 286 - protein, 10 aa, synthetic construct
  SITE 4: note = 7-methyltryptophan
  SITE 7: note = S-beta-aminobutanoic acid
  SITE 10: note = amidated norleucine
GQHWAVXHLX

SEQ ID NO: 287 - protein, 10 aa, synthetic construct
  SITE 2: note = (S)-2,3-diaminopropanoic acid
  SITE 7: note = Beta-Alanine
  SITE 10: note = amidated norleucine
GXHWAVAHLX

SEQ ID NO: 288 - protein, 10 aa, synthetic construct
  SITE 2: note = (S)-6-((2-amino-2-carboxyethyl)amino)-N,N,N-trimeth yl-6-oxohexan-1-aminium
  SITE 7: note = Beta-Alanine
  SITE 10: note = amidated norleucine
GXHWAVAHLX

SEQ ID NO: 289 - protein, 10 aa, synthetic construct
  SITE 2: note = (S)-2-(2-(3-((2-amino-2-carboxyethyl)amino)-3-oxopr opoxy)ethoxy)-N,N,N-trimethylethan-1-aminium
  SITE 7: note = Beta-Alanine
  SITE 10: note = amidated norleucine
GXHWAVAHLX

SEQ ID NO: 290 - protein, 10 aa, synthetic construct
  SITE 4: note = 7-methyltryptophan
  SITE 7: note = (S)-beta-aminoisobutyric acid
  SITE 10: note = amidated norleucine
GQHWAVXHLX

SEQ ID NO: 291 - protein, 10 aa, synthetic construct
  SITE 4: note = 7-methyltryptophan
  SITE 7: note = 3-amino-3-methylbutanoic acid
  SITE 10: note = amidated norleucine
GNHWAVXHLX

SEQ ID NO: 292 - protein, 10 aa, synthetic construct
  SITE 7: note = Beta-Alanine
  SITE 10: note = O-methyl-L-homoserine
GQHWAVAHLS

SEQ ID NO: 293 - protein, 12 aa, synthetic construct
  SITE 9: note = Beta-Alanine
  SITE 12: note = amidated norleucine
GEGEHWAVAHLX

SEQ ID NO: 294 - protein, 10 aa, synthetic construct
  SITE 7: note = Beta-Alanine
  SITE 8: note = N-methyl-L-histidine
  SITE 10: note = amidated norleucine
GQHWAVAHLX

SEQ ID NO: 295 - protein, 10 aa, synthetic construct
  SITE 7: note = Beta-Alanine
  SITE 10: note = amidated norleucine
PQHWAVAHLX

SEQ ID NO: 296 - protein, 10 aa, synthetic construct
  SITE 1: note = (2S,4R)-4-hydroxyproline
  SITE 7: note = Beta-Alanine
  SITE 10: note = amidated norleucine
PQHWAVAHLX

SEQ ID NO: 297 - protein, 10 aa, synthetic construct
  SITE 4: note = 7-methyltryptophan
  SITE 7: note = S-beta-aminobutanoic acid
  SITE 8: note = N-methyl-L-histidine
  SITE 10: note = amidated norleucine
GQHWAVXHLX

SEQ ID NO: 298 - protein, 10 aa, synthetic construct
  SITE 7: note = (1S,2S)-2-aminocyclopentane-1-carboxylic acid
  SITE 10: note = amidated norleucine
GQHWAVXHLX

SEQ ID NO: 299 - protein, 10 aa, synthetic construct
  SITE 7: note = 2-(1-aminocyclopropyl)acetic acid
  SITE 10: note = amidated norleucine
GQHWAVXHLX

SEQ ID NO: 300 - protein, 10 aa, synthetic construct
  SITE 7: note = 2-(1-aminocyclobutyl)acetic acid
  SITE 10: note = amidated norleucine
GQHWAVXHLX

SEQ ID NO: 301 - protein, 10 aa, synthetic construct
  SITE 4: note = alpha-MeTryptophan
  SITE 7: note = Beta-Alanine
  SITE 10: note = amidated norleucine
GNHWAVAHLX

SEQ ID NO: 302 - protein, 10 aa, synthetic construct
  SITE 4: note = 7-methyltryptophan
  SITE 7: note = S-beta-aminobutanoic acid
  SITE 10: note = amidated norleucine
GEHWAVXHLX

SEQ ID NO: 303 - protein, 10 aa, synthetic construct
  SITE 7: note = Beta-Alanine
  SITE 10: note = Amidated (2S)-2-amino-4-(methylsulfinyl)butanoic acid
GQHWAVAHLM

SEQ ID NO: 304 - protein, 10 aa, synthetic construct
  SITE 4: note = 7-methyltryptophan
  SITE 7: note = S-beta-aminobutanoic acid
  SITE 8: note = N-methyl-L-histidine
  SITE 10: note = amidated norleucine
SEHWAVXHLX

SEQ ID NO: 305 - protein, 10 aa, synthetic construct
  SITE 4: note = 7-methyltryptophan
  SITE 7: note = S-beta-aminobutanoic acid
  SITE 8: note = N-methyl-L-histidine
  SITE 10: note = amidated norleucine
GDHWAVXHLX

SEQ ID NO: 306 - protein, 11 aa, synthetic construct
  SITE 5: note = 7-methyltryptophan
  SITE 8: note = Beta-Alanine
  SITE 11: note = amidated norleucine
EGRHWAVAHLX

SEQ ID NO: 307 - protein, 5 aa, synthetic construct
  SITE 3: note = trityl-histidine
  SITE 5: note = norleucine-rink-amide MBHA resin
AVHLX

SEQ ID NO: 308 - protein, 6 aa, synthetic construct
  SITE 4: note = trityl-histidine
  SITE 6: note = norleucine-rink-amide MBHA resin
AVAHLX

SEQ ID NO: 309 - protein, 7 aa, synthetic construct
  SITE 1: note = (tert-Butoxycarbonyl)-L-tryptophan
  SITE 5: note = trityl-histidine
  SITE 7: note = norleucine-rink-amide MBHA resin
WAVAHLX

SEQ ID NO: 310 - protein, 8 aa, synthetic construct
  SITE 1: note = trityl-histidine
  SITE 2: note = (tert-Butoxycarbonyl)-L-tryptophan
  SITE 6: note = trityl-histidine
  SITE 8: note = norleucine-rink-amide MBHA resin
HWAVAHLX

SEQ ID NO: 311 - protein, 9 aa, synthetic construct
  SITE 1: note = trityl-asparagine
  SITE 2: note = trityl-histidine
  SITE 3: note = (tert-Butoxycarbonyl)-L-tryptophan
  SITE 7: note = trityl-histidine
  SITE 9: note = norleucine-rink-amide MBHA resin
NHWAVAHLX

SEQ ID NO: 312 - protein, 10 aa, synthetic construct
  SITE 2: note = trityl-asparagine
  SITE 3: note = trityl-histidine
  SITE 4: note = (tert-Butoxycarbonyl)-L-tryptophan
  SITE 8: note = trityl-histidine
  SITE 10: note = norleucine-rink-amide MBHA resin
GNHWAVAHLX

SEQ ID NO: 313 - protein, 11 aa, synthetic construct
  SITE 1: note = H2N-(CH2CH2O)2
  SITE 3: note = trityl-asparagine
  SITE 4: note = trityl-histidine
  SITE 5: note = (tert-Butoxycarbonyl)-L-tryptophan
  SITE 9: note = trityl-histidine
  SITE 11: note = norleucine-rink-amide MBHA resin
XGNHWAVAHLX

SEQ ID NO: 314 - protein, 12 aa, synthetic construct
  SITE 1: note = H2N-NH-CH2CH2O-CH2CH2O-CH2C(=O)-
  SITE 2: note = -NH-CH2CH2O-CH2CH2O-CH2C(=O)-
  SITE 4: note = trityl-asparagine
  SITE 5: note = trityl-histidine
  SITE 6: note = (tert-Butoxycarbonyl)-L-tryptophan
  SITE 10: note = trityl-histidine
  SITE 12: note = norleucine-rink-amide MBHA resin
XXGNHWAVAHLX

SEQ ID NO: 315 - protein, 13 aa, synthetic construct
  SITE 1: note = glutamic-acid(O-t-butyl)
  SITE 2: note = NH-NH-CH2CH2O-CH2CH2O-CH2C(=O)-
  SITE 3: note = -NH-CH2CH2O-CH2CH2O-CH2C(=O)-
  SITE 5: note = trityl-asparagine
  SITE 6: note = trityl-histidine
  SITE 7: note = (tert-Butoxycarbonyl)-L-tryptophan
  SITE 11: note = trityl-histidine
  SITE 13: note = norleucine-rink-amide MBHA resin
EXXGNHWAVAHLX

SEQ ID NO: 316 - protein, 13 aa, synthetic construct
  SITE 1: note = palmitic acid-Glu(OtBu)
  SITE 2: note = NH-NH-CH2CH2O-CH2CH2O-CH2C(=O)-
  SITE 3: note = -NH-CH2CH2O-CH2CH2O-CH2C(=O)-
  SITE 5: note = trityl-asparagine
  SITE 6: note = trityl-histidine
  SITE 7: note = (tert-Butoxycarbonyl)-L-tryptophan
  SITE 11: note = trityl-histidine
  SITE 13: note = norleucine-rink-amide MBHA resin
EXXGNHWAVAHLX

SEQ ID NO: 317 - protein, 10 aa, synthetic construct
  SITE 1: note = N-Acetylglycine
  SITE 2: note = trityl-asparagine
  SITE 3: note = trityl-histidine
  SITE 4: note = (tert-Butoxycarbonyl)-L-tryptophan
  SITE 7: note = D-Alanine
  SITE 8: note = trityl-histidine
  SITE 10: note = norleucine
GNHWAVAHLX

SEQ ID NO: 318 - protein, 14 aa, synthetic construct
  SITE 1: note = Octadecanedioyl(OtBu)-gamma-Glutamic acid(O-t-butyl)
  SITE 2: note = -NH-CH2CH2O-CH2CH2O-CH2C(=O)-
  SITE 3: note = -NH-CH2CH2O-CH2CH2O-CH2C(=O)-
  SITE 5: note = Lysine(Alloc)
  SITE 7: note = trityl-histidine
  SITE 8: note = (tert-Butoxycarbonyl)-L-tryptophan
  SITE 11: note = D-Alanine
  SITE 12: note = trityl-histidine
  SITE 14: note = Amidated norleucine
EXXGKNHWAVAHLX

SEQ ID NO: 319 - protein, 14 aa, synthetic construct
  SITE 1: note = octadecanedioic(OtBu) Acid-Glutamic acid(O-t-butyl)
  SITE 2: note = -NH-CH2CH2O-CH2CH2O-CH2C(=O)-
  SITE 3: note = -NH-CH2CH2O-CH2CH2O-CH2C(=O)-
  SITE 7: note = trityl-histidine
  SITE 8: note = (tert-Butoxycarbonyl)-L-tryptophan
  SITE 11: note = D-Alanine
  SITE 12: note = trityl-histidine
  SITE 14: note = norleucine-rink-amide MBHA resin
EXXGKNHWAVAHLX

SEQ ID NO: 320 - protein, 14 aa, synthetic construct
  SITE 1: note = octadecanedioyl(OtBu)-gamma-Glutamic acid(O-t-butyl)
  SITE 2: note = -NH-CH2CH2O-CH2CH2O-CH2C(=O)-
  SITE 3: note = -NH-CH2CH2O-CH2CH2O-CH2C(=O)-
  SITE 7: note = trityl-histidine
  SITE 8: note = (tert-Butoxycarbonyl)-L-tryptophan
  SITE 11: note = D-Alanine
  SITE 12: note = trityl-histidine
  SITE 14: note = norleucine-rink-amide MBHA resin
EXXGKNHWAVAHLX

SEQ ID NO: 321 - protein, 14 aa, synthetic construct
  SITE 1: note = octadecanedioic(OtBu) Acid-Glutamic acid(O-t-butyl)
  SITE 2: note = -NH-CH2CH2O-CH2CH2O-CH2C(=O)-
  SITE 3: note = -NH-CH2CH2O-CH2CH2O-CH2C(=O)-
  SITE 5: note = Lysine(Octadecanedioic(OtBu) Acid-Glu(OtBu)-NH-CH2C H2O-CH2CH2O-CH2C(=O)-NH-CH2CH2O-CH2CH2O-CH2C(=O)-)
  SITE 7: note = trityl-histidine
  SITE 8: note = (tert-Butoxycarbonyl)-L-tryptophan
  SITE 11: note = D-Alanine
  SITE 12: note = trityl-histidine
  SITE 14: note = norleucine-rink-amide MBHA resin
EXXGKNHWAVAHLX

SEQ ID NO: 322 - protein, 14 aa, synthetic construct
  SITE 1: note = octadecanedioyl(OtBu)-gamma-Glu(OtBu)
  SITE 2: note = -NH-CH2CH2O-CH2CH2O-CH2C(=O)-
  SITE 3: note = -NH-CH2CH2O-CH2CH2O-CH2C(=O)-
  SITE 5: note = epsilon-Lys(Octadecanedioyl(OtBu)-gamma-Glu(OtBu)-N H-CH2CH2O-CH2CH2O-CH2C(=O)-NH-CH2CH2O-CH2CH2O-CH2C(=O)-)
  SITE 7: note = trityl-histidine
  SITE 8: note = (tert-Butoxycarbonyl)-L-tryptophan
  SITE 11: note = D-Alanine
  SITE 12: note = trityl-histidine
  SITE 14: note = norleucine-rink-amide MBHA resin
EXXGKNHWAVAHLX

SEQ ID NO: 323 - protein, 13 aa, synthetic construct
  SITE 1: note = octadecanedioyl(OtBu)-gamma-Glu(OtBu)
  SITE 2: note = -NH-CH2CH2O-CH2CH2O-CH2C(=O)-
  SITE 3: note = -NH-CH2CH2O-CH2CH2O-CH2C(=O)-
  SITE 5: note = Arginine(Pbf)
  SITE 6: note = trityl-histidine
  SITE 7: note = (tert-Butoxycarbonyl)-L-tryptophan
  SITE 10: note = D-Alanine
  SITE 11: note = trityl-histidine
  SITE 13: note = norleucine 2-chlorotrityl chloride resin
EXXGRHWAVAHLX

SEQ ID NO: 324 - protein, 14 aa, synthetic construct
  SITE 1: note = Eicosanedioyl(OtBu)-gamma-Glutamic acid(O-t-butyl)
  SITE 2: note = gamma-Glutamic acid(O-t-butyl)
  SITE 3: note = -NH-CH2CH2O-CH2CH2O-CH2C(=O)-
  SITE 4: note = -NH-CH2CH2O-CH2CH2O-CH2C(=O)-
  SITE 6: note = Glutamic acid(O-t-butyl)
  SITE 7: note = trityl-histidine
  SITE 8: note = 7-methyltryptophan
  SITE 11: note = S-beta-aminobutanoic acid
  SITE 12: note = trityl-histidine
  SITE 14: note = norleucine-rink-amide MBHA resin
EEXXGEHWAVXHLX

SEQ ID NO: 325 - protein, 15 aa, synthetic construct
  SITE 1: note = Eicosanedioyl(OtBu)-gamma-Glutamic acid(O-t-butyl)
  SITE 2: note = gamma-Glutamic acid(O-t-butyl)
  SITE 3: note = -NH-CH2CH2O-CH2CH2O-CH2C(=O)-
  SITE 4: note = -NH-CH2CH2O-CH2CH2O-CH2C(=O)-
  SITE 5: note = Glutamic acid(O-t-butyl)
  SITE 6: note = D-Serine
  SITE 7: note = Glutamic acid(O-t-butyl)
  SITE 8: note = trityl-histidine
  SITE 9: note = 7-methyltryptophan
  SITE 12: note = Beta-Alanine
  SITE 13: note = trityl-histidine
  SITE 15: note = norleucine-rink-amide MBHA resin
EEXXESEHWAVAHLX